SEQUENCE LISTING (37 sequences)

SEQ ID NO: 1 - DNA, 2321 bp, Homo sapiens
  misc_feature: GUSB
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 2 - DNA, 1435 bp, Homo sapiens
  misc_feature: HPRT1
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 3 - DNA, 5365 bp, Homo sapiens
  misc_feature: IPO8
gttttccgtacagcagcatggcggccgccgacgggaggcggtcatagcatcacgcccgggggaagaggccgccgtaaaggaagctctgcttcctcttcttccttctcccgcctcccaccggctgtcgtaaaacggtgaatggagagcgagttgtgggggggaaaaagggaggacagggggcgcggagtcagagtggcgcagcaagtggccgcaggtggcgacggtggcggggggtggggtgtgaggtaatccaggggtcgcggaagaggaggctgagagggtcaaaagaaaactaaagctgcagtccggcctactgttccgggggccgcggagcccccacccggggagatggacctcaaccggatcatccaggcgctgaagggcaccatcgacccgaagttgcggattgcagccgagaacgagctcaaccagtcctacaagattatcaattttgcccccagtttacttcggattatagtctctgaccatgtggaattcccagtacgacaggcagctgccatttacctgaagaacatggtgacacaatactggccagatcgagaacctccaccaggagaagcaatatttccattcaacattcacgaaaacgatcgccagcaaatacgtgataacattgtggaaggaataattcggtctccagatttagtgagagtccaattaacaatgtgtctccgtgccatcataaaacatgattttcctggtcactggccaggagtggtcgacaagatagactattacttgcaatcacagagcagtgcaagctggcttggcagtttattatgcctgtatcaactggtgaagacatatgaatataagaaagcagaagagagagaacctcttataatagcaatgcagatattcctgcctcgtattcagcaacaaattgttcagctccttcctgattcctcctattattctgtattactgcagaaacaaattctgaaaatcttttatgcacttgttcagtatgcattgcctcttcagctagtgaataaccaaaccatgacaacatggatggagatcttccgaactattatcgacaggaccgttcctcctgagactctgcacattgatgaggatgatagaccagaactggtatggtggaagtgtaagaagtgggcactgcatattgtagctcggctctttgaacgatatggaagcccaggaaatgtcacaaaagaatactttgaattttctgaattctttttgaaaacctatgcagtgggcattcagcaggtgctactaaaaattttagatcaatatagacagaaagaatatgtagctccccgtgttcttcagcaagcattcaactatctcaaccaaggggtggttcattctataacctggaagcagatgaagccacacatacagaatatctctgaagatgtgattttttctgtgatgtgttataaagatgaggatgaagagctgtggcaagaagatccatatgagtatataaggatgaaatttgatatttttgaagattatgcttctcccaccacagcagcccagactctcttatatactgctgcaaagaaaagaaaagaggtgttgccaaaaatgatggcattctgttatcaaatcctgacagacccgaactttgaccctaggaagaaagatggagccctgcatgtgattggttccctagctgagattttactgaagaagagtttattcaaggaccaaatggagctgtttctacaaaatcatgtatttccattattattgtctaacctgggatatcttcgagctagatcttgctgggtacttcatgcatttagttctttgaagttccataatgagctcaatctaagaaatgccgttgaattagcgaagaagagcctgattgaagataaagagatgcctgtcaaagttgaagctgcccttgctcttcagtctttaatttctaaccagatacaagctaaggaatatatgaagccacatgtgaggcctattatgcaggaactgttgcacattgttagagagacagaaaatgatgatgttactaatgtcatccagaagatgatatgtgaatacagtcaagaggtagcctcaattgctgttgatatgacccaacacttggctgagatatttggcaaagttcttcaaagtgatgaatatgaagaagttgaagacaaaacagtaatggctatgggaattttacataccattgatactatcttaacagttgtagaagatcataaagagattacccagcagttagagaatatctgtctacggatcattgatcttgttctgcagaaacatgtaattgaattctatgaagaaattctttccctggcatacagtttaacctgccacagtatttcccctcaaatgtggcagcttctaggtatactatatgaagtgtttcagcaggattgctttgaatactttacagacatgatgcctctcctgcataattatgtgacaatagatacagataccttactatcaaatgcaaaacatttagaaattctttttacaatgtgtaggaaggtactatgtggagatgcaggagaagatgcagagtgtcatgcagctaaacttctggaagtcatcattcttcagtgcaaaggaaggggaattgatcagtgcattccactcttcgttcaacttgttttggagagattaactcgaggggtcaaaactagtgagcttcgtactatgtgtcttcaggttgcaattgctgccttgtactacaaccctgatttgctgctacatactttagaacgaattcagttgcctcacaaccctggacctatcactgtacagtttataaatcaatggatgaatgatacagattgttttcttgggcatcatgaccggaagatgtgtataataggactgagtatccttttggaattgcaaaatcgacctcctgcagtagatgctgtggtgggacagattgttccctcaattcttttccttttccttggcctaaagcaggtctgtgctactagacaactggtaaaccgggaagatcgttcaaaagcagagaaagctgatatggaagaaaatgaggagatttcaagtgatgaagaggagacaaatgtaactgctcaagcaatgcagtcaaataatggaagaggtgaagatgaggaggaggaagatgatgactgggatgaagaagtattggaagaaaccgcgcttgaggggttcagtactccacttgaccttgacaatagtgtggatgaatatcagttttttacacaagctctgataactgtgcagagtcgagatgcagcctggtaccagctgctgatggcaccactcagcgaggatcagaggacagcactgcaggaggtgtacacactggcagagcaccgacggacggtggcagaggcaaagaagaagattgaacaacagggaggcttcacctttgaaaacaaaggagtcctctccgcatttaattttgggactgtgcccagcaacaactgaaggaaagaacatcagctgaccaaatgtcatcgctgcattttatttcacaagaggagtgtgagggtcaaggggatgaaatgaggggctgcttttagggccctcctgctgtgccagttaccatctggcattaggcagcacttttatctactctttcccctttgacctttgtcaccctgaaatatatattttaaacagctactgtaagtatgaaatgaaagaaaaacaatcattggacggaaaaggacaacccatatgttccaaaggctgaatgcccaaggttgttttagaggattggatagacttgcacgtctcaggtttttgccatgcagaatcaatggatttatgcggataacagtgccttctgttgtacatgaattatcagaaaaaaatttttggagtgcattgcaattttttttaaagcataaaacatatttctagatacaacataaaccttggttatatgtcaactattctgcattttactctgtgaatttattgttaggcagttactgcaagtcactctggtttcaaaatcttcacggctccctctgctcaccctgctgctggggggcttttttcaggggttgtattataaaatatgcactggctgtggtttttcataagatgttttgtggctttttaaagaagtgtcttactccctcttctctcatttttttctgccttgaaaaggggtggtatttcttttggggtcaacaaatacacatatcagtttcactcctaaccttgtaagttcaggactcattttcttggcagcaagtggcaggggctctttagtcactggagttaacagtaagtctgagtatattctgaataatgtaattatgcaattaattgataatcatacctaaagcacattgaacttctaagagacaggtgctacgtaagcacactgtctttctggatggggacttgtattttaaataacttatcattccagctatgttggactagggtccaaagcattttataatatttttttttaatctaggaaaaagacccaaacaaatctaacttcttgctttctcacctatttgaattttctcctacctaatttgtttgtgtctttattatagctcagtttgacttctcaaacttttcaagggttggggcagctgcacttttaggttgcctataggaaatattgcatatggagattacagtgtttttctgacctagttcaagaccagaacatgggtcatagggtttttattcagcaaaatgaaaacgtatcttcagaacttaacatattactggatgtgatacagattttgctttctgtggaattgaaaattcacaaaaattcacagcttaaatttcccatcagtgactggaggaatttttttcaggtgcttcctatattaccatccctcatgcatattaactctttagaattttaggttaagtgatgtctatagaagtagcctggaaaaccatgagttttggagttcagtgaccccctgctttcctctgctcctcccttcccaaggcattgaagctgaatgtgccaactggcagttagaagcgaaagatggcattgggagaaattttagagagctttcaaactctttattctcatgttccacatggtctaattttaacataaataatatgccttcacactggattgtaaaaggcatgtgatttttgagattttattttgtgatgtattgtcttctgcagtattaaagggaaagagatattaatgtgcattaccctatcttgtttttgaagccagggtagttgtatgattttgttaccagcagtgctaacctgaatgtgacctggttaccttggaaatgcaggaacttatatgaatgtactataaaataaaatgcggactgattcccaggattctgaaaaaaaaaaaaaaaaaaaaaaaaaa

SEQ ID NO: 4 - DNA, 6738 bp, Homo sapiens
  misc_feature: POLR2A
gagagcgcggccgggacggttggagaagaaggcggctcccggaagggggagagacaaactgccgtaacctctgccgttcaggaacccggttacttatttattcgttaccctttttcttcttcctcccccaaaaaccttttccttttcccttctttttttttcctttttgggagctgaaaaatttccggtaagggaaagaagggctcctttcgctccttatttccccgcctccttccctcccccaccttcccctcctccggctttttcctcccaactcggggaggtccttcccggtggccgccctgacgaggtctgagcacctaggcggaggcggcgcaggctttttgtagtgaggtttgcgcctgcgcagcgcgcctgcctccgccatgcacgggggtggccccccctcgggggacagcgcatgcccgctgcgcaccatcaagagagtccagttcggagtcctgagtccggatgaactgaagcgaatgtctgtgacggagggtggcatcaaatacccagagacgactgagggaggccgccccaagcttggggggctgatggacccgaggcagggggtgattgagcggactggccgctgccaaacatgtgcaggaaacatgacagagtgtcctggccactttggccacattgaactggccaagcctgtgtttcacgtgggcttcctggtgaagacaatgaaagttttgcgctgtgtctgcttcttctgctccaaactgcttgtggactctaacaacccaaagatcaaggatatcctggctaagtccaagggacagcccaagaagcggctcacacatgtctacgacctttgcaagggcaaaaacatatgcgagggtggggaggagatggacaacaagttcggtgtggaacaacctgagggtgacgaggatctgaccaaagaaaagggccatggtggctgtgggcggtaccagcccaggatccggcgttctggcctagagctgtatgcggaatggaagcacgttaatgaggactctcaggagaagaagatcctgctgagtccagagcgagtgcatgagatcttcaaacgcatctcagatgaggagtgttttgtgctgggcatggagccccgctatgcacggccagagtggatgattgtcacagtgctgcctgtgcccccgctctccgtgcggcctgctgttgtgatgcagggctctgcccgtaaccaggatgacctgactcacaaactggctgacatcgtgaagatcaacaatcagctgcggcgcaatgagcagaacggcgcagcggcccatgtcattgcagaggatgtgaagctcctccagttccatgtggccaccatggtggacaatgagctgcctggcttgccccgtgccatgcagaagtctgggcgtcccctcaagtccctgaagcagcggttgaagggcaaggaaggccgggtgcgagggaacctgatgggcaaaagagtggacttctcggcccgtactgtcatcacccccgaccccaacctctccattgaccaggttggcgtgccccgctccattgctgccaacatgacctttgcggagattgtcacccccttcaacattgacagacttcaagaactagtgcgcagggggaacagccagtacccaggcgccaagtacatcatccgagacaatggtgatcgcattgacttgcgtttccaccccaagcccagtgaccttcacctgcagaccggctataaggtggaacggcacatgtgtgatggggacattgttatcttcaaccggcagccaactctgcacaaaatgtccatgatggggcatcgggtccgcattctcccatggtctacctttcgcttgaatcttagtgtgacaactccgtacaatgcagactttgacggggatgagatgaacttgcacctgccacagtctctggagacgcgagcagagatccaggagctggccatggttcctcgcatgattgtcaccccccagagcaatcggcctgtcatgggtattgtgcaggacacactcacagcagtgcgcaaattcaccaagagagacgtcttcctggagcggggtgaagtgatgaacctcctgatgttcctgtcgacgtgggatgggaaggtcccacagccggccatcctaaagccccggcccctgtggacaggcaagcaaatcttctccctcatcatacctggtcacatcaattgtatccgtacccacagcacccatcccgatgatgaagacagtggcccttacaagcacatctctcctggggacaccaaggtggtggtggagaatggggagctgatcatgggcatcctgtgtaagaagtctctgggcacgtcagctggctccctggtccacatctcctacctagagatgggtcatgacatcactcgcctcttctactccaacattcagactgtcattaacaactggctcctcatcgagggtcatactattggcattggggactccattgctgattctaagacttaccaggacattcagaacactattaagaaggccaagcaggacgtaatagaggtcatcgagaaggcacacaacaatgagctggagcccaccccagggaacactctgcggcagacgtttgagaatcaggtgaaccgcattcttaacgatgcccgagacaagactggctcctctgctcagaaatccctgtctgaatacaacaacttcaagtctatggtcgtgtccggagctaaaggttccaagattaacatctcccaggtcattgctgtcgttggacagcagaacgtcgagggcaagcggattccatttggcttcaagcaccggactctgcctcacttcatcaaggatgactacgggcctgagagccgtggctttgtggagaactcctacctagccggcctcacacccactgagttctttttccacgccatggggggtcgtgaggggctcattgacacggctgtcaagactgctgagactggatacatccagcggcggctgatcaagtccatggagtcagtgatggtgaagtacgacgcgactgtgcggaactccatcaaccaggtggtgcagctgcgctacggcgaagacggcctggcaggcgagagcgttgagttccagaacctggctacgcttaagccttccaacaaggcttttgagaagaagttccgctttgattataccaatgagagggccctgcggcgcactctgcaggaggacctggtgaaggacgtgctgagcaacgcacacatccagaacgagttggagcgggaatttgagcggatgcgggaggatcgggaggtgctcagggtcatcttcccaactggagacagcaaggtcgtcctcccctgtaacctgctgcggatgatctggaatgctcagaaaatcttccacatcaacccacgccttccctccgacctgcaccccatcaaagtggtggagggagtcaaggaattgagcaagaagctggtgattgtgaatggggatgacccactaagtcgacaggcccaggaaaatgccacgctgctcttcaacatccacctgcggtccacgttgtgttcccgccgcatggcagaggagtttcggctcagtggggaggccttcgactggctgcttggggagattgagtccaagttcaaccaagccattgcgcatcccggggaaatggtgggggctctggctgcgcagtcccttggagaacctgccacccagatgaccttgaataccttccactatgctggtgtgtctgccaagaatgtgacgctgggtgtgccccgacttaaggagctcatcaacatttccaagaagccaaagactccttcgcttactgtcttcctgttgggccagtccgctcgagatgctgagagagccaaggatattctgtgccgtctggagcatacaacgttgaggaaggtgactgccaacacagccatctactatgaccccaacccccagagcacggtggtggcagaggatcaggaatgggtgaatgtctactatgaaatgcctgactttgatgtggcccgaatctccccctggctgttgcgggtggagctggatcggaagcacatgactgaccggaagctcaccatggagcagattgctgaaaagatcaatgctggttttggtgacgacttgaactgcatctttaatgatgacaatgcagagaagctggtgctccgtattcgcatcatgaacagcgatgagaacaagatgcaagaggaggaagaggtggtggacaagatggatgatgatgtcttcctgcgctgcatcgagtccaacatgctgacagatatgaccctgcagggcatcgagcagatcagcaaggtgtacatgcacttgccacagacagacaacaagaagaagatcatcatcacggaggatggggaattcaaggccctgcaggagtggatcctggagacggacggcgtgagcttgatgcgggtgctgagtgagaaggacgtggaccccgtacgcaccacgtccaatgacattgtggagatcttcacggtgctgggcattgaagccgtgcggaaggccctggagcgggagctgtaccacgtcatctcctttgatggctcctatgtcaattaccgacacttggctctcttgtgtgataccatgacctgtcgtggccacttgatggccatcacccgacacggagtcaaccgccaggacacaggaccactcatgaagtgttcctttgaggaaacggtggacgtgcttatggaagcagccgcacacggtgagagtgaccccatgaagggggtctctgagaatatcatgctgggccagctggctccggccggcactggctgctttgacctcctgcttgatgcagagaagtgcaagtatggcatggagatccccaccaatatccccggcctgggggctgctggacccaccggcatgttctttggttcagcacccagtcccatgggtggaatctctcctgccatgacaccttggaaccagggtgcaacccctgcctatggcgcctggtcccccagtgttgggagtggaatgaccccaggggcagccggcttctctcccagtgctgcgtcagatgccagcggcttcagcccaggttactcccctgcctggtctcccacaccgggctccccggggtccccaggtccctcaagcccctacatcccttcaccaggtggtgccatgtctcccagctactcgccaacgtcacctgcctacgagccccgctctcctgggggctacacaccccagagtccctcttattcccccacttcaccctcctactcccctacctctccatcctattctccaaccagtcccaactatagtcccacatcacccagctattcgccaacgtcacccagctactcaccgacctctcccagctactcacccacctctcccagctactcgcccacctctcccagctactcgcccacctctcccagctactcacccacttcccctagctactcgcccacttcccctagctactcgccaacgtctcccagctactcgccgacatctcccagctactcgccaacttcacccagctattctcccacttctcccagctactcacctacctctccaagctattcacccacctcccccagctactcacccacttccccaagttactcacccaccagcccgaactattctccaaccagtcccaattacaccccaacatcacccagctacagcccgacatcacccagctattcacctactagtcccaactacacacctaccagccctaactacagcccaacctctccaagctactctccaacatcacccagctattccccgacctcaccaagttactccccttccagcccacgatacacaccacagtctccaacctataccccaagctcacccagctacagccccagctcgcccagctacagcccaacctcacccaagtacaccccaaccagtccttcttacagtcccagctccccagagtataccccaacctctcccaagtactcacctaccagtcccaaatattcacccacctctcccaagtactcgcctaccagtcccacctattcacccaccaccccaaaatactccccaacatctcctacttattccccaacctctccagtctacaccccaacctctcccaagtactcacctactagccccacttactcgcccacttcccccaagtactcgcccaccagccccacctactcgcccacctcccccaaaggctcaacctactctcccacttcccctggttactcgcccaccagccccacctacagtctcacaagcccggctatcagcccggatgacagtgacgaggagaactgagggcacgtggggtgcggcagcgggctagggcccagggcagcttgcccgtgctgctgtgcagttcttgcctccctcacggggcgtcacccccagcccagctccgttgtacataaatgccttgtggcagagctcccggtgaacttctggatcccgtttctgatgcagactcttgtcttgttctccacttgtgctgttagaactcactggcccagtggtgttctcactcctaccccacccaccccctgcctgtccccaaattgaagatccttccttgcctgtggcttgatgcggggcgggtaaagggtattttaacttaggggtagttcctgctgtgagtggttacagctgatcctcgggaagaacaaagctaaagctgccttttgtctgttattttatttttttgaagtttaaataaagtttactaattttgaccaaaagt

SEQ ID NO: 5 - DNA, 1921 bp, Homo sapiens
  misc_feature: TBP
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 6 - DNA, 1464 bp, Homo sapiens
  misc_feature: KLK3
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 7 - DNA, 2653 bp, Homo sapiens
  misc_feature: FOLH1
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 8 - DNA, 1992 bp, Homo sapiens
  misc_feature: FOLH1B
agcaaatactcactaccacaaataagaacatttccaaatctgatgttctgaggatttttagagcttatagtagcaaaaagaaaagggaaattctctctgagatgtccttttttgtaggcctaatgacaaaaggttgaagataaagttctagtactcatttaagtgtaatattgaaaattgatattaccaaatctggaacaaccaatttaaaataaggaaagaaagacactgtgttttctaggttaaaaatgcccagctggcaggggccaaaggagtcattctctactcagaccctgctgactactttgctcctggggtgaagtcctatccagacggttggaatcttcctggaggtggtgtccagcgtggaaatatcctaaatctgaatggtgcaggagaccctctcacaccaggttacccagcaaatgaatacgcttataggcatggaattgcagaggctgttggtcttccaagtattcctgttcatccagttggatactatgatgcacagaagctcctagaaaaaatgggtggctcagcaccaccagatagcagctggagaggaagtctcaaagtgtcctacaatgttggacctggctttactggaaacttttctacacaaaaagtcaagatgcacatccactctaccaatgaagtgacgagaatttacaatgtgataggtactctcagaggagcagtggaaccagacagatatgtcattctgggaggtcaccgggactcatgggtgtttggtggtattgaccctcagagtggagcagctgttgttcatgaaactgtgaggagctttggaacactgaaaaaggaagggtggagacctagaagaacaattttgtttgcaagctgggatgcagaagaatttggtcttcttggttctactgagtgggcagaggataattcaagactccttcaagagcgtggcgtggcttatattaatgctgactcatctatagaaggaaactacactctgagagttgattgtacaccactgatgtacagcttggtatacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacaatatttctatgaaacatccacaggaaatgaagacatacagtttatcatttgattcacttttttctgcagtaaaaaattttacagaaattgcttccaagttcagcgagagactccaggactttgacaaaagcaacccaatattgttaagaatgatgaatgatcaactcatgtttctggaaagagcatttattgatccattagggttaccagacagacctttttataggcatgtcatctatgctccaagcagccacaacaagtatgcaggggagtcattcccaggaatttatgatgctctgtttgatattgaaagcaaagtggacccttccaaggcctggggagatgtgaagagacagatttctgttgcagccttcacagtgcaggcagctgcagagactttgagtgaagtagcctaagaggattctttagagactctgtattgaatttgtgtggtatgtcactcaaagaataataatgggtatattgataaattttaaaattggtatatttgaaataaagttgaatattatatataaaaaaaaaaaaaaaaaaa

SEQ ID NO: 9 - DNA, 3130 bp, Homo sapiens
  misc_feature: OR51E1
gggagtaggcggagacagagaggctgtatttcagtgcagcctgccagacctcttctggaggaagactggacaaagggggtcacacattccttccatacggttgagcctctacctgcctggtgctggtcacagttcagcttcttcatgatggtggatcccaatggcaatgaatccagtgctacatacttcatcctaataggcctccctggtttagaagaggctcagttctggttggccttcccattgtgctccctctaccttattgctgtgctaggtaacttgacaatcatctacattgtgcggactgagcacagcctgcatgagcccatgtatatatttctttgcatgctttcaggcattgacatcctcatctccacctcatccatgcccaaaatgctggccatcttctggttcaattccactaccatccagtttgatgcttgtctgctacagatgtttgccatccactccttatctggcatggaatccacagtgctgctggccatggcttttgaccgctatgtggccatctgtcacccactgcgccatgccacagtacttacgttgcctcgtgtcaccaaaattggtgtggctgctgtggtgcggggggctgcactgatggcaccccttcctgtcttcatcaagcagctgcccttctgccgctccaatatcctttcccattcctactgcctacaccaagatgtcatgaagctggcctgtgatgatatccgggtcaatgtcgtctatggccttatcgtcatcatctccgccattggcctggactcacttctcatctccttctcatatctgcttattcttaagactgtgttgggcttgacacgtgaagcccaggccaaggcatttggcacttgcgtctctcatgtgtgtgctgtgttcatattctatgtacctttcattggattgtccatggtgcatcgctttagcaagcggcgtgactctccgctgcccgtcatcttggccaatatctatctgctggttcctcctgtgctcaacccaattgtctatggagtgaagacaaaggagattcgacagcgcatccttcgacttttccatgtggccacacacgcttcagagccctaggtgtcagtgatcaaacttcttttccattcagagtcctctgattcagattttaatgttaacattttggaagacagtattcagaaaaaaaatttccttaataaaaatacaactcagatccttcaaatatgaaactggttggggaatctccattttttcaatattattttcttctttgttttcttgctacatataattattaataccctgactaggttgtggttggagggttattacttttcattttaccatgcagtccaaatctaaactgcttctactgatggtttacagcattctgagataagaatggtacatctagagaacatttgccaaaggcctaagcacggcaaaggaaaataaacacagaatataataaaatgagataatctagcttaaaactataacttcctcttcagaactcccaaccacattggatctcagaaaaatgctgtcttcaaaatgacttctacagagaagaaataatttttcctctggacactagcacttaaggggaagattggaagtaaagccttgaaaagagtacatttacctacgttaatgaaagttgacacactgttctgagagttttcacagcatatggaccctgtttttcctatttaattttcttatcaaccctttaattaggcaaagatattattagtaccctcattgtagccatgggaaaattgatgttcagtggggatcagtgaattaaatggggtcatacaagtataaaaattaaaaaaaaaagacttcatgcccaatctcatatgatgtggaagaactgttagagagaccaacagggtagtgggttagagatttccagagtcttacattttctagaggaggtatttaatttcttctcactcatccagtgttgtatttaggaatttcctggcaacagaactcatggctttaatcccactagctattgcttattgtcctggtccaattgccaattacctgtgtcttggaagaagtgatttctaggttcaccattatggaagattcttattcagaaagtctgcatagggcttatagcaagttatttatttttaaaagttccataggtgattctgataggcagtgaggttagggagccaccagttatgatgggaagtatggaatggcaggtcttgaagataacattggccttttgagtgtgactcgtagctggaaagtgagggaatcttcaggaccatgctttatttggggctttgtgcagtatggaacagggactttgagaccaggaaagcaatctgacttaggcatgggaatcaggcatttttgcttctgaggggctattaccaagggttaataggtttcatcttcaacaggatatgacaacagtgttaaccaagaaactcaaattacaaatactaaaacatgtgatcatatatgtggtaagtttcattttctttttcaatcctcaggttccctgatatggattcctataacatgctttcatccccttttgtaatggatatcatatttggaaatgcctatttaatacttgtatttgctgctggactgtaagcccatgagggcactgtttattattgaatgtcatctctgttcatcattgactgctctttgctcatcattgaatcccccagcaaagtgcctagaacataatagtgcttatgcttgacaccggttatttttcatcaaacctgattccttctgtcctgaacacatagccaggcaattttccagccttctttgagttgggtattattaaattctggccattacttccaatgtgagtggaagtgacatgtgcaatttctatacctggctcataaaaccctcccatgtgcagcctttcatgttgacattaaatgtgacttgggaagctatgtgttacacagagtaaatcaccagaagcctggatttctgaaaaaactgtgcagagccaaacctctgtcatttgcaactcccacttgtatttgtacgaggcagttggataagtgaaaaataaagtactattgtgtcaagtctctgaaaaaaaaaaaaaaaaaaaaaaaaaaaa

SEQ ID NO: 10 - DNA, 2785 bp, Homo sapiens
  misc_feature: OR51E2
gaatctccacaccctgaagacacagtgagttagcaccaccaccaggaattggcctttcagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagtggtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaactttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacttgagtataagataaaataaaattttattttaaatttt

SEQ ID NO: 11 - DNA, 1603 bp, Homo sapiens
  misc_feature: PCGEM1
aaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaaaaaaaaaaaaaaa

SEQ ID NO: 12 - DNA, 4934 bp, Homo sapiens
  misc_feature: PMEPA1
aaacccgatctccttggacttgaatgaggaggaggaggcggcggcggcggcggcggcggaggcgctcggctggggaaagctagcggcagaggctcagccccggcggcagcgcgcgccccgctgccagcccattttccggacgccacccgcgggcactgccgacgcccccggggctgccgaggggaggccgggggggcgcagcggagcgcggtcccgcgcactgagccccgcggcgccccgggaacttggcggcgacccgagcccggcgagccggggcgcgcctcccccgccgcgcgcctcctgcatgcggggccccagctccgggcgccggccggagccccccccggccgcccccgagccccccgcgccccgcgccgcgccgccgcgccgtccatgcaccgcttgatgggggtcaacagcaccgccgccgccgccgccgggcagcccaatgtctcctgcacgtgcaactgcaaacgctctttgttccagagcatggagatcacggagctggagtttgttcagatcatcatcatcgtggtggtgatgatggtgatggtggtggtgatcacgtgcctgctgagccactacaagctgtctgcacggtccttcatcagccggcacagccaggggcggaggagagaagatgccctgtcctcagaaggatgcctgtggccctcggagagcacagtgtcaggcaacggaatcccagagccgcaggtctacgccccgcctcggcccaccgaccgcctggccgtgccgcccttcgcccagcgggagcgcttccaccgcttccagcccacctatccgtacctgcagcacgagatcgacctgccacccaccatctcgctgtcagacggggaggagcccccaccctaccagggcccctgcaccctccagcttcgggaccccgagcagcagctggaactgaaccgggagtcggtgcgcgcacccccaaacagaaccatcttcgacagtgacctgatggatagtgccaggctgggcggcccctgcccccccagcagtaactcgggcatcagcgccacgtgctacggcagcggcgggcgcatggaggggccgccgcccacctacagcgaggtcatcggccactacccggggtcctccttccagcaccagcagagcagtgggccgccctccttgctggaggggacccggctccaccacacacacatcgcgcccctagagagcgcagccatctggagcaaagagaaggataaacagaaaggacaccctctctagggtccccaggggggccgggctggggctgcgtaggtgaaaaggcagaacactccgcgcttcttagaagaggagtgagaggaaggcggggggcgcagcaacgcatcgtgtggccctcccctcccacctccctgtgtataaatatttacatgtgatgtctggtctgaatgcacaagctaagagagcttgcaaaaaaaaaaagaaaaaagaaaaaaaaaaaccacgtttctttgttgagctgtgtcttgaaggcaaaagaaaaaaaatttctacagtagtctttcttgtttctagttgagctgcgtgcgtgaatgcttattttcttttgtttatgataatttcacttaactttaaagacatatttgcacaaaacctttgtttaaagatctgcaatattatatatataaatatatataagataagagaaactgtatgtgcgagggcaggagtatttttgtattagaagaggcctattaaaaaaaaaagttgttttctgaactagaagaggaaaaaaatggcaatttttgagtgccaagtcagaaagtgtgtattaccttgtaaagaaaaaaattacaaagcaggggtttagagttatttatataaatgttgagattttgcactattttttaatataaatatgtcagtgcttgcttgatggaaacttctcttgtgtctgttgagactttaagggagaaatgtcggaatttcagagtcgcctgacggcagagggtgagcccccgtggagtctgcagagaggccttggccaggagcggcgggctttcccgaggggccactgtccctgcagagtggatgcttctgcctagtgacaggttatcaccacgttatatattccctaccgaaggagacaccttttcccccctgacccagaacagcctttaaatcacaagcaaaataggaaagttaaccacggaggcaccgagttccaggtagtggttttgcctttcccaaaaatgaaaataaactgttaccgaaggaattagtttttcctcttcttttttccaactgtgaaggtccccgtggggtggagcatggtgcccctcacaagccgcagcggctggtgcccgggctaccagggacatgccagagggctcgatgacttgtctctgcagggcgctttggtggttgttcagctggctaaaggttcaccggtgaaggcaggtgcggtaactgccgcactggaccctaggaagccccaggtattcgcaatctgacctcctcctgtctgtttcccttcacggatcaattctcacttaagaggccaataaacaacccaacatgaaaaggtgacaagcctgggtttctcccaggataggtgaaagggttaaaatgagtaaagcagttgagcaaacaccaacccgagcttcgggcgcagaattcttcaccttctcttcccctttccatctcctttccccgcggaaacaacgcttcccttctggtgtgtctgttgatctgtgttttcatttacatctctcttagactccgctcttgttctccaggttttcaccagatagatttggggttggcgggacctgctggtgacgtgcaggtgaaggacaggaaggggcatgtgagcgtaaatagaggtgaccagaggagagcatgaggggtggggctttgggacccaccggggccagtggctggagcttgacgtctttcctccccatgggggtgggagggcccccagctggaagagcagactcccagctgctaccccctcccttcccatgggagtggctttccattttgggcagaatgctgactagtagactaacataaaagatataaaaggcaataactattgtttgtgagcaacttttttataacttccaaaacaaaaacctgagcacagttttgaagttctagccactcgagctcatgcatgtgaaacgtgtgctttacgaaggtggcagctgacagacgtgggctctgcatgccgccagcctagtagaaagttctcgttcattggcaacagcagaacctgcctctccgtgaagtcgtcagcctaaaatttgtttctctcttgaagaggattctttgaaaaggtcctgcagagaaatcagtacaggttatcccgaaaggtacaaggacgcacttgtaaagatgattaaaacgtatctttcctttatgtgacgcgtctctagtgccttactgaagaagcagtgacactcccgtcgctcggtgaggacgttcccggacagtgcctcactcacctgggactggtatcccctcccagggtccaccaagggctcctgcttttcagacaccccatcatcctcgcgcgtcctcaccctgtctctaccagggaggtgcctagcttggtgaggttactcctgctcctccaacctttttttgccaaggtttgtacacgactcccatctaggctgaaaacctagaagtggaccttgtgtgtgtgcatggtgtcagcccaaagccaggctgagacagtcctcatatcctcttgagccaaactgtttgggtctcgttgcttcatggtatggtctggatttgtgggaatggctttgcgtgagaaaggggaggagagtggttgctgccctcagccggcttgaggacagagcctgtccctctcatgacaactcagtgttgaagcccagtgtcctcagcttcatgtccagtggatggcagaagttcatggggtagtggcctctcaaaggctgggcgcatcccaagacagccagcaggttgtctctggaaacgaccagagttaagctctcggcttctctgctgagggtgcaccctttcctctagatggtagttgtcacgttatctttgaaaactcttggactgctcctgaggaggccctcttttccagtaggaagttagatgggggttctcagaagtggctgattggaaggggacaagcttcgtttcaggggtctgccgttccatcctggttcagagaaggccgagcgtggctttctctagccttgtcactgtctccctgcctgtcaatcaccacctttcctccagaggaggaaaattatctcccctgcaaagcccggttctacacagatttcacaaattgtgctaagaaccgtccgtgttctcagaaagcccagtgtttttgcaaagaatgaaaagggaccccatatgtagcaaaaatcagggctgggggagagccgggttcattccctgtcctcattggtcgtccctatgaattgtacgtttcagagaaattttttttcctatgtgcaacacgaagcttccagaaccataaaatatcccgtcgataaggaaagaaaatgtcgttgttgttgtttttctggaaactgcttgaaatcttgctgtactatagagctcagaaggacacagcccgtcctcccctgcctgcctgattccatggctgttgtgctgattccaatgctttcacgttggttcctggcgtgggaactgctctcctttgcagccccatttcccaagctctgttcaagttaaacttatgtaagctttccgtggcatgcggggcgcgcacccacgtccccgctgcgtaagactctgtatttggatgccaatccacaggcctgaagaaactgcttgttgtgtatcagtaatcattagtggcaatgatgacattctgaaaagctgcaatacttatacaataaattttacaattctttggaatgagaaaaaaaaaaaaaaaa

SEQ ID NO: 13 - DNA, 1038 bp, Homo sapiens
  misc_feature: PSCA
atttgaggccatataaagtcacctgaggccctctccaccacagcccaccagtgaccacgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctaggctctggggggccccgctgcagcccacactgggtgtggtgccccaggcctctgtgccactcctcacacacccggcccagtgggagcctgtcctggttcctgaggcacatcctaacgcaagtctgaccatgtatgtctgcgcccctgtcccccaccctgaccctcccatggccctctccaggactcccacccggcagatcggctctattgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccggtaaaggctgagatgaagtggactgagtagaactggaggacaggagtcgacgtgagttcctgggagtctccagagatggggcctggaggcctggaggaaggggccaggcctcacattcgtggggctccctgaatggcagcctcagcacagcgtaggcccttaataaacacctgttggataagccagaaaaaaaaaaaaaaaa

SEQ ID NO: 14 - DNA, 4934 bp, Homo sapiens; identical to SEQ ID NO: 12
  misc_feature: KRT15

SEQ ID NO: 15 - DNA, 7771 bp, Homo sapiens
  misc_feature: CACNA1D
tttctgttatttgtccccgtccctccccacccccctgctgaagcgagaataagggcagggaccgcggctcctacctcttggtgatccccttccccattccgcccccgcctcaacgcccagcacagtgccctgcacacagtagtcgctcaataaatgttcgtggatgatgatgatgatgatgatgaaaaaaatgcagcatcaacggcagcagcaagcggaccacgcgaacgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggagtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagatgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctggtgctggtggagacggagaggcgcggccaaggcggggccctctgggtgtcggcggtggggtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcttcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagacccaactgaaagtgaaaatgtccctgtcccaactgctacacctgggaactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgatgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaagaacccgctattatgaaacttacattaggtccgactcaggagatgaacagctcccaactatttgccgggaagacccagagatacatggctatttcagggacccccactgcttgggggagcaggagtatttcagtagtgaggaatgctacgaggatgacagctcgcccacctggagcaggcaaaactatggctactacagcagatacccaggcagaaacatcgactctgagaggccccgaggctaccatcatccccaaggattcttggaggacgatgactcgcccgtttgctatgattcacggagatctccaaggagacgcctactacctcccaccccagcatcccaccggagatcctccttcaactttgagtgcctgcgccggcagagcagccaggaagaggtcccgtcgtctcccatcttcccccatcgcacggccctgcctctgcatctaatgcagcaacagatcatggcagttgccggcctagattcaagtaaagcccagaagtactcaccgagtcactcgacccggtcgtgggccacccctccagcaacccctccctaccgggactggacaccgtgctacacccccctgatccaagtggagcagtcagaggccctggaccaggtgaacggcagcctgccgtccctgcaccgcagctcctggtacacagacgagcccgacatctcctaccggactttcacaccagccagcctgactgtccccagcagcttccggaacaaaaacagcgacaagcagaggagtgcggacagcttggtggaggcagtcctgatatccgaaggcttgggacgctatgcaagggacccaaaatttgtgtcagcaacaaaacacgaaatcgctgatgcctgtgacctcaccatcgacgagatggagagtgcagccagcaccctgcttaatgggaacgtgcgtccccgagccaacggggatgtgggccccctctcacaccggcaggactatgagctacaggactttggtcctggctacagcgacgaagagccagaccctgggagggatgaggaggacctggcggatgaaatgatatgcatcaccaccttgtagcccccagcgaggggcagactggctctggcctcaggtggggcgcaggagagccaggggaaaagtgcctcatagttaggaaagtttaggcactagttgggagtaatattcaattaattagacttttgtataagagatgtcatgcctcaagaaagccataaacctggtaggaacaggtcccaagcggttgagcctggcagagtaccatgcgctcggccccagctgcaggaaacagcaggccccgccctctcacagaggatgggtgaggaggccagacctgccctgccccattgtccagatgggcactgctgtggagtctgcttctcccatgtaccagggcaccaggcccacccaactgaaggcatggcggcggggtgcaggggaaagttaaaggtgatgacgatcatcacacctgtgtcgttacctcagccatcggtctagcatatcagtcactgggcccaacatatccatttttaaaccctttcccccaaatacactgcgtcctggttcctgtttagctgttctgaaatacggtgtgtaagtaagtcagaacccagctaccagtgattattgcgagggcaatgggacctcataaataaggttttctgtgatgtgacgccagtttacataagagaatatcactccgatggtcggtttctgactgtcacgctaagggcaactgtaaactggaataataatgcactcgcaaccaggtaaacttagatacactagtttgtttaaaattatagatttactgtacatgacttgtaatatactataatttgtatttgtaaagagatggtctatattttgtaattactgtattgtatttgaactgcagcaatatccatgggtcctaataattgtagttccccactaaaatctagaaattattagtatttttactcgggctatccagaagtagaagaaatagagccaattctcatttattcagcgaaaatcctctggggttaaaattttaagtttgaaagaacttgacactacagaaatttttctaaaatattttgagtcactataaacctatcatctttccacaagataaaa

SEQ ID NO: 16 - DNA, 4945 bp, Homo sapiens
  misc_feature: ERG
ttcatttcccagacttagcacaatctcatccgctctaaacaacctcatcaaaactactttctggtcagagagaagcaataattattattaacatttattaacgatcaataaacttgatcgcattatggccagcactattaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaatacaccaaaaaaaa

SEQ ID NO: 17 - DNA, 4093 bp, Homo sapiens
  misc_feature: LAMB3
cagaggtgaggctgttgtttaaaaacctggagccgggaggggagacccccacattcaagaggagctttcaggcgatctggagaaagaacggcagaacacacagcaaggaaaggtcctttctggggatcaccccattggctgaagatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcacagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgtaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgatgctacagcttccagcccgttgccccactcatctgccgcctttgcttttggttgggggcagattgggttggaatgctttccatctccaggagactttcatgcagcctaaagtacagcctggaccacccctggtgtgtagctagtaagattaccctgagctgcagctgagcctgagccaatgggacagttacacttgacagacaaagatggtggagattggcatgccattgaaactaagagctctcaagtcaaggaagctgggctgggcagtatcccccgcctttagttctccactggggaggaatcctggaccaagcacaaaaacttaacaaaagtgatgtaaaaatgaaaagccaaataaaaatctttggaaaagagcctggaggttcaacgaggaaaaaaaaaaaaaaaaaaaaaaaaaaaaaaaaaa

SEQ ID NO: 18 - DNA, 9149 bp, Homo sapiens
  misc_feature: FLNC
ccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatagacagcaaggccatcgtggatgggaacctgaagctgatcctgggcctgatctggacgctgatcctgcactactccatctccatgcccatgtgggaggatgaagatgatgaggatgcccgcaaacagacgcccaagcagcggctgcttggctggatccagaacaaggtgccccagctgcccatcaccaacttcaaccgtgactggcaggacggcaaagctctgggcgccctggtggacaactgcgcccccggtctctgccccgactgggaggcctgggaccccaaccagcccgtggagaacgcccgggaggccatgcagcaggccgacgactggcttggggtgccccaggtcattgcccctgaggagattgtggaccccaacgtggatgagcattctgttatgacctacctgtcccagttccccaaggccaagctcaaacctggtgcccctgttcgatccaagcagctgaaccccaagaaagccatcgcctatgggcctggcatcgagccacagggcaacaccgtgctgcagcctgcccacttcaccgtgcagacggtggacgcgggcgtgggcgaggtgctggtctacatcgaggaccctgaaggccacaccgaggaggctaaggtggttcccaacaatgacaaggatcgcacctatgctgtctcctatgtgcccaaggtcgctgggttacacaaggtgaccgtgctctttgctggccagaacattgaacgcagtccctttgaggtgaacgtgggcatggccctgggagatgccaacaaggtgtcagcccgtggccctggcctggaacctgtgggcaatgtggccaacaaacccacctactttgacatctacactgcgggggccggcactggcgatgttgctgtggtgatcgtggacccacagggccggcgggacacagtggaggtggccctggaggacaagggtgacagcacgttccgctgcacatacagacctgccatggaggggccacataccgtgcatgtggcctttgcgggtgcccccatcacccgcagtcccttccctgtccatgtgtcggaagcctgtaaccccaacgcctgccgcgcctctgggcgaggcctgcagcccaagggtgttcgcgtgaaagaggtggctgacttcaaggtgtttaccaagggtgccggcagcggggagctcaaggtcacggtcaaggggccaaagggcacagaggagccagtgaaggtgcgggaggctggggatggtgtgttcgagtgcgagtactacccggtggtgcctgggaagtatgtggtgaccatcacgtggggcggctacgccatccctcgcagcccctttgaggtacaggtgagcccagaggcaggagtgcaaaaggtccgggcctggggtcctggtttggagactggccaggtgggcaagtcagccgattttgtggtggaagccattggcaccgaggtggggacactgggcttctccatcgaggggccctcacaagccaagatcgaatgtgacgacaagggggatggctcctgcgatgtgcggtactggcccacggagcctggggagtacgctgtgcacgtcatctgtgacgatgaggacatccgagactcacccttcattgcccacatcctgcccgccccacctgactgcttcccagataaggtgaaggcctttgggcctggcctggagcctaccggctgcatcgtggacaagcccgctgagttcaccattgatgctcgtgcagctggcaagggagacctgaagctctatgcccaggacgccgacggctgtcccatcgacatcaaggtgatccccaacggcgacggcaccttccgctgctcctacgtgcccaccaagcccattaagcacaccatcatcatctcctggggaggcgtaaacgtgcccaagagccccttccgggtgaacgtgggcgagggcagccaccccgagcgggtaaaggtgtacggccccggagtggagaagacaggcctcaaggccaatgagcccacctacttcacggtggactgcagcgaggcggggcaaggcgacgtgagcatcggcatcaagtgcgccccaggcgtggtgggccctgcagaggctgacattgacttcgacatcatcaagaatgacaacgacaccttcaccgtcaagtacacgccaccaggggcgggccgctacaccatcatggtgctgtttgccaaccaggagatccccgccagccccttccacatcaaggtggacccatcccacgatgccagcaaagtcaaggccgagggccctgggctgaatcgcacaggtgtggaagtcgggaagcccacccacttcacggtgctgaccaagggagccggcaaggccaagctggatgtgcagtttgcagggacagccaagggcgaggttgtgcgggactttgagatcatagacaaccatgactactcctacactgtcaagtacaccgctgtccagcagggcaacatggcagtgacagtgacttatggcggggaccctgtccccaagagcccctttgtggtgaatgtggcacccccgctggacctcagcaaaatcaaagttcagggccttaatagcaaggtggctgtgggacaggaacaagcattctctgtgaacacacgaggggctggcggtcagggccaactggatgtgcggatgacttcgccctctcgccggcccatcccctgcaagctggagccaggcggtggagcggaagcccaggctgtgcgctacatgcccccggaggaggggccctacaaggtggatatcacctacgatggtcacccggtgcctggcagcccgtttgctgtggagggtgtcctgccccctgatccctccaaggtctgtgcttatggcccgggtctcaagggtggactggtaggcacccccgcgccattctccatcgacaccaagggggctggcacaggtggcctggggctgaccgtagagggcccctgcgaggccaagatcgagtgccaggacaatggtgatggctcatgtgctgtcagctacctgcccacggagcctggcgagtacaccatcaacatcctgtttgctgaggcccacatccctggctcgcccttcaaagccaccattcggcctgtgtttgacccgagcaaggtgcgggccagtggaccgggcctggagcgcggcaaggtcggtgaggcagccaccttcactgtggactgctcagaggcaggcgaggcggagctgaccattgagatcctgtcggatgccggggtcaaggccgaggtgctgatccacaacaacgcggatggcacctaccacatcacctacagccctgccttccctggcacctacaccattaccatcaagtatggcgggcatcccgtgcccaaattccccacccgtgtccatgtgcagcctgcggtcgataccagtggcgtcaaggtctcagggcctggtgttgagccacacggtgtcctgcgggaggtgaccactgagttcactgtggatgcaagatccctaacagccacaggcggcaaccacgtgacggctcgtgtgctcaacccctcgggggccaagacagacacctatgtgacagacaatggggacggcacctaccgagtgcagtacaccgcctacgaggagggcgtgcatctggtggaggtcctgtatgatgaggtcgctgtgcccaagagccccttccgagtgggcgtgaccgagggctgtgatcccacccgcgtccgagccttcgggccaggcctggagggtggcttggtcaacaaggccaaccgattcactgtggagaccaggggagcgggcaccgggggccttggcctagccatcgagggtccctcggaagccaagatgtcctgcaaggacaacaaggatggtagctgcaccgtggagtacatccccttcactcctggagactatgacgtcaacatcaccttcggggggcggcccatcccagggagcccgttccgcgtgccagtgaaggatgtggtggaccctgggaaggtgaagtgctcagggccagggctgggggctggtgtcagggcccgggttcctcagaccttcacagtggactgcagtcaagctggccgggcgcccctgcaggtggctgtgctgggccccacaggtgtggccgagcctgtggaggtgcgggacaatggagatggcacccacactgtccactacaccccagccactgacgggccctacacggtagccgtcaagtatgctgaccaggaggtgccacgcagccccttcaagatcaaggtcctcccagctcatgatgccagcaaggtgcgggccagcggcccaggcctcaacgcctctggcatccctgccagcctgcctgtggagttcaccatcgacgcacgggacgcgggcgaggggttgctcactgtccagatcttggaccccgagggtaagcccaagaaggccaacatccgggacaatggggatggcacgtacactgtgtcctacctgccggacatgagtggccggtacaccatcaccatcaagtatggcggtgatgagatcccctactcgcccttccgcatccatgctctgcccactggggatgccagcaagtgcctcgtcacagtgtccattggaggccatggcctgggtgcctgcctgggccctcgaatccagattgggcaggagacggtgatcacggtggatgccaaggcagccggtgaggggaaggtgacatgcacggtgtccacgccggatggggcagagctcgatgtggatgtggttgagaaccatgacggtacctttgacatctactacacagcgcccgagccgggcaagtacgtcatcaccatccgcttcgggggtgagcacatccccaacagccccttccacgtgctggcgtgtgaccccctgccgcacgaggaggagccctctgaagtgccacagctgcgccagccctacgctcctccccggcccggcgcccgccccacacactgggccacagaggagccagtggtgcctgtggagccaatggagtccatgctgaggcccttcaacctggtcatccccttcgcggtgcagaaaggggagctcacaggagaggtgcggatgccctcggggaagacggcacggcccaacatcaccgacaacaaggacggcaccatcacggtgaggtatgcacccactgagaaaggcctgcaccagatggggatcaagtatgacggcaaccacatccctgggagccccttacagttctatgtggatgccatcaacagccgccatgtcagtgcctatgggccaggcctgagccatggcatggtcaacaagccagccaccttcactattgtcaccaaagatgctggagaagggggtctgtcactggccgtggagggcccatccaaggcagagatcacctgtaaggacaacaaggatggcacctgcaccgtgtcctatctgccgactgcgcctggagactacagcatcatcgtgcgcttcgatgacaagcacatcccggggagccccttcacagccaagatcacaggtgatgactccatgaggacctcacagctgaatgtgggcacctccacggacgtgtcactgaagatcaccgagagtgatctgagccagctgaccgccagcatccgtgccccctcgggcaacgaggagccctgcctgctgaagcgcctgcccaaccggcacattgggatctccttcacccccaaggaggtcggggagcacgtggtgagcgtgcgcaagagtggcaagcatgtcaccaacagccccttcaagatcctggtggggccatctgagatcggggacgccagcaaggtgcgggtctggggcaaggggctttccgagggacacacattccaggtggcagagttcatcgtggacactcgcaatgcaggttatgggggcttggggctgagtattgaaggcccaagcaaggtggacatcaactgtgaggacatggaggacgggacatgcaaagtcacctactgccccaccgagcccggcacctacatcatcaacatcaagtttgctgacaagcacgtgcctggaagccccttcactgtgaaggtgaccggcgagggccgcatgaaggagagcatcacccggcggagacaggcaccttccatcgccaccatcggcagcacctgtgacctcaacctcaagatcccaggaaactggttccagatggtgtctgcccaggagcgcctgacacgcaccttcacacgcagcagccacacctacacccgcacggagcgcacggagatcagcaagacgcggggcggggagacaaagcgcgaggtgcgggtggaggagtccacccaggtcggcggggaccccttccctgctgtgtttggggacttcctgggccgggagcgcctgggatccttcggcagcatcacccggcagcaggagggtgaggccagctctcaggacatgactgcacaggtgaccagcccatcgggcaaggtggaagccgcagagatcgtcgagggcgaggacagcgcctacagcgtgcgctttgtgccccaggaaatggggccccatacggtcgctgtcaagtaccgtggccagcacgtgcccggcagcccctttcagttcactgtggggccgctgggtgaaggtggtgcccacaaggtgcgggccggaggcacagggctggagcgaggtgtggccggcgtgccagccgagttcagcatctggacccgggaggctggcgctgggggcctgtccattgctgtggagggtcctagcaaagcggagattgcatttgaggatcgcaaagatggctcctgcggcgtctcctatgtcgtccaggaaccaggtgactatgaggtctccatcaagttcaatgatgagcacatcccagacagcccctttgtggtgcctgtggcctccctctcggatgacgctcgccgtctcactgtcaccagcctccaggagacggggctcaaggtgaaccagccagcgtcctttgccgtgcagctgaacggtgcccggggcgtgattgatgcccgggtgcacacaccctcgggggctgtggaggagtgctacgtctctgagctggacagtgacaagcacaccatccgcttcatcccccacgagaatggcgtccactccatcgatgtcaagttcaacggtgcccacatccctggaagtcccttcaagatccgcgttggggagcagagccaggctggggacccaggcttggtgtcagcctacggtcctgggctcgagggaggcactaccggtgtgtcatcagagttcatcgtgaacaccctgaatgccggctcgggggccttgtctgtcaccattgatggcccctccaaggtgcagctggactgtcgggagtgtcctgagggccatgtggtcacttatactcccatggcccctggcaactacctcattgccatcaagtacggtggcccccagcacatcgtgggcagccccttcaaggccaaggtcactggtccgaggctgtccggaggccacagccttcacgaaacatccacggttctggtggagactgtgaccaagtcctcctcaagccggggctccagctacagctccatccccaagttctcctcagatgccagcaaggtggtgactcggggccctgggctgtcccaggccttcgtgggccagaagaactccttcaccgtggactgcagcaaagcaggcaccaacatgatgatggtgggcgtgcacggccccaagaccccctgtgaggaggtgtacgtgaagcacatggggaaccgggtgtacaatgtcacctacactgtcaaggagaaaggggactacatcctcattgtcaagtggggtgacgaaagtgtccctggaagccccttcaaagtcaaggtcccttgaatcccaaaagtgcctccccagcctcagcccccacctccagccacacacacattacacacacacacacacacacacaaatgtgccacacccagacacgcacagaatcagacactacaaacacctgccttgggggtgaagtgaaggcccagcctccccaccccaccgcgccccaggggttggaggaccttgtctgtgtcaggacagtgtccctccctgggaatgtgacatgagggccgactggggccaggctcaggggcagaggctgggacacaaggggctggcgagggctgcgaggccagggaagccctgagtttctggcggggctgagcagtgggggagcattgtgttgtgggtgtctgtgtgtgaggtcaccctcaaactgcaccgccggccagataccctcctgaccccgaggacttggtctggtctctctggtggctacaaccccagagttttaaggacttggaaaggaaagcacaatcagagaagaaaacagcccccgaaccagcaggagtggcctggcacatggaccggcctgagcgatgtgcactccacccaagccaggctcccagggggcctgatttctctctcactgtctctttttttaaaatggttgcacggctctgccccatggggggccttttttacacactgcgaggcccagctttctaggggacttttgcacatgtcatgcagctcagctgggagctgcttaggtggaaaactccaaataaagtgcggctgtcgcagaaaaaaaaaaaa

SEQ ID NO: 19 - DNA, 1968 bp, Homo sapiens
  misc_feature: RASSF1A
tctcctcagctccttcccgccgcccagtctggatcctgggggaggcgctgaagtcggggcccgccctgtggccccgcccggcccgcgcttgctagcgcccaaagccagcgaagcacgggcccaaccgggccatgtcgggggagcctgagctcattgagctgcgggagctggcacccgctgggcgcgctgggaagggccgcacccggctggagcgtgccaacgcgctgcgcatcgcgcggggcaccgcgtgcaaccccacacggcagctggtccctggccgtggccaccgcttccagcccgcggggcccgccacgcacacgtggtgcgacctctgtggcgacttcatctggggcgtcgtgcgcaaaggcctgcagtgcgcgcattgcaagttcacctgccactaccgctgccgcgcgctcgtctgcctggactgttgcgggccccgggacctgggctgggaacccgcggtggagcgggacacgaacgtggacgagcctgtggagtgggagacacctgacctttctcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttgggtgacctcttgtacccccaggtggaaggcagacagcaggcagcgccaagtgcgtgccgtgtgagtgtgacagggccagtggggcctgtggaatgagtgtgcatggaggccctcctgtgctgggggaatgagcccagagaacagcgaagtagcttgctccctgtgtccacctgtgggtgtagccaggtatggctctgcacccctctgccctcattactgggccttagtgggccagggctgccctgagaagctgctccaggcctgcagcaggagtggtgcagacagaagtctcctcaatttttgtctcagaagtgaaaatcttggagaccctgcaaacagaacagggtcatgtttgcaggggtgacggccctcatctatgaggaaaggttttggatcttgaatgtggtctcaggatatccttatcagagctaagggtgggtgctcagaataaggcaggcattgaggaagagtcttggtttctctctacagtgccaactcctcacacaccctgaggtcagggagtgctggctcacagtacagcatgtgccttaatgcttcatatgaggaggatgtccctgggccagggtctgtgtgaatgtgggcactggcccaggttcataccttatttgctaatcaaagccagggtctctccctcaggtgttttttatgaagtgcgtgaatgtatgtaatgtgtggtggcctcagctgaatgcctcctgtggggaaaggggttggggtgacagtcatcatcagggcctggggcctgagagaattggctcaataaagatttcaagatcctcaaaaaaaaaaaaaaaaaaa

SEQ ID NO: 20 - DNA, 1703 bp, Homo sapiens
  misc_feature: TMEM178
gggcggagagctggggccaagtgcattgtgtctggcggcggcgcgcgagcccaccggcggctgcggcggggcgggaagccatggagccgcgggcgctcgtcacggcgctcagcctcggcctcagcctgtgctccctggggctgctcgtcacggccatcttcaccgaccactggtacgagaccgacccccggcgccacaaggagagctgcgagcgcagccgcgcgggcgccgaccccccggaccagaagaaccgcctgatgccgctgtcgcacctgccgctgcgggactcgcccccgctggggcgccggctgctcccgggcggcccggggcgcgccgaccccgagtcctggcgctcgctcctggggctcggcgggctggacgccgagtgcggccggcccctcttcgccacctactcgggcctctggaggaagtgctacttcctgggcatcgaccgggacatcgacaccctcatcctgaaaggtattgcgcagcgatgcacggccatcaagtaccacttttctcagcccatccgcttgcgaaacattccttttaatttaaccaagaccatacagcaagatgagtggcacctgcttcatttaagaagaatcactgctggcttcctcggcatggccgtagccgtccttctctgcggctgcattgtggccacagtcagtttcttctgggaggagagcttgacccagcacgtggctggactcctgttcctcatgacagggatattttgcaccatttccctctgtacttatgccgccagtatctcgtatgatttgaaccggctcccaaagctaatttatagcctgcctgctgatgtggaacatggttacagctggtccatcttttgcgcctggtgcagtttaggctttattgtggcagctggaggtctctgcatcgcttatccgtttattagccggaccaagattgcacagctaaagtctggcagagactccacggtatgactgtcctcactgggcctgtccacagtgcgagcgactcctgaggggaacagcgcggagttcaggagtccaagcacaaagcggtcttttacattccaacctgttgcctgccagccctttctggattactgatagaaaatcatgcaaaacctcccaacctttctaaggacaagactactgtggattcaagtgctttaatgactatttatgcgttgactgtgagaatagggagcagtgccatgggacatttctaggtgtagagaaagaagaaactgcaatggaaaaatttgtatgatttccatttatttcagaaagtttgtatgtaacaattacccgagagtcatttctacttgcaaaaggattcgtaacaaagcgagtataattttcttgtcattgtatcatgcttgttaaattttaatgcagcatcttcagaacttgtcctgatggtgtcttattgtgtcagcaccaaatatttgtgcattatttgtggacgttccttgtcacaggaagattcttcttctgttgccttattgtttttttttttttaagtctcttctctgtctttgtactggaatcgaaatcataagataaacagatcaaacgtgcttaagagctaactcgtgacactatgcagtattgtttgaagacctgttgttcaacctctgtctctttatgttaactggatttctgcattaaatgactgcccccttgttaaaaaaaaaaaaaaaaaaa

SEQ ID NO: 21 - DNA, 2300 bp, Homo sapiens
  misc_feature: HOXC4
ttattgtggtttgtccgttccgagcgctccgcagaacagtcctccctgtaagagcctaaccattgccagggaaacctgccctgggcgctcccttcattagcagtattttttttaaattaatctgattaataattatttttcccccatttaattttttttcctcccaggtggagttgccgaagctgggggcagctggggagggtggggatgggaggggagagacagaagttgagggcatctctctcttccttcccgaccctctggcccccaaggggcaggaggaatgcaggagcaggagttgagcttgggagctgcagatgcctccgcccctcctctctcccaggctcttcctcctgcccccttcttgcaactctccttaattttgtttggcttttggatgattataattatttttatttttgaatttatataaagtatatgtgtgtgtgtgtggagctgagacaggctcggcagcggcacagaatgagggaagacgagaaagagagtgggagagagagaggcagagagggagagagggagagtgacagcagcgctcgcgggggctcaacccccagacctccagaaatgacgtcagaatcatttgcatcccgctgcctctacctgcctggtccagctgggaccctgcctcgccggccgcatggccagagggttggaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagtttta

SEQ ID NO: 22 - DNA, 1978 bp, Homo sapiens
  misc_feature: RPL22L1
cgctctcagcgcgtgacgcagcacgctttgatataaatgcagaccgcgcggccgtagcttcctctctgctctcgcggccgactcgcaagatggcgccgcagaaagacaggaagcccaagaggtcaacctggaggtttaatttggaccttactcatccagtagaagatggaatttttgattctggaaattttgagcaatttctacgggagaaggttaaagtcaatggcaaaactggaaatctcgggaatgttgttcacattgaacgcttcaagaataaaatcacagttgtttctgagaaacagttctctaaaaggtatttgaaataccttaccaagaaataccttaagaagaacaatcttcgtgattggcttcgagtggttgcatctgacaaggagacctacgaacttcgttacttccagattagtcaagatgaagatgaatcagagtcggaggactaggcaaaggctccccttacagggctttgcttattaataaaataaatgaagtatacatgagaaataccaagaaattggcttttagtttatcagtgaataaaaaatattatactcttgaacttttgtctcatttttttgagtatgctgtttatatgattttgatttccctctgataactatcaacagtatttaaatagcttatagctggtataattttttcccacgatttccaaaatcttttatgtactcaggtaaaagtagcgttatataggaaatcttttttttagacactctcgttctgtcacccaggctggagtgcagtgactcagcttcctaaatagctggaattacaggtgtgagccaccatgcccggctaattttttgtacttttagtagagtagggtttggccatgttggccaggctggtttcaaactcctgacctcaagtgatctacccacctcggcttcccaaagtgctgattatagctgtgaaccaccatgcccggccaggaaatcttactgtagaacaattttttatatagctgtataaaatgtatatgattgtcttgacagtctcaaatactgtttttaatagcttgtaaatgtaatctcaagtgcttagaacagttcttacatataagttgctctgtagtttgctcttatagttagcccaaagactctgggtgtgaggcctgctgtaaaccaatgttaaactgcttattagaaagccctaaccacctgctttgtaggcaccagaaactcaaaaccaaatctcaactcagctacagaatctactgtggtccttgtctgaaaaaattagttcactcggttggaatcttgtctcagagcatcctcatctctttctcaaaagcccctaccccaacaccggcgtgttggttgtctattgaaacttacaagtggatggaccctttctcccgaataaactggcctttgaaagctctaatcgaaatggtttggcaaaatccatactgcaggagattagggaggacaagaatgatgtgcctttttgtactgctgagcctgatggtggtgccactacttcaggtacttagatgagtcttgatgctaatagaattgtgtcgccaaacatatctggacagttacaacctaatctatgcattaattggtttgggaattgcttgaaattattgtttaattcaatgttttaattcgttttcctaaaaatttaagtgcccccatcatcgtgcaatacctcagtgcagcaactccttgattcttggatgactgaacttcctaacttggctctgccccattgttcccatttttcatgtttttcacaaatagttaaccaggtacctactactgtgcaccgctgcagagcattgaggatgtatgtgatgagtaaaaacacccagcctgctctgctgtgttagtattatgacggaaactgatcaaatcacatgtgaacaaatttactgctacaaaagggagggcttaataaaaggaatttcatctgggaaggcaaaaaaaaaaaaaaaaaa

SEQ ID NO: 23 - DNA, 5335 bp, Homo sapiens
  misc_feature: EFNA5
gcttctctccatcttgtgattcctttttcctcctgaaccctccagtgggggtgcgagtttgtctttatcaccccccatcccaccgccttcttttcttctcgctctcctacccctccccagcttggtgggcgcctctttcctttctcgccccctttcatttttatttattcatatttatttggcgcccgctctctctctgtccctttgcctgcctccctccctccggatccccgctctctccccggagtggcgcgtcgggggctccgccgctggccaggcgtgatgttgcacgtggagatgttgacgctggtgtttctggtgctctggatgtgtgtgttcagccaggacccgggctccaaggccgtcgccgaccgctacgctgtctactggaacagcagcaaccccagattccagaggggtgactaccatattgatgtctgtatcaatgactacctggatgttttctgccctcactatgaggactccgtcccagaagataagactgagcgctatgtcctctacatggtgaactttgatggctacagtgcctgcgaccacacttccaaagggttcaagagatgggaatgtaaccggcctcactctccaaatggaccgctgaagttctctgaaaaattccagctcttcactcccttttctctaggatttgaattcaggccaggccgagaatatttctacatctcctctgcaatcccagataatggaagaaggtcctgtctaaagctcaaagtctttgtgagaccaacaaatagctgtatgaaaactataggtgttcatgatcgtgttttcgatgttaacgacaaagtagaaaattcattagaaccagcagatgacaccgtacatgagtcagccgagccatcccgcggcgagaacgcggcacaaacaccaaggatacccagccgccttttggcaatcctactgttcctcctggcgatgcttttgacattatagcacagtctcctcccatcacttgtcacagaaaacatcagggtcttggaacaccagagatccacctaactgctcatcctaagaagggacttgttattgggttttggcagatgtcagatttttgttttctttctttcagcctgaattctaagcaacaacttcaggttgggggcctaaacttgttcctgcctccctcaccccaccccgccccacccccagccctggcccttggcttctctcacccctcccaaattaaatggactccagatgaaaatgccaaattgtcatagtgacaccagtggttcgtcagctcctgtgcattctcctctaagaactcacctccgttagcgcactgtgtcagcgggctatggacaaggaagaatagtggcagatgcagccagcgctggctagggctgggagggttttgctctcctatgcaatatttatgccttctcattcagaactgtaagatgatcgcgcagggcatcatgtcaccatgtcaggtccggaggggaggtattaagaatagatacgatattacaccatttcctataggagtatgtaaatgaacaggcttctaaaaggttgagacactggtttttttttttaatatgactgtcttaaagcattcttgacagcaaaacttgtgctctctaaaagaagccttttttttttttctaggaggcaggttgggtgtggaatgctaatacagagcaggtgtgaaaacagagaaaactacaggtttgctgggggtgtgtatgtgtgagtgcctctaatttttttggtgactgggcagtgcacaccagatattttttctttgaatacagatcaccatggtgctacaactttttttttttttttttttttttttttttttttttttaagaaactcaaagaggcatttttatgaataaagtgaccttccccaaggctgacaagccagggttgatgagtgcatagtggaatagctttggatactcctctgggggatgacatgtaccaaggagaggaccgcagtggccagaggagacatgatttggctttgctggagcgccagtgtgctgtggcctttccccgcctcccaccctagtacccacgttttgctccacactccttgaccgcaggggctcggacacaaacccctgtcaccaggagagtcagtcagcactacttgggagggctaaagggaaatttggaaataaaattccaaagtttggagtaaaaaaattcaagtgttgattttatattctttccctttctgacacagcctaaagcgtagggggaacatgtgtttatctgtgggagataaacaagatggagtcccaaagactttaacaaaatatttttttaaaaatccactagaatagaaaatacattatttagatatactttatgctgagagtgagtatatatgcttgtcctatttaaacttgtgagaaaaagtggtatcccttgatacatttagaaatatgggggctatcttgtttcattgtgggggtggggcagaaggagaataaatgcaggatgaccctgttgaaggaatcttagcatggccaacaggggacgtttccagtcgattaccaggaaatgcaagccttggggtttctactggtggtggggctgtcatgaactttaaaatccaaagcctagacaaggaaaagtgttagaccaattgaaaagcaatccagccctttttttttttttttttttggctttgcacgacatgtcaacagaaaccatgcctttcaatataagaaataaatgtgatgatcatgtaaaatgtgaaaaattgaaagcattccagcaaaataagaattttttatatatttgttttttaagatgtatatgttaaaaaaagagaaggtcgcattatggacagacttcgtgaatgggaatttgcttagaattgtgagtagttctgaattagaaaagtatgtgaaggaaaggcagctgtaaacgtattgtgccctggagagttgtacacatgttgaaatgtaatctgggcttacctgatccatttggagtggatgtcactgccgagtctgttctcacatggaaccatgtgtgtggggttgccagcctcacagatacaatcaatcctattcccctctgacataaggaactcctctggagtggcagagtcttatcacagaaggcagccaccatttcaccaaaacaaaagttcacggcattcaattcctttttcctttagctatttatatatgcagtactctcagtcatatgcagaaatacttttttttttttaattaatagttacaggcttgttggtccagtgggatttgggtagggggagaaagataccttctaaaatggatcaatagaaccaaaataatacagcatgttctataaccacaaggaaatcaaatgatcctgtcatgattccagttagtcataactatgttagcagtgctaaatgcattttagaaatggtgacttctgtggttttcctagcatttgtctctaacaaatggtgaaataattactcatggccctctctgccattgtctttcattttttcacagtgaaattagacccctttacttcaccattctgccactgcaaattaagtataaagaaaatagcaagagtgtccacaccagtagacagtaagcttctctacctgtaagtgatgaaatcatagctaatgcacttgccatggagttttcaagatgattggtgtcagacagttttcactttgtttaaaaagtgttggtggccttttgtggtggtgttacaatcctctgggggcttaggaggatgttgatgcaacttttagaagcttttaatttcaaaaacaactcaaaaatctgaaggacagtcatagctgccactcagccccagttagtcaaaccccagtgacctttgcccctggttgccaagggctttgcaacatcaagcagggaaataaggatctgtctgtttagtggataccgtgtatcctttaatagaccaggtaacagttcgtgttagtttagactattgttttgtactgtactttcttgggtggcagaggaaagaaaagtaaaacattaaaaaaaaaaaaaaaactgcgttctttaaattctgtattattagcaacctctgttgtacatagtgtttgataataaagtattaatttgattcttatgtcttttgtaagtgagaacaatagactttcaggatacaaaacatgcattgaggctttgaaacatccaatgtgtaccatggctgaaaaaaagagtcacaagtggctgagacactgctccatacaggactcatgtgtggtcattgcccacccaatcttatgactccatcatcttgggacctggaacaacatgacttttttgatcgatattttgtcctcggtgttttcaaggtctgatgttggagccctgtggcccaccctcccttctccaccagcatgttggtttgaaaaggatagggaatttagaaacagttctgtactttggtttggtttggttttctgtttgtgattgttttcatggactgttttattttttcccaggaagagtctttatcaatatcatgtgcagctcactcatggaaatggttgcaaaccaatcagtgtaggaagcttaaatggggctgtttccctctctgtgtcgttgtgaaaggaagagtcacacagtacctgtggattttcagggactctgtttttctccggtgccttagcaacggcagcagccatttgtattattttcaaataattagaaaaacagttttcaactcctcctttccattcattgctctcagagttgtggtcactgacttttcttttgaaaaccgcctccaccaacacccccgtttgcctacaccaccccccttttacttagtatgtttattttttgtgtgtctcttgccttcctcccacgttttatttcccctcagagctgtgaatgggcaggtctgtctctggtttggcatcactgagtttttcccatgcattggccccagggctgctaggatgtgagacaaatctccctacaatgggcttgctcccattgtctgtacagtttaatagatgctggcatgtcggaggttacccatgagtcaaaatccgctctccatgcttactcttgacaccccattgaagccactcattgtgtgtgcgtctgggtgtgaagtccagctccgtgtggtcctgtgcttgtactgccctgctttgcagttcctttgcacttactcatcgagtgctgttttgaaatgctgacattatataaacgtaaaagaaaatgtaaaaaaaaaaaacccacacacaaacaaacccatacgatctgtatttgtatatacacgtgtccgtacaagtataactaaataaaaattaaagattttcatcattttaattgga

SEQ ID NO: 24 - DNA, 3037 bp, Homo sapiens
  misc_feature: TRIM29
ctcctcacaggtgtgtctctagtcctcgtggttgcctgccccactccctgccgagacgcctgccagaaaggtcacctatcctgaaccccagcaagcctgaaacagctcagccaagcaccctgcgatggaagctgcagatgcctccaggagcaacgggtcgagcccagaagccagggatgcccggagcccgtcgggccccagtggcagcctggagaatggcaccaaggctgacggcaaggatgccaagaccaccaacgggcacggcggggaggcagctgagggcaagagcctgggcagcgccctgaagccaggggaaggtaggagcgccctgttcgcgggcaatgagtggcggcgacccatcatccagtttgtcgagtccggggacgacaagaactccaactacttcagcatggactctatggaaggcaagaggtcgccgtacgcagggctccagctgggggctgccaagaagccacccgttacctttgccgaaaagggcgagctgcgcaagtccattttctcggagtcccggaagcccacggtgtccatcatggagcccggggagacccggcggaacagctacccccgggccgacacgggccttttttcacggtccaagtccggctccgaggaggtgctgtgcgactcctgcatcggcaacaagcagaaggcggtcaagtcctgcctggtgtgccaggcctccttctgcgagctgcatctcaagccccacctggagggcgccgccttccgagaccaccagctgctcgagcccatccgggactttgaggcccgcaagtgtcccgtgcatggcaagacgatggagctcttctgccagaccgaccagacctgcatctgctacctttgcatgttccaggagcacaagaatcatagcaccgtgacagtggaggaggccaaggccgagaaggagacggagctgtcattgcaaaaggagcagctgcagctcaagatcattgagattgaggatgaagctgagaagtggcagaaggagaaggaccgcatcaagagcttcaccaccaatgagaaggccatcctggagcagaacttccgggacctggtgcgggacctggagaagcaaaaggaggaagtgagggctgcgctggagcagcgggagcaggatgctgtggaccaagtgaaggtgatcatggatgctctggatgagagagccaaggtgctgcatgaggacaagcagacccgggagcagctgcatagcatcagcgactctgtgttgtttctgcaggaatttggtgcattgatgagcaattactctctccccccacccctgcccacctatcatgtcctgctggagggggagggcctgggacagtcactaggcaacttcaaggacgacctgctcaatgtatgcatgcgccacgttgagaagatgtgcaaggcggacctgagccgtaacttcattgagaggaaccacatggagaacggtggtgaccatcgctatgtgaacaactacacgaacagcttcgggggtgagtggagtgcaccggacaccatgaagagatactccatgtacctgacacccaaaggtggggtccggacatcataccagccctcgtctcctggccgcttcaccaaggagaccacccagaagaatttcaacaatctctatggcaccaaaggtaactacacctcccgggtctgggagtactcctccagcattcagaactctgacaatgacctgcccgtcgtccaaggcagctcctccttctccctgaaaggctatccctccctcatgcggagccaaagccccaaggcccagccccagacttggaaatctggcaagcagactatgctgtctcactaccggccattctacgtcaacaaaggcaacgggattgggtccaacgaagccccatgagctcctggcggaaggaacgaggcgccacacccctgctcttcctcctgaccctgctgctcttgccttctaagctactgtgcttgtctgggtgggagggagcctggtcctgcacctgccctctgcagccctctgccagcctcttgggggcagttccggcctctccgacttccccactggccacactccattcagactcctttcctgccttgtgacctcagatggtcaccatcattcctgtgctcagaggccaacccatcacaggggtgagataggttggggcctgccctaacccgccagcctcctcctctcgggctggatctgggggctagcagtgagtacccgcatggtatcagcctgcctctcccgcccacgccctgctgtctccaggcctatagacgtttctctccaaggccctatcccccaatgttgtcagcagatgcctggacagcacagccacccatctcccattcacatggcccacctcctgcttcccagaggactggccctacgtgctctctctcgtcctacctatcaatgcccagcatggcagaacctgcagcccttggccactgcagatggaaacctctcagtgtcttgacatcaccctacccaggcggtgggtctccaccacagccactttgagtctgtggtccctggagggtggcttctcctgactggcaggatgaccttagccaagatattcctctgttccctctgctgagataaagaattcccttaacatgatataatccacccatgcaaatagctactggcccagctaccatttaccatttgcctacagaatttcattcagtctacactttggcattctctctggcgatggagtgtggctgggctgaccgcaaaaggtgccttacacactgcccccaccctcagccgttgccccatcagaggctgcctcctccttctgattaccccccatgttgcatatcagggtgctcaaggattggagaggagacaaaaccaggagcagcacagtggggacatctcccgtctcaacagccccaggcctatgggggctctggaaggatgggccagcttgcaggggttggggagggagacatccagcttgggctttcccctttggaataaaccattggtctgtcaaaaaaaaaaaaaaaaaaa

SEQ ID NO: 25 - DNA, 1412 bp, Homo sapiens
  misc_feature: HLA-DMB
atttgtccctgcctacctagccaatctgtccctgtttgggacactggactcccgtgagctggaaggaacagatttaatatctaggggctgggtatccccacatcactcatttggggggtcaagggacccgggcaatatagtattctgctcagtgtctggagatcatctacccaggctggggcttctgggacaggcgaggacccacggaccctggaagagctggtccaggggactgaactcccggcatctttacagagcagagcatgatcacattcctgccgctgctgctggggctcagcctgggctgcacaggagcaggtggcttcgtggcccatgtggaaagcacctgtctgttggatgatgctgggactccaaaggatttcacatactgcatctccttcaacaaggatctgctgacctgctgggatccagaggagaataagatggccccttgcgaatttggggtgctgaatagcttggcgaatgtcctctcacagcacctcaaccaaaaagacaccctgatgcagcgcttgcgcaatgggcttcagaattgtgccacacacacccagcccttctggggatcactgaccaacaggacacggccaccatctgtgcaagtagccaaaaccactccttttaacacgagggagcctgtgatgctggcctgctatgtgtggggcttctatccagcagaagtgactatcacgtggaggaagaacgggaagcttgtcatgcctcacagcagtgcgcacaagactgcccagcccaatggagactggacataccagaccctctcccatttagccttaaccccctcttacggggacacttacacctgtgtggtagagcacactggggctcctgagcccatccttcgggactggacacctgggctgtcccccatgcagaccctgaaggtttctgtgtctgcagtgactctgggcctgggcctcatcatcttctctcttggtgtgatcagctggcggagagctggccactctagttacactcctcttcctgggtccaattattcagaaggatggcacatttcctagaggcagaatcctacaacttccactccaagtgagaaggagattcaaactcaatgatgctaccatgcctctccaacatcttcaaccccctgacattatcttggatcctatggtttctccatccaattctttgaatttcccagtctcccctatgtaaaacttagcaacttgggggacctcattcctgggactatgctgtaaccaaattattgtccaaggctatatttctgggatgaatataatctgaggaagggagttaaagaccctcctggggctctcagtgtgccatagaggacagcaactggtgattgtttcagagaaataaactttggtggaaatattgttaaaaaaaaaaaaaaa

SEQ ID NO: 26 - DNA, 1681 bp, Homo sapiens
  misc_feature: HOXC6
ttttgtctgtcctggattggagccgtccctataaccatctagttccgagtacaaactggagacagaaataaatattaaagaaatcatagaccgaccaggtaaaggcaaagggatgaattcctacttcactaacccttccttatcctgccacctcgccgggggccaggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta

SEQ ID NO: 27 - DNA, 3233 bp, Homo sapiens
  misc_feature: THBS4
cagcagccagctccccagcaccgcacggcggggacgcgagcgcgcccccgacggcagcccggacgccgagcacgggtcacctgcggcgccggcccgggcgccgaccgaggttcaacgcacggcccggggacccccaggcggggccaacgccgccgtcgcccccggcctcgcggggagcaggaagagccaacatgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaaaaaaaaaaa

SEQ ID NO: 28 - DNA, 2219 bp, Homo sapiens
  misc_feature: CRISP3
gcacaaccagaatttgccaaaacaggaaataggtgtttcatatatacggctctaaccttctctctctgcaccttccttctgtcaatagatgaaacaaatacttcatcctgctctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaa

SEQ ID NO: 29 - DNA, 10397 bp, Homo sapiens
  misc_feature: SDK1
cctcagcgctgggcggccgctcacctcgggccggggggcgccgcgcctcccgcggagtggccgcgcccgctcggagccgtcccgcctgtcctgcccgcccgtccgtccggcgcggcgctcggggtggcggctgctcggcatggcccggggcgcccggccctcggcggccggtggcggcggcggcggcgcggagccccctgagcgcgcgggccccgggcggccgcggggatccccgcccggccgcgcccgcccctcgctggcgccgcgccccggcccggagccctcgcgaccccgggcggcgcccgagacctccggcggggacacggcgggcgcggggcggtgcggcgggcggcgggcggcaaagttggggccgggccgccgcggctggtgggcgctgctggcgctgcagctgcacttgctccgggcgctggcgcaagatgatgttgctccatattttaaaacggagccaggcctaccacagatccacctggaagggaaccgccttgttctcacctgccttgccgaagggagctggcctttggagttcaagtggatgcgcgatgacagtgagctcaccacctacagcagcgaatataagtacattattccatctttgcagaagctcgatgctgggttttaccgctgcgtggtgcgaaacagaatgggagcactcctgcaaagaaaatcagaagttcaagtcgcatatatgggaagtttcatggatacggaccagaggaaaacagtttctcaaggacgtgcagcgattctaaacctgctgcccatcaccagctaccccagacctcaagtgacttggtttagagaagggcacaagattattccaagcaacagaatagccatcacattggagaatcagctggtgatcctcgccaccacaaccagtgatgccggggcatactacgtgcaggccgtgaatgagaaaaatggagaaaacaagacaagcccattcattcatttgagcatagcaagagatgttggcacacctgaaaccatggccccaaccattgtggttcccccgggcaacagaagtgtggtggctggatccagtgagaccaccttggaatgtatagccagtgccaggcctgtggaggacctgagtgtgacctggaagaggaatggagtgagaatcaccagtggcctccacagctttggaagacgcctcaccatcagcaacccgacgtccgcggacaccgggccatacgtctgcgaggcggcgctgccggggagcgcttttgaaccggccagggcgacggcctttcttttcatcatagagccaccatattttactgctgagcccgagagtcggatttcagctgaagtagaagaaactgtggacatcggatgtcaagccatgggggtcccccttcccaccctccagtggtacaaggatgccatctccatcagcaggctccagaatcctcgatacaaagtgctcgccagcggaggcctgcgcatccagaagctgcgtccagaggactccggaatcttccagtgcttcgccagcaatgaaggaggggagatccagacccacacctacctggatgtaaccaatatcgctccagtgttcacccagcggccagtggacaccacagttactgacgggatgacagccattctaaggtgtgaggtgtccggggctcccaaacccgccatcacctggaaaagagaaaaccacattctggccagtggctctgtccggattcctaggttcatgcttcttgaatcggggggtctacagatcgcgcccgtcttcatccaggatgccggcaactacacctgctatgcggccaacacagagggctccctgaatgcatcggccacgctcactgtgtggaatcggacgtccatcgtccaccctcctgaggaccacgtggtgattaaggggaccacggccacgctgcactgtggtgccacacatgacccccgggtttcactccgctacgtttggaagaaggacaacgtggccctgactccatcgagcacgtctaggatcgtggtggagaaggacgggtcccttctcatcagccagacgtggtcaggcgacatcggtgactacagctgcgagattgtttctgaaggagggaatgactccaggatggcccggctggaagtgattgaactgcctcattcacctcagaacctcctggtcagccctaattcttcccacagccacgccgtggtgctctcttgggtccggccctttgatggaaacagtcctattctttattacatcgtggagctctctgaaaacaactctccatggaaggtgcatctgtcaaacgttggccctgagatgacaggcgtcaccgtgagtggcctgactccggctcgtacctatcaattccgggtgtgcgcggtgaatgaagtgggcaggggccagtacagcgccgagacaagcaggttgatgctacctgaagaaccacccagtgctcccccgaaaaatatagtggccagtgggcggactaatcagtccattatggtccagtggcagccacccccagaaacagagcacaacggggtgttgcgtggatacatcctcaggtaccgcctggctggccttcccggagagtaccagcagcggaacatcaccagcccggaggtgaactactgcctggtgacagacctgatcatctggacacagtatgagatacaggtggcggcgtacaacggggccggtctgggcgtcttcagcagggcagtgaccgagtacaccttgcagggagtgcccaccgcgcccccgcagaacgtgcagacggaagccgtgaactccaccaccattcagttcctgtggaaccctccgcctcagcagtttatcaatggcatcaaccagggatacaagcttctggcatggccggcagatgcccccgaggctgtcactgtggtcactattgccccagatttccacggagtccaccatggacacataacgaacctgaagaagtttaccgcctacttcacttccgttctgtgcttcaccacccctggggacgggcctcccagcacacctcagctggtctggactcaggaagacaaaccaggagctgtgggacatctgagtttcacagagatcttggacacatctctcaaggtcagctggcaggagcccctggagaaaaatggcatcattactggctatcagatctcttgggaagtgtacggcaggaacgactctcgtctcacgcacaccctgaacagcacgacgcacgagtacaagatccaaggcctctcatctctcaccacctacaccatcgacgtggccgctgtgactgccgtgggcactggcctggtgacttcatccaccatttcttctggagtgcccccagaccttcctggtgccccatccaacctggtcatttccaacatcagccctcgctccgccacccttcagttccggccaggctatgacgggaaaacgtccatctccaggtggattgttgaggggcaggtgggagctatcggcgacgaggaggagtgggtcaccctctatgaagaggagaatgagcctgatgcccagatgctggagatcccaaacctcacaccctacactcactacagatttcgaatgaagcaagtgaacattgttgggccgagcccctacagtccgtcttcccgggtcatccagaccctgcaggccccacccgacgtggctccaaccagcgtcacggtccgtactgccagtgagaccagcctgcggcttcgctgggtgcccctgccggattctcagtacaacgggaaccccgagtccgtgggctacaggattaagtactggcgctcagacctccagtcctcagcagtggcccaagtcgtcagtgaccggctggagagagaattcaccatcgaggagctggaggagtggatggaatacgagctgcagatgcaggccttcaacgccgtcggggctgggccgtggagcgaggtggtgcggggccggacgcgggagtcagttccttcagccgcccctgagaacgtgtcagccgaggctgtcagctcgacccagattttactgacatggacatccgtgccggaacaggaccagaatgggctcatactgggctacaagatcctgttccgggccaaagacctggatcccgagcccaggagccacatcgtgcgagggaaccacacgcagtcggccctgctggcaggcctgcgcaagttcgtgctctacgagctccaggtgctggcgttcacccgcatcgggaacggggtccccagcacgcccctcatcctggagcgcaccaaagacgatgccccaggcccaccagtgaggctcgtgttccccgaagtgagactcacctccgtgcggatagtgtggcaacctccggaggagcccaacggcatcatcctggggtaccagattgcctaccgcctggccagcagcagcccccacaccttcaccaccgtggaggtcggcgccacagtgaggcagttcacagccaccgacctggccccggagtccgcatacatcttcaggctgtccgccaagacgaggcagggctggggggagccactggaggccaccgtcatcaccaccgagaagagagagcggccggcaccccccagagagctcctggtgccccaggcagaagtgaccgcacgcagcctccggctccagtgggtcccgggcagcgacggggcctcccccatccggtacttcaccatgcaggtgcgagagctgcctcggggtgagtggcagacctactcctcgtccatcagccatgaggcgacagcatgcgtcgttgacagactgaggcccttcacctcctacaagctgcgcctgaaagccaccaacgacattggggacagtgacttcagttcagagacagaggcggtgaccacgctgcaggatgttccaggagagcccccgggatctgtctcagcgacgccacacaccacgtcctctgtcctgatacagtggcagcctccgagggacgaaagcctgaatggccttcttcagggatacaggatctactacagggagctggagtatgaagccgggtcaggcactgaggccaagacgctcaaaaaccctatagctttacatgctgagctcacagcccaaagcagcttcaagacggtgaacagcagctccacatcgacgatgtgtgaactaacacatttaaagaagtaccggcgctatgaagtaataatgaccgcctataacatcatcggcgagagcccagccagcgcgcccgtggaggtctttgtcggcgaggctgccccggccatggccccgcagaacgtgcaggtgaccccactcacggccagccagctggaggtcacgtgggacccaccacccccggagagccagaatgggaacatccaaggctacaagatttactactgggaggcagacagccagaacgaaacggagaaaatgaaggtcctcttcctccccgagcccgtggtgaggctgaagaacctgaccagccataccaagtacctggtcagcatatcagccttcaacgccgccggagatggacctaagagtgacccccagcaggggcgcacccaccaggccgcccctggggcccccagctttctggcgttctcagaaataacctccaccacgctcaacgtgtcctggggcgagcctgcggcggccaacggcatcctgcagggctatcgggtggtgtacgagcccttggcccctgtacaaggggtgagcaaggtggtgaccgtggaagtgagagggaactggcagcgctggctgaaggtgcgggacctcaccaagggagtgacctatttcttccgtgtccaagcgcggaccatcacctacgggcccgagctccaagccaatatcacagccgggccagccgagggatccccgggctcgcctagagatgtcctggtcaccaagtccgcctctgaactgacgctgcagtggactgagggacactctggcgacacacctaccacgggctatgtgatcgaggcccggccctcagatgaaggcttatgggacatgtttgtgaaggacatcccgcggagcgccacatcctacaccctcagcctggataagctccggcaaggagtgacttacgagttccgggtggtggctgtgaatgaggcgggctacggggagcccagcaacccctccacggctgtgtcagctcaagtggaagccccattctacgaggagtggtggttcctcctggtgatggctctgtccagcctgatcgtcatcctgctggtggtgttcgccctcgtcctgcacgggcagaataagaagtataagaactgcagcacaggaaaggggatctccaccatggaggagtctgtgaccctggacaacggaggatttgctgccctggagctcagcagccgccacctcaatgtcaagagcaccttctccaagaagaacgggaccaggtccccaccccggcctagccccggcggcctgcactactcagacgaggacatctgcaacaagtacaacggcgccgtgctgaccgagagcgtgagcctcaaggagaagtcggcagatgcatcagaatctgaggccacggactctgactacgaggacgcgctgcccaagcactccttcgtgaaccactacatgagcgaccccacctactacaactcatggaagcgcagggcccagggccgcgcacctgcgccgcacaggtacgaggcggtggcgggctccgaggcgggcgcgcagctgcacccggtcatcaccacgcagagcgcgggcggcgtctacacccccgctggccccggcgcgcgaactccgctcaccggcttctcctccttcgtgtgagcaaagcgccgcgcctccctcagggcggaacggaggcaactttccggagtctatttttgttaagacaatcaactccaataactgagctgaagtttttgtttaaaaagaaaaaaatctgataagtgatgattttacctacttgtggacactagatttcaattaggaaggtttttttaaacggctttttgtaacttcgctgcaggaagcaggtttgtttctttttcttttctttttaagagaaggtgtatttcactggtgcaatggcttggcacctccggggcctgggaggacctcagacctccccagccctgggtttctccgtcttcaagaccaactaggaagggtcaagcggggagagggagtggagggtcaggtgagatctcagagctgccccggccggcccccgtctctttctacctcctcttccagagaaccagcggctcacacccttctcaacgcaggacatcctcggcggctcctggggtttgaagagcaaacgtttttccctgggctcagtgcgttttgtcccaacttcatctgtttctgaaatgttctcacttggcagtgtctagtcaaggagtcggctttcaggttcctgacggccaggcagggatgctaaggtgtggctcagccgtcactgtctgtgtcactgcagtggtgcggtcctcaggcttttctgcctgtctttcccccttccttctcacctgacagcgagggagagggaagcctcttagggctggaagccaccacgctggccctctccttccccgaacacagcacacggtcaactccgcgggacacgaggacacgggacggcgtctccagaattgcttgttacgtaggaagcgtgcattgttaaccagagtatttttaaaatctttttatctttttttaaactatgtcacatgaaatgaatgcgtctttgctgtctccaggtgcctttttattaattgttcagctttgtacatgggaaagatgaaaagcaacagtgtctgcaaataaagcaaaacagctctgagaacacacgctcccgactcgcctcgtgcacaccaggccgtcccctccctctgtcctggcttctgctggctgctcgcagcagccaccgcttctccaccactggcgctgctgctgccccctctcccagtccgaggccagcttttagccttaacaggttttttggaaatgtttcttttttttatttaaaattgtcattgtttggtttaaatttttcagctagatgaaaagagtatgaactactttggaaaacttaacagctcagagatggccatgcctccagcccctcacgtcatctttgcaacagacgactgggctgccatggtccacccctcagcccgggtcccgggtctggatggaacgggagcactgctggtgcccactggcgtgtgtgccccgggtccctgtaagtgccccctcaccagcagcagcgtgacacacacaagactcaagaccaccctgtcagtgccccccagtgcacggcaaacgggcaggtgccgttcccccagtgacctgagggtaggggacaactgagcagtatctgaccagtgccacccaggagccagtctcctggccacatgcagaaagtgtggcccctgcttacctagatgttttgtgcacctccatgggcagagggtgtggatattgcctggattctgtgctgtcagcgttgctgagtatggccccaggagaccaaggagagttttgtataggctggaaaaccccttttcagtctttccaaaattagagggtatggcaagtttccttttttctctcctcccttccttcccctccttcctttcctttacccctcctttccttccttcctcccttcctctcttctttcctccctccctccctccttccctcccttccttcctctctttcctccttccttccctcccttctttccctccctcccttcctctctcccctattccttcttccttttctcctcctttttctgagtggagggggaaatattctaaaccaaaaatcctagatgctctgcccaaagccacttctgcatgagaatcgcaacccacagttccccggatgagactcaccacagtggacagtgccacctccttcccctcggccccggagagggcgaagtgggcgggaagccaggatgtgagcactggaatttcttggaagagaagcgataaatggagaccatggccagcgctgctttctgtgcactctgatgactgctctctgcagccatgaggatgtggctttacatgccagggagagtgttgagacgtcttaggttgaggatgagcagattcgagatatgtttgttgctctcgggttttcgatacaacatcatgacacttctgtttcaagctcatgttttccgtctcccctccactcttagtaaaccttgatctgtacggagcggcctgtccgaggctacgccggcctcctggctgctgctggactgtgcttaggacagcgcccatgcctcggagggactctgtcccatgagaaccacctgtgcaaaggaacagagctggatgtttccaggtagattttggcctcccagagcaatgcggcatttgagaagcaacagttcctaactccttatcttcagggaaggaaaagaaaatcacagcctaggaagatggaggttggattttaatctcggttttaaaaagaggacaaacaaaatgtctctaagccaggctagatggaatgtgctcccgctctctcctgccgtgctgaaagtcatgccttgcggatgcctcatgacagcagtggctgagtctccccacccacccccaacgtggctcatttcagattgcttcggccccaccctgcaaggatgtggtcacggagtggccaggaggctccgtctgagccacagggatgggtgtgcagagctccctcctcctggggtgccagggcagagattccaggcaggtgagcccagagagagctgccaggccacaccccctcggcctcctgcacggccaccttctgggtgaatcggtccagcccaagcccctctccccagcctcgccttcagcctctctcccagcctgcttttataaggcgcacttcactcaatgctgtagccaaaaaacgaggggccccagggagaggggacccagatggccacacacggaacgcgcctccacagccccgggaggtggctcactctgtacaggtcttcggaggccgtgtttgtatctaactgtgactgggctgaagcatgatgtttgcctaatggttcgtagcatggtttttatttcttacgcattcttggcacacagtgtagctatcctcctgacgagcaacccgtctgcgtacctaagtgtggctccccgtgggtcagcgtcctggtagcatggatccagtctgaaaggtgaggacaacgtggaaactcatgagctgagcctgcccgctgggacacgtctccttcccgcgtcaccttctggtttagggagccgtcaggtccctaaacgttccctacaactttttctgaaattgtgcagaaaaacagatctcattaaaagaaaaaaagaaacaacttgtaggaagacagagaggtgctatgggtacaatttttaataaaaacattattttgttccttaaa

SEQ ID NO: 30 - DNA, 2446 bp, Homo sapiens
  misc_feature: SDR5A2
tccataaaggggttgcgggggccgcgctctcttctgggagggcagcggccaccggcgaggaacacggcgcgatgcaggttcagtgccagcagagcccagtgctggcaggcagcgccactttggtcgcccttggggcactggccttgtacgtcgcgaagccctccggctacgggaagcacacggagagcctgaagccggcggctacccgcctgccagcccgcgccgcctggttcctgcaggagctgccttccttcgcggtgcccgcggggatcctcgcccggcagcccctctccctcttcgggccacctgggacggtacttctgggcctcttctgcctacattacttccacaggacatttgtgtactcactgctcaatcgagggaggccttatccagctatactcattctcagaggcactgccttctgcactggaaatggagtccttcaaggctactatctgatttactgtgctgaataccctgatgggtggtacacagacatacggtttagcttgggtgtcttcttatttattttgggaatgggaataaacattcatagtgactatatattgcgccagctcaggaagcctggagaaatcagctacaggattccacaaggtggcttgtttacgtatgtttctggagccaatttcctcggtgagatcattgaatggatcggctatgccctggccacttggtccctcccagcacttgcatttgcatttttctcactttgtttccttgggctgcgagcttttcaccaccataggttctacctcaagatgtttgaggactaccccaaatctcggaaagcccttattccattcatcttttaaaggaaccaaattaaaaaggagcagagctcccacaatgctgatgaaaactgtcaagctgctgaaactgtaattttcatgatataatagtcccgtatatatgtaatagtaggtctcctggcgttctgccagctggcctggggattctgagtggtgtctgcttagagtttactcctacccttccagggacccctatcctgatccccaactgaagcttcaaaaagccacttttccaaatggcgacagttgcttcttagctattgctctgagaaagtacaaacttctcctatgtctttcaccgggcaatccaagtacatgtggcttcatacccactccctgtcaatgcaggacaactctgtaatcaagaattttttgacttgaaggcagtacttatagaccttattaaaggtatgcattttatacatgtaacagagtagcagaaatttaaactctgaagccacaaagacccagagcaaacccactcccaaatgaaaaccccagtcatggcttcctttttcttggttaattaggaaagatgagaaattattaggtagaccttgaatacaggagccctctcctcatagtgctgaaaagatactgatgcattgacctcatttcaaatttgtgcagtgtcttagttgatgagtgcctctgttttccagaagatttcacaatccccggaaaactggtatggctattcttgaaggccaggttttaataaccacaaacaaaaaggcatgaacctgggtggcttatgagagagtagagaacaacatgaccctggatggctactaagaggatagagaacagttttacaatagacattgcaaactctcatgtttttggaaactagtggcaatatccaaataatgagtagtgtaaaacaaagagaattaatgatgaggttacatgctgcttgcctccaccagatgtccacaacaatatgaagtacagcagaagccccaagcaactttcctttcctggagcttcttccttgtagttctcaggacctgttcaagaaggtgtctcctaggggcagcctgaatgcctccctcaaaggacctgcaggcagagactgaaaattgcagacagaggggcacgtctgggcagaaaacctgttttgtttggctcagacatatagttttttttttttttacaaagtttcaaaaacttaaaaatcaggagattccttcataaaactctagcattctagtttcatttaaaaagttggaggatctgaacatacagagcccacatttccacaccagaactggaactacgtagctagtaagcatttgagtttgcaaactcttgtgaaggggtcaccccagcatgagtgctgagatatggactctctaaggaaggggccgaacgcttgtaattggaatacatggaaatatttgtcttctcaggcctatgtttgcggaatgcattgtcaatatttagcaaactgttttgacaaatgagcaccagtggtactaagcacagaaactcactatataagtcacataggaaacttgaaaggtctgaggatgatgtagattactgaaaaatgcaaattgcaatcatataaataagtgtttttgttgttcattaaatacctttaaatcatg

SEQ ID NO: 31 - DNA, 1177 bp, Homo sapiens
  misc_feature: TAGLN
tcaccacggcggcagccctttaaacccctcacccagccagcgccccatcctgtctgtccgaacccagacacaagtcttcactccttcctgcgagccctgaggaagccttctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaaaaaaaaaaaaaaaaaa

SEQ ID NO: 32 - DNA, 3640 bp, Homo sapiens
  misc_feature: WFS1
gtgcagaaggccgcgctagccggctcttcagcagcgagtgcagattgctcccccgcggccgcagatctcccgtttgcgccgcgttcagctgctcccgaacaacttttctgccggcccagaggccccagggcgtcgcagcgccgcgtgcggcccactcacgggccggcaggatggactccaacactgctccgctgggcccctcctgcccacagcccccgccagcaccgcagccccaggcgcgttcccgactcaatgccacagcctcgttggagcaggagaggagcgaaaggccccgagcacccggaccccaggctggccctggccctggtgttagagacgcagcggcccccgctgaaccccaggcccagcataccaggagccgggaaagagcagacggcaccgggcctacaaagggagacatggaaatcccctttgaagaagtcctggagagggccaaggccggggaccccaaggcacagactgaggtggggaagcactacctgcagttggccggcgacacggatgaagaactcaacagctgcaccgctgtggactggctggtcctcgccgcgaagcagggccgtcgcgaggctgtgaagctgcttcgccggtgcttggcggacagaagaggcatcacgtccgagaacgaacgggaggtgaggcagctctcctccgagaccgacctggagagggccgtgcgcaaggcagccctggtcatgtactggaagctcaaccccaagaagaagaagcaggtggccgtggcggagctgctggagaatgtcggccaggtcaacgagcacgatggaggggcgcagccaggccccgtgcccaagtccctgcagaagcagaggcgcatgctggagcgcctggtcagcagcgagtccaagaactacatcgcgctggatgactttgtggagatcactaagaagtacgccaagggcgtcatccccagcagcctgttcctgcaggacgacgaagatgatgacgagctggcggggaagagccctgaggacctgccactgcgtctgaaggtggtcaagtaccccctgcacgccatcatggagatcaaggagtacctgattgacatggcctccagggcaggcatgcactggctgtccaccatcatccccacgcaccacatcaacgcgctcatcttcttcttcatcgtcagcaacctcaccatcgacttcttcgccttcttcatcccgctggtcatcttctacctgtccttcatctccatggtgatctgcaccctcaaggtgttccaggacagcaaggcctgggagaacttccgcaccctcaccgacctgctgctgcgcttcgagcccaacctggatgtggagcaggccgaggtcaacttcggctggaaccacctggagccctatgcccatttcctgctctctgtcttcttcgtcatcttctccttccccatcgccagcaaggactgcatcccctgctcggagctggctgtcatcaccggcttctttaccgtgaccagctacctgagcctgagcacccatgcagagccctacacgcgcagggccctggccaccgaggtcaccgccggcctgctatcgctgctgccctccatgcccttgaattggccctacctgaaggtccttggccagaccttcatcaccgtgcctgtcggccacctggtcgtcctcaacgtcagcgtcccgtgcctgctctatgtctacctgctctatctcttcttccgcatggcacagctgaggaatttcaagggcacctactgctaccttgtgccctacctggtgtgcttcatgtggtgtgagctctccgtggtcatcctgctggagtccaccggcctggggctgctccgcgcctccatcggctacttcctcttcctctttgccctccccatcctggtggccggcctggccctggtgggcgtgctgcagttcgcccggtggttcacgtctctggagctcaccaagatcgcagtcaccgtggcggtctgtagtgtgcccctgctgttgcgctggtggaccaaggccagcttctctgtggtggggatggtgaagtccctgacgcggagctccatggtcaagctcatcctggtgtggctcacggccatcgtgctgttctgctggttctatgtgtaccgctcagagggcatgaaggtctacaactccacactgacctggcagcagtatggtgcgctgtgcgggccacgcgcctggaaggagaccaacatggcgcgcacccagatcctctgcagccacctggagggccacagggtcacgtggaccggccgcttcaagtacgtccgcgtgactgacatcgacaacagcgccgagtctgccatcaacatgctcccgttcttcatcggcgactggatgcgctgcctctacggcgaggcctaccctgcctgcagccctggcaacacctccacggccgaggaggagctctgtcgccttaagctgctggccaagcacccctgccacatcaagaagttcgaccgctacaagtttgagattaccgtgggcatgccattcagcagcggcgctgacggctcgcgcagccgcgaggaggacgacgtcaccaaggacatcgtgctgcgggccagcagcgagttcaagagcgtgctgctcagcctgcgccagggcagcctcatcgagttcagcaccatcctggagggccgcctgggcagcaagtggcctgtcttcgagctcaaggccatcagctgcctcaactgcatggcccagctctcacccaccaggcggcacgtgaagatcgagcacgactggcgcagcaccgtgcatggcgccgtgaagttcgccttcgacttctttttcttcccattcctgtcggcggcctgaggatggtccgccacgaggagcttccagtgcatgttgccatgaggcctttccccagtgtggccccagcccgacaggcatgcaccagtgccgcctgtgcccacgtgtgcagactgtggctgcagagaccttgcgaccatgtgtagattgcgtggaccccgacaaagggaaggctgctgtgtagctctgtccactctgaataccaagtgtgttgggaattgcatgccatctccaccctgagcctgacctttctgagtgacatgggtgtgccaggctagactaggaggttccggtgtctggaaaagcactttacagatgagattccctctcctcccccaccttcaagcaccctgttccctctttctttcttttgtgttggatttgtttaaaaaccaaataagcatctgtgtaacctccacagtagcatttcttatttgtttggtcactgctacaccttagcagctcttcccctttcctgggggatgtgcacggcagcttgagcctgtcacgtggtcaaggcccggccccatcagaggctgggggaggcggcacattggcagtgtgtcacactgagctgggcaccacaggctgcctcatgaccctcctgtccagcaggtagtgggtgaatgtgtgaaggtcttgcctgaatccatcaggacttgggaaacagagaaccctgtgggggcggctgtgggggaggtccctgccagtgtttagaagagcctgactgtgttcagtgccttggagcagaaagccagggtcctgagtggctgaaataaaagcctctggtggaacctgca

SEQ ID NO: 33 - DNA, 2112 bp, Homo sapiens
  misc_feature: SNAI2
aaaacgggctcagttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaa

SEQ ID NO: 34 - DNA, 3296 bp, Homo sapiens
  misc_feature: GDPD1
ccggcaccgacaagtgcgctgcaccagtggcaccggctgggggcgagccgacctcgagcagccgccgccgccgccgtcgttgctactgccgcagcggagttcagagggcccggaggtgggagacttcccacacggtgactgagatgtcgtccactgcggctttttaccttctctctacgctaggaggatacttggtgacctcattcttgttgcttaaatacccgaccttgctgcaccagagaaagaagcagcgattcctcagtaaacacatctctcaccgcggaggtgctggagaaaatttggagaatacaatggcagcctttcagcatgcggttaaaatcggaactgatatgctagaattggactgccatatcacaaaagatgaacaagttgtagtgtcacatgatgagaatctaaagagagcaactggggtcaatgtaaacatctctgatctcaaatactgtgagctcccaccttaccttggcaaactggatgtctcatttcaaagagcatgccagtgtgaaggaaaagataaccgaattccattactgaaggaagtttttgaggcctttcctaacactcccattaacatcgatatcaaagtcaacaacaatgtgctgattaagaaggtttcagagttggtgaagcggtataatcgagaacacttaacagtgtggggtaatgccaattatgaaattgtagaaaagtgctacaaagagaattcagatattcctatactcttcagtctacaacgtgtcctgctcattcttggccttttcttcactggcctcttgccctttgtgcccattcgagaacagttttttgaaatcccaatgccttctattatactgaagctaaaagaaccacacaccatgtccagaagtcaaaagtttctcatctggctttctgatctcttactaatgaggaaagctttgtttgaccacctaactgctcgaggcattcaagtgtatatttgggtattaaatgaagaacaagaatacaaaagagcttttgatttgggagcaactggggtgatgacagactatccaacaaagcttagggattttttacataacttttcagcatagaaaaagaggtacttagaagtattgaaggaaaaaatgaagacctaagaaaaaaatatttcatgatcatttccctaagccatttccagaatggtaaaaggtttaatcagtttttattacctcatttttaagcctgtatgagaatgtagaaactatatattatatgtatatttattttaaataatattgtatattttatgtttgtaaattgtttagaaagataattggttatgagatgtaagttttaatttcttaatgtgcatttttgtttctagatcttatacagaaatcttgattaataacatacacagaaatgtacatactacatcatctacagaaatcttgatcaataacctagaaactaggttatctaggttattgatcaagatttctgtagatgatgcagtgttctatcataagtaattctggaatcaaagactattggatacatttggcattgggctgagtgtggtggctcatgcctgtaatcccagcactttgggaggctgagacaggcggatcatctgaagtcaggagttaaagaccagcctggccaacatggcaaaaccccatctctaccaaaaatacaaaaattaaccatgcgtggtggtacacgtctgtcatcccagcagctcttaaggctgaggcacaagaattgcttgaacccgggaggcagaggctgtagtgagccaagatagcaccactgcactccagcctgggagacagagtgagactccgtctcaaaaaaaaaaaaaaaaaaaaaaaatgggaggccgaggcgggcggatcacgaggtcaggagatcgagaccatcctggctaacatggtgaaaccccgtctctactaaaaatacaaaaaattagctgggcgtggtggtgggcaccttagtcccagctactcgggaggctgagtcaggagaatggcgtgaacccgggaggcggagcttgcagtgagccgagatcgcgccactgcactccagcctgggctacagagcaagactccgtctcaaaaaaagaaaaaaagaaaaaaaattggcaatagtcttcactggaatacaatcaattagtaaaagattttttttttttttttgacatgtagtcttgctctgtcgccgaggccggagtgcagtggtgcgatcttggctcactgcaacctctgcctcccaggttccagcaattctcctgcctctgcctcccgagtacctgggattacaggtgcctgctaccatgcccagctaatttttgtatttttagtagagacagggttttgccacgttggccagactggtctcgaactcctgacctcaggtgatccacccacctcggcctctcaaagtgctgggattacaggtttgaaccactgcacccggccagtaaaagaaattttgaaggccattgcagctatttggtagtgtcttgttatttctaggtgtaccttagttaaagaggaaaaataaaacggaaaaaagcttggaaatcagtgatgtgtagttatttggcaagttatacataatcagcagcagccaggctcaagaaaataaaagttgattagttgatcagaaataaaatctgtagagtgaattagatttctgagttgttgttgttaatggaacattctatttgagacctttttcaggtgtgtagcaattctaccatgtccatttttttaagcattaaaaaggaacttaccagttgtaaattaagacaagatccaaatagtcatatttttgtgtttcctctaaaaaaatgtaagatttccatttttggcactgactaactgagcctacatctagattttaaataccatcttgaatcctaataattaaactgatgaaagtgcatatcattgttcctaacatttatgaacccccataaagatgttcctgatctttaaaactcattaatctgagtattaagtagaaacagaattttccaaagcattagacatcactttctcagtttatctgaggtgacttcgtgtacatctgtttctaatatatttgactaattttcatgatctcagattgtgaggtaaatgtaatctggaataataagtgtcttttacctagattacatctctcatttggagtttggcaatgaaactgctatgaagaatgactgtactctcctatctgtccctggatgacataaatatcatttgctttgttgtttaaactgaaataaagttttccaagaacaaaaaaaaaaaaaa

SEQ ID NO: 35 - DNA, 3010 bp, Homo sapiens
agcattgaccaataggagaccgtagtgatagcgacggggaaattcaaacgtgtttgcggaaaggagtttgggttccatcttttcatttccccagcgcagctttctgtagaaatggaatccgaggatttaagtggcagagaattgacaattgattccataatgaacaaagtgagagacattaaaaataagtttaaaaatgaagaccttactgatgaactaagcttgaataaaatttctgctgatactacagataactcgggaactgttaaccaaattatgatgatggcaaacaacccagaggactggttgagtttgttgctcaaactagagaaaaacagtgttccgctaagtgatgctcttttaaataaattgattggtcgttacagtcaagcaattgaagcgcttcccccagataaatatggccaaaatgagagttttgctagaattcaagtgagatttgctgaattaaaagctattcaagagccagatgatgcacgtgactactttcaaatggccagagcaaactgcaagaaatttgcttttgttcatatatcttttgcacaatttgaactgtcacaaggtaatgtcaaaaaaagtaaacaacttcttcaaaaagctgtagaacgtggagcagtaccactagaaatgctggaaattgccctgcggaatttaaacctccaaaaaaagcagctgctttcagaggaggaaaagaagaatttatcagcatctacggtattaactgcccaagaatcattttccggttcacttgggcatttacagaataggaacaacagttgtgattccagaggacagactactaaagccaggtttttatatggagagaacatgccaccacaagatgcagaaataggttaccggaattcattgagacaaactaacaaaactaaacagtcatgcccatttggaagagtcccagttaaccttctaaatagcccagattgtgatgtgaagacagatgattcagttgtaccttgttttatgaaaagacaaacctctagatcagaatgccgagatttggttgtgcctggatctaaaccaagtggaaatgattcctgtgaattaagaaatttaaagtctgttcaaaatagtcatttcaaggaacctctggtgtcagatgaaaagagttctgaacttattattactgattcaataaccctgaagaataaaacggaatcaagtcttctagctaaattagaagaaactaaagagtatcaagaaccagaggttccagagagtaaccagaaacagtggcaatctaagagaaagtcagagtgtattaaccagaatcctgctgcatcttcaaatcactggcagattccggagttagcccgaaaagttaatacagagcagaaacataccacttttgagcaacctgtcttttcagtttcaaaacagtcaccaccaatatcaacatctaaatggtttgacccaaaatctatttgtaagacaccaagcagcaataccttggatgattacatgagctgttttagaactccagttgtaaagaatgactttccacctgcttgtcagttgtcaacaccttatggccaacctgcctgtttccagcagcaacagcatcaaatacttgccactccacttcaaaatttacaggttttagcatcttcttcagcaaatgaatgcatttcggttaaaggaagaatttattccatattaaagcagataggaagtggaggttcaagcaaggtatttcaggtgttaaatgaaaagaaacagatatatgctataaaatatgtgaacttagaagaagcagataaccaaactcttgatagttaccggaacgaaatagcttatttgaataaactacaacaacacagtgataagatcatccgactttatgattatgaaatcacggaccagtacatctacatggtaatggagtgtggaaatattgatcttaatagttggcttaaaaagaaaaaatccattgatccatgggaacgcaagagttactggaaaaatatgttagaggcagttcacacaatccatcaacatggcattgttcacagtgatcttaaaccagctaactttctgatagttgatggaatgctaaagctaattgattttgggattgcaaaccaaatgcaaccagatacaacaagtgttgttaaagattctcaggttggcacagttaattatatgccaccagaagcaatcaaagatatgtcttcctccagagagaatgggaaatctaagtcaaagataagccccaaaagtgatgtttggtccttaggatgtattttgtactatatgacttacgggaaaacaccatttcagcagataattaatcagatttctaaattacatgccataattgatcctaatcatgaaattgaatttcccgatattccagagaaagatcttcaagatgtgttaaagtgttgtttaaaaagggacccaaaacagaggatatccattcctgagctcctggctcatccatatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcagataccacctataaaatatattggactgttatactcttgaatccctgtggaaatctacatttgaagacaacatcactctgaagtgttatcagcaaaaaaaattcagtagattatctttaaaagaaaactgtaaaaatagcaaccacttatggcactgtatatattgtagacttgttttctctgttttatgctcttgtgtaatctacttgacatcattttactcttggaatagtgggtggatagcaagtatattctaaaaaactttgtaaataaagttttgtggctaaaatgacactaacattt

SEQ ID NO: 36 - DNA, 11659 bp, Homo sapiens
  misc_feature: TTK
gctggagggatcctccattcctgtgtcatttgcatgggtcctgctgtgaaatgaacctggcagggacttgttagacacttccttccttccctcattgagcactccagtgccattgttccacagttgttctaattgggtcctagcttcctcctgccaaggcaaacagcatagtctcgagtaggtgtccctaggctcatctgccagcctgaacatgaacacaggcaaagctgatgatggccagggaccccaggggacgtggggccctgtggggtctggcccccaggagcaagacctctgatgatgctggtgtctgggagtgagcaccatgcccatcacccaggacaatgccgtgctgcacctgcccctcctctaccagtggctgcagaacagcctgcaggaaggtggggatgggccggagcagcggctctgccaggcggccatccagaagctgcaggagtacatccagctgaactttgctgtggatgagagtacggtcccacctgatcacagcccccccgaaatggagatctgtactgtgtacctcaccaaggagctgggggacacagagactgtgggcctgagttttgggaacatccctgttttcggggactatggtgaaaagcgcagggggggcaagaagaggaaaacccaccagggtcctgtgctggatgtgggctgcatctgggtgacagagctgaggaagaacagcccagcagggaagagtgggaaggtccgactgcgggatgagatcctctcactgaatgggcagctgatggttggagttgatgtcagtggggccagttacctggctgagcagtgctggaatggcggctttatctacctgatcatgctgcgtcgctttaagcacaaagcccactccacttataatggcaacagtagcaacagctctgaaccaggagaaacacctaccttggagctgggtgaccgaactgcgaaaaaggggaaacgaaccagaaagtttggggtcatctccaggcctcctgccaacaaggcccctgaagaatccaagggcagcgctggctgtgaggtgtccagtgaccccagcactgagctggagaacggccctgaccctgaacttggaaacggccatgtctttcagctagaaaatggcccagattctctcaaggaggtggctggaccccatctagagaggtcagaagtggacagagggacagagcatagaattccaaagacagatgctcctctgaccacaagcaatgacaaacgccgcttctcaaaaggtgggaagacggacttccaatcgagtgactgcctggcacgggaggaagttggccgaatatggaagatggagctgctcaaagaatcggatgggctgggaattcaggttagtggaggccgaggatcaaagcgctcacctcacgctatcgttgtcactcaagtgaaggaaggaggtgccgctcacagggatggcaggctgtccttaggagatgagctgctggtaatcaatggtcatttactggtcgggctctcccacgaggaagcagtggccattcttcgctccgccacgggaatggtgcagcttgtggtggccagcaaggaaaactccgcagaggacctcctcaggttaacatctaagagcttgccagatctgaccagctcggtagaagatgtgtcctcctggactgataacgaagaccaggaggcagacggggaagaggacgaaggaaccagctcttctgtccagagagcaatgcctgggacagatgaaccccaagatgtgtgcggtgctgaggaatccaaggggaacttggaaagtcccaaacagggcagcaataaaatcaagctcaagagtcgcctttcagggggtgtacaccgccttgagtcagttgaagaatataacgagctgatggtgcggaatggggacccccggatccggatgttggaggtctcccgagatggccggaaacactccctcccgcagctgctggactcttccagtgcctcacaggaataccacattgtgaagaagtctacccgctccttaagcacgactcaggtggaatctccttggaggctcattcggccatccgtcatctcgatcattgggttgtacaaagaaaaaggcaagggccttggctttagtattgctggaggtcgagactgcattcgtggacagatggggatttttgtcaagaccatcttcccaaatggatcagctgcagaggacggaagacttaaagaaggggatgaaatcctagatgtaaatggaataccaataaagggcttgacatttcaagaagccattcatacctttaagcaaatccggagtggattatttgttttaacggtacgcacaaagttggtgagccccagcctcacaccctgctcgacacccacacacatgagcagatccgcctccccgaacttcaataccagtgggggagcctcagcgggaggttccgatgaaggcagttcttcatccctgggtcggaagacccctgggcccaaggacaggatcgtcatggaagtaacactcaacaaagagccaagagttggattaggcattggtgcctgctgcttggctctggaaaacagtcctcctggcatctacattcacagccttgctccaggatcagtggccaagatggagagcaacctgagccgcggggatcaaatcctggaagtgaactccgtcaacgtccgccatgctgctttaagcaaagtccacgccatcttgagtaaatgccctccaggacccgttcgccttgtcatcggccggcaccctaatccaaaggtttccgagcaggaaatggatgaagtcatagcacgcagcacttatcaggagagcaaagaggccaattcctctcctggcttaggtacccccttgaagagtccctctcttgcaaaaaaggactcccttatttctgaatctgaactctcccagtactttgcccacgatgtccctggccccttgtcagacttcatggtggccggttctgaggacgaggatcacccgggaagtggctgcagcacgtcggaggagggcagcctgcctcccagcacctccactcacaaggagcctggaaaacccagagccaacagcctcgtgactcttgggagccatcgggcttctgggctcttccacaagcaggtgacagttgccagacaagccagtctccccggaagcccacaggccctccgaaaccctctcctccgccagaggaaggtaggctgctacgatgccaacgatgccagtgatgaggaagagtttgacagagaaggggactgcatttcactcccaggggccctcccgggtcccatcaggcctctgtcagaggatgacccgaggcgtgtctcaatttcctcttccaagggcatggacgtccacaaccaagaggaacgaccccggaaaacactggtgagcaaggccatctcggcacctcttcttggtagctcagtggacttagaggagagtatcccagagggcatggtggatgctgcgtcctatgcagccaacctcacggactctgcagaggcccccaaggggagccctggaagctggtggaagaaggaactgtcaggatcaagtagcgcacccaaattggaatacacagtccgtacagacacccagagtccgacgaacactgggagccccagttccccccagcagaaaagtgaaggcctgggctccaggcacagaccagtggccagggtaagcccccactgcaagagatccgaggctgaggccaagcccagtggctcacagacagtgaacctgactggcagagccaatgatccatgcgatctggactcgagagtccaggccacttctgtcaaagtgactgtcgctggctttcagccaggtggagctgtggagaaggaatctctgggaaagctgaccactggagatgcttgtgtctctaccagctgtgaactagccagtgctctgtcccatctggatgccagccacctcacagagaacctgcccaaagctgcatcagagctggggcaacaacccatgactgaactggacagctcctcagacctcatctcttccccagggaagaagggggccgctcatcctgaccccagcaagacctctgtagacacagggcaagtcagtcggccagagaatcccagccagcctgcatcgcccagggtcaccaagtgcaaggccaggtctccagtcaggctcccccatgagggcagcccctccccgggggagaaagcagcggctccccctgactacagcaagactcgatcagcatcggaaaccagcacaccccacaataccaggagggtggctgccctcaggggagcgggacctggagcagagggaatgacaccagctggtgctgtcctgccaggagaccccctcacatcccaggagcagagacagggagctccaggtaaccacagtaaggctctggaaatgacaggaatccatgcacctgaaagctcccaggagccttccctgctggagggagcagattctgtgtcctcaagggcaccgcaggccagcctctccatgctgccatccactgacaacaccaaagaagcatgtggccatgtctcggggcactgctgcccaggggggagtagagagagccctgtgacggacattgacagcttcatcaaggagctggatgcttctgcagcaaggtctccgtcttcccagacgggggacagtggctctcaggagggcagtgctcagggccacccaccagccggggctggaggtgggagctcctgccgtgccgaaccagtcccggggggccagacctcctccccgaggagggcctgggctgctggtgcccccgcctacccacaatgggcctcccagccttcggttttagattcaattaatcccgacaaacattttactgtgaacaaaaactttctgagcaactactctagaaattttagcagttttcatgaagacagcacctccctatcaggcctgggtgacagcacggagccgtctctgtcatccatgtatggcgatgctgaggattcttcttctgaccctgagtcactcactgaagccccacgagcttctgccagggacggctggtcccctcctcgttcccgtgtgtctttgcacaaggaagatccttcggagtcagaagaggaacagattgagatttgttccacacgtggctgccccaatccaccctcgagtcctgctcatcttcccacccaggctgccatctgtcctgcctcagccaaagttctgtcattaaaatacagcactccgagagagtcggtggccagtccccgtgagaaggccgcctgcttgccaggctcatacacttcaggcccagactcttcccagccatcatcactcttggagatgagctctcaggagcatgaaactcatgcggacataagcacttcacagaaccacaggccctcgtgtgcagaagaaaccacagaagtcaccagcgctagctcagccatggaaaacagtccgctgtctaaagtagccaggcattttcacagtccgcccatcattctcagctcccccaacatggtaaatggcttggaacatgacctgctagatgacgaaaccctgaatcaatacgaaacaagcattaatgcagctgccagtctgtcctccttcagtgtggatgtccctaagaatggagaatctgttttggaaaacctccacatctctgaaagtcaagacctggatgacttgctacagaaaccaaaaatgatcgctaggaggcccatcatggcctggtttaaagaaataaataaacataaccaaggcacacatttgaggagcaaaaccgagaaggaacaacctctaatgcctgccagaagtcccgactccaagattcagatggtgagttcaagccaaaaaaagggcgttactgtgcctcatagccctcctcagccgaaaacaaacctggaaaataaggacctgtctaagaagagtccggcagaaatgcttctgactaatggtcagaaggcaaagtgtggtccgaagctgaagaggctcagcctcaagggcaaggccaaagtcaactctgaggcccctgctgcgaatgctgtgaaggctggggggacggaccacaggaaacccttgatctcaccccagacctcccacaaaacactttctaaggcagtgtcacagcggctccatgtagccgaccacgaggaccctgacagaaacaccacagctgcccccaggtccccccagtgtgtgctggaaagcaagccacctcttgccacctctgggccactgaaaccctcagtgtctgacacgagcatcaggacatttgtctcgcccctgacctctcccaagcctgttcctgagcaaggcatgtggagcaggttccacatggctgtcctctctgaacccgacagaggttgcccaaccacccctaaatctcctaagtgtagagcagagggcagggcgccccgtgctgactccgggccggtgagtccggcagcgtctaggaacggcatgtccgtggcagggaacagacagagtgagccgcgcctggccagccatgtggcagcagacacagcccaacccaggccgactggcgaaaaaggaggcaacataatggccagcgatcgcctcgaaagaacaaaccagctgaaaatcgtggagatttctgctgaagcagtgtcagagactgtatgtggtaacaagccagctgaaagcgacagacggggagggtgcttggcccagggcaactgtcaggagaagagtgaaatcaggctctatcgccaggtcgcagaatcatccacaagtcatccatcctcactcccatctcatgcctcccaggcagagcaggaaatgtcacgatcattcagcatggcaaaactggcgtcctcctcctcctcccttcaaacagccattagaaaggcagaatactcccagggaaaatcaagcctgatgtcagactcccgaggggtgcccagaaacagcattccagggggcccctcgggggaggaccatctctacttcaccccaaggccagcgaccaggacctactccatgccagcccagttctcaagccattttggacgggagggtcaccccccacacagcctgggtcgctctcgggacagccaggtccctgtgacaagcagtgttgtccccgaggcaaaggcatccagaggtggtcttcccagcctggctaatggacagggcatatatagtgtaaagccgctgctggacacatcgaggaatcttccagccacagatgaaggggatatcatttcagtccaggagacgagctgcctagtcacagacaaaatcaaagtcaccagacgacactactgctatgagcagaactggccccatgaatctacctcatttttctctgtgaagcagcggatcaagtcttttgagaacctggccaatgctgaccggcctgtagccaagtccggggcttccccatttttgtcggtgagctccaagcctcccattgggaggcggtcttccggcagcattgtttccgggagcctgggccacccaggtgacgcagcagcaaggttgttgagacgcagcttgagttcctgcagcgaaaaccaaagcgaagccggcaccctcctgccccagatggccaagtctccctcaatcatgacactgaccatctctcggcagaacccaccagagaccagtagcaagggctctgattcggaactaaagaaatcacttggtcctttgggaattcccaccccaacgatgaccctggcttctcctgttaagaggaacaagtcctcggtacgccacacgcagccctcgcccgtgtcccgctccaagctccaggagctgagagccttgagcatgcctgaccttgacaagctctgcagcgaggattactcagcagggccgagcgccgtgctcttcaaaactgagctggagatcacccccaggaggtcacctggccctcctgctggaggcgtttcgtgtcccgagaagggcgggaacagggcctgtccaggaggaagtggccctaaaaccagtgctgctgagacacccagttcagccagtgatacgggtgaagctgcccaggatctgccttttagaagaagctggtcagttaatttggatcaacttctagtctcagcgggggaccagcaaagattacagtctgttttatcgtcagtgggatcgaaatctaccatcctaactctcattcaggaagcgaaagcacaatcagagaatgaagaagatgtttgcttcatagtcttgaatagaaaagaaggctcaggtctgggattcagtgtggcaggagggacagatgtggagccaaaatcaatcacggtccacagggtgttttctcagggggcggcttctcaggaagggactatgaaccgaggggatttccttctgtcagtcaacggcgcctcactggctggcttagcccacgggaatgtcctgaaggttctgcaccaggcacagctgcacaaagatgccctcgtggtcatcaagaaagggatggatcagcccaggccctctgcccggcaggagcctcccacagccaatgggaagggtttgctgtccagaaagaccatccccctggagcctggcattgggagaagtgtggctgtacacgatgctctgtgtgttgaagtgctgaagacctcggctgggctgggactgagtctggatgggggaaaatcatcggtgacgggagatgggcccttggtcattaaaagagtgtacaaaggtggtgcggctgaacaagctggaataatagaagctggagatgaaattcttgctattaatgggaaacctctggttgggctcatgcactttgatgcctggaatattatgaagtctgtcccagaaggacctgtgcagttattaattagaaagcataggaattcttcatgaattttaacaagaatcattttctcagttctcttctttctttagcaaatcagagtgacttctttaaaccacaggttgttgaaatggccaacactggtacagacacggactataaaaatctccaagcttgtgcttacacatgaagcctgacttaactgtatgtgcaacagcaatgaaattaactccagaagccttccacctgcgtcacccaggccgggagggttccttcgttccagtgcctgtcccctacctttatgttatgtttactgatggggatacaagatgtgacacacccttctttatttgaaacaaacaaacatttagctagacctttgcttccttcttgccagctctcccaacatacccaatcctggtgatcagggaactaaaagtctgagggggacacaaatgtcacacctaagaggacaatcaatcattttgtatgattttgtaagtaaatgacagaatgcttttaggcacattcaatggaaggaggagatgtaggtctgtatatgttaccctgaaaagagaataagacttacttaaaaaaatgaattatgacctgttaggctgagctcaggaattgtccaaaaaggaaaaagcaaaataattaattgagagtattttttagtgagtgtaatgtataatgtacgtatgcaaagttcaactcaataggttattgatcaccatgaagtattgatcattttctatctcaaaagtgtaagccataaggctgttttacagaatagcacttctgataagctgtattaaatagccatgagcttcactgcttagagggagcagaaaggtcaacatctaaaagcaccttacaactagtttttgaacctgtcttgataagtgcttgaattcaagactggtcagtccaagagcagacaaaaatatcacaagtcagtcagtcactgggtttccatttctgaattttatgcactccaaccatgaatttaaactaaatttttagaaatcaagtatctttctaagtgtccttggatttatagacaatgtatgtacaatccaaatagaggagcttaatggaatccttttaggagactggttggtttttttccctctttcccaacatgtttaagaaatgtaacattctaagtattggatctcttttcttgacctagtataatgacaactgcagtgacttaagtttttgctgttttcgttttcccgctttgcaatttcctccttttgccaaaaatgttttcctacagaagactgtcgtgactcacgctacttgggaaactcactctggccactcctcctctggtggcatgagctgcttcccagtagctattccgattggatattccgttcgtcgtcacatagctggcttttctctcctcatgatgtaccttattttcttaggtaaataattccaaactctcatcgggtcataaagaggaggagaaacagggtgagtcaaggtaaaggagcagaaatgtagttacaagccaggtcgtcttcagtggcacaaaccaacccgttgagccctgacaacatgagtggagagtgcatttgccatacctgtgtgcatgacactaagattttatgttggagatacttctttaaataacctacagcttgggtctatggctgtgacccccagattcatggaggggctttagccatcagctttgtacatcatcatttttctgaatgaccaatcccactaaacatctttgaagtcggcctagagaggtccttcagatgagagagaaatagctggcttgtctgagtccagatttctcatcaactggcaatacaaaggaaaatatggtacaggagttagttagaaaggtcttattgattttacttctacttttcactacagttacaggtagaatactgtaggaagtcagtgcaaggtgcatgcttgattgatagatattgattgattgtttttcagtctctggggtcagttttgtggtttctgctttcttgcctaaatcaaagactatttcaagtcaacaacactgaaaactgcttttcgcctccactcttacagctgtgcctaataataattaattaataaacgcacagccctatgtgaacagacaggaatttcttgtgcaatgtggagcaaatggaatggtctccttccgcaagtctttttaatcctcatatctggagtacaagggtagacctctggcttaccacatacactatgctaaagtcatcagccactgctactacatcttgccagaaggtttccctcgccaacaaacagttgaaatttaagggaagaagcaaaagctaaactgtctttgaccctaagatagatagaaagctatttatttgtcttcagtgttcaaggcatgactagtatttctaattagcctaataaattcccacactttctgaagtgaacactaatggtattgtcctactaaaactgtcattgtttctttttttttaactggtcagtcattcacaataagctatgagggtaaataaatatgtgttataacaagtaaaccgtagttgcaagaatataccatgaagattaaagtaggctgggtttcatttccatcttcccacacatctcattgaatttgatggttgacttaattggcaccataactttgtatgatattatacattaacctttatttatgtaaagtaaaatgccttatatattaaagagtaagtgcaataatatgaaatagcctgtacattttaaaaatgttgtcaccaagttatataaatccacatctctgtaaacaaccttttttaagtaattttaaaaaaaataaacactctgcttactacttgaaaaaaaaaaaaaaaaaa

SEQ ID NO: 37 - DNA, 4510 bp, Homo sapiens
  misc_feature: TDRD1
gctgaggccaggagggcgcactggggattggaggcgagggaagtgcagggcgcatcccaggcggcagggctcccagcatcggcagtcgccatcaccgccagaccgcagagacaggttcggatccgcggtcctcttgcctctttccaggcctcgatgagtgttaaatcgccatttaatgtgatgtcaagaaataatttggaagcacctccttgtaagatgacagagccatttaattttgagaaaaatgaaaacaagcttccaccacatgagtctttaagaagtcctggaacacttcctaaccaccctaatttcaggctgaaaagctcagagaatggaaataaaaagaacaattttttgctttgtgagcaaaccaaacaatatttggctagtcaggaagacaattcagtttcttcaaacccgaatggcatcaacggagaagtagttggctccaaaggagacaggaaaaaattgccagcaggaaactcagtgtcaccaccaagtgctgaaagtaattcaccacccaaagaagtgaatattaagcctggaaataatgtacgtcctgcaaaatcaaaaaaactaaacaagttggtcgagaattccttgtccataagtaatccagggctcttcacctccttaggacctcctcttcggtccacaacttgccatcgctgtggcctatttggatcgctgaggtgctctcagtgcaagcagacctactattgctccacagcatgtcaaagaagagactggtctgcacacagcatcgtgtgcaggcctgttcagccaaatttccacaaacttgaaaataaatcatctattgaaacaaaggatgtggaggtaaacaataagagtgactgtccacttggagttactaaggaaatagccatttgggctgagagaataatgttttctgatttgagaagtctacaactcaagaaaaccatggaaataaagggtacggttaccgaattcaaacacccaggggacttctacgtgcagttatattcttcagaagttttagaatacatgaaccaactctctgccagcttaaaagaaacatatgcaaatgtgcatgaaaaagactatattcctgttaagggggaagtttgtattgccaagtacactgttgatcagacctggaacagagcaatcatacaaaacgttgatgtgcagcaaaagaaggcacatgtcttatatattgattatggaaatgaagaaataattccattaaacagaatttaccacctcaacaggaacattgacttgtttcctccttgtgccataaagtgctttgtagccaatgttatcccagcagaagggaattggagcagtgattgtatcaaagctactaaaccactgttaatggagcagtactgctccataaagattgtcgacatcttggaagaggaagtggttacctttgctgtagaagttgagctgccaaattcaggaaaacttttagaccatgtgcttatagaaatgggatatggcttgaaacccagtggacaagattctaagaaggaaaatgcagatcaaagtgatcctgaagatgttggaaaaatgacaactgaaaacaacattgtcgtagacaaaagtgacctaatcccaaaagtgttaactttgaatgtaggtgatgagttttgtggtgtggttgcccacattcaaacaccagaagacttcttttgtcaacaactgcaaagtggccgaaagcttgctgaacttcaggcatcccttagcaagtactgtgatcagttgcctccacgctctgatttttatccagccattggtgatatatgttgtgctcagttctcagaggatgatcagtggtaccgtgcctctgttttggcttacgcttctgaagaatctgtactggtcggatatgtagattatggaaactttgaaatccttagtttgatgagactttgtcccataatcccaaagttgttggaattgccaatgcaagctataaagtgtgtactagcaggagtaaagccatcattaggaatttggactccagaagctatttgtctcatgaaaaaacttgtacagaacaaaataatcacagtgaaagtggtggacaagttggaaaacagttccctggtggagcttattgataaatccgagacgcctcatgtcagtgttagcaaagttctcctagatgcaggctttgctgtgggagaacagagtatggtgacagataaacccagtgacgtgaaagaaaccagtgttcccttgggtgtggaaggaaaagtaaatccattggagtggacatgggttgaacttggtgttgaccaaacagtagatgttgtggtctgtgtgatatatagtcctggagaattttattgccatgtgcttaaagaggatgctttaaagaaactcaatgatttgaacaagtcattagcagaacactgccagcagaagttacctaatggtttcaaggcagagataggacaaccttgttgtgctttttttgcaggtgatggtagttggtatcgtgctttagtcaaggaaatcttaccaaatggacatgttaaagtacattttgtggattatggaaacatcgaagaagttactgcagatgaactccgaatgatatcatcaacatttttaaaccttccctttcagggaatacggtgccagttagcagatatacagtctagaaacaaacattggtctgaagaagccataacaagattccagatgtgtgttgctgggataaaattgcaagccagagtggttgaagtcactgaaaatgggataggagttgaactcaccgatctctccacttgttatcccagaataattagtgatgttctgattgatgaacatctggttttaaaatctgcttcaccacataaagacttaccaaatgacagacttgttaataaacatgagcttcaagttcatgtacagggacttcaagctacctcttcagctgagcaatggaagacgatagaattgccagtggataaaactatacaagcaaatgtattagaaatcataagcccaaacttgttttatgctctaccaaaagggatgccagaaaatcaggaaaagctgtgcatgttgacagctgaattattagaatactgcaatgctccgaaaagtcgaccaccctatagaccaagaattggagacgcatgctgtgccaaatacacaagtgatgatttttggtatcgtgcagttgttctggggacatcagacactgatgtggaagtgctctatgcagactatggaaacattgaaaccctgcctctttgcagagtgcaaccaatcacctctagccacctggcgcttcctttccaaattattagatgttcacttgaaggattaatggaattgaatggaagctcttctcaattaataataatgctattaaaaaatttcatgttgaatcagaatgtaatgctttctgtgaaaggaattacaaagaatgtccatacagtgtcagttgagaaatgttctgagaatgggactgtcgatgtagctgataagctagtgacatttggtctggcaaaaaacatcacacctcaaaggcagagtgctttaaatacagaaaagatgtataggatgaattgctgctgcacagagttacagaaacaagttgaaaaacatgaacatattcttctcttcctcttaaacaattcaaccaatcaaaataaatttattgaaatgaaaaaactgttaaaaaaaacagcatctcttggaggtaaacccttatgagacaggaaacagcaaaggctagctttaggagagaaagtacagcacctggtgtttttatttatgagaaccttttctttgtccactttctctgtaatgaccttctatccctccgtttttgcctgcctgccattctcctattaggttggtggtttttattttcctctaagttccttccaccaaataaatattacgtaaaaaattcataccaaatcaatgagaatactggcaaggaatacatagggactttctgctatatatgtaactttttattacttaaaggtaccgaaggaaggccaggtgcagtggctcacgcccagcactttgggaggctgaggtgggaggatcccttgaggccaggagttcaaggttacagtgagctatgatagtgccactgcactccagcctgggtgacagattttgtcttaaaaaaaaaaaaaaaaaagttgatatgagttttattttctgtccgtttgaaatattttgtaatattccctgcattctctgtcgtctgcctcttccacataatgtcctttgctttcatgtttgttatcttctttttctgttcactcagaggtcatcaatttctttctctccgtccttaattggattatttttcttttggcctttgggcacagagtctgacctctggaccactctaactggagaaggaactttatgttccctctcctgctgtgtccacaaccttagaaatctgtagctagatttttgttgttatagatagaatttactgtttctgaaacccaaatacagttatcagtttaaggtt

(19) United States
(12) Patent Application Publication
(10) Pub. No.: US 2015/0218646 A1
(43) Pub. Date: Aug. 6, 2015

(54) METHODS, KITS AND COMPOSITIONS FOR PROVIDING A CLINICAL ASSESSMENT OF PROSTATE CANCER

(71) Applicant: DIAGNOCURE INC., Québec (CA)

(72) Inventors: Jean-François Haince, Quebec (CA); Guillaume Beaudry, Quebec (CA); Yves Fradet, Quebec (CA); Éric Paquet, Quebec (CA)

(73) Assignee: DIAGNOCURE INC, Quebec QC, (CA)

(21) Appl. No.: 14/416,036

(22) PCT Filed: Jun. 14, 2013
(86) PCT No.: PCT/CA2013/050452

§ 371 Date: Jan. 20, 2015

Related U.S. Application Data
(60) Provisional application No. 61/674,079, filed on Jul. 20, 2012.

(51) Int. Cl. C12Q 1/68; G06F 19/00

(52) U.S. Cl. CPC C12Q 1/6886; G06F 19/3431; C12Q 2600/16; C12Q 2600/158; C12Q 2600/166; C12Q 2600/118

(57) ABSTRACT

The present invention relates to prostate cancer signatures which are useful for providing a clinical assessment of prostate cancer from a biological sample of a subject. By performing initial gene expression studies on urine samples from prostate cancer and non-prostate cancer subjects, and using the PCA3/PSA prostate cancer test as a performance benchmark, the present inventors have surprisingly discovered multiple signatures that are informative in urine-based prostate cancer tests, as well as in tissue-based tests. The signatures relate to combinations of at least two prostate cancer markers whose expression pattern in urine has been validated as being associated (either positively or negatively) with a clinical assessment of prostate cancer. The prostate cancer markers can be used in conjunction with bioinformatics approaches to generate a prostate cancer score, which correlates with a clinical assessment of prostate cancer. Methods, kits and compositions relating to the aforementioned signatures are also described.

### FIELD OF THE INVENTION

The present invention relates to prostate cancer. More specifically, the present invention relates to methods, kits and compositions for providing a clinical assessment of prostate cancer in a subject based on a biological sample therefrom. In particular, the present invention relates to prostate cancer signatures comprising at least two prostate cancer markers for providing a clinical assessment of prostate cancer.

### BACKGROUND OF THE INVENTION

Prostate cancer is the most common form of cancer affecting men. In the United States, more than 241,000 men are diagnosed with prostate cancer each year, and nearly 28,000 die from this disease annually. While the lifetime risk of developing prostate cancer is estimated at 16% (and the risk of dying from this disease is estimated at 2.9%), autopsies reveal that prostate cancer is actually present in about two thirds of men over 80 years old. These results highlight a striking problem in the field of prostate cancer diagnosis, where many cases go undetected and do not become clinically evident. Thus, an improved screening program that can identify, in particular, asymptomatic men with aggressive localized tumors would be useful in reducing prostate cancer morbidity and mortality.

Prostate cancer survival is related to many factors, especially tumor extent at the time of diagnosis. Due to current limitations in methods for prostate cancer diagnosis, prostate tumors which are progressive in nature are likely to have metastasized by the time of detection, and survival rates for individuals with metastatic prostate cancer are quite low. For patients with prostate tumors that will metastasize but have not yet done so, surgical prostate removal is often curative. Determining tumor extent is thus important for selecting optimal treatment and improving patient survival rates.

Currently, a diagnosis of prostate cancer is generally made as a result of an elevated prostate specific antigen (PSA) blood test or, less frequently, based upon an abnormal digital rectal examination (DRE). PSA is a glycoprotein produced by prostate epithelial cells and the PSA test measures the amount of PSA in a sample of blood. Most men with prostate cancer have an elevated PSA concentration (e.g., greater than 4 ng/mL), although an elevated PSA level does not necessarily indicate the presence of prostate cancer, and there is no PSA level at which the risk of having prostate cancer is zero. In fact, the most common cause for an elevated PSA is benign prostatic hyperplasia (BPH), a non-cancerous enlargement of the prostate.

There are a number of factors that can transiently elevate or reduce PSA levels independent of prostate cancer, some of which are significant enough to affect the diagnostic performance of the PSA blood test. For example, bacterial prostatitis can elevate PSA levels until infection symptoms resolve after six to eight weeks. Ejaculation can increase PSA levels (e.g., by up to 0.8 ng/mL) before they return to normal within 48 hours. Asymptomatic prostate inflammation, which is generally diagnosed via prostate biopsy, can also elevate PSA levels. Furthermore, PSA levels tend to increase with age and it has been suggested that the PSA blood test may be improved by setting higher normal PSA levels for older men. On the other hand, drugs such as five-alpha reductase inhibitors (e.g., finasteride, dutasteride) have been shown to lower PSA levels.

In view of the above, only about 30% of men with an elevated PSA actually have prostate cancer. The majority of these newly-diagnosed cancers are clinically localized, which leads to an increase in radical prostatectomy and radiation therapy, which are aggressive treatments intended to cure these early-stage cancers. While the utility of early prostate cancer diagnosis/screening was demonstrated in a multi-center study where PSA-based screening significantly reduced prostate cancer specific mortality (Schroder et al., Prostate-cancer mortality at 11 years of follow-up, N Engl J Med 2012; 366:981-90), this reduction was not without consequence since the very high false positive rate of PSA drove the number of unnecessary prostate biopsies as high as 75%. These unnecessary biopsies create morbidity, especially in terms of infection following the intervention, creating hospital readmission rates as high as 4% in the month following the biopsy (Nam et al., Increasing hospital admission rates for urological complications after transrectal ultrasound guided prostate biopsy, J Urol 2010; 183: 963-8). This situation creates another dilemma: the group of patients with an elevated PSA but with a negative prostate biopsy result increases every year. Since prostate biopsy is not 100% accurate at detecting prostate cancer—as much as 25% of prostate cancer could be missed by a first biopsy—this situation creates much anxiety to patients and, until recently, there was no clinical solution to this dilemma except to perform follow-up biopsies.

The inadequacies of the PSA blood test were further brought to light on May 22, 2012, when the U.S. Preventive Services Task Force issued a final recommendation against PSA-screening for prostate cancer. Based on its review of research studies, the Task Force concluded that the expected harms of PSA screening are greater than the potential benefit. The recommendation is based on the following facts. On one hand, the reduction in prostate cancer deaths from PSA screening is very small as one man in 1,000, at most, avoids death from prostate cancer because of screening. On the other hand, the Task Force considers that most prostate cancers found by PSA screening are slow growing, not life threatening, and will not cause a man any harm during his lifetime and that there is currently no way to determine which cancers are likely to threaten a man's health and which will not. As a result, almost all men with PSA-detected prostate cancer will opt to receive treatment, which in some cases may be unnecessary or not recommended.

Determining an accurate diagnosis and prognosis of prostate cancer is critical in selecting the most appropriate treatment. All of the potentially curative therapies carry inherent risks of serious complications; and these risks can be justified only if the treatment has a reasonable chance of achieving significantly improved clinical outcomes including, for example, long-term survival and improved quality of life. Numerous forms of therapy are available to treat prostate cancer, including but not limited to: surgery such as prostatectomy; tumor destruction therapy such as cryotherapy; radiation therapy such as brachytherapy; and drug and other agent therapies such as hormone therapy and chemotherapy. Clinical assessments that have improved accuracy, or are otherwise enhanced as compared to currently available diagnostic and prognostic methods, will provide better selection for therapy and yield improved clinical outcomes for the prostate cancer patient.

Prostate cancer antigen 3 (PCA3) is a non-coding RNA whose spliced isoform is specific to prostate tissue and is highly over expressed in prostate cancer, but is not over expressed in hyperplastic (BPH) or normal prostate tissue. Although PCA3 is widely considered as a superior prostate cancer marker to PSA, it has thus far only been approved by the US FDA as a tool to help physicians determine the need for a repeat biopsy in men who have had a previous negative biopsy (Summary of Safety and Effectiveness Data (SSED) issued by the US FDA for PROGENSA® PCA3 Assay; http://www.accessdata.fda.gov/cdrh_docs/pdf10/P100033b.pdf). Thus, an improved prostate cancer marker to PCA3 is desirable.

Over the years, many single molecular markers have been evaluated with the goal of identifying one that can surpass the performance of PCA3 for prostate cancer diagnosis. Some of these markers detect a loss of gene expression through hypermethylation detection (e.g., GSTP1), genetic translocation through expression of gene fusion (e.g., TMPRSS2 and ETS transcription factors like ERG, ETV1 or ETV4) or other overexpressed genes in prostate cancer (e.g., GOLPH2 or SPINK1). Unfortunately, the vast majority of these markers identified by tissue analysis were not subsequently validated as efficient or accurate prostate cancer markers. In fact, these markers usually are shown not to be usable as targets in non-invasive biological samples. For instance, Laxman et al. (Cancer Res., 2008, 68: 645-649) demonstrated that AMACR and TFF3 mRNAs, which had previously been shown to be specific biomarkers for prostate cancer in tissues, were not statistically significant predictors of prostate cancer in urine samples (P=0.450 and 0.189, respectively). In any event, none of these molecular markers have yet been validated to a point where they outperform PCA3, which to this day, is the only prostate cancer marker that can be reliably measured in a urine-based test. Thus, with the exception of the PCA3 assay, there is no reliable method for providing a clinical assessment of prostate cancer using non-invasive clinical samples such as urine. In addition, the vast majority of previous studies, seeking to identify prostate cancer markers focused on gene expression profiling in tissue samples first, as opposed to gene expression profiling in urine. Another issue has been the lack of robust control markers that can be used to normalize and/or validate prostate cancer marker detection.

Accordingly, there remains an urgent need for improved prostate cancer markers that can provide a superior clinical assessment of prostate cancer in men, including, without being limited to, improved diagnosis, prognosis, and/or tumor grading/staging. There also remains a need for the identification of one or more control markers to be used in conjunction with the new prostate cancer markers for clinical assessment of prostate cancer in a patient's sample. The present invention seeks to address at least some of the deficiencies of the prostate cancer markers of the prior art.

The present description refers to a number of documents, the content of which is herein incorporated by reference in their entirety.

### SUMMARY OF THE INVENTION

The present invention relates to prostate cancer signatures comprising combinations of at least two prostate cancer markers whose expression pattern in urine has been validated herein to be associated (either positively or negatively) with a clinical assessment of prostate cancer. Traditionally, prostate cancer markers have been identified by performing differential expression analysis on cancerous and non-cancerous prostate tissue samples. However, few prostate cancer markers identified in this way have been successfully translated into urine-based prostate cancer tests, possibly due to a number of confounding factors associated with the use of urine (e.g., acidic environment and/or contaminating background urinary tract cells). By performing initial gene expression studies on urine samples from prostate cancer and non-prostate cancer subjects, and using the PCA3/PSA prostate cancer test as a performance benchmark, the present inventors have surprisingly discovered multiple prostate cancer signatures that are robustly informative in urine-based prostate cancer tests, as well as in tissue-based tests. More particularly, the prostate cancer markers of the present invention can be used in conjunction with bioinformatics approaches (e.g., machine-learning) to generate a score, which correlates with a clinical assessment of prostate cancer.

Accordingly, the present invention generally relates to methods, kits and compositions for providing a clinical assessment of prostate cancer in a subject based on a biological sample therefrom. More particularly, a clinical assessment of prostate cancer can include diagnosis, grading, staging and prognosis, based on a biological sample from a subject.

In one aspect of the present invention, a biological sample is obtained from a subject (e.g., urine, tissue or blood sample), and normalized expression levels of at least two prostate cancer markers in a prostate cancer signature of the present invention are determined. A mathematical correlation of the normalized expression levels of the at least two prostate cancer markers is then performed to obtain a score, which is used to provide a clinical assessment of prostate cancer in the subject.

In one embodiment, the prostate cancer signatures of the present invention are able to outperform PCA3 (or PCA3/PSA ratio) for providing a clinical assessment of prostate cancer. This represents a significant advancement in the field of prostate cancer, since PCA3 is widely regarded as the best prostate cancer marker to date. Thus, a prostate cancer signature capable of outperforming PCA3 (particularly in the context of a non-invasive sample such as urine) is highly desirable. In some cases, it may be useful to employ a prostate cancer diagnostic tool that does not rely on PCA3 per se. For example, if a clinical assessment of prostate cancer is made on a subject using a PCA3-based test, it may be desirable to have a separate, independent clinical assessment of prostate cancer performed which does not rely on PCA3. In this way, the prostate cancer signatures of the present invention may be used to independently validate a PCA3-based test result, or vice versa. Accordingly, in a particular embodiment, the prostate cancer signatures of the present invention do not include PCA3.

In another aspect, the present invention relates to a method for providing a clinical assessment of prostate cancer in a subject, said method comprising:
(a) determining the expression of at least two prostate cancer markers listed in Table 5 or 6A, or a marker co-regulated therewith in prostate cancer, in a biological sample from said subject;
(b) normalizing the expression of said at least two prostate cancer markers using one or more control markers;
(c) performing a mathematical correlation of the normalized expression levels of said at least two prostate cancer markers;
(d) deriving a score from said mathematical correlation; and
(e) providing said clinical assessment of prostate cancer based on said derived score.

In another aspect, the present invention relates to a method for providing a clinical assessment of prostate cancer in a subject, said method comprising:
(a) selecting at least two prostate cancer markers validated as such, based on their expression profile in urines of a population of patients known to have or lack prostate cancer;
(b) determining the expression of said at least two prostate cancer markers in a biological sample from said subject;
(c) normalizing the expression of said at least two prostate cancer markers using one or more control markers;
(d) performing a mathematical correlation of the normalized expression of said at least two prostate cancer markers;
(e) deriving a score from said mathematical correlation; and
(f) providing said clinical assessment of prostate cancer based on said derived score.

In another aspect, the present invention relates to a prostate cancer diagnostic composition comprising:
(a) urine, or a fraction thereof having markers of prostate origin, from a subject having or suspected of having prostate cancer; and
(b) reagents enabling the detection and/or amplification of at least two prostate cancer markers from Table 5 or 6A, or a marker co-regulated therewith.

In another aspect, the present invention relates to a kit for providing a clinical assessment of prostate cancer in a subject from a biological sample therefrom, said kit comprising:
(a) reagents enabling the detection and/or amplification of at least two prostate cancer markers from Table 5 or 6A, or a marker co-regulated therewith; and
(b) a suitable container.

In particular embodiments, the above mentioned at least two prostate cancer markers is at least three prostate cancer markers; at least four prostate cancer markers; at least five prostate cancer markers; at least six prostate cancer markers; at least seven prostate cancer markers; at least eight prostate cancer markers; or at least nine prostate cancer markers.

In another embodiment, the above mentioned at least two prostate cancer markers are selected from:

(1) CACNA1D or a marker co-regulated therewith in prostate cancer;

(2) ERG or a marker co-regulated therewith in prostate cancer;

(3) HOXC4 or a marker co-regulated therewith in prostate cancer;

(4) ERG-SNAI2 prostate cancer marker pair;

(5) ERG-RPL22L1 prostate cancer marker pair;

(6) KRT 15 or a marker co-regulated therewith in prostate cancer;

(7) LAMB3 or a marker co-regulated therewith in prostate cancer;

(8) HOXC6 or a marker co-regulated therewith in prostate cancer;

(9) TAGLN or a marker co-regulated therewith in prostate cancer;

(10) TDRD1 or a marker co-regulated therewith in prostate cancer;

(11) SDK1 or a marker co-regulated therewith in prostate cancer;

(12) EFNA5 or a marker co-regulated therewith in prostate cancer;

(13) SRD5A2 or a marker co-regulated therewith in prostate cancer;

(14) maxERG CACNA1D prostate cancer marker pair;

(15) TRIM29 or a marker co-regulated therewith in prostate cancer;

(16) OR51E1 or a marker co-regulated therewith in prostate cancer; and

(17) HOXC6 or a marker co-regulated therewith in prostate cancer.

In another embodiment, the above mentioned at least two prostate cancer markers comprise CACNA1 D or a prostate cancer marker co-regulated therewith in prostate cancer. In another embodiment, the above mentioned at least two prostate cancer markers comprise CACNA1D, or a prostate cancer marker co-regulated therewith in prostate cancer, and ERG, or a prostate cancer marker co-regulated therewith in prostate cancer. In another embodiment, the above mentioned at least two prostate cancer markers are combined in classifiers as defined in Tables 7-9.

In another embodiment, one or more of the above mentioned marker co-regulated therewith in prostate cancer is as defined in Table 6B.

In another embodiment, the above mentioned one or more control markers comprise endogenous reference genes. In another embodiment, the above mentioned one or more control markers further comprise at least one prostate-specific control marker. In another embodiment, the above mentioned one or more control markers are as defined in Table 2, Table 7A and/or Table 7B. In another embodiment, the above mentioned prostate-specific control marker comprises one or more of KLK3, FOLH1, FOLH1B, PCGEM1, PMEPA1, OR51E1, OR51E2, and PSCA. In another embodiment, the above mentioned control markers comprise KLK3, IPO8, and POLR2A. In another embodiment, the above mentioned one or more control markers comprise IPO8, POLR2A, GUSB, TBP, and KLK3. In another embodiment, the above mentioned control markers comprise at least one of the above prostate-specific control markers plus IPO8 and POLR2A. In another embodiment, the above mentioned control markers comprise at least one of the above prostate-specific control markers, as well as IPO8, POLR2A, GUSB, and TBP.

In another embodiment, the above mentioned clinical assessment of prostate cancer comprises: (i) a diagnosis of prostate cancer; (ii) a prognosis of prostate cancer; (iii) a staging assessment of prostate cancer; (iv) a prostate cancer aggressiveness classification; (v) an assessment of therapy effectiveness; (vi) as assessment of the need for a prostate biopsy; or (vii) any combination of (i) to (vi).

In another embodiment, the above mentioned marker is a gene. In another embodiment, the above mentioned marker is a protein.

In another embodiment, the above mentioned determining the expression of said at least two prostate cancer markers comprises determining RNA expression and/or protein expression. In another embodiment, the above mentioned determining RNA expression comprises performing a hybridization and/or amplification reaction. In another embodiment, the above mentioned hybridization and/or amplification reaction comprises: (a) polymerase chain reaction (PCR); (b) nucleic acid sequence-based amplification assay (NASBA); (c) transcription mediated amplification (TMA); (d) ligase chain reaction (LCR); or (e) strand displacement amplification (SDA).

In another embodiment, the above mentioned determining RNA expression comprises a direct sequencing of at least two prostate cancer markers.

In another embodiment, the above mentioned biological sample is urine, prostate tissue resection, prostate tissue biopsy, ejaculate or bladder washing. In another embodiment, the above mentioned biological sample is whole or crude urine. In another embodiment, the above mentioned biological sample is a urine fraction such as urine supernatant or urine cell pellets (e.g., urine sediment). In another embodiment, the above mentioned urine is obtained with or without prior digital rectal examination.

In another embodiment, the above mentioned mathematical correlation performed can be any one of linear and quadratic discriminant analysis (LDA and QDA), Support Vector Machine (SVM), Naïve Bayes or Random Forest. In a particular embodiment, the statistical method used to generate the score associating the level of expression of the at least two prostate cancer markers to a clinical assessment of prostate cancer is Naïve Bayes.

Other objects, advantages and features of the present invention will become more apparent upon reading of the following non-restrictive description of illustrative embodiments thereof, given by way of example only with reference to the accompanying drawings.

### BRIEF DESCRIPTION OF THE DRAWINGS

In the appended drawings:

FIG. 1 shows the average expression stability values of control markers between subjects harboring or not prostate cancer.

FIG. 2A shows the determination of the optimal number of control markers for normalization between subjects harboring or not prostate cancer.

FIG. 2B shows the distribution of mRNA expression values (Ct) of selected control markers in 261 whole urine samples from normal individuals (n=152) and prostate cancer subjects (n=109).

FIG. 2C shows the normalized gene expression level of PCA3 and five (5) prostate specific markers in prostate tissue samples (Normal and Tumor) as compared to other tumor and non-tumor tissues of the male genitourinary tract.

FIG. 3 shows the ordering of candidate genes from Table 1 based on AUC as a function of normalization techniques (Exo: using the level of expression (Ct) of an exogenous control; Mean Endo: using the mean Ct of 5 control markers from Table 2 (HPRT1, IPO8, POLR2A, TBP and GUSB); PSA: using the Ct of PSA (KLK3); Exo+PSA: using the Ct of PSA and the Ct of an exogenous control).

FIG. 4 (A-F) represents ROC curve analyses of 261 whole urine samples from subjects scheduled for prostate biopsy using the level of expression (Ct) of the prostate cancer markers and control markers of each classifier listed in Table 7A.

FIG. 5 shows altered gene expression for the prostate cancer markers of classifier 1, its interacting network in prostate cancer and effects on disease-free survival. A) OncoPrint™ of the total number of RNA expression altered in the 150 cases of primary and metastatic prostate cancer cases. B) Graph view of the neighborhood network of the prostate cancer markers (indicated with thick border) of classifier 1 and genes reported as belonging to a common pathway. C) Survival analysis of prostate cancer patients with altered versus not altered gene expression value (Z-value ≧1.25). Log rank p-value <0.05 was considered statistically significant.

FIG. 6 shows altered gene expression for the prostate cancer markers of classifier 3, its interacting network in prostate cancer and effects on disease-free survival. A) OncoPrint™ of the total number of RNA expression altered in the 150 cases of primary and metastatic prostate cancer cases. B) Graph view of the neighborhood network of the prostate cancer markers (indicated with thick border) of classifier 3 and genes reported as belonging to a common pathway. C) Survival analysis of prostate cancer patients with altered versus not altered gene expression value (Z-value ≧3.5). Log rank p-value <0.05 was considered statistically significant.

FIG. 7 shows altered gene expression for the prostate cancer markers of classifier 4, its interacting network in prostate cancer and effects on disease-free survival. A) OncoPrint™ of the total number of RNA expression altered in the 150 cases of primary and metastatic prostate cancer cases. B) Graph view of the neighborhood network of the prostate cancer markers (indicated with thick border) of classifier 4 and genes reported as belonging to a common pathway. C) Survival analysis of prostate cancer patients with altered versus not altered gene expression value (Z-value ≧3.5). Log rank p-value <0.05 was considered statistically significant.

FIG. 8 shows altered gene expression for the prostate cancer markers of classifier 5, its interacting network in prostate cancer and effects on disease-free survival. A) OncoPrint™ of the total number of RNA expression altered in the 150 cases of primary and metastatic prostate cancer cases. B) Graph view of the neighborhood network of the prostate cancer markers (indicated with thick border) of classifier 5 and genes reported as belonging to a common pathway. C) Survival analysis of prostate cancer patients with altered versus not altered gene expression value (Z-value ≧3.5). Log rank p-value <0.05 was considered statistically significant.

FIG. 9 shows altered gene expression for the prostate cancer markers of classifier 6, its interacting network in prostate cancer and effects on disease-free survival. A) OncoPrint™ of the total number of RNA expression altered in the 150 cases of primary and metastatic prostate cancer cases. B) Graph view of the neighborhood network of the prostate cancer markers (indicated with thick border) of classifier 6 and genes reported as belonging to a common pathway. C) Survival analysis of prostate cancer patient with altered versus not altered gene expression value (Z-value ≧3.75) versus not altered. Log rank p-value <0.05 was considered statistically significant.

FIG. 10 shows ROC curve comparison of classifier 3 normalized with 5 control markers, and the PCA3/PSA ratio for A) the training set (n=174; 101N/73T), B) the validation set (n=87; 51N/36T), C) the total cohort (n=261; 152N/109T) and D) a subset of cancer patients with high Gleason (≧7) score (n=204; 152N/52T).

FIG. 11 shows stratified performances analysis of classifier 3 normalized with 5 control markers per quintile for A) the total cohort (n=261; 152N/109T) and B) a group of patients before the first prostate biopsy (n=220; 122N/98T). In the total cohort (FIG. 11A), when considering all patients with multigene score below 0.4 (groups 1 and 2), only 17.3% of men with a positive biopsy will not be detected with the classifier 3, which translates into a negative predictive value (NPV) of 82.7% and a 6.59 times higher risk of positive biopsy for the group of men with a score over 0.4 (p-value <0.0001). In the group of patients before the first prostate biopsy (FIG. 11B), when considering all patients with multigene score below 0.4 (groups 1 and 2), 22.4% of men with a positive biopsy will not be detected with the classifier 3, which translates into a negative predictive value (NPV) of 77.6% and a 6.56 times higher risk of positive biopsy for the group of men with a score over 0.4 (p-value <0.0001).

FIG. 12 shows ROC curve comparison for the PCA3/PSA ratio, the classifier 3 and the classifier 3 with the addition of PCA3 for A) the total cohort (n=261; 152N/109T) and B) a subset of cancer patients with high Gleason (7) score (n=204; 152N/52T). In both the total cohort (FIG. 12A) and the subset of high Gleason (7) score (FIG. 12B), the difference between areas for the classifier alone and the classifier including the PCA3 marker was not statistically significant (p=0.3040 and 0.4224, respectively).

FIG. 13 shows stratified performances analysis of classifier 3 combined with PCA3 per quintile for the total cohort (n=261; 152N/109T). For the classifier 3, we observed equivalent sensitivity, specificity and negative predictive value (NPV) with or without the PCA3 marker. The only difference was the higher proportion of men with a positive biopsy in the group of men with score >0.8.

### DESCRIPTION OF ILLUSTRATIVE EMBODIMENTS

### Definitions

In the present description, a number of terms are extensively utilized. In order to provide a clear and consistent understanding of the specification and claims, including the scope to be given such terms, the following definitions are provided.

The use of the word “a” or an when used in conjunction with the term “comprising” in the claims and/or the specification may mean one but it is also consistent with the meaning of one or more“, at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”.

An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized. Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission.

As used herein, “gene” is meant to broadly include any nucleic acid sequence transcribed into an RNA molecule, whether the RNA is coding (e.g., mRNA) or non-coding (e.g., ncRNA). A number of gene/protein names and/or accession numbers are referred to herein. Accessing the corresponding sequence information based on gene/protein names and/or accession numbers can be readily done by any person of ordinary skill in the art from a number of publicly available gene databanks. Furthermore, while certain gene/protein names are used to refer to specific markers of the present invention, the skilled person will understand that other names/designations relating to the same markers (i.e., genes and proteins) can also be used.

As used herein, the term “marker” (used either alone or in combination with other qualifying terms such as prostate cancer marker, prostate-specific marker, control marker, exogenous marker, endogenous marker, etc.) relates to a measurable, calculable or otherwise obtainable parameter associated with any molecule, or combination of molecules, that is useful as an indicator of a biological and/or chemical state. In one embodiment, “marker” relates to a parameter associated with one or more biological molecules (i.e., “biomarkers”) such as naturally or synthetically produced nucleic acids (i.e., individual genes, as well as coding and non-coding DNA and RNA) and proteins (e.g., peptides, polypeptides). In another embodiment, “marker” relates to a single parameter which is calculated or otherwise obtained by considering expression data from two or more different markers (e.g., which are co-regulated in the context of prostate cancer and are considered together as a “marker pair” as defined herein). Markers can be further categorized into particular groups, depending on the type of indication that is sought, as discussed below. The skilled person would understand that these groups can be, but are not necessarily, mutually exclusive. For example, a prostate cancer marker can also be a prostate-specific marker, with the cancer distinguishing aspect being the expression level of the marker.

As used herein, “target” refers to a specific sub-region of a marker (e.g., exon-exon junction in the case of an RNA marker, or a specific epitope in the case of a protein marker) that is targeted for detection, amplification and/or hybridization in accordance with a method of the present invention.

“Prostate cancer marker” refers to a particular type of marker that is useful (either individually or when combined with other markers) as an indicator of prostate cancer in a subject in accordance with the methods of the present invention. In a particular embodiment, prostate cancer markers include those which are useful for providing (either individually or when combined with other markers) a clinical assessment of prostate cancer in a subject. In certain embodiments, the prostate cancer markers of the present invention include those listed in Table 5 or Table 6A, as well as markers which are co-regulated therewith (as shown in Table 6B) in accordance with the present invention. While specific accession numbers may be recited in certain sections of this application, other accession numbers relating to the same targets are nevertheless encompassed.

“Prostate-specific marker” refers to a particular type of marker that is useful (either individually or when combined with other markers) as an indicator of the presence or absence of prostate cells (both cancerous and non-cancerous) or a marker therefrom in a sample. Such markers can help distinguish prostate cells from non-prostate cells, or help assess the amount of prostate cells present in the sample. In some embodiments, the prostate-specific marker can be a molecule that is normally found in prostate cells and is not normally found in other tissues which could potentially “contaminate” the particular sample being analyzed. In fact, markers which are solely expressed in one organ or tissue are very rare. Accordingly, the fact that a prostate-specific marker is also expressed in a non-prostate tissue should not jeopardize the specificity of this marker provided that the non-prostate expression of this marker occurs in cells of tissues/organs which are not normally present in the particular sample being analyzed (e.g., urine). For example, when urine is the sample being analyzed, the prostate-specific marker should not be normally expressed in other types of cells (e.g., cells from the urinary tract system) expected to be found in the urine sample. Similarly, if another type of sample is used (e.g., sperm), the prostate-specific marker should not be expressed in other cell types that are normally encountered within such a sample. In one embodiment, a prostate-specific marker can be used as a control marker (i.e., prostate-specific control marker) for example to make sure that a sample contains a sufficient amount of prostate cells (e.g., in order to validate a negative result).

“Endogenous marker” refers to a marker (e.g., nucleic acid or polypeptide) that originates from the same subject as the sample being analyzed. More particularly, an “endogenous control marker” refers to a marker which is both useful as a control marker (either individually or when combined with other control markers) and originates from the same subject as the sample being analyzed. In one embodiment, an endogenous control marker can include one or more endogenous genes (i.e., “control gene” or “reference gene”) whose expression is relatively stable, e.g., in prostate-cancer versus non-prostate cancer samples, and/or from subject to subject.

“Exogenous marker” refers to a marker (e.g., nucleic acid or polypeptide) that does not originate from the same subject as the sample being analyzed. More particularly, an “exogenous control marker” refers to a marker which is both useful as a control marker (either individually or when combined with other control markers) and does not originate from the same subject as the sample being analyzed. For example, an exogenous control marker can be used to control for the steps of a method itself (e.g., amount of cells/starting material present in the sample, cell extraction, capture, hybridization/amplification/detection reaction, combinations thereof or any step which could be monitored to positively validate that the absence of a signal is not the result of a defect in one or more of the steps). In one embodiment, the exogenous marker or exogenous control marker can be isolated from a different subject, or can be synthetically produced, and may be added to the sample being analyzed. In another embodiment, the exogenous control marker can be a molecule that is added or spiked into the samples being analyzed for use as an internal positive or negative control. Exogenous control markers may be used together with the detection of one or more prostate cancer markers to distinguish between a “true negative” result (e.g., non-prostate cancer diagnosis), and a “false-negative” or “non-informative” result (e.g., due to a problem with an amplification reaction).

“Control marker” or “reference marker” refers to a particular type of marker that is useful (either individually or when combined with other control markers) to control for potential interfering factors and/or to provide one or more indications about sample quality, effective sample preparation, and/or proper reaction assembly/execution (e.g., of an RT-PCR reaction). In some embodiments, a control marker can be an endogenous control marker, an exogenous control marker, and/or a prostate-specific control marker, as described herein. A control marker may either be co-detected or detected separately from prostate cancer markers of the present invention. Control markers may be a combination of one or more endogenous genes such as housekeeping genes or prostate-specific control markers or genes.

In some embodiments, single markers (e.g., RNA) can be detected individually. In other embodiments, multiple primer sets and probes can be used within a single amplification reaction to produce amplicons of varying sizes that are specific to different markers. In another embodiment, at least two prostate cancer markers of the present invention are detected and measured. Amplicons typically have a length of at least 50 nucleotides to more than 200 nucleotides. However, it is also possible to produce amplicons of between 1000 to 2000 nucleotides, or amplicons of up to 10 kb or more. The person of skill in the art to which the present invention pertains can adapt the amplification reaction so as to enable a more efficient production of amplicons of a chosen size, as well known in the art.

In addition to considering markers of the present invention individually, in some embodiments, diagnostic or prognostic performance may be increased by considering the expression data from two or more different markers to yield a new parameter, which can then be treated as a new marker in itself. When the expression data from two different markers are considered, this is referred to herein as a “marker pair” (or “biomarker pair”, when the markers are biological molecules). More particularly, a “prostate cancer marker pair” relates to a single parameter obtained by considering the expression data from two different prostate cancer markers to improve the performance (e.g., the diagnostic/prognostic performance) of the methods of the present invention. In one embodiment, the single parameter can be obtained by considering the normalized expression value (e.g., deltaCt) of two different prostate cancer markers, determining which of these markers is the most over-expressed, and selecting the normalized expression value of the most over-expressed marker. For brevity, this type of prostate cancer marker pair is referred to herein by inserting the term “max” immediately preceding the names of the two prostate cancer markers being considered (e.g., “maxERG CACNA1D”). In another embodiment, the single parameter can be obtained by calculating the difference in the normalized expression values (e.g., delta Ct) between the most up-regulated marker and the most down-regulated marker among the tested dataset. For brevity, this type of prostate cancer marker pair is referred to herein by inserting a “-” between the names of the two prostate cancer markers being considered. For example, in the marker pair “ERG-SNAI2”, the single parameter is calculated by subtracting the expression value of SNAI2, which is the most down-regulated gene in the cohort, from the expression value of ERG, which is the most up-regulated gene in the cohort.

As used herein, the terms “classifier” or “prostate cancer classifier” includes a subset or ensemble of prostate cancer markers of the present invention (preferably used in combination), which enable classification of biological samples as originating from subjects having or lacking prostate cancer (e.g., the classifiers (“class 1-6”) listed in each of Tables 7-9). In one embodiment, the prostate cancer markers comprised in the classifier can be normalized or validated using one or more control markers (e.g., prostate-specific control markers, endogenous control markers, etc.) before being subjected to a mathematical correlation to generate a score associated with a clinical assessment of prostate cancer. In a particular embodiment, the classifier can include the means for providing the mathematical correlation (e.g., the statistical method or machine-learning algorithm that can be “trained”), and thus the clinical assessment score.

As used herein, “prostate cancer signature” includes the prostate cancer markers of a classifier of the present invention, along with one or more control markers. In one embodiment, each particular combination of prostate cancer markers and control marker(s) of the present invention (e.g., the 18 signatures listed in each of Tables 7-9) represent distinct prostate cancer signatures. When one or more prostate cancer markers in a prostate cancer signature of the present invention relate to gene expression values, the prostate cancer signature can be referred to herein as a “multi-gene signature” or a “multi-gene prostate cancer signature”.

“Hybridization” or “nucleic acid hybridization” or “hybridization” refers generally to the hybridization of two single stranded nucleic acid molecules having complementary base sequences, which under appropriate conditions will form a thermodynamically favored double stranded structure. The term “hybridizes” as used herein may relate to hybridizations under stringent or non-stringent conditions. The setting of conditions is well within the skill of the artisan and can be determined according to protocols described in the art. The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 2000, supra). Other protocols or commercially available hybridization kits (e.g., ExpressHyb™ from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands. By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) or may contain one or more residues (including abasic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes.

The term “identical” or “percent identity” in the context of two or more nucleic acid or amino acid sequences as used herein, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

A “probe” is meant to include a nucleic acid oligomer or aptamer that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

Methods of gene expression profiling include methods based on hybridization analysis of oligonucleotides, methods based on sequencing of polynucleotides, and proteomic-based methods determining protein level of the oligonucleotide. Exemplary methods known in the art for the quantification of RNA expression in a sample include without being limited to Southern blots, Northern blots, Microarray, Polymerase chain reaction (PCR), NASBA, and TMA.

Nucleic acid sequences may be detected by using hybridization with a complementary sequence (e.g., oligonucleotide probes) (see U.S. Pat. No. 5,503,980 (Cantor), U.S. Pat. No. 5,202,231 (Drmanac et al.), U.S. Pat. No. 5,149,625 (Church et al.), U.S. Pat. No. 5,112,736 (Caldwell et al.), U.S. Pat. No. 5,068,176 (Vijg et al.), and U.S. Pat. No. 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ one nucleotide (see U.S. Pat. Nos. 5,837,832 and 5,861,242 (Chee et al.)).

A detection step may use any of a variety of known methods to detect the presence of nucleic acid by hybridization to a probe oligonucleotide. One specific example of a detection step uses a homogeneous detection method such as described in detail previously in Arnold et al., Clinical Chemistry 35:1588-1594 (1989), and U.S. Pat. No. 5,658,737 (Nelson et al.), and U.S. Pat. Nos. 5,118,801 and 5,312,728 (Lizardi et al.).

The types of detection methods in which probes can be used include Southern blots (DNA detection), dot or slot blots (DNA, RNA), and Northern blots (RNA detection). Labeled proteins could also be used to detect a particular nucleic acid sequence to which it binds (e.g., protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g., an array) of different probes.

A “label” refers to a molecular moiety or compound that can be detected or can lead to a detectable signal. A label can be joined, directly or indirectly, to a probe/primer or the nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as additional oligonucleotide(s), which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label. Other methods of labeling nucleic acids are known whereby a label is attached to a nucleic acid strand as it is fragmented, which is useful for labeling nucleic acids to be detected by hybridization to an array of immobilized DNA probes (e.g., see PCT No. PCT/IB99/02073).

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criteria like stability for example. Chimeras of deoxyribonucleotides and ribonucleotides may also be within the scope of the present invention.

The term “microarray” refers to an orderly arrangement of hybridizable molecules (e.g., oligonucleotide or polypeptide) attached to a solid support. The principle aim of using microarray technology as a gene expression profiling tool is to study the effects of certain treatments, diseases, and developmental stages on the expression levels of thousands of genes simultaneously. For example, microarray-based gene expression profiling can be used to identify genes whose expression is up- or down-regulated in tumor samples as compared to samples from normal individuals.

An “immobilized probe” or “immobilized nucleic acid” refers to a nucleic acid that joins, directly or indirectly, a capture oligomer to a solid support. An immobilized probe is an oligomer joined to a solid support that facilitates separation of bound target sequence from unbound material in a sample. Any known solid support may be used, such as matrices and particles free in solution, made of any known material (e.g., nitrocellulose, nylon, glass, polyacrylate, mixed polymers, polystyrene, silane polypropylene and metal particles, preferably paramagnetic particles). Preferred supports are monodisperse paramagnetic spheres (i.e., uniform in size ±about 5%), thereby providing consistent results, to which an immobilized probe is stably joined directly (e.g., via a direct covalent linkage, chelation, or ionic interaction), or indirectly (e.g., via one or more linkers), permitting hybridization to another nucleic acid in solution.

“Complementary DNA (cDNA)”. Refers to recombinant nucleic acid molecules synthesized by reverse transcription of RNA (e.g., mRNA).

“Amplification” or “amplification reaction” refers to any in vitro procedure for obtaining multiple copies (“amplicons”) of a target nucleic acid sequence or its complement, or fragments thereof. In vitro amplification refers to production of an amplified nucleic acid that may contain less than the complete target region sequence or its complement. In vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as QR-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. No. 6,087,133 and U.S. Pat. No. 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14 25 and (Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., 2000, Molecular Cloning—A Laboratory Manual, Third Edition, CSH Laboratories). As commonly known in the art, the oligos are designed to bind to a complementary sequence under selected conditions.

As used herein, a “primer” defines an oligonucleotide which is capable of annealing to a target sequence, thereby creating a double stranded region which can serve as an initiation point for nucleic acid synthesis under suitable conditions. Primers can be, for example, designed to be specific for certain alleles so as to be used in an allele-specific amplification system. For example, a primer can be designed so as to be complementary to a differentially expressed RNA which is associated with a malignant state of the prostate, whereas another differentially expressed RNA form the same gene is associated with a non-malignant state (benign) thereof. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Of course the design of a primer from a known nucleic acid sequence is well known in the art. Oligos can comprise a number of types of different nucleotides. Skilled artisans can easily assess the specificity of selected primers and probes by performing computer alignments/searches using well-known databases (e.g., Genbank™). Primers and probes can be designed based upon exon or intron sequences present in the mRNA transcript using publicly available sequence database such as the NCBI Reference Sequence (RefSeq) database. Where necessary or desired, primers and probes are designed to detect the maximum number of transcripts for the gene of interest without detecting gene products with similar sequence such as homologs. Those skilled in the art will recognize that primers and probes design required several steps such as mapping the target sequence to the genome, identify exon-exon junctions and designing a primer at each junction, identifying SNP and transcript variant that can be detected simultaneously or separately with a set of primers. Other factors that can influence primer design include without being restricted to: primer length, melting temperature (Tm), G/C content, specificity, complementary primer sequence, primer dimers and 3′ sequence. For general use, optimal primer and probes can be designed using any commercially or otherwise publicly available primer/probe design software, such as PrimerExpress™ (Applied Biosystem) or Primer3™ (http://primer3.sourceforge.net). Each assay associated with the examples disclosed herein used a fluorescently-labeled TaqMan® Minor Groove Binder (MGB) probe and two unlabeled PCR primers. Because they are designed to perform under universal thermal cycling conditions for two-step RT-PCR, primers used in examples herein are generally 17-30 bases in length and contain about 50-60% G+C bases and exhibit Tm's between 50 and 80° C. TaqMan® assays use 5′ nuclease chemistry and probe that incorporate the MGB technology. The MGB technology enhances the probe Tm by binding in the minor groove of a DNA duplex. This Tm enhancement enables the use of probes as short as 13 bases. Shorter probes allow superior specificity and shorter amplicon size. Table 1, Table 2 and Table 5 provide further information concerning the primer, probe and amplicon sequences associated with the present invention.

The terminology “amplification pair” or “primer pair” refers herein to a pair of oligonucleotides (oligos) of the present invention, which are selected to be used together for amplifying a selected nucleic acid sequence (e.g., a marker) by one of a number of types of amplification processes.

The following technologies are included within the scope of an “amplification and/or hybridization reaction”.

Polymerase Chain Reaction (PCR).

Polymerase chain reaction can be carried out in accordance with known techniques. See, e.g., U.S. Pat. Nos. 4,683,195; 4,683,202; 4,800,159; and 4,965,188 (the disclosures of all three U.S. Patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following Ethidium Bromide (EtBr) staining of the DNA following gel electrophoresis, or using a detectable label in accordance with known techniques, and the like. For a review on PCR techniques (see PCR Protocols, A Guide to Methods and Amplifications, Michael et al., Eds, Acad. Press, 1990).

Nucleic Acid Sequence Based Amplification (NASBA).

NASBA can be carried out in accordance with known techniques (Malek et al., Methods Mol Biol, 28:253-260, U.S. Pat. Nos. 5,399,491 and 5,554,516). In an embodiment, the NASBA amplification starts with the annealing of an antisense primer P1 (containing the T7 RNA polymerase promoter) to the mRNA target. Reverse transcriptase (RTase) then synthesizes a complementary DNA strand. The double stranded DNA/RNA hybrid is recognized by RNase H that digests the RNA strand, leaving a single-stranded DNA molecule to which the sense primer P2 can bind. P2 serves as an anchor to the RTase that synthesizes a second DNA strand. The resulting double-stranded DNA has a functional T7 RNA polymerase promoter recognized by the respective enzyme. The NASBA reaction can then enter in the phase of cyclic amplification comprising six steps: (1) Synthesis of short antisense single-stranded RNA molecules (101 to 103 copies per DNA template) by the T7 RNA polymerase; (2) annealing of primer P2 to these RNA molecules; (3) synthesis of a complementary DNA strand by RTase; (4) digestion of the RNA strand in the DNA/RNA hybrid; (5) annealing of primer P1 to the single-stranded DNA; and (6) generation of double stranded DNA molecules by RTase. Because the NASBA reaction is isothermal (41° C.), specific amplification of ssRNA is possible if denaturation of dsDNA is prevented in the sample preparation procedure. It is thus possible to pick up RNA in a dsDNA background without getting false positive results caused by genomic dsDNA.

Transcription-Mediated Amplification (TMA).

TMA is an isothermal nucleic-acid-based method that can amplify RNA or DNA targets a billion-fold in only a few hours. Developed at Gen-Probe (e.g., see U.S. Pat. Nos. 5,399,491, 5,480,784, 5,824,818 and 5,888,779), TMA technology uses two primers and two enzymes: RNA polymerase and reverse transcriptase. One primer contains a promoter sequence for RNA polymerase. In the first step of amplification, this primer hybridizes to the target rRNA at a defined site. Reverse transcriptase creates a DNA copy of the target rRNA by extension from the 3′end of the promoter primer. The RNA in the resulting RNA:DNA duplex is degraded by the RNase activity of the reverse transcriptase. Next, a second primer binds to the DNA copy. A new strand of DNA is synthesized from the end of this primer by reverse transcriptase, creating a double-stranded DNA molecule. RNA polymerase recognizes the promoter sequence in the DNA template and initiates transcription. Each of the newly synthesized RNA amplicons reenters the TMA process and serves as a template for a new round of replication. The amplicons produced in these reactions are detected by a specific gene probe in hybridization protection assay, a chemiluminescence detection format or using other probe specific technologies (e.g., molecular beacons).

Sequencing technologies such as Sanger sequencing, pyrosequencing, sequencing by ligation, massively parallel sequencing, also called “Next-generation sequencing” (NGS), and other high-throughput sequencing approaches with or without sequence amplification of the target can also be used to detect and quantify the presence of target nucleic acid in a sample. Sequence-based methods can provide further information regarding alternative splicing and sequence variation in previously identified genes. Sequencing technologies include a number of steps that are grouped broadly as template preparation, sequencing, detection and data analysis. Current methods for template preparation involve randomly breaking genomic DNA into smaller sizes from which each fragment is immobilized to a support. The immobilization of spatially separated fragment allows thousands to billions of sequencing reaction to be performed simultaneously. A sequencing step may use any of a variety of methods that are commonly known in the art. One specific example of a sequencing step uses the addition of nucleotides to the complementary strand to provide the DNA sequence. The detection steps range from measuring bioluminescent signal of a synthesized fragment to four-color imaging of single molecule. The voluminous amount of data produced by NGS technologies demands substantial informatics support in term of data storage to be able to perform genome alignment and assembly from billions of sequencing reads. Validation of this assembly also requires rigorous tracking and quality control.

Ligase chain reaction (LCR) can be carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392 396; and ibid, 1992, Nucleic Acids Res. 20:1691 1696).

Target Capture.

In one embodiment, target capture is included in the method to increase the concentration or purity of the target nucleic acid before in vitro amplification. Preferably, target capture involves a relatively simple method of hybridizing and isolating the target nucleic acid, as described in detail elsewhere (e.g., see U.S. Pat. Nos. 6,110,678, 6,280,952, and 6,534,273). Generally speaking, target capture can be divided in two family, sequence specific and non-sequence specific. In the non-specific method, a reagent (e.g., silica beads) is used to capture non-specifically nucleic acids. In the sequence specific method an oligonucleotide attached to a solid support is contacted with a mixture containing the target nucleic acid under appropriate hybridization conditions to allow the target nucleic acid to be attached to the solid support to allow purification of the target from other sample components. Target capture may result from direct hybridization between the target nucleic acid and an oligonucleotide attached to the solid support, but preferably results from indirect hybridization with an oligonucleotide that forms a hybridization complex that links the target nucleic acid to the oligonucleotide on the solid support. The solid support is preferably a particle that can be separated from the solution, more preferably a paramagnetic particle that can be retrieved by applying a magnetic field to the vessel. After separation, the target nucleic acid linked to the solid support is washed and amplified when the target sequence is contacted with appropriate primers, substrates and enzymes in an in vitro amplification reaction.

Generally, capture oligomer sequences include a sequence that specifically binds to the target sequence, when the capture method is indeed specific, and a “tail” sequence that links the complex to an immobilized sequence by hybridization. That is, the capture oligomer includes a sequence that binds specifically to a marker of the present invention, PSA or to another prostate specific marker (e.g., hK2/KLK2, PMSA, transglutaminase 4, acid phosphatase, PCGEM1) target sequence and a covalently attached 3′ tail sequence (e.g., a homopolymer complementary to an immobilized homopolymer sequence). The tail sequence which is, for example, 5 to 50 nucleotides long, hybridizes to the immobilized sequence to link the target-containing complex to the solid support and thus purify the hybridized target nucleic acid from other sample components. A capture oligomer may use any backbone linkage, but some embodiments include one or more 2′-methoxy linkages. Of course, other capture methods are well known in the art. The capture method on the cap structure (Edery et al., 1988, gene 74(2): 517-525, U.S. Pat. No. 5,219,989) and the silica-based method are two non-limiting examples of capture methods.

As used herein, the term “purified” refers to a molecule (e.g., nucleic acid) having been separated from a component of the composition in which it was originally present. Thus, for example, a “purified nucleic acid” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). In contrast, the term “crude” means molecules that have not been separated from the components of the original composition in which it was present. For the sake of brevity, the units (e.g., 66, 67 . . . 81, 82, 83, 84, 85, . . . 91, 92% . . . ) have not been specifically recited but are considered nevertheless within the scope of the present invention.

Herein the terminology “Gleason Score”, as well known in the art, is the most commonly used system for the grading/staging and prognosis of adenocarcinoma. The system describes a score between 2 and 10, with 2 being the least aggressive and 10 being the most aggressive. The score is the sum of the two most common patterns (grade 1-5) of tumor growth found. To be counted a pattern (grade) needs to occupy more than 5% of the biopsy sample. The scoring system requires biopsy material (core biopsy or operative sample) in order to be accurate; cytological preparations cannot be used. If the biopsy confirms the presence of cancer, the extent of cancer and aggressiveness of the tumor (termed the Gleason grade) are determined. The pathologist typically identifies two architectural patterns of the prostate tumor, and assigns a Gleason grade to each: a primary grade, related to how the cells look, between 1 to 5 and a secondary grade, related to how the cells are arranged, also between 1 and 5. The primary grade is determined by the appearance of the cancerous cells in the biopsy sample; if the tissue appears similar to normal prostate tissue, a grade of 1 is assigned. If the tissue has none of the normal features and cancer cells are seen throughout the sample, a grade of 5 is assigned. Grades 2 through 4 are assigned to tissues whose appearance is between 1 and 5. Secondary grade numbers pertaining to arrangement of cells are similarly assigned.

The primary and secondary grade numbers are then combined together to form the Gleason score. The higher the Gleason score, the more aggressive (fast-growing) the tumor appears. If the cancerous tissue shows primary grade 3 and secondary grade 4 areas of tumor involvement, the combined Gleason score is “3 plus 4” or 7. Currently, about 90 percent of men with newly diagnosed prostate cancer have a Gleason score of 6 or 7. Gleason scores between less than 6 are typically referred to as low grade or well-differentiated. Gleason scores between 6 and 7 are referred to as intermediate grade. Gleason scores between 8 and 10 tumors are high grade or poorly differentiated.

In developing his system, Dr. Gleason discovered that by giving a combination of the grades of the two most common patterns he could see in any particular patients samples, he was better able to predict the likelihood that a particular patient would do well or badly. Therefore, although it may seem confusing, the Gleason score which a physician usually gives to a patient is actually a combination or sum of two numbers which is accurate enough to be very widely used. These combined Gleason sums or scores may be determined as follows:
The lowest possible Gleason score is 2 (1+1), where both the primary and secondary patterns have a Gleason grade of 1 and therefore when added together their combined sum is 2.
Very typical Gleason scores might be 5 (2+3), where the primary pattern has a Gleason grade of 2 and the secondary pattern has a grade of 3, or 6 (3+3), a pure pattern.
Another typical Gleason score might be 7 (4+3), where the primary pattern has a Gleason grade of 4 and the secondary pattern has a grade of 3.
Finally, the highest possible Gleason score is 10 (5+5), when the primary and secondary patterns both have the most disordered Gleason grades of 5.

Another way of staging prostate cancer is by using the “TNM System”, as described by the American Joint Committee on Cancer (AJCC) in the AJCC Seventh Edition Cancer Staging Manual. It describes the extent of the primary tumor (T stage), the absence or presence of spread to nearby lymph nodes (N stage) and the absence or presence of distant spread, or metastasis (M stage). Each category of the TNM classification is divided into subcategories representative of its particular state. For example, primary tumors (T stage) may be classified into:
T1: The tumor cannot be felt during a digital rectal exam, or seen by imaging studies, but cancer cells are found in a biopsy sample;
T2: The tumor can be felt during a DRE and the cancer is confined within the prostate gland;
T3: The tumor has extended through the prostatic capsule (a layer of fibrous tissue surrounding the prostate gland) and/or to the seminal vesicles (two small sacs next to the prostate that store semen), but no other organs are affected;
T4: The tumor has spread or attached to tissues next to the prostate (other than the seminal vesicles).

Lymph node involvement is divided into the following 2 categories:
N0: Cancer has not spread to any lymph nodes;
N1: Cancer has spread to regional lymph node (inside the pelvis).

Metastasis is generally divided into the following two categories:
M0: The cancer has not metastasized (spread) beyond the regional lymph nodes; and
M1: The cancer has metastasized to distant lymph nodes (outside of the pelvis), bones, or other distant organs such as lungs, liver, or brain.

In addition, the T stage is further divided into subcategories T1a-c T2a-c, T3a-b and T4. The characteristics of each of these subcategories are well known in the art and can be found in a number of textbooks.

Control Sample.

The terms “control sample”, “normal sample”, or “reference sample” refer herein to a sample that is indicative or representative of a non-cancerous status (e.g., non-prostate cancer status). Control samples can be obtained from patients/individuals not afflicted with prostate cancer. Other types of control samples may also be used. Once a cut-off value is determined, a control sample giving a signal characteristic of the predetermined cut-off value can also be designed and used in the methods of the present invention. Diagnosis/prognosis tests are commonly characterized by the following 4 performance indicators: sensitivity (Se), specificity (Sp), positive predictive value (PPV), and negative predictive value (NPV). The following table presents the data used in calculating the 4 performance indicators.

Disease/condition

| | | Presence (+) | Absence (−) | |
|---|---|---|---|---|
| Test | (+) | a | b | a + b |
| | (−) | c | d | c + d |
| | | a + c | b + d | |

Sensitivity corresponds to the proportion of subjects having a positive diagnostic test who truly have the disease or condition (Se=a/a+c). Specificity relates to the proportion of subjects having a negative diagnostic test and who do not have the disease or condition (Sp=d/b+d). The positive predictive value concerns the probability of actually having the disease or condition (e.g., prostate cancer) when the diagnostic test is positive (PPV=a/a+b). Finally, the negative predictive value is indicative of the probability of truly not having the disease/condition when the diagnostic test is negative (NPV=c/c+d). The values are generally expressed in %. Se and Sp generally relate to the precision of the test, while PPV and NPV concern its clinical utility.

The terminologies “level” and “amount” are used herein interchangeably when referring to a marker which is measured.

It should be understood by a person of ordinary skill, that numerous statistical methods can be used in the context of the present invention to determine if the test is positive or negative or to determine the particular stage, grade, volume of the prostate tumor or aggressiveness thereof.

The term “variant” refers herein to a protein or nucleic acid molecule which is substantially similar in structure and biological activity to the protein or nucleic acid of the present invention, to maintain at least one of its biological activities. Thus, provided that two molecules possess a common activity and can substitute for each other, they are considered variants as that term is used herein even if the composition, or secondary, tertiary or quaternary structure of one molecule is not identical to that found in the other, or if the amino acid sequence or nucleotide sequence is not identical.

As used herein, the terms “subject” and “patient” refer to a mammal, preferably a human, having a prostate gland. Specific examples of subjects and patients include, but are not limited to individuals requiring medical assistance, and in particular, patients with cancer such as prostate cancer, patients suspected of having prostate, or patients being monitored to assess the state of their prostate.

As used herein, the term “up-regulated” or “over-expressed” refers to a gene that is expressed (e.g., RNA and/or protein expression) at a higher level in cancer tissue (e.g., in prostate cancer tissue) relative to the level in other corresponding tissues (e.g., normal or non-cancerous prostate tissue). In some embodiments, genes up-regulated in cancer are expressed at a level at least 10%, preferably at least 25%, even more preferably at least 50%, still more preferably 100%, yet more preferably at least 200%, and most preferably 300% higher than the level of expression in other corresponding tissues (e.g., normal or non-cancerous prostate tissue). In some embodiments, genes up-regulated in prostate cancer are “androgen regulated genes”. Conversely, as used herein, the term “down regulated” refers to a gene that is expressed (e.g., mRNA or protein expression) at a lower level in cancer tissue (e.g., in prostate cancer) relative to the level in other corresponding tissues (e.g., normal or non-cancerous prostate tissue). In some embodiments, genes down-regulated in cancer are expressed at a level at least 10%, preferably at least 25%, even more preferably at least 50%, still more preferably 100%, yet more preferably at least 200%, and most preferably 300% lower than the level of expression in other corresponding tissues (e.g., normal or non-cancerous prostate tissue).

Establishing whether one or more genes is up or down regulated in cancer tissue (e.g., prostate cancer tissue) can be done by comparing the expression level of the one or more gene to that of a subject lacking prostate cancer. In one embodiment, this can be done by comparing the expression level to one or more predetermined values that are indicative of the expression of a subject lacking cancer (e.g., lacking prostate cancer). As used herein, the phrase “determining the expression” refers to the measuring of any expression product (e.g., coding RNA, non-coding RNA, or an expressed polypeptide) of the preset invention.

Gene “co-regulation”, “co-occurrence” or “co-occurrence regulation”. Genes often work together and thus their expression may be “co-regulated” in a concerted way, a process also referred as “co-expression regulation” or “co-regulation”. “Co-regulated genes” or “co-expressed genes” identified for a disease process like cancer (e.g., prostate cancer) can serve as biomarkers for tumor status, and can thus be useful in lieu of, or in addition to, another marker with which it is co-regulated. As used herein, the terminology “co-regulated genes”, or the like, refers to sets of connected genes that are up- or down-regulated in a concerted fashion and belong to the same biological process, such as cancer, across multiple subjects. For example, co-regulated genes can be up-regulated or down-regulated together in cancer (e.g., prostate cancer) tissue. Also encompassed within the meaning of co-regulated genes are genes which are co-regulated in an opposite fashion. For example, one gene of among the co-regulated genes may be up-regulated in cancer tissue, while the other gene may be correspondingly down-regulated in the cancer tissue. Co-regulation also encompasses instances of mutual exclusivity, for example, where the detection of one gene correlates with the absence of detection of another gene. Co-regulation can be determined using an algorithm accessible via the cBio Cancer Genomics Portal (http://cbioportal.org) which computes mutual exclusivity or co-occurrence between all pairs of gene and generates a binary matrix with p-values for all target genes by applying the Fisher Exact test to each individual gene pair. The strength of co-regulation between two genes can be represented in terms of p-values. In one embodiment, “strongly co-regulated genes” can refer to genes that are co-regulated with a p-value of <0.00001. In another embodiment, “moderately co-regulated genes” can refer to genes that are co-regulated with a p-value of <0.001. In another embodiment, “co-regulated genes” can refer to genes that are co-regulated with a p-value of <0.05. In another embodiment, “strong mutually exclusive genes” can refer to genes that are not co-regulated with a p-value <0.005. In another embodiment, “mutually exclusive genes” refer to genes that are not co-regulated with a p-value <0.05. It should be understood that the present invention should not be limited to the above-listed p-values, as others could be chosen to suit particular needs of a skill artisan. Such other p-values are also encompassed by the present invention.

A “biological sample”, “sample of a patient” or “sample of a subject” is meant to include any tissue or material derived from a living or dead mammal (preferably a living human) which may contain a marker of the present invention.

As used herein the term “parameters”, also known as “process parameters”, include one or more variables used in the methods of the present invention to determine one or more of: the amount of marker/target detected in a sample; the expression level of one or more markers/targets; and the value of the clinical assessment that correlates with an expression level of one or more markers/targets. Parameters include but are not limited to: primer type; probe type; amplicon length; concentration of a substance; mass or weight of a substance; time for a process; temperature for a process; activity during a process such as centrifugation, rotating, shaking, cutting, grinding, liquefying, precipitating, dissolving, electrically modifying, chemically modifying, mechanically modifying, heating, cooling, preserving (e.g., for days, weeks, months and even years) and maintaining in a still (unagitated) state. Parameters may further include a variable in one or more mathematical formulas used in the method of the present invention. Parameters may include a threshold used to determine the value of one or more parameters or outputs used or created in a subsequent step of the method of the present invention. In a preferred embodiment, the threshold is a minimum or maximum amount of target detected. Of course, such parameters can be adjusted by the person of skill in the art to which the present invention pertains, so as to more particularly suit particular needs of sensitivity, specificity, efficiency and the like.

As used herein the phrase “signal detection”, refers to a measured quantity of one or more markers detected in sample or sub-sample, such as a quantity of mass, volume or concentration (e.g., concentration of light emission from fluorescent dyes). The amount of target detected may be an indirect or surrogate measure of the quantity of the target, such as a Ct or Copy number measurement from a PCR reaction, or a deltaCt or deltaCopy number result when normalizing such as to one or more reference or housekeeping genes or other known internal standards.

As used herein the phrase “expression level” refers to a potential range of continuous or discrete values for a determined expression level of a target. An expression level can be a discrete value or determined relatively to a level in normal cells such as prostate cells, such as for example, an increase in level relative to a prior time point, or an increase in level relative to a pre-established threshold level.

As used herein the term “nomogram” refers to an algorithm or other means of deriving a result taking into account a combination of disease factors or clinical factors such as: age; race; stage of the cancer; PSA level; biopsy; pathology; use of hormone therapy; radiation dosage; heredity; and so on. The terminology “nomogram” is widely used where prostate cancer is of concern.

As used herein the term “clinical assessment” refers to an evaluation of a patients physical condition and prediction of the presence and/or degree of severity of prostate cancer and its evolution, as well as the prospect of recovery as anticipated from usual course of the disease and is based on information gathered from physical and laboratory examinations and the patients medical history. As used herein the phrase “clinical assessment range of outcomes” refers to a potential range of continuous or discrete values for a clinical assessment of the patient.

As used herein the term “screening” refers to a type of clinical assessment wherein the presence of cancer or lack of cancer is first identified. Detection of cancer at an early stage is believed to improve therapeutic benefit and the clinical outcomes that result.

As used herein the term “diagnosis” refers to another type of clinical assessment where the presence of cancer or lack of cancer is confirmed.

As used herein the term “staging” refers to a further type of clinical assessment. Staging typically is the determination of the extent and location of the tumor to develop appropriate treatment strategies and estimate a prognosis. Staging is one way of predicting the degree of severity of prostate cancer and of its evolution, as well as the prospect of recovery as anticipated from the usual course of the disease.

As used herein the term “prognosis” refers to yet another further type of clinical assessment. Prognosis typically involves establishing the prospect of recovery as anticipated from the usual course of disease or peculiarities of the case such as determining likelihood of developing prostate cancer, determining the likelihood of developing aggressive prostate cancer, determining the likelihood of developing metastatic prostate cancer and/or determining long-term survival outcome.

As used herein, the term “determination of aggressiveness” refers to an additional type of clinical assessment. The determination of aggressiveness is often made by establishing the Gleason Score for prostate cancer, which in turn can guide the choice of appropriate treatment method(s).

As used herein the term “treatment planning” refers to yet an additional type of clinical assessment. Treatment planning typically refers to the recommendation for or ruling out of one or more treatment options including but not limited to: observation (watchful waiting); surgery such as radical prostatectomy; radiation therapy such as external beam radiation or brachytherapy; pharmaceutical or other agent therapy such as hormonal therapy or chemotherapy; testosterone lowering therapy such as via medication or surgical removal of the testis; and combinations of these.

As used herein the term “monitoring response to treatment” refers to another type of clinical assessment. Monitoring response to treatment typically refers to one or more patient condition monitoring options that are directly or indirectly related to a current patient treatment such as routine (e.g., of planned frequency) diagnostic and prognostic procedures. Applicable diagnostic procedures include but are not limited to: routine performance of one or more tests made on a sample obtained from the patient such as a blood or urine test; routine imaging tests; and routine biopsies.

As used herein the term “surveillance” refers to a further type of clinical assessment. Surveillance typically refers to one or more patient condition monitoring options such as routine (e.g., of planned frequency) diagnostic and prognostic procedures. Surveillance is not necessarily related to a current patient treatment (e.g., may be in an observation only period). Applicable diagnostic procedures include but are not limited to: routine performance of one or more tests made on a sample obtained from the patient such as a blood or urine test; routine imaging tests; and routine biopsies.

Methods, Kits and Compositions for Providing a Clinical Assessment of Prostate Cancer

The present invention relates to methods, kits and compositions for providing a clinical assessment of prostate cancer in a subject based on a biological sample therefrom. Briefly, in one particular embodiment, a biological sample is obtained from a subject (e.g., urine, tissue or blood sample), and normalized expression levels of at least two prostate cancer markers in a prostate cancer signature of the present invention are determined. A mathematical correlation of the normalized expression levels of the at least two prostate cancer markers is performed to obtain a score, and this score is used to provide a clinical assessment of prostate cancer in the subject.

Prostate Cancer Signatures

Prostate cancer signatures of the present invention relate to combinations of at least two prostate cancer markers whose expression pattern in urine is associated (e.g., either positively or negatively) with a clinical assessment of prostate cancer.

In one embodiment, the prostate cancer signatures of the present invention can include at least two prostate cancer markers selected from Table 5 or Table 6A. In another embodiment, prostate cancer signatures of the present invention can include at least two prostate cancer markers selected from: (1) CACNA1D or a marker co-regulated therewith in prostate cancer; (2) ERG or a marker co-regulated therewith in prostate cancer; (3) HOXC4 or a marker co-regulated therewith in prostate cancer; (4) ERG-SNAI2 prostate cancer marker pair; (5) ERG-RPL22L1 prostate cancer marker pair; (6) KRT 15 or a marker co-regulated therewith in prostate cancer; (7) LAMB3 or a marker co-regulated therewith in prostate cancer; (8) HOXC6 or a marker co-regulated therewith in prostate cancer; (9) TAGLN or a marker co-regulated therewith in prostate cancer; (10) TDRD1 or a marker co-regulated therewith in prostate cancer; (11) SDK1 or a marker co-regulated therewith in prostate cancer; (12) EFNA5 or a marker co-regulated therewith in prostate cancer; (13) SRD5A2 or a marker co-regulated therewith in prostate cancer; (14) maxERG CACNA1D prostate cancer marker pair; (15) TRIM29 or a marker co-regulated therewith in prostate cancer; (16) OR51E1 or a marker co-regulated therewith in prostate cancer; and (17) HOXC6 or a marker co-regulated therewith in prostate cancer.

In another embodiment, the prostate cancer signatures of the present invention can comprise as least two prostate cancer markers, wherein one of the markers is CACNA1 D or a prostate cancer marker co-regulated therewith in prostate cancer. In another embodiment, the prostate cancer signatures of the present invention can comprise at least two prostate cancer markers being CACNA1D or a prostate cancer marker co-regulated therewith in prostate cancer, and ERG or a prostate cancer marker co-regulated therewith in prostate cancer.

In a particular embodiment, a marker that is co-regulated with a prostate cancer marker mentioned above is as set forth in Table 6B. In other particular embodiments, the co-regulated markers set forth in Table 6B show co-regulation with: a p-value <0.05 (“co-regulation”); a p-value of <0.001 (“moderate co-regulation”); a p-value of <0.05 (“strong co-regulation”); a p-value <0.05 (“mutually exclusive”); or a p-value of <0.005 (“strongly mutually exclusive”).

In another embodiment, the prostate cancer signatures of the present invention can include at least two prostate cancer markers of the present invention, combined with one or more control markers. In another embodiment, the one or more control markers are selected from those listed in Table 2 or Tables 7-9.

In another embodiment, the expression data from two or more different markers of the present invention can be considered together to yield a new parameter, which can then be treated as a new marker in itself (i.e., a “marker pair”, as explained above). In particular embodiments, the marker pair can be a prostate cancer marker pair, such as the maximum expression level between two different prostate cancer markers (e.g., “maxERG CACNA1D”), or the difference in the expression levels between two different prostate cancer markers (e.g., “ERG-SNAI2”). For brevity, the former is referred to herein by inserting the term “max” immediately preceding the names of the two prostate cancer markers being considered, and the latter is referred to herein by inserting a “-” between the names of the two prostate cancer markers being considered. The skilled person would be able to derive other types of informative marker pairs based on the prostate cancer markers and control markers disclosed herein.

In another embodiment, the prostate cancer signatures of the present invention provide a clinical assessment of prostate cancer which is superior (i.e., better able to discriminate between prostate cancer and non-prostate cancer) to PCA3 (e.g., PCA3/PSA ratio). In another embodiment, it may be useful to employ a prostate cancer diagnostic tool that does not rely on PCA3 per se. For example, if a clinical assessment of prostate cancer is made on a subject using a PCA3-based test, it may be desirable to have a separate, independent clinical assessment of prostate cancer performed which does not rely on PCA3. In this way, the prostate cancer signatures of the present invention may be used to independently validate a PCA3-based test result, or vice versa. Accordingly, on a particular embodiment, the prostate cancer signatures of the present invention do not include PCA3.

Biological Samples

A biological sample is generally obtained from a subject having or suspected of having prostate cancer. In various embodiments, the subject may have or be suspected to have cancer (e.g., primary prostate cancer); may have a family history of prostate cancer; may be followed for prostate cancer progression (e.g., to monitor cancer progression and/or effectiveness of cancer therapy); may have one or more conditions other than prostate cancer, or exhibit symptoms related to benign prostatic hyperplasia (BPH), high grade prostatic intraepithelial neoplasia (HGPIN), or atypical small acinar proliferation (ASAP). In other embodiments, the methods of the present invention may be performed on a biological sample from a subject subsequent to a previous diagnostic test, such as a PSA test in which the PSA level was higher than 10 ng/mL, 4 ng/mL, 2.5 ng/mL, 2 ng/mL, or some other diagnostically useful value.

In one embodiment, samples may be tumor or non-tumor tissue, and can include, for example, any tissue or material that may contain cells or markers therefrom associated with prostatic tissue such as: urine; prostate biopsy; semen/ejaculate; bladder washings; blood; lymph nodes; lymphatic tissue; lymphatic fluid; transurethral resection of the prostate (TURP); other bodily fluids, tissues or materials; cell lines; histological slides; preserved tissue such as formalin fixed, frozen or dehydrated tissue; paraffin-embedded tissue; laser capture microdissection; or any combination thereof as long as they contain or are thought to contain nucleic acids or polypeptides of prostatic origin. Samples may be obtained by methods such as withdrawing fluid with a syringe or by a swab. One skilled in the art would readily recognize other methods of obtaining samples.

In another embodiment, samples of the present invention can also comprise multiple sub-samples, which can be obtained at the same time or spread over a period of time (e.g., urine or blood collected at different times, or multiple biopsy samples (e.g., multiple individual biopsy cores)). These sub-samples can then be processed at the same time or together (e.g., “pooled”).

Samples may be processed prior to analysis as long as the ability to detect the markers of the present invention is preserved. Sample processing may include preservation and storage, as well as treating the samples to physically disrupt tissue or cell structure, thus releasing intracellular components into a solution which may further contain enzymes, buffers, salts, detergents, and the like, which are used to prepare the sample for analysis. Cells may be isolated from a fluid sample such as with centrifugation, filtration or sedimentation. Body fluids such as urine and blood may require the addition of one or more stabilizing agents, such as when further testing is to be performed hours or days after sample collection. Further processing of the sample may require one or more storage or preservation steps to be reversed, such as the removal of stabilizing and preserving agents. Tissue samples may be homogenized or otherwise prepared for analysis by well-known techniques including but not limited to: sonication; mechanical disruption; chemical lysis such as detergent lysis; and combinations thereof. Samples may also be physically divided; exposed to a chemical reaction such as a deparaffinization and/or a precipitation procedure; exposed to a separation process such as separation in a centrifuge; exposed to a washing procedure; preserved; fixed; frozen; or the like. Samples, such as tissue may be frozen, dehydrated, or preserved with a chemical agent such as formalin. Fixed tissue samples may be embedded in paraffin which eases storage and transportation, as well as facilitates the creation of slides used by a pathologist to visually inspect and assess the sample, or frozen in a medium such as RNALater® or Trizol®. Tissue section preparation for surgical pathology may be frozen and prepared using standard techniques. Immunohistochemistry and in situ hybridization binding assays on tissue sections can be performed on fixed cells. The skilled person would readily appreciate the variety of samples that may be examined for a prostate cancer marker of the present invention, and recognize methods of obtaining, storing and preserving (if needed) the samples.

In accordance with the present invention, RNA may be extracted from biological sample in a number of ways, e.g., using an organic extraction or a solid surface target capture method. In one embodiment, the sample is urine and the RNA is extracted using one of the following extraction kits: ZR Urine RNA Isolation Kit™ (Zymo Research); Trizol™ LS (Invitrogen); Urine (Exfoliated Cell) RNA Purification Kit (Norgen Biotek cat.22500); Ribo-Sorb RNA/DNA extraction kit (Sacace); RNeasy™ mini kit (Qiagen). In another embodiment, the sample is human tissue and Trizol® reagent is used for the extraction process.

The preferred biological sample of the present invention is urine, although other samples (e.g., tissue) have been tested herein and are also envisioned. The fact that urine is so easy to collect and is herein validated for enabling clinical assessment such as diagnosis, prognosis, grade, etc., clearly supports the importance and power of the present invention. Urine samples may or may not be collected following an event such as a digital rectal exam, ejaculation, prostate massage, biopsy, or any other means which increase the content of prostate cells in the urine. The present can also be carried out using crude, unprocessed whole urine. As used herein, “crude urine” refers to urine that has been collected from a subject but has not been substantially further processed for example by centrifugation, filtration or sedimentation. Of course, urine fractions such as urine supernatant or urine cell pellets (e.g., urine sediments) can also be used in accordance with the present invention.

For a urine-based assay in which the prostate cancer markers of interest include nucleic acids (RNA or DNA), the urine may be stabilized as soon as possible after collection. Cellular components (including nucleic acids) can then be isolated from the urine for example, by filtering, centrifugation or sedimentation, followed by lysis of the isolated cells and stabilization of the RNA and/or DNA, such as through the use of a chaotropic agent like guanidium thiocyanate. The nucleic acids can then be removed, for example, via binding to a silica matrix.

In an assay using a blood sample, the whole blood or serum may be used or the blood plasma may be separated from the blood cells. The blood plasma may be screened for a prostate cancer marker of the present invention, including truncated proteins which are released into the blood when one or more prostate cancer markers of the present invention are cleaved from or sloughed off from tumor cells. In one embodiment, blood cell fractions are screened for the presence of prostate tumor cells. In another embodiment, lymphocytes present in the blood cell fraction can be screened by lysing the cells and detecting the presence of a marker of the present invention (e.g., a protein or a gene transcript), which may be present as a result of prostate tumor cells engulfed by the white blood cells.

Marker Expression Level Detection

In accordance with the present invention, a suitable biological sample is obtained from a subject having or suspected of having prostate cancer and the expression level of at least two prostate cancer markers of the present invention is determined. Briefly, the expression level can be obtained by detecting an amount of a target present in the sample, which is indicative of the expression level of the prostate cancer marker, and then processing or converting this raw target detection data (e.g., mathematically, statistically or otherwise) to produce an expression level of the prostate cancer marker in the sample, or some expression-related score.

As alluded to above, “target” refers to a specific sub-region of a marker of the present invention (non-limiting examples thereof comprising a chosen exon-exon junction in the case of an RNA marker, or chosen epitope in the case of a protein marker) that is targeted for detection, amplification and/or hybridization in accordance with a method of the present invention. Thus, in one embodiment, the determination of the expression level of a marker may begin with the detection of an amount of a target which is indicative/representative of the presence of the marker in the biological sample. That is, the amount of target detected can represent a surrogate to a quantity of the corresponding marker whose expression level is sought. The amount of target detected may be represented by one or more of the following: number of molecules/cells detected (e.g., cycle threshold (Ct) or Copy Number); mass detected; the concentration detected such as the ratio of the mass detected compared to sample mass or the ratio of mass detected compared to a patient parameter such as patient body mass or surface area; or any combination thereof.

The amount of target can be determined by measuring fluorescence output. The amount of target detected can also represent a surrogate to a quantity of the corresponding marker detected, such as a Ct (cycle threshold) value or Copy Number from a test measuring fluorescence output as a correlation to the target amount detected.

In one non-limiting embodiment, the marker of the present invention that is to be detected is a gene. Determination of the expression level of a gene target of the present invention can be done by quantifying an expression product of the gene (e.g., RNA or a polypeptide resulting therefrom). An RNA target can be quantified using any hybridization and/or amplification reaction or related technology known in the art. In another embodiment, the hybridization and/or amplification reaction (e.g., sequencing or amplification (e.g., PCR)) may utilize one or more oligonucleotides which are sufficiently complimentary to the RNA marker (or cDNA generated therefrom) to bind specifically thereto. In another embodiment, the oligonucleotide can be an amplification primer or a detection probe. Suitable oligonucleotides (e.g., amplification primers and probes) and amplification/hybridization reactions can be designed routinely by those having ordinary skill in the art using available sequence information. In another embodiment, the present invention includes labeled oligonucleotides (e.g., labeled with radiolabeled nucleotides or are otherwise detectable by readily available nonradioactive detection systems).

In fact, numerous detection and quantification technologies may be used to determine the expression level of the targets of the present invention, including but not limited to: PCR, RT-PCR; RT-qPCR; NASBA; Northern blot technology; a hybridization array; branched nucleic acid amplification/technology; TMA; LCR; High-throughput sequencing; in situ hybridization technology; and amplification process followed by HPLC detection or MALDI-TOF mass spectrometry. In a particular embodiment, an amplification process is performed by PCR. The marker detection methods described herein are meant to exemplify how the present invention may be practiced and are not meant to limit the scope of invention. It is contemplated that other sequence-based methodologies for detecting the presence of a marker of the present invention in a subject sample may be employed according to the invention. The foregoing is meant to be included within the scope of “amplification and/or hybridization reaction”.

In a typical PCR reaction, the RNA or cDNA is combined with the primers, free nucleotides and enzyme following standard PCR protocols and the mixture undergoes a series of temperature changes. If a marker of present invention or cDNA generated therefrom is present, that is, if both primers hybridize to target sequences on the same molecule, the molecule comprising the primers and the intervening complementary sequences will be exponentially amplified. The amplified DNA can be easily detected by a variety of well-known means. If the marker is absent, no PCR product will be exponentially amplified. The PCR technology therefore provides a reliable method of detecting a marker of the present invention.

In an embodiment, the PCR reaction may be configured or designed to amplify a specific exon-exon junction.

In some instances, such as when unusually small amounts of RNA are recovered and only small amounts of cDNA are generated therefrom, it may be desirable or necessary to perform a PCR reaction on the first PCR reaction product. That is, if it is difficult to detect quantities of amplified DNA produced by the first reaction, a second PCR can be performed to make multiple copies of DNA sequences of the first amplified DNA. A nested set of primers can be used in the second PCR reaction.

In situ hybridization technology is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express markers of the present invention. Probes preferably hybridize to a nucleotide sequence that corresponds to such markers. Hybridization conditions can be routinely optimized to minimize background signal by non-fully complementary hybridization. The probes are preferably fully complementary to their target sequence. Since probes do not hybridize as well to partially complementary sequences, full complementarity is often preferred. For in situ hybridization according to the invention, it is also preferred that the probes are labeled with fluorescent dye attached to the probes to be readily detectable by fluorescence.

In another embodiment, target detection may be accomplished by detection of a protein (or an epitope thereof) encoded by a gene or RNA marker of the present invention. Proteins and polypeptides can be quantified using methods routinely available in the art, as would be recognized by the skilled person. In another embodiment, an immunoassay can be used to determine the expression level of a polypeptide marker of the present invention. Techniques such as immunohistochemistry assays may be performed to determine whether markers of the present invention are present in cells in the sample. In another embodiment, protein markers of the present invention can be detected using marker-specific antibodies. In particular embodiment, the antibodies can be monoclonal antibodies, polyclonal antibodies, humanized antibodies or antibody fragments. Antibodies against the polypeptide markers of the present are available or can be readily produced by a person of ordinary skill in the art.

Once the amount of target of the present invention is obtained, the expression level of a corresponding marker can be determined for example to produce an expression level of the prostate cancer marker in the sample.

In one embodiment, determining the expression level of a marker of the present invention can include merely determining the presence (or lack thereof) of the marker (i.e., “yes” or “no”).

In another embodiment, determining the expression level of a marker of the present invention can include processing or converting the raw target detection data (e.g., mathematically, statistically or otherwise) into an expression level (or normalized expression level) of the prostate cancer marker using a statistical method (e.g., logistic regression) that takes into account subject data or other data. Subject data may include (but is not limited to): age; race; cancer stage, such as stage determined by histopathology; Gleason score (as determined by biopsies) or Gleason grade (as determined by a pathologist after prostatectomy); PSA level such as preoperative PSA level; PCA3 ratio, or other diagnosis such as HGPIN; BPH; or ASAP; or of course to different combinations of such subject data or other data. The algorithm may be or include a nomogram, as defined hereinabove. The algorithm may also take into account factors such as the presence, diagnosis and/or prognosis of a subjects condition other than (or in addition to) prostate cancer. In a particular embodiment, where the sample obtained from the subject is urine, the algorithm may take into account the timing of the urine sample collection relative to another event, such as digital rectal exam; prostate massage; biopsy; surgical prostate removal; first diagnosis of cancer; or any combination thereof. In another embodiment, the statistical method may process target amounts that represent levels for: number of cells detected; number of molecules detected; mass detected; concentration detected such as mass of marker detected compared to the mass of the sample or a sub-sample; and combinations of these. In another embodiment, the algorithm may be configured to determine a concentration of the target (e.g., amount of marker detected compared to another parameter). As will be clear to the skilled artisan to which the present invention pertains, from above and below, numerous combinations of data parameters and/or factors may be used by the algorithm or algorithms encompassed herein, to obtain the desired output.

In another embodiment, determination of expression level of a prostate cancer marker can involve determining the expression level of one or more alternative splice variants of this prostate cancer marker. In this embodiment, the presence or absence of an alternative splice variant is typically detected by RT-PCR using primers which bind specifically to the nucleotide sequences which flank the region or regions where alternative splicing occurs.

In another embodiment, determining the expression level of a marker of the present invention can include a comparison to one or more threshold values (e.g., above or below the threshold). In another embodiment, the expression level represents a quantitative or qualitative level or value, such as a value selected from a continuous range of values or a value selected from a range of multiple discrete values. The expression level may be based on a direct measurement of a marker of the present invention, or be based on the measurement of a normalized value.

Normalization with Control Markers

Following the expression level determination of markers of the present invention, the expression level can then be normalized for example using a normalization algorithm, mathematical process, or other data manipulation tool or method that uses one or more control markers (e.g., prostate-specific control marker, endogenous control marker, exogenous control marker). The normalized expression level of the prostate cancer marker may then be processed, e.g., through comparison to one or more thresholds including: classification into one or more discrete levels or groups; comparison to another method or clinical parameter of the sample or the subject; and/or other mathematical or non-mathematical transformations.

Generally, an expression level of a prostate cancer marker of the present invention is normalized to one or more control markers to produce a normalized expression level, as well-known to those of skill in the art. As used herein and as alluded above, a “control marker” refers to a particular type of marker that is useful (either individually or when combined with one or more control markers) to control for potential interfering factors and/or to provide one or more indications about sample quality, effective sample preparation, and/or proper reaction assembly/execution (e.g., of an RT-PCR reaction).

In one embodiment, suitable control markers of the present invention have an expression not affected by the presence of cancer cells in the sample, a behavior similar to the prostate cancer markers in samples somehow degraded because of long storage periods, poor storage conditions or other stress factors. The approach of normalizing prostate cancer markers with suitable control markers as shown herein provides a useful adjunct to current methods for enabling a clinical assessment of prostate cancer as early detection is desirable for effective treatment and management of cancer.

In one embodiment, control markers can be one or more of endogenous control markers, an exogenous control markers, and/or a prostate-specific control markers (e.g., PSA), as described herein. Control markers can be a combination of one or more endogenous genes such as housekeeping genes or prostate-specific control markers or genes.

In one embodiment, an endogenous control marker can include one or more endogenous genes (i.e., “endogenous control gene” or “reference gene”) whose expression is relatively stable (e.g., does not significantly vary in prostate-cancer versus non-prostate cancer samples, and/or from subject to subject) in the particular sample that is being tested (e.g., urine), as well as when the sample/markers are subjected to various processing steps, depending on the method used to determine the marker expression levels. The expression stability of endogenous control genes can be analyzed using for example a software (e.g., geNorm™), which uses a pair-wise comparison model to select a gene pair showing the least variation in expression ratio across samples.

In another embodiment, control markers used for normalization can include one or more prostate-specific control markers such as PSA, which can be useful for example for controlling for, or validating the presence of, prostate cells in the sample being tested. Examples of other control markers that can be included are ones that provide information relating to providing a clinical assessment to the subject, such as one or more control markers that are useful confirming or ruling out a disease/disorder other than prostate cancer (e.g., a non-prostate cancer cell proliferative disorder) as has been listed in Table 7B.

In one particular embodiment, the expression level of at least two prostate cancer markers of the present invention is determined from a urine sample, and the expression levels are normalized using one or more control markers that are substantially stable in urine (e.g., between urine from subjects having or lacking prostate cancer). In one such embodiment, the one or more control markers are selected from those listed in Table 2 or Tables 7-9. In another such embodiment, the one or more control markers comprise IPO8, POLR2A, GUSB, TBP, KLK3, or any combination thereof.

Prostate Cancer Score

Following data normalization, a mathematical correlation of the normalized expression levels of the at least two prostate cancer markers of the present invention is performed to obtain a “score” or “prostate cancer score”, which is then used to provide a clinical assessment of prostate cancer in the subject. In one embodiment, different scores can be obtained from multiple samples or sub-samples, which can be obtained at the same time or spread over a period of time (e.g., urine or blood collected at different times, or multiple biopsy samples (e.g., multiple individual biopsy cores)). The different scores can then be compared to provide a clinical assessment of prostate cancer.

In accordance with the present invention, performing a “mathematical correlation”, “mathematical transformation”, “statistical method”, or “clinical assessment algorithm” refers to any computational method or machine learning approach (or combinations thereof) that help associate the level of expression of at least two markers from a biological sample (e.g., urine) with a clinical assessment of prostate cancer, such as predicting, for example, the result of a prostate biopsy or assessing the need to perform a prostate biopsy. A person of ordinary skill in the art will appreciate that different computational methods/tools may be selected for providing the mathematical correlations of the present invention, such as logistic regression, top scoring pairs, neural network, linear and quadratic discriminant analysis (LQA and QDA), Naïve Bayes, Random Forest and Support Vector Machines. Some statistical methods require hyperparameters tuned prior to launching the final model on the training data. In Bayesian statistics, a hyperparameter is a parameter of a prior distribution (e.g., number of layers, number of nodes or the C parameter in SVM) whose numbers are left to be tuned manually using basic procedures such as a cross-validated grid search. The selection of parameters, such as normalized gene expression values or delta Cts, to be used in the models of the present invention, was performed by incrementally adding the top scoring genes defined by their discriminative p-values on the cross-validated training set and stopping adding the features when either the maximal number of genes was reached or the performance (AUC) stops improving.

As used herein the term “Naives Bayes” refers to a computational method where there is no covariance assumed between the delta Ct of gene A and delta Ct of gene B. The different weights given to the genes used in such a model are assumed to be independent of each other and are weighted equally. The parameters are estimated directly from the training set and consist of the mean and variance for each of the selected genes times two for the two classes. The likelihood that sample X belongs to class Y is estimated using the Gaussian distribution from the mean and variance estimated from the training set. The Naive Bayes method selects the most likely classification V_{nb} (e.g., Normal or Tumor) given the attribute values a₁; a₂; . . . aₙ in the corresponding function:

V_{nb}=(a₁, a₂, . . . , aₙ)=argmaxᵥ_{ⱼ}_{ε}_{^{v}}P(vⱼ)ΠP(aᵢ|vⱼ)

Where P(aᵢ|vⱼ) is generally estimated using normal distribution for which mean μᵥⱼ and standard deviation σᵥⱼ are estimated from the training set for every class and gene as in:

$$P ( {a}_{i} \vert {v}_{j} ) = \frac{1}{\sqrt{2 {πσ}_{vj}^{2}}} {e}^{\frac{- {( {a}_{i} - {μ}_{vj} )}^{2}}{2 {σ}_{vj}^{2}}}$$

and

aᵢ=the delta Ct of gene i

vⱼ=either tumor or normal

μᵥⱼ=the mean of class vⱼ and gene i

σᵥⱼ=the standard deviation of class vⱼ and gene i

As used herein, the term “Linear Discriminant Analysis (LDA)” refers to a computational method that is a subclass of “Quadratic Discriminant Analysis (QDA)”. The quadratic form, from which the linear case could be extrapolated, consists of a 2-dimension (2-D) plot in which the first dimension represents the delta Ct for gene A and the second dimension the delta Ct for gene B. For all the samples in the training set, an “X” is placed on the 2-D plot at coordinate (delta Ct gene A, delta Ct gene B) in the case of a normal sample and an “O” in the case of a tumor sample. The goal is to find a quadratic function ax²+by+c (where “+c” appears only in the linear form) that will separate the “X” from the “O”. This function is obtained by computing the mean delta CT for gene A and B for the two classes respectively as well as the covariance matrices for every class. In the case of the linear discriminant analysis, only one covariance matrix is computed for all the classes instead of two (e.g., one for each class). There is no hyperparameter for this approach.

As used herein the term “Random Forest” refers to a computational method that is based on the idea of using multiple different decision trees to compute the overall most predicted class (the mode). In a specific application, the mode will be either tumor or normal based on how many decision trees predicted the samples as tumor or normal. The class (tumor or normal) predicted by the majority is selected as the predicted class for the sample. The different decision trees used in this algorithm are trained on a randomly generated subset of the training set and on a randomly selected set of the variables. This is why this algorithm relies on two hyperparameters: the number of random trees to use, and the number of random variables used to train the different trees.

As used herein the term “Support Vector Machine (SVM)” refers to a computational method with a goal, contrary to other linear classification approaches like LDA, to find a line that will best separate the two classes (e.g., tumor or normal), this line being the farthest from any training points (maximum margin). This definition of the problem leads to a completely different cost function with interesting generalization property (the property of being as good on untested samples). SVM are sometimes used in combination with kernel function that transform the data in a way that could simplify the discrimination of the samples (finding a line that will discriminate the samples). The linear kernel, which is the default scheme using the data as is, as well as the Gaussian radial-kernel, that transforms the data using radial basis Gaussian function, can both be used, as shown herein. In the SVM approach, mislabeled training data C and the gamma of the Gaussian function of the radial-kernel are the hyperparameters. Those hyperparameters could be selected using a 2-D grid search and cross-validation.

In one embodiment, the mathematical correlation can produce a range of output clinical assessment values that comprise a continuous or near-continuous range of values, such as has been described above in reference to the expression level algorithm of the present invention. Alternatively, the clinical assessment algorithm may produce a range of output clinical assessment values that comprise a range of discrete values. In a particular embodiment, the range of output clinical assessment values is two discrete values, such as two clinical assessment values selected from or clinically similar to the following group: “yes” and “no”; “low” and “high”; “present” and “not present” such as in reference to the presence of cancer; “no prostate cancer cells detected” and “at least one prostate cancer cell detected”; “mild” and “severe” such as in reference to aggressiveness of cancer; “likely” and “unlikely” such as in reference to potential recurrence or initial onset of cancer; and other two level output clinical assessment relevant to a clinical assessment of a prostate cancer subject. Of course, it will be understood that other such two clinical assessment values can be easily chosen by the skilled artisan using the methods and kits of the present invention.

In a particular embodiment, the clinical assessment algorithm produces a range of output clinical assessment values comprising three or more discrete values, such as three or more values related to one or more of: aggressiveness of cancer; prognosis of success for a future therapy such as a future chemotherapy; a diagnosis and/or prognosis of success of a current therapy such as a current chemotherapy; likelihood of future cancer onset; likelihood of cancer recurrence; and likelihood of long term survival. In another particular embodiment, the range of output values is three or more discrete values, such as values selected from or clinically similar to the following group: aggressiveness values such as not aggressive, mildly aggressive and very aggressive; future onset or recurrence values such as unexpected, moderate chance and strong chance; success of therapy values such as unlikely, moderately likely and very likely; and other multi-level outputs relevant to the clinical assessment of a prostate cancer subject. Multiple discrete values can be qualitative assessments as described above, or quantitative ranges such as 0-100, where the maximum and minimum values represent the limits of the clinical assessment values.

In another embodiment, the clinical assessment algorithm may compare the (normalized) expression levels of the prostate cancer markers of the present invention to one or more thresholds (e.g., to classify them into two or more discrete clinical assessment values). In a particular embodiment, the threshold can enable classification into two or more discrete clinical assessment values relating to: presence of cancer or not; aggressiveness of cancer; stages of cancer; locations of cancer; Gleason scores; likelihood of developing cancer such as the likelihood of developing an aggressive cancer; likelihood of a therapy being successful such as a therapy involving one or more chemotherapeutic drugs; likelihood of achieving long-term survival; and other clinical assessment values. For example, a first clinical assessment value of “likely to respond” to a particular chemotherapeutic, may correspond to prostate cancer marker expression levels below a first threshold, and a second clinical assessment value of “moderately likely to respond” to that chemotherapeutic, may correspond to prostate cancer marker expression levels above a first threshold but below a second threshold. Accordingly, a third clinical assessment value of “unlikely to respond” to that chemotherapeutic agent may correspond to prostate cancer marker expression levels which are above the second threshold.

In particular embodiments, the threshold values of the present invention are preferably based on previous, and potentially current, testing of samples, known as positive or negative “control samples” or “training samples” from individuals with a confirmed diagnosis of prostate cancer, and from other individuals such as those with other non-prostate cancer diseases/disorders as well as healthy individuals. Determining the expression level(s) of prostate cancer markers by testing known healthy individuals and subjects with a confirmed diagnosis of prostate cancer allows the clinical assessment algorithm to identify the deterministic values for one or more thresholds, particularly as they relate to thresholds for determining the presence or absence prostate cancer. Thresholds may also be determined based on testing of control samples from individuals with a known history of one or more of: onset of cancer; presence of high grade cancer; recurrence of cancer; clinical success with one or more specific therapies such as a specific chemotherapeutic; and other known clinical outcomes. Alternatively or additionally, thresholds may be determined by testing a control sample from the same subject as is being tested according to the present invention, such as a sample taken at an earlier time. Preferably, testing of these types of control samples to determine one or more thresholds includes normalization of the expression level of the detected prostate cancer markers, such as normalization using one or more control markers.

In other embodiments, the threshold may be a quantity of zero, such as when any non-zero expression level of the prostate cancer markers correlates to a particular clinical assessment value, such as the presence of cancer. The threshold may be a non-zero minimum value, such as a value determined by testing of one or more control markers of the present invention. In further embodiments, one or more thresholds can be used to determine two or more clinical assessment values, respectively. In an alternative embodiment, two or more thresholds can be compared to the normalized expression levels of the prostate cancer markers and/or control markers of the present invention. In other embodiments, the same or different thresholds can be used for each marker.

Clinical Assessment of Prostate Cancer

A “score” or “prostate cancer score” (or comparison of various scores) of the present invention provides information to a clinician about prostate cancer status in a subject. As used herein, “clinical assessment” can include an evaluation of a patient's physical condition and prediction of the presence and/or degree of severity of prostate cancer and its evolution, as well as the prospect of recovery as anticipated from usual course of the disease and is based on information gathered from physical and laboratory examinations and the patient's medical history. In various embodiments, a clinical assessment of prostate cancer includes one or more of: prostate cancer screening, diagnosis, staging, prognosis, determination of aggressiveness, treatment planning, monitoring response to treatment, surveillance, and other clinical assessments of prostate cancer. More particularly, the clinical assessment may represent one or more of: a diagnosis such as a cancer screening assessment, a staging assessment or a cancer aggressiveness classification; a prognosis such as a treatment planning assessment, a cancer onset prognosis including differentiation between aggressiveness of the cancer, a cancer recurrence prognosis, an effectiveness of therapy prognosis, prognosis of long term survival; other clinical assessments for prostate cancer subjects or potential prostate cancer subjects; and any combination thereof. In another embodiment, the clinical assessment can include providing a stratified or otherwise differentiated assessment of benign prostate hyperplasia (BPH), or one or more cell proliferative disorders, such as prostate cancer; prostatic intraepithelial neoplasia (PIN), and small acinar proliferation (ASAP). In another embodiment, the clinical assessment can be used to determine a clinical course of prostate cancer care, including but not limited to: observation (watchful waiting); surgery such as prostatectomy; radiation therapy such as external beam radiation or brachytherapy; pharmaceutical or other agent therapy such as hormonal therapy or chemotherapy; testosterone lowering therapy such as via medication or surgical removal of the testis; and combinations of these.

In one embodiment, the clinical assessment of the present invention may be transferred or otherwise provided to an entity separate from the entity performing the test, such as a clinical assessment provided to a hospital or doctor's office by a Clinical Laboratory Improvement Amendments (CLIA) laboratory. In particular embodiment, the clinical assessment may be provided in one or more communicative forms, including verbal, electronic and tangible forms. In a preferred embodiment, the clinical assessment is provided in paper and/or electronic form, such as electronic form provided over wired or wireless communication means such as the Internet. In addition to the clinical assessment, the expression level of the prostate cancer markers of Table 5 or Table 6A of the present invention as well as the co-regulated markers of Table 6B may also be provided. In another embodiment, the score generated by the mathematical correlation of the present invention used to classify the expression level of the prostate cancer markers listed in Table 5 or Table 6A can be provided. In another embodiment, the clinical assessment can enable or include screening of individuals who are at high risk of developing prostate cancer, or who have been diagnosed with localized disease and/or metastasized disease, and/or those who are genetically linked to the disease. In another embodiment, the present invention can be used to monitor individuals who are undergoing and/or have been treated for primary prostate cancer to determine if the cancer has metastasized. In another embodiment, the present invention can also be used to monitor individuals who are undergoing and/or have been treated for prostate cancer to determine if the cancer has been eliminated. All of these uses are included within the scope of providing a clinical assessment.

In another embodiment, the present invention can be used to monitor individuals who are otherwise susceptible, i.e., individuals who have been identified as genetically predisposed to prostate cancer (e.g., by genetic screening and/or family histories). Advancements in the understanding of genetics and developments in technology/epidemiology enable improved probabilities and risk assessments relating to prostate cancer. Using family health histories and/or genetic screening, it is possible to estimate the probability that a particular individual has for developing certain types of cancer including prostate cancer. Those individuals that have been identified as being predisposed to developing a particular form of cancer can be monitored or screened to detect evidence of prostate cancer. Upon discovery of such evidence, early treatment can be undertaken to combat the disease. Accordingly, individuals who are at risk of developing prostate cancer may be identified and samples may be obtained from such individuals. In another embodiment, the present invention is also useful to monitor individuals who have been identified as having family medical histories which include relatives who have suffered from prostate cancer. Likewise, the invention is useful to monitor individuals who have been diagnosed as having prostate cancer and, particularly those who have been treated and had tumors removed and/or are otherwise experiencing remission including those who have been treated for prostate cancer. Moreover, in another embodiment, the present invention can be used to monitor individuals who have been diagnosed as having prostate cancer and, more particularly, those who are closely monitored for disease progression before receiving a treatment for the disease. All of these uses are included within the scope of providing a clinical assessment.

In another embodiment, the clinical assessment of prostate cancer in accordance with the present invention can further enable or include determining the particular or more suitable therapy that is to be given to a subject after the clinical assessment has been provided. Examples of applicable therapies include but are not limited to: surgery (e.g., prostatectomy); tumor destruction therapy (e.g., cryotherapy); radiation therapy (e.g., brachytherapy); and drug and other agent therapies (e.g., chemotherapy and hormone therapy).

Kits and Compositions

In various embodiments, numerous kits configurations are to be considered within the scope of the present invention. A kit may include one or more components, substances or pieces of equipment as has been described herein. The present invention further includes reagents and compositions useful as components in these kits. In other embodiments, the present invention relates to diagnostic compositions comprising reagents for detecting prostate cancer signatures of the present invention. In particular embodiments, the diagnostic composition further comprises urine, blood, tissue or a nucleic acid extract therefrom.

In one embodiment, the kit or compositions can include at least one oligonucleotide (e.g., probe or primer) that hybridizes to one or more of:

(1) a nucleic acid sequence according to a prostate cancer marker of the present invention;

(2) a polynucleotide encoding a protein of a prostate cancer marker of the present invention;

(3) a sequence which is fully complementary to (1) or (2); or

(4) a sequence which hybridizes under high stringency conditions to (1), (2) or (3);

In another embodiment, the present invention relates to a kit or composition comprising reagents enabling the detection of at least two prostate cancer markers (e.g., RNA markers) of the present invention.

In another embodiment, the kits of the present invention preferably include a container for transporting the sample, such as a container for transporting urine or blood.

In another embodiment, the kits or compositions of the present invention preferably also include at least one oligonucleotide (e.g., probe or primer) that hybridizes to one or more of:

(1) a nucleic acid sequence according to a control marker of the present invention;

(2) a polynucleotide encoding a protein of a control marker of the present invention;

(3) a sequence which is fully complementary to (1) or (2); or

(4) a sequence which hybridizes under high stringency conditions to (1), (2) or (3).

It should be understood that numerous other configurations of the methods, reagents and kits described herein can be employed without departing from the spirit or scope of this application. Portions of the methods described above may individually be considered a unique invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed and/or combine one or more steps, and it is intended that the particular steps of the method or procedure claim set forth herein below not be construed as being order-specific unless such order specificity is expressly stated in the claim.

TABLE 1
List of Candidate Markers Selected for Gene Expression Profiling

| Gene | Official | | Accession | Amplicon | Associated |
|---|---|---|---|---|---|
| ID | Symbol | Gene Name | Number | Size | SNP(s) |
| 23461 | ABCA5 | ATP-binding cassette, sub-family A (ABC1), | NM_018672 | 100 | |
| | | member 5 | | | |
| 10257 | ABCC4 | ATP-binding cassette, sub-family C | NM_005845 | 63 | |
| | | (CFTR/MRP), member 4 | | | |
| 116285 | ACSM1 | acyl-CoA synthetase medium-chain family | NM_052956 | 74 | |
| | | member 1 | | | |
| 59 | ACTA2 | actin, alpha 2, smooth muscle, aorta | NM_001141945 | 64 | |
| 70 | ACTC1 | actin, alpha, cardiac muscle 1 | NM_005159 | 70 | |
| 2515 | ADAM2 | ADAM metallopeptidase domain 2 | NM_001464 | 78 | |
| 177 | AGER | advanced glycosylation end product-specific | NM_172197 | 70 | |
| | | receptor | | | |
| 221120 | ALKBH3 | alkylation repair homolog 3 | NM_139178 | 74 | |
| 23600 | AMACR | alpha-methylacyl-CoA racemase | NM_014324 | 97 | rs76184600 |
| 272 | AMPD3 | adenosine monophosphate deaminase 3 | NM_000480 | 83 | |
| 301 | ANXA1 | annexin A1 | NM_000700 | 66 | |
| 9411 | ARHGAP29 | Rho GTPase activating protein 29 | NM_004815 | 69 | rs79740616 |
| 26084 | ARHGEF26 | Rho guanine nucleotide exchange factor | NM_015595 | 76 | |
| | | (GEF) 26 | | | |
| 51309 | ARMCX1 | armadillo repeat containing, X-linked 1 | NM_016608 | 75 | |
| 477 | ATP1A2 | ATPase, Na+/K+ transporting, alpha 2 | NM_000702 | 57 | |
| | | polypeptide | | | |
| 493 | ATP2B4 | ATPase, Ca++ transporting, plasma | NM_001684 | 84 | |
| | | membrane 4 | | | |
| 540 | ATP7B | ATPase, Cu++ transporting, beta | AB209461 | 83 | |
| | | polypeptide | | | |
| 389206 | BEND4 | BEN domain containing 4 | NM_207406 | 52 | |
| 387882 | C12orf75 | chromosome 12 open reading frame 75 | NM_001145199 | 81 | |
| 776 | CACNA1D | calcium channel, voltage-dependent, L type, | NM_000720 | 69 | rs72556363 |
| | | alpha 1D subunit | | | |
| 10645 | CAMKK2 | calcium/calmodulin-dependent protein | NM_006549 | 93 | |
| | | kinase kinase 2, beta | | | |
| 822 | CAPG | capping protein (actin filament), gelsolin-like | NM_001747 | 58 | |
| 857 | CAV1 | caveolin 1 | NM_001753 | 66 | |
| 1066 | CES1 | carboxylesterase 1 | NM_001025195 | 69 | |
| 10370 | CITED2 | Cbp/p300-interacting transactivator 2 | NM_006079 | 80 | |
| 1191 | CLU | clusterin (non-protein coding) | NR_038335 | 65 | |
| 1308 | COL17A1 | collagen, type XVII, alpha 1 | NM_000494 | 64 | |
| 1280 | COL2A1 | collagen, type II, alpha 1 | NM_001844 | 70 | rs11168349 |
| 148327 | CREB3L4 | cAMP responsive element binding protein | NM_130898 | 95 | rs41308369, |
| | | 3-like 4 | | | rs34612917 |
| 10321 | CRISP3 | cysteine-rich secretory protein 3 | NM_006061 | 111 | |
| 1410 | CRYAB | crystallin, alpha B | NM_001885 | 66 | |
| 1465 | CSRP1 | cysteine and glycine-rich protein 1 | NM_004078 | 77 | rs34504522 |
| 1475 | CSTA | cystatin A | NM_005213 | 114 | rs34145621 |
| 1501 | CTNND2 | catenin (cadherin-associated protein), delta | NM_001332 | 102 | |
| | | 2 | | | |
| 51700 | CYB5R2 | cytochrome b5 reductase 2 | NM_016229 | 81 | |
| 55510 | DDX43 | DEAD (Asp-Glu-Ala-Asp) box polypeptide | NM_018665 | 60 | |
| | | 43 | | | |
| 1745 | DLX1 | distal-less homeobox 1 | NM_178120 | 95 | |
| 54431 | DNAJC10 | DnaJ (Hsp40) homolog, subfamily C, | NM_018981 | 65 | rs34783249 |
| | | member 10 | | | |
| 1855 | DVL1 | dishevelled, dsh homolog 1 (Drosophila) | NM_004421 | 51 | |
| 1871 | E2F3 | E2F transcription factor 3 | NM_001949 | 139 | |
| 2202 | EFEMP1 | EGF containing fibulin-like extracellular | NM_001039348 | 86 | |
| | | matrix protein 1 | | | |
| 1946 | EFNA5 | ephrin-A5 | NM_001962 | 98 | |
| 10278 | EFS | embryonal Fyn-associated substrate | NM_005864 | 66 | rs2231798 |
| 2000 | ELF4 | E74-like factor 4 | NM_001421 | 65 | |
| 4072 | EPCAM | epithelial cell adhesion molecule | NM_002354 | 64 | |
| 2078 | ERG | v-ets erythroblastosis virus E26 oncogene | NM_182918 | 60 | |
| | | homolog | | | |
| 51290 | ERGIC2 | ERGIC and golgi 2 | NM_016570 | 96 | |
| 2146 | EZH2 | enhancer of zeste homolog 2 | NM_152998.2 | 75 | |
| 2171 | FABP5 | fatty acid binding protein 5 | NM_001444 | 91 | rs541099, |
| | | | | | rs61744912 |
| 2194 | FASN | fatty acid synthase | NM_004104 | 62 | |
| 2203 | FBP1 | fructose-1,6-bisphosphatase 1 | NM_000507 | 81 | |
| 2253 | FGF8 | fibroblast growth factor 8 (androgen- | NM_033165 | 76 | |
| | | induced) | | | |
| 2263 | FGFR2 | fibroblast growth factor receptor 2 | NM_000141 | 77 | |
| 2316 | FLNA | filamin A, alpha | NM_001456 | 73 | |
| 2318 | FLNC | filamin C, gamma | NM_001458 | 71 | |
| 2330 | FMO5 | flavin containing monooxygenase 5 | NM_001461 | 80 | |
| 57600 | FNIP2 | folliculin interacting protein 2 | NM_020840 | 64 | |
| 2346 | FOLH1 | folate hydrolase (prostate-specific | NM_004476 | 110 | rs79155991, |
| | | membrane antigen) 1 | | | rs75111588 |
| 219595 | FOLH1B | folate hydrolase 1B | NM_153696 | 102 | |
| 3169 | FOXA1 | forkhead box A1 | NM_004496 | 74 | rs80196093 |
| 2294 | FOXF1 | forkhead box F1 | NM_001451 | 69 | |
| 2295 | FOXF2 | forkhead box F2 | NM_001452.1 | 77 | rs11759800 |
| 122786 | FRMD6 | FERM domain containing 6 | NM_001042481 | 67 | rs78316801 |
| 2591 | GALNT3 | UDP-N-acetyl-alpha-D- | NM_004482 | 66 | |
| | | galactosamine:polypeptide N- | | | |
| | | acetylgalactosaminyltransferase 3 (GalNAc- | | | |
| | | T3) | | | |
| 51809 | GALNT7 | UDP-N-acetyl-alpha-D- | NM_017423 | 78 | |
| | | galactosamine:polypeptide N- | | | |
| | | acetylgalactosaminyltransferase 7 | | | |
| 284161 | GDPD1 | glycerophosphodiester phosphodiesterase | NM_182569 | 69 | |
| | | domain containing 1 | | | |
| 2762 | GMDS | GDP-mannose 4,6-dehydratase | NM_001500 | 99 | |
| 2768 | GNA12 | guanine nucleotide binding protein (G | NM_007353 | 69 | rs12721531 |
| | | protein) alpha 12 | | | |
| 51280 | GOLM1 | golgi membrane protein 1 | NM_016548 | 88 | rs77104922 |
| 26996 | GPR160 | G protein-coupled receptor 160 | NM_014373 | 77 | |
| 2950 | GSTP1 | glutathione S-transferase 1 | NM_000852 | 54 | rs45458200, |
| | | | | | rs45485891, |
| | | | | | rs8191444, |
| | | | | | rs8191439 |
| 2982 | GUCY1A3 | guanylate cyclase 1, soluble, alpha 3 | NM_000856 | 75 | |
| 2990 | GUSB | glucuronidase, beta | NM_000181 | 96 | |
| 3092 | HIP1 | huntingtin interacting protein 1 | AF365404 | 69 | |
| 3109 | HLA-DMB | major histocompatibility complex, class II, | NM_002118 | 75 | |
| | | DM beta | | | |
| 3221 | HOXC4 | homeobox C4 | NM_014620 | 85 | rs17854635, |
| | | | | | rs75256744 |
| 3222 | HOXC5 | homeobox C5 | NM_018953.2 | 76 | |
| 3223 | HOXC6 | homeobox C6 | NM_004503 | 87 | |
| 3224 | HOXC8 | homeobox C8 | NM_022658 | 80 | |
| 3249 | HPN | hepsin (TMPRSS1) | NM_182983 | 89 | |
| 3251 | HPRT1 | hypoxanthine phosphoribosyltransferase 1 | NM_000194 | 72 | |
| 3257 | HPS1 | Hermansky-Pudlak syndrome 1 | NM_000195 | 74 | |
| 51170 | HSD17B11 | hydroxysteroid (17-beta) dehydrogenase 11 | NM_016245 | 60 | |
| 8630 | HSD17B6 | hydroxysteroid (17-beta) dehydrogenase 6 | NM_003725 | 84 | |
| 7923 | HSD17B8 | hydroxysteroid (17-beta) dehydrogenase 8 | NM_014234 | 90 | |
| 3400 | ID4 | inhibitor of DNA binding 4, dominant | NM_001546 | 54 | |
| | | negative helix-loop-helix protein | | | |
| 3611 | ILK | integrin-linked kinase | NM_004517 | 70 | rs56057203 |
| 10526 | IPO8 | importin 8 | NM_006390 | 71 | |
| 9903 | KLHL21 | kelch-like 21 (Drosophila) | AB007938 | 70 | rs2232460 |
| 354 | KLK3 | kallikrein-related peptidase 3 | NM_001648 | 83 | rs11573 |
| 3866 | KRT15 | keratin 15 | NM_002275 | 81 | rs2305556 |
| 3852 | KRT5 | keratin 5 | NM_000424 | 133 | |
| 3914 | LAMB3 | laminin, beta 3 | NM_000228 | 69 | |
| 55353 | LAPTM4B | lysosomal protein transmembrane 4 beta | NM_018407 | 80 | |
| 3964 | LGALS8 | lectin, galactoside-binding, soluble, 8 | NM_006499 | 86 | rs2737713, |
| | | | | | rs1041934, |
| | | | | | rs74151924 |
| 4008 | LMO7 | LIM domain 7 | NM_005358 | 62 | rs75375399 |
| 7216 | MAGED3 | trophinin | NM_001039705 | 76 | |
| 728239 | MAGED4 | melanoma antigen family D, 4 | NM_001098800 | 58 | |
| 4129 | MAOB | monoamine oxidase B | NM_000898 | 65 | |
| 9053 | MAP7 | microtubule-associated protein 7 | NM_001198608 | 91 | |
| 64087 | MCCC2 | methylcrotonoyl-CoA carboxylase 2 | NM_022132 | 76 | rs34253895 |
| 4212 | MEIS2 | Meis homeobox 2 | NM_001220482 | 59 | |
| 744 | MPPED2 | metallophosphoesterase domain containing | NM_001584 | 71 | |
| | | 2 | | | |
| 10205 | MPZL2 | myelin protein zero-like 2 | NM_005797 | 82 | |
| 4638 | MYLK | myosin light chain kinase | NM_053025 | 69 | rs75383538 |
| 4646 | MY06 | myosin VI | NM_004999 | 70 | |
| 26509 | MYOF | myoferlin | NM_013451 | 68 | |
| 89797 | NAV2 | neuron navigator 2 | NM_182964 | 56 | |
| 8204 | NRIP1 | nuclear receptor interacting protein 1 | BE792046 | 127 | |
| 143503 | OR51E1 | olfactory receptor, family 51, subfamily E, | NM_152430 | 97 | rs1873974 |
| | | member 1 | | | |
| 81285 | OR51E2 | olfactory receptor, family 51, subfamily E, | NM_030774 | 61 | |
| | | member 2 | | | |
| 9506 | PAGE4 | P antigen family, member 4 (prostate | NM_007003 | 88 | |
| | | associated) | | | |
| 25849 | PARM1 | prostate androgen-regulated mucin-like | NM_015393 | 62 | |
| | | protein 1 | | | |
| 50652 | PCA3 | prostate cancer antigen 3 (non-protein | NR_015342 | 52 | |
| | | coding) | | | |
| 64002 | PCGEM1 | prostate-specific transcript 1 (non-protein | NR_002769 | 94 | rs13404783, |
| | | coding) | | | rs13418130 |
| 23037 | PDZD2 | PDZ domain containing 2 | NM_178140 | 83 | |
| 5192 | PEX10 | peroxisomal biogenesis factor 10 | NM_153818 | 98 | rs61752096 |
| 5300 | PIN1 | peptidylprolyl cis/trans isomerase, NIMA- | NM_006221 | 118 | rs79067653 |
| | | interacting 1 | | | |
| 7941 | PLA2G7 | phospholipase A2, group VII (platelet- | NM_005084 | 71 | |
| | | activating factor acetylhydrolase, plasma) | | | |
| 56937 | PMEPA1 | prostate transmembrane protein, androgen | NM_020182 | 77 | |
| | | induced 1 | | | |
| 5425 | POLD2 | polymerase (DNA directed), delta 2, | NM_001127218 | 70 | |
| | | regulatory subunit | | | |
| 5430 | POLR2A | polymerase (RNA) II (DNA directed) | NM_000937 | 61 | |
| | | polypeptide A | | | |
| 5457 | POU4F1 | POU class 4 homeobox 1 | NM_006237 | 104 | |
| 5507 | PPP1R3C | protein phosphatase 1, regulatory (inhibitor) | NM_005398 | 61 | |
| | | subunit 3C | | | |
| 5530 | PPP3CA | protein phosphatase 3, catalytic subunit, | NM_000944 | 88 | |
| | | alpha isozyme | | | |
| 8000 | PSCA | prostate stem cell antigen | NM_005672 | 82 | |
| 11156 | PTP4A3 | protein tyrosine phosphatase type IVA, | NM_032611 | 112 | |
| | | member 3 | | | |
| 83871 | RAB34 | RAB34, member RAS oncogene family | NM_031934 | 99 | rs11545697 |
| 57186 | RALGAPA2 | Ral GTPase activating protein, alpha | NM_020343 | 67 | rs6112935 |
| | | subunit 2 | | | |
| 5909 | RAP1GAP | RAP1 GTPase activating protein | NM_001145658 | 96 | rs61014678 |
| 11186 | RASSF1A | Ras association domain family member 1 | NM_007182 | 55 | |
| 83998 | REG4 | regenerating islet-derived family, member 4 | NM_001159352 | 58 | rs77250186 |
| 200916 | RPL22L1 | ribosomal protein L22-like 1 | NM_001099645 | 136 | |
| 6277 | S100A6 | S100 calcium binding protein A6 | NM_014624 | 94 | |
| 6279 | S100A8 | S100 calcium binding protein A8 | NM_002964 | 70 | |
| 6280 | S100A9 | S100 calcium binding protein A9 | NM_002965 | 83 | |
| 221935 | SDK1 | sidekick homolog 1, cell adhesion molecule | NM_152744 | 57 | |
| 6401 | SELE | selectin E | NM_000450 | 83 | |
| 57630 | SH3RF1 | SH3 domain containing ring finger 1 | NM_020870 | 59 | |
| 6493 | SIM2 | single-minded homolog 2 | NM_005069 | 69 | |
| 8501 | SLC43A1 | solute carrier family 43, member 1 | NM_003627 | 58 | |
| 6546 | SLC8A1 | solute carrier family 8 (sodium/calcium | NM_021097 | 73 | |
| | | exchanger), member 1 | | | |
| 84189 | SLITRK6 | SLIT and NTRK-like family, member 6 | NM_032229 | 144 | rs12863734, |
| | | | | | rs9566107 |
| 6591 | SNAI2 | snail homolog 2 (Drosophila) | NM_003068 | 79 | rs11544360 |
| 6690 | SPINK1 | serine peptidase inhibitor, Kazal type 1 | NM_003122 | 85 | rs35877720, |
| | | | | | rs17107315 |
| 10417 | SPON2 | spondin 2, extracellular matrix protein | NM_012445 | 104 | |
| 6715 | SRD5A1 | steroid-5-alpha-reductase, alpha | NM_001047 | 75 | |
| | | polypeptide 1 | | | |
| 6716 | SRD5A2 | steroid-5-alpha-reductase, alpha | NM_000348 | 83 | |
| | | polypeptide 2 | | | |
| 26872 | STEAP1 | six transmembrane epithelial antigen of the | NM_012449 | 78 | |
| | | prostate 1 | | | |
| 23336 | SYNM | synemin, intermediate filament protein | NM_145728 | 92 | rs5030691 |
| 6876 | TAGLN | transgelin | NM_003186 | 82 | |
| 6908 | TBP | TATA box binding protein | NM_003194 | 65 | |
| 140597 | TCEAL2 | transcription elongation factor A (SII)-like 2 | NM_080390 | 117 | |
| 56165 | TDRD1 | tudor domain containing 1 | NM_198795 | 67 | |
| 7031 | TFF1 | trefoil factor 1 | NM_003225 | 79 | |
| 7033 | TFF3 | trefoil factor 3 | NM_003226 | 64 | |
| 7060 | THBS4 | thrombospondin 4 | NM_003248 | 96 | |
| 130733 | TMEM178 | transmembrane protein 178 | NM_152390 | 62 | |
| 84899 | TMTC4 | transmembrane and tetratricopeptide repeat | NM_032813 | 84 | |
| | | containing 4 | | | |
| 10188 | TNK2 | tyrosine kinase, non-receptor, 2 | NM_005781 | 95 | rs56161912 |
| 8626 | TP63 | tumor protein p63 | NM_003722 | 75 | |
| 7163 | TPD52 | tumor protein D52 | NM_001025252 | 60 | |
| 7169 | TPM2 | tropomyosin 2 | NM_003289 | 56 | |
| 10221 | TRIB1 | tribbles homolog 1 | NM_025195 | 78 | |
| 23650 | TRIM29 | tripartite motif containing 29 | NM_012101 | 82 | |
| 79054 | TRPM8 | transient receptor potential cation channel, | NM_024080 | 77 | |
| | | subfamily M, member 8 | | | |
| 10103 | TSPAN1 | tetraspanin 1 | NM_005727 | 87 | |
| 27075 | TSPAN13 | tetraspanin 13 | NM_014399 | 63 | |
| 7272 | TTK | TTK protein kinase | NM_003318 | 64 | rs16891423, |
| | | | | | rs1801379 |
| 7291 | TWIST1 | twist homolog 1 | NM_000474 | 115 | |
| 6675 | UAP1 | UDP-N-acteylglucosamine | NM_003115 | 99 | |
| | | pyrophosphorylase 1 | | | |
| 7316 | UBC | ubiquitin C | NM_021009 | 71 | rs73417486, |
| | | | | | rs12302110, |
| | | | | | rs8397, |
| | | | | | rs41276688 |
| 7371 | UCK2 | uridine-cytidine kinase 2 | NM_012474 | 72 | |
| 9341 | VAMP3 | vesicle-associated membrane protein 3 | NM_004781 | 82 | rs57351330 |
| 115825 | WDFY2 | WD repeat and FYVE domain containing 2 | NM_052950 | 70 | |
| 10406 | WFDC2 | WAP four-disulfide core domain 2 | NM_006103 | 60 | rs6017577 |
| 7466 | WFS1 | Wolfram syndrome 1 | NM_006005 | 81 | |
| 25937 | WWTR1 | WW domain containing transcription | NM_015472 | 72 | |
| | | regulator 1 | | | |
| 92822 | ZNF276 | zinc finger protein 276 | NM_152287 | 79 | rs17719249 |
| 7551 | ZNF3 | zinc finger protein 3 | NM_017715 | 82 | |

TABLE 2
List of Endogenous Control Markers Evaluated for Gene Expression Normalization

| | Official | | Accession | Amplicon | |
|---|---|---|---|---|---|
| | Symbol | Gene Name | Number | Size | TaqMan Assay |
| Endogenous | GUSB | glucuronidase, beta | NM_000181 | 96 | Hs00939627_m1 |
| control | HPRT1 | hypoxanthine phosphoribosyltransferase 1 | NM_000194 | 72 | Hs01003267_m1 |
| markers | IPO8 | importin 8 | NM_006390 | 71 | Hs00183533_m1 |
| | POLR2A | polymerase (RNA) II (DNA directed) polypeptide A | NM_000937 | 61 | Hs00172187_m1 |
| | TBP | TATA box binding protein | NM_003194 | 65 | Hs00427621_m1 |
| Prostate Specific | KLK3 | kallikrein-related peptidase 3 | NM_001648 | 83 | Hs02576345_m1 |
| Control | FOLH1 | folate hydrolase (prostate-specific membrane antigen) 1 | NM_004476 | 110 | Hs00379515_m1 |
| Markers | FOLH1B | folate hydrolase 1B | NM_153696 | 102 | Hs00189528_m1 |
| | OR51E1 | olfactory receptor, family 51, subfamily E, member 1 | NM_152430 | 97 | Hs00379183_m1 |
| | OR51E2 | olfactory receptor, family 51, subfamily E, member 2 | NM_030774 | 61 | Hs04231197_m1 |
| | PCGEM1 | prostate-specific transcript 1 (non-protein coding) | NR_002769 | 94 | Hs01369007_m1 |
| | PMEPA1 | prostate transmembrane protein, androgen induced 1 | NM_020182 | 77 | Hs00375306_m1 |
| | PSCA | prostate stem cell antigen | NM_005672 | 82 | Hs00194665_m1 |

TABLE 3A
Expression Characteristics of Candidate Markers in Whole Urine Samples

| | Official | Mean DeltaCt | | Difference | t-test p | |
|---|---|---|---|---|---|---|
| Rank | Symbol | Normal (n = 45) | Tumor (n = 45) | in means | value | AUC |
| 1 | ERG | 4.9593 | 2.5004 | 2.4589 | 0.0002 | 0.7205 |
| 2 | PCA3 | −0.6432 | −1.8375 | 1.1943 | 0.0015 | 0.6775 |
| 3 | CACNA1D | 5.4588 | 4.0689 | 1.3899 | 0.0084 | 0.6869 |
| 4 | AMACR | 1.2009 | 0.5896 | 0.6113 | 0.0114 | 0.6721 |
| 5 | ADAM2 | 0.0746 | −0.8439 | 0.9186 | 0.0131 | 0.6825 |
| 6 | HPN | −0.1870 | −0.7806 | 0.5936 | 0.0134 | 0.6449 |
| 7 | SPON2 | 0.5864 | −0.3950 | 0.9813 | 0.0166 | 0.6780 |
| 8 | ACTA2 | 4.3714 | 3.4700 | 0.9014 | 0.0186 | 0.6193 |
| 9 | OR51E2 | 0.3373 | −0.7410 | 1.0783 | 0.0197 | 0.6711 |
| 10 | HOXC6 | 5.2894 | 4.1389 | 1.1505 | 0.0346 | 0.6311 |
| 11 | COL2A1 | 7.8097 | 6.5850 | 1.2247 | 0.0385 | 0.6030 |
| 12 | GOLM1 | 2.1220 | 1.4886 | 0.6333 | 0.0412 | 0.6351 |
| 13 | SDK1 | 6.0585 | 4.9567 | 1.1018 | 0.0419 | 0.6089 |
| 14 | TAGLN | 4.6389 | 3.4788 | 1.1601 | 0.0451 | 0.6040 |
| 15 | TDRD1 | 4.1210 | 2.9354 | 1.1856 | 0.0454 | 0.6622 |
| 16 | FMO5 | 1.7495 | 1.0971 | 0.6524 | 0.0481 | 0.6281 |
| 17 | LAMB3 | 2.5609 | 1.5388 | 1.0221 | 0.0483 | 0.6025 |
| 18 | HPRT1 | 0.8885 | 0.3233 | 0.5652 | 0.0555 | 0.6217 |
| 19 | TSPAN1 | 2.2670 | 1.7738 | 0.4932 | 0.0652 | 0.6311 |
| 20 | GUCY1A3 | −0.0444 | −0.7551 | 0.7107 | 0.0652 | 0.6479 |
| 21 | TPM2 | 6.4831 | 5.7103 | 0.7728 | 0.0822 | 0.6030 |
| 22 | LAPTM4B | 0.7354 | 0.0108 | 0.7247 | 0.0942 | 0.5911 |
| 23 | SLITRK6 | 7.7499 | 8.4941 | −0.7442 | 0.0948 | 0.5773 |
| 24 | MAOB | 3.1672 | 2.3322 | 0.8350 | 0.0964 | 0.5822 |
| 25 | DVL1 | 0.7329 | 0.1238 | 0.6091 | 0.0974 | 0.5560 |
| 26 | KRT15 | 0.0300 | −0.9135 | 0.9434 | 0.0997 | 0.5916 |
| 27 | TFF3 | 1.1851 | 0.2327 | 0.9524 | 0.1007 | 0.6000 |
| 28 | S100A8 | −5.1128 | −4.4034 | −0.7094 | 0.1173 | 0.5778 |
| 29 | GALNT7 | 0.6889 | 0.1107 | 0.5782 | 0.1233 | 0.5931 |
| 30 | FNIP2 | 1.0091 | 0.5104 | 0.4987 | 0.1283 | 0.5857 |
| 31 | HSD17B6 | 2.4826 | 1.8337 | 0.6489 | 0.1295 | 0.6010 |
| 32 | EPCAM | 3.0116 | 2.5774 | 0.4343 | 0.1360 | 0.6193 |
| 33 | HOXC4 | 5.8213 | 5.0013 | 0.8200 | 0.1373 | 0.6163 |
| 34 | TNK2 | 1.7087 | 1.1240 | 0.5848 | 0.1403 | 0.5862 |
| 35 | POLR2A | −1.9575 | −1.6047 | −0.3527 | 0.1450 | 0.5630 |
| 36 | RASSF1A | 1.0152 | 1.8134 | −0.7982 | 0.1528 | 0.5941 |
| 37 | SNAI2 | 2.8972 | 3.8911 | −0.9938 | 0.1539 | 0.5783 |
| 38 | FRMD6 | 2.0580 | 1.4153 | 0.6428 | 0.1704 | 0.5170 |
| 39 | FBP1 | −1.1715 | −1.3830 | 0.2116 | 0.1870 | 0.5699 |
| 40 | OR51E1 | 3.2073 | 2.4405 | 0.7668 | 0.1881 | 0.5975 |
| 41 | WWTR1 | 0.3107 | −0.1570 | 0.4677 | 0.2040 | 0.5862 |
| 42 | NRIP1 | 1.0006 | 0.4750 | 0.5256 | 0.2146 | 0.6079 |
| 43 | S100A9 | −2.8768 | −2.2992 | −0.5776 | 0.2159 | 0.5541 |
| 44 | TWIST1 | 5.6257 | 4.8625 | 0.7632 | 0.2166 | 0.5748 |
| 45 | MYO6 | 0.4742 | 0.1037 | 0.3705 | 0.2197 | 0.5640 |
| 46 | ARHGEF26 | 2.9437 | 2.3977 | 0.5460 | 0.2214 | 0.5822 |
| 47 | TSPAN13 | −0.0471 | −0.5568 | 0.5097 | 0.2326 | 0.5432 |
| 48 | GUSB | 0.5015 | 0.7722 | −0.2707 | 0.2450 | 0.5788 |
| 49 | PTP4A3 | 1.3806 | 1.1068 | 0.2738 | 0.2591 | 0.6247 |
| 50 | RAP1GAP | 1.1255 | 0.7497 | 0.3758 | 0.2626 | 0.5921 |
| 51 | NAV2 | 3.0146 | 2.5470 | 0.4676 | 0.2676 | 0.5798 |
| 52 | SRD5A1 | 0.4345 | 0.0397 | 0.3949 | 0.2688 | 0.5615 |
| 53 | GALNT3 | 1.2496 | 1.0449 | 0.2047 | 0.2738 | 0.5467 |
| 54 | WFDC2 | −0.5945 | −1.0906 | 0.4961 | 0.3091 | 0.5388 |
| 55 | TFF1 | 0.9095 | 0.5238 | 0.3857 | 0.3203 | 0.5664 |
| 56 | PLA2G7 | −0.7933 | −0.4972 | −0.2961 | 0.3284 | 0.5738 |
| 57 | MEIS2 | 1.2066 | 0.7786 | 0.4280 | 0.3353 | 0.5531 |
| 58 | TMEM178 | 1.9390 | 1.4260 | 0.5130 | 0.3354 | 0.6030 |
| 59 | MPPED2 | 1.1804 | 1.5077 | −0.3273 | 0.3372 | 0.5348 |
| 60 | TBP | 1.3446 | 1.5182 | −0.1737 | 0.3379 | 0.5551 |
| 61 | FLNC | 6.3375 | 5.8175 | 0.5200 | 0.3605 | 0.5714 |
| 62 | TRIB1 | 0.3232 | 0.6123 | −0.2891 | 0.3613 | 0.5185 |
| 63 | FOXF1 | 8.6553 | 8.9445 | −0.2892 | 0.3637 | 0.5328 |
| 64 | SYNM | 1.8368 | 1.5249 | 0.3118 | 0.3685 | 0.5832 |
| 65 | FOLH1 | −0.4049 | −0.6757 | 0.2708 | 0.3686 | 0.5798 |
| 66 | ERGIC2 | 1.8474 | 2.0856 | −0.2382 | 0.3718 | 0.5521 |
| 67 | ABCC4 | −1.3192 | −1.5287 | 0.2095 | 0.3756 | 0.5299 |
| 68 | FGF8 | 8.8134 | 8.4956 | 0.3179 | 0.3760 | 0.5422 |
| 69 | SPINK1 | 0.0973 | −0.3275 | 0.4248 | 0.3794 | 0.5802 |
| 70 | SRD5A2 | 5.2191 | 5.8110 | −0.5919 | 0.3795 | 0.5427 |
| 71 | CYB5R2 | 0.1912 | 0.4528 | −0.2616 | 0.3887 | 0.5728 |
| 72 | MYLK | 4.0279 | 3.5761 | 0.4518 | 0.3908 | 0.5669 |
| 73 | IPO8 | −0.7472 | −0.9454 | 0.1982 | 0.3992 | 0.5062 |
| 74 | CAV1 | 3.3959 | 3.7827 | −0.3867 | 0.4103 | 0.5353 |
| 75 | ELF4 | 0.2466 | 0.4879 | −0.2413 | 0.4231 | 0.5570 |
| 76 | COL17A1 | 7.7942 | 7.4205 | 0.3736 | 0.4276 | 0.5822 |
| 77 | CAMKK2 | −0.6919 | −0.8700 | 0.1782 | 0.4396 | 0.5580 |
| 78 | GPR160 | −1.1870 | −0.9274 | −0.2596 | 0.4457 | 0.5190 |
| 79 | PPP3CA | −0.5808 | −0.8989 | 0.3182 | 0.4544 | 0.5798 |
| 80 | EFNA5 | 3.6065 | 3.1541 | 0.4523 | 0.4773 | 0.5867 |
| 81 | HPS1 | 1.2172 | 1.4100 | −0.1928 | 0.4803 | 0.5393 |
| 82 | RALGAPA2 | −0.6274 | −0.9311 | 0.3037 | 0.4809 | 0.5956 |
| 83 | MCCC2 | 0.0629 | −0.1568 | 0.2196 | 0.4825 | 0.5491 |
| 84 | TCEAL2 | −0.4801 | −0.1753 | −0.3049 | 0.4835 | 0.5240 |
| 85 | DNAJC10 | 0.1806 | 0.3683 | −0.1877 | 0.4837 | 0.5812 |
| 86 | EZH2 | 2.3134 | 2.0548 | 0.2585 | 0.4875 | 0.5625 |
| 87 | TPD52 | −3.6078 | −3.3571 | −0.2507 | 0.4963 | 0.5027 |
| 88 | ACTC1 | 8.8134 | 9.0153 | −0.2019 | 0.5128 | 0.5240 |
| 89 | AGER | 8.8134 | 9.0153 | −0.2019 | 0.5128 | 0.5240 |
| 90 | CLU | 1.8531 | 1.6642 | 0.1889 | 0.5196 | 0.5338 |
| 91 | SLC43A1 | 0.7544 | 0.4921 | 0.2623 | 0.5259 | 0.5160 |
| 92 | POU4F1 | 8.7474 | 8.9350 | −0.1876 | 0.5297 | 0.5274 |
| 93 | MYOF | 0.7912 | 0.9667 | −0.1755 | 0.5360 | 0.5373 |
| 94 | SIM2 | 1.1007 | 0.8271 | 0.2736 | 0.5424 | 0.5699 |
| 95 | ARMCX1 | 0.1294 | −0.0358 | 0.1651 | 0.5431 | 0.5343 |
| 96 | ATP7B | 1.8904 | 1.7452 | 0.1452 | 0.5438 | 0.5664 |
| 97 | HLA.DMB | −0.8523 | −1.2019 | 0.3497 | 0.5463 | 0.5294 |
| 98 | UBC | −5.1870 | −5.0254 | −0.1616 | 0.5554 | 0.5111 |
| 99 | TRIM29 | 4.4984 | 4.0775 | 0.4209 | 0.5620 | 0.5240 |
| 100 | HSD17B11 | 1.2821 | 1.4725 | −0.1904 | 0.5686 | 0.5373 |
| 101 | FASN | −2.2334 | −2.4163 | 0.1830 | 0.5756 | 0.5333 |
| 102 | STEAP1 | 0.5492 | 0.7354 | −0.1862 | 0.5813 | 0.5111 |
| 103 | FOXA1 | −2.0167 | −2.1748 | 0.1581 | 0.5816 | 0.5393 |
| 104 | CREB3L4 | 0.0609 | 0.2618 | −0.2009 | 0.5824 | 0.5156 |
| 105 | CSTA | 0.3872 | 0.5739 | −0.1867 | 0.5851 | 0.5462 |
| 106 | MPZL2 | 1.6739 | 1.4457 | 0.2281 | 0.5877 | 0.5077 |
| 107 | MAP7 | −0.1789 | −0.3477 | 0.1688 | 0.6110 | 0.5225 |
| 108 | TTK | 3.5626 | 3.8478 | −0.2851 | 0.6114 | 0.5373 |
| 109 | CTNND2 | 0.9868 | 0.7791 | 0.2078 | 0.6199 | 0.5363 |
| 110 | RPL22L1 | 4.7274 | 5.0035 | −0.2761 | 0.6271 | 0.5319 |
| 111 | RAB34 | −0.0272 | −0.1395 | 0.1123 | 0.6305 | 0.5427 |
| 112 | DDX43 | 4.1737 | 3.9226 | 0.2511 | 0.6331 | 0.5496 |
| 113 | EFS | −0.6773 | −0.4926 | −0.1847 | 0.6335 | 0.5151 |
| 114 | UCK2 | 0.9319 | 0.7428 | 0.1892 | 0.6346 | 0.5259 |
| 115 | C12orf75 | 1.8173 | 1.9985 | −0.1812 | 0.6361 | 0.5343 |
| 116 | TRPM8 | −0.3203 | −0.1452 | −0.1751 | 0.6399 | 0.5086 |
| 117 | ARHGAP29 | 0.9249 | 0.7959 | 0.1290 | 0.6474 | 0.5249 |
| 118 | HOXC8 | 8.8134 | 8.9511 | −0.1376 | 0.6540 | 0.5086 |
| 119 | KRT5 | 4.1253 | 3.8503 | 0.2750 | 0.6549 | 0.5383 |
| 120 | SLC8A1 | 0.4700 | 0.2736 | 0.1964 | 0.6550 | 0.5215 |
| 121 | SELE | 8.7440 | 8.8823 | −0.1382 | 0.6635 | 0.5175 |
| 122 | PDZD2 | 3.3259 | 3.0725 | 0.2534 | 0.6800 | 0.5659 |
| 123 | HOXC5 | 8.2479 | 7.9784 | 0.2695 | 0.6854 | 0.5072 |
| 124 | ILK | 0.8396 | 0.7413 | 0.0983 | 0.6951 | 0.5062 |
| 125 | GNA12 | 1.6216 | 1.5299 | 0.0917 | 0.7060 | 0.5027 |
| 126 | HIP1 | 1.6854 | 1.5549 | 0.1305 | 0.7103 | 0.5299 |
| 127 | MAGED3 | 4.1239 | 3.9335 | 0.1904 | 0.7131 | 0.5319 |
| 128 | SH3RF1 | 0.2626 | 0.1094 | 0.1532 | 0.7179 | 0.5319 |
| 129 | PCGEM1 | −0.9065 | −0.7397 | −0.1668 | 0.7214 | 0.5101 |
| 130 | PARM1 | 1.4234 | 1.5361 | −0.1127 | 0.7292 | 0.5348 |
| 131 | GMDS | 1.1881 | 1.0779 | 0.1102 | 0.7406 | 0.5215 |
| 132 | GSTP1 | −2.3478 | −2.2977 | −0.0501 | 0.7532 | 0.5328 |
| 133 | BEND4 | −0.3342 | −0.4322 | 0.0980 | 0.7563 | 0.5417 |
| 134 | TMTC4 | 0.8539 | 0.7245 | 0.1294 | 0.7571 | 0.5590 |
| 135 | PMEPA1 | −2.4577 | −2.5409 | 0.0832 | 0.7620 | 0.5151 |
| 136 | FABP5 | −0.1850 | −0.2879 | 0.1028 | 0.7661 | 0.5160 |
| 137 | PPP1R3C | 1.6833 | 1.5799 | 0.1034 | 0.7764 | 0.5289 |
| 138 | ALKBH3 | 0.0385 | −0.0732 | 0.1117 | 0.7792 | 0.5333 |
| 139 | PEX10 | −1.0387 | −0.9567 | −0.0821 | 0.7800 | 0.5210 |
| 140 | WFS1 | 2.5732 | 2.7225 | −0.1493 | 0.7847 | 0.5393 |
| 141 | PSCA | −1.2920 | −1.4146 | 0.1226 | 0.7914 | 0.5338 |
| 142 | CES1 | 0.4058 | 0.5316 | −0.1258 | 0.7973 | 0.5042 |
| 143 | LMO7 | 1.8604 | 1.7620 | 0.0985 | 0.7977 | 0.5531 |
| 144 | AMPD3 | 4.4065 | 4.4961 | −0.0896 | 0.7991 | 0.5215 |
| 145 | CAPG | −4.7961 | −4.7066 | −0.0895 | 0.8041 | 0.5595 |
| 146 | FLNA | 1.2994 | 1.3950 | −0.0956 | 0.8070 | 0.5249 |
| 147 | ABCA5 | 0.2573 | 0.2972 | −0.0399 | 0.8267 | 0.5160 |
| 148 | PIN1 | −1.3256 | −1.2847 | −0.0409 | 0.8415 | 0.5047 |
| 149 | CITED2 | −1.5571 | −1.5019 | −0.0553 | 0.8437 | 0.5072 |
| 150 | UAP1 | −1.3395 | −1.4249 | 0.0855 | 0.8502 | 0.5294 |
| 151 | GDPD1 | 4.5863 | 4.4831 | 0.1032 | 0.8564 | 0.5116 |
| 152 | CRYAB | −0.4127 | −0.3447 | −0.0680 | 0.8593 | 0.5457 |
| 153 | VAMP3 | 0.5205 | 0.5815 | −0.0610 | 0.8601 | 0.5437 |
| 154 | ATP1A2 | 8.7406 | 8.6811 | 0.0595 | 0.8631 | 0.5062 |
| 155 | E2F3 | 1.4213 | 1.4627 | −0.0414 | 0.8639 | 0.5348 |
| 156 | FOXF2 | 8.3384 | 8.2730 | 0.0654 | 0.8685 | 0.5012 |
| 157 | ATP2B4 | 1.7438 | 1.6793 | 0.0645 | 0.8715 | 0.5081 |
| 158 | FOLH1B | 0.7295 | 0.7809 | −0.0514 | 0.8848 | 0.5304 |
| 159 | PAGE4 | 8.4394 | 8.4923 | −0.0530 | 0.8864 | 0.5012 |
| 160 | KLHL21 | −1.0755 | −1.0287 | −0.0469 | 0.8931 | 0.5042 |
| 161 | EFEMP1 | 1.9780 | 2.0314 | −0.0534 | 0.9096 | 0.5378 |
| 162 | KLK3 | −3.1769 | −3.2132 | 0.0363 | 0.9101 | 0.5126 |
| 163 | HSD17B8 | 1.9487 | 1.9118 | 0.0369 | 0.9103 | 0.5057 |
| 164 | ZNF3 | 1.0811 | 1.0428 | 0.0384 | 0.9220 | 0.5264 |
| 165 | ACSM1 | 1.7029 | 1.6506 | 0.0522 | 0.9222 | 0.5022 |
| 166 | ANXA1 | −2.9108 | −2.9271 | 0.0163 | 0.9367 | 0.5269 |
| 167 | MAGED4 | 2.9869 | 3.0242 | −0.0373 | 0.9389 | 0.5521 |
| 168 | CSRP1 | −1.2859 | −1.2678 | −0.0181 | 0.9426 | 0.5042 |
| 169 | LGALS8 | −0.3414 | −0.3268 | −0.0147 | 0.9685 | 0.5254 |
| 170 | ZNF276 | 0.5132 | 0.5258 | −0.0126 | 0.9793 | 0.5067 |
| 171 | CRISP3 | −0.2822 | −0.2703 | −0.0119 | 0.9841 | 0.5007 |
| 172 | DLX1 | 6.4728 | 6.4614 | 0.0114 | 0.9850 | 0.5121 |
| 173 | WDFY2 | 2.1927 | 2.1990 | −0.0063 | 0.9854 | 0.5269 |
| 174 | FGFR2 | 0.9312 | 0.9373 | −0.0061 | 0.9868 | 0.5185 |
| 175 | S100A6 | −1.2939 | −1.3000 | 0.0062 | 0.9874 | 0.5319 |
| 176 | THBS4 | 4.8180 | 4.8116 | 0.0064 | 0.9903 | 0.5279 |
| 177 | REG4 | 2.7771 | 2.7818 | −0.0047 | 0.9921 | 0.5067 |
| 178 | ID4 | 0.7066 | 0.7093 | −0.0028 | 0.9926 | 0.5180 |

TABLE 3B
Expression Characteristics of Candidate Markers in Urine Sediments

| | Official | Mean DeltaCt | | Difference | t-test | |
|---|---|---|---|---|---|---|
| Rank | Symbol | Normal (n = 50) | Tumor (n = 27) | in Means | p-value | AUC |
| 1 | OR51E2 | 5.3146 | 3.3055 | 2.0091 | 0.0014 | 0.6785 |
| 2 | TMEM178 | 5.7793 | 4.1774 | 1.6019 | 0.0016 | 0.6408 |
| 3 | HOXC4 | 6.0017 | 4.6367 | 1.3650 | 0.0017 | 0.6331 |
| 4 | ARHGEF26 | 5.1998 | 3.3905 | 1.8093 | 0.0020 | 0.6632 |
| 5 | CACNA1D | 6.1800 | 4.7580 | 1.4220 | 0.0032 | 0.6299 |
| 6 | FOLH1 | 3.9355 | 1.9305 | 2.0049 | 0.0033 | 0.6807 |
| 7 | PCA3 | 2.8204 | 0.5102 | 2.3102 | 0.0034 | 0.7056 |
| 8 | TBP | 0.3385 | −0.4277 | 0.7662 | 0.0069 | 0.5681 |
| 9 | ERG | 5.9606 | 4.7118 | 1.2489 | 0.0075 | 0.6282 |
| 10 | TWIST1 | 6.2414 | 4.9820 | 1.2593 | 0.0086 | 0.6162 |
| 11 | SDK1 | 6.2414 | 4.9834 | 1.2579 | 0.0086 | 0.6162 |
| 12 | PDZD2 | 5.4484 | 3.8359 | 1.6125 | 0.0091 | 0.6435 |
| 13 | ADAM2 | 5.9164 | 4.4842 | 1.4322 | 0.0107 | 0.6315 |
| 14 | FOXA1 | −0.7121 | −1.9536 | 1.2415 | 0.0115 | 0.6315 |
| 15 | TTK | 5.7120 | 4.6134 | 1.0986 | 0.0123 | 0.6124 |
| 16 | COL17A1 | 6.1157 | 4.9344 | 1.1813 | 0.0138 | 0.6102 |
| 17 | FLNC | 5.9588 | 4.7478 | 1.2110 | 0.0138 | 0.6129 |
| 18 | HOXC6 | 5.8600 | 4.7612 | 1.0988 | 0.0150 | 0.6091 |
| 19 | TRIM29 | 3.8267 | 2.4014 | 1.4254 | 0.0151 | 0.6681 |
| 20 | FGF8 | 6.1812 | 5.0922 | 1.0890 | 0.0161 | 0.6058 |
| 21 | SLITRK6 | 6.1849 | 5.0922 | 1.0927 | 0.0167 | 0.6058 |
| 22 | FOLH1B | 5.8848 | 4.7602 | 1.1246 | 0.0182 | 0.6113 |
| 23 | COL2A1 | 6.1830 | 5.0922 | 1.0908 | 0.0186 | 0.6063 |
| 24 | POU4F1 | 6.1778 | 5.0922 | 1.0856 | 0.0210 | 0.6036 |
| 25 | TRPM8 | 4.9268 | 3.5297 | 1.3971 | 0.0243 | 0.6386 |
| 26 | REG4 | 5.7627 | 4.6972 | 1.0655 | 0.0243 | 0.6091 |
| 27 | CTNND2 | 5.7481 | 4.4276 | 1.3206 | 0.0274 | 0.6214 |
| 28 | RASSF1A | 2.0557 | 3.4078 | −1.3521 | 0.0274 | 0.5839 |
| 29 | SRD5A1 | 0.9943 | 2.2221 | −1.2278 | 0.0276 | 0.5817 |
| 30 | NRIP1 | 2.2159 | 3.5335 | −1.3175 | 0.0279 | 0.5905 |
| 31 | STEAP1 | 5.1299 | 3.8983 | 1.2315 | 0.0345 | 0.6151 |
| 32 | MYLK | 5.4961 | 4.4078 | 1.0883 | 0.0361 | 0.6118 |
| 33 | TFF1 | 1.7934 | 0.2493 | 1.5441 | 0.0365 | 0.6340 |
| 34 | ACSM1 | 6.0583 | 5.0922 | 0.9661 | 0.0372 | 0.5932 |
| 35 | EPCAM | 2.0475 | 0.6475 | 1.4000 | 0.0381 | 0.6441 |
| 36 | MAGED3 | 5.9152 | 5.0004 | 0.9148 | 0.0401 | 0.6031 |
| 37 | ARHGAP29 | 3.2513 | 1.8786 | 1.3726 | 0.0417 | 0.6244 |
| 38 | DNAJC10 | 3.2357 | 4.2363 | −1.0006 | 0.0460 | 0.5517 |
| 39 | WWTR1 | 3.0809 | 1.8749 | 1.2060 | 0.0507 | 0.6512 |
| 40 | GNA12 | 1.3404 | 2.2782 | −0.9378 | 0.0507 | 0.5905 |
| 41 | WDFY2 | 1.7244 | 0.6958 | 1.0287 | 0.0534 | 0.6514 |
| 42 | AMPD3 | 5.8667 | 5.0922 | 0.7746 | 0.0547 | 0.5921 |
| 43 | KRT15 | 0.2788 | −1.0953 | 1.3741 | 0.0562 | 0.6583 |
| 44 | ELF4 | 1.0343 | 2.0991 | −1.0648 | 0.0597 | 0.6052 |
| 45 | EFNA5 | 5.6967 | 4.7402 | 0.9565 | 0.0622 | 0.5938 |
| 46 | THBS4 | 5.9510 | 5.0922 | 0.8589 | 0.0622 | 0.5927 |
| 47 | HPN | 2.7187 | 1.5646 | 1.1541 | 0.0774 | 0.6446 |
| 48 | TFF3 | 3.8921 | 2.7895 | 1.1025 | 0.0781 | 0.6052 |
| 49 | TDRD1 | 5.8406 | 4.9915 | 0.8492 | 0.0822 | 0.5927 |
| 50 | MAGED4 | 5.5494 | 4.6425 | 0.9069 | 0.0955 | 0.5981 |
| 51 | FMO5 | 1.9558 | 2.7912 | −0.8354 | 0.0969 | 0.5358 |
| 52 | TPD52 | −0.2720 | −1.1235 | 0.8515 | 0.1012 | 0.6441 |
| 53 | CLU | −0.7026 | −1.3801 | 0.6775 | 0.1022 | 0.6408 |
| 54 | HSD17B6 | 5.2060 | 4.2928 | 0.9131 | 0.1046 | 0.6020 |
| 55 | MYO6 | 2.8433 | 1.7246 | 1.1187 | 0.1057 | 0.6479 |
| 56 | HLA-DMB | −2.8507 | −1.7567 | −1.0940 | 0.1064 | 0.5309 |
| 57 | SRD5A2 | 5.9209 | 5.0922 | 0.8288 | 0.1090 | 0.5735 |
| 58 | CRISP3 | 0.6908 | −0.4277 | 1.1186 | 0.1125 | 0.6791 |
| 59 | FLNA | 0.3058 | 1.3502 | −1.0445 | 0.1188 | 0.5506 |
| 60 | FABP5 | −3.3083 | −4.0045 | 0.6962 | 0.1299 | 0.6140 |
| 61 | RAB34 | 1.9869 | 2.9130 | −0.9261 | 0.1359 | 0.5686 |
| 62 | ANXA1 | −3.4823 | −3.9273 | 0.4450 | 0.1415 | 0.6408 |
| 63 | DDX43 | 5.6988 | 5.0922 | 0.6066 | 0.1490 | 0.5856 |
| 64 | OR51E1 | 5.8005 | 5.0922 | 0.7083 | 0.1509 | 0.5708 |
| 65 | HSD17B8 | 1.6142 | 0.7701 | 0.8440 | 0.1580 | 0.6036 |
| 66 | POLR2A | −0.7121 | −0.3264 | −0.3857 | 0.1612 | 0.5107 |
| 67 | PSCA | −1.6439 | −2.4417 | 0.7978 | 0.1650 | 0.6192 |
| 68 | ZNF276 | 1.5986 | 2.5212 | −0.9226 | 0.1664 | 0.5631 |
| 69 | SNAI2 | 0.1743 | −0.9787 | 1.1530 | 0.1744 | 0.5686 |
| 70 | FNIP2 | 2.5732 | 3.3186 | −0.7454 | 0.1751 | 0.5249 |
| 71 | PARM1 | 1.9736 | 2.8660 | −0.8923 | 0.1769 | 0.5467 |
| 72 | CES1 | 0.5269 | 1.3586 | −0.8318 | 0.1810 | 0.5391 |
| 73 | PPP1R3C | 0.3639 | −0.4781 | 0.8419 | 0.1892 | 0.6047 |
| 74 | GUCY1A3 | 2.3073 | 1.4051 | 0.9022 | 0.1941 | 0.6107 |
| 75 | PPP3CA | 1.8086 | 0.9719 | 0.8367 | 0.1969 | 0.6137 |
| 76 | TCEAL2 | 4.2810 | 3.5334 | 0.7476 | 0.1969 | 0.5757 |
| 77 | PCGEM1 | 2.7562 | 1.7239 | 1.0323 | 0.1988 | 0.5656 |
| 78 | PMEPA1 | −1.3734 | −1.9732 | 0.5998 | 0.2013 | 0.6121 |
| 79 | TRIB1 | 0.8147 | 1.5543 | −0.7396 | 0.2037 | 0.5014 |
| 80 | SIM2 | 5.0847 | 4.3812 | 0.7035 | 0.2076 | 0.5719 |
| 81 | MAOB | 3.6854 | 2.9033 | 0.7822 | 0.2111 | 0.5845 |
| 82 | GOLM1 | 1.3003 | 0.6326 | 0.6677 | 0.2161 | 0.5960 |
| 83 | PLA2G7 | 0.8370 | 1.6501 | −0.8131 | 0.2252 | 0.5025 |
| 84 | SLC8A1 | 2.4864 | 3.0633 | −0.5769 | 0.2388 | 0.5566 |
| 85 | SPINK1 | 0.3111 | 1.1019 | −0.7907 | 0.2501 | 0.5465 |
| 86 | KLHL21 | 2.3548 | 3.0099 | −0.6550 | 0.2593 | 0.5090 |
| 87 | ERGIC2 | 1.4801 | 2.1579 | −0.6778 | 0.2725 | 0.5074 |
| 88 | KLK3 | −0.3973 | −1.1647 | 0.7674 | 0.2827 | 0.5919 |
| 89 | UCK2 | 2.3282 | 2.9290 | −0.6008 | 0.2893 | 0.5478 |
| 90 | S100A8 | −5.2699 | −5.7515 | 0.4816 | 0.2981 | 0.6124 |
| 91 | PIN1 | −0.2502 | 0.2306 | −0.4808 | 0.3053 | 0.5120 |
| 92 | FRMD6 | 4.9901 | 4.5268 | 0.4633 | 0.3102 | 0.5703 |
| 93 | MEIS2 | 3.9643 | 3.2363 | 0.7280 | 0.3109 | 0.5891 |
| 94 | SH3RF1 | 3.9595 | 3.4100 | 0.5494 | 0.3148 | 0.5894 |
| 95 | E2F3 | 2.1070 | 2.6585 | −0.5516 | 0.3214 | 0.5314 |
| 96 | EZH2 | 3.2873 | 3.7912 | −0.5039 | 0.3306 | 0.5112 |
| 97 | NAV2 | 4.9308 | 4.4232 | 0.5075 | 0.3328 | 0.5522 |
| 98 | TSPAN1 | 1.4614 | 0.9523 | 0.5091 | 0.3388 | 0.5681 |
| 99 | S100A9 | −4.1992 | −4.6212 | 0.4220 | 0.3406 | 0.6167 |
| 100 | CREB3L4 | 3.4376 | 3.9070 | −0.4694 | 0.3427 | 0.5137 |
| 101 | CRYAB | −1.0017 | −1.6088 | 0.6071 | 0.3436 | 0.5765 |
| 102 | HIP1 | 3.4126 | 3.8441 | −0.4314 | 0.3477 | 0.5014 |
| 103 | CITED2 | −1.9781 | −2.3339 | 0.3558 | 0.3487 | 0.5571 |
| 104 | HPS1 | −0.4384 | 0.0692 | −0.5075 | 0.3493 | 0.5090 |
| 105 | RALGAPA2 | 3.4965 | 2.9276 | 0.5688 | 0.3571 | 0.6102 |
| 106 | S100A6 | −2.6451 | −2.1477 | −0.4974 | 0.3622 | 0.5396 |
| 107 | VAMP3 | −0.7627 | −1.3236 | 0.5609 | 0.3649 | 0.5837 |
| 108 | ZNF3 | 2.2777 | 1.7880 | 0.4897 | 0.3681 | 0.5872 |
| 109 | EFS | 3.9783 | 3.3004 | 0.6779 | 0.3896 | 0.5495 |
| 110 | TNK2 | 3.1807 | 3.6257 | −0.4451 | 0.3923 | 0.5112 |
| 111 | SPON2 | 4.6987 | 4.2122 | 0.4865 | 0.3943 | 0.5681 |
| 112 | AMACR | 0.7973 | 1.2929 | −0.4956 | 0.3946 | 0.5200 |
| 113 | TAGLN | 5.0253 | 4.5434 | 0.4819 | 0.3981 | 0.5571 |
| 114 | LMO7 | −0.2115 | −0.6596 | 0.4481 | 0.4127 | 0.5596 |
| 115 | DVL1 | 1.0029 | 1.3897 | −0.3868 | 0.4141 | 0.5331 |
| 116 | GMDS | 3.7594 | 3.2973 | 0.4621 | 0.4157 | 0.5910 |
| 117 | SYNM | 4.5867 | 4.1660 | 0.4207 | 0.4192 | 0.5675 |
| 118 | CSTA | −2.1048 | −2.4380 | 0.3332 | 0.4253 | 0.6167 |
| 119 | MAP7 | 1.5734 | 1.0723 | 0.5011 | 0.4326 | 0.5850 |
| 120 | MCCC2 | 1.3798 | 0.8787 | 0.5011 | 0.4423 | 0.6003 |
| 121 | ACTA2 | 4.5680 | 4.2171 | 0.3508 | 0.4538 | 0.5724 |
| 122 | HPRT1 | −0.2581 | 0.0263 | −0.2843 | 0.4716 | 0.6124 |
| 123 | ATP7B | 5.2411 | 4.9058 | 0.3353 | 0.4936 | 0.5626 |
| 124 | RAP1GAP | 4.2886 | 3.8566 | 0.4320 | 0.5110 | 0.5440 |
| 125 | WFS1 | 2.3291 | 1.8079 | 0.5211 | 0.5149 | 0.5697 |
| 126 | FGFR2 | 3.0887 | 2.6564 | 0.4323 | 0.5263 | 0.5560 |
| 127 | ABCC4 | 2.4497 | 2.0811 | 0.3687 | 0.5282 | 0.5708 |
| 128 | CAMKK2 | 1.0014 | 1.3353 | −0.3338 | 0.5446 | 0.5030 |
| 129 | CAV1 | 4.4190 | 4.0733 | 0.3458 | 0.5549 | 0.5375 |
| 130 | C12orf75 | 2.4185 | 1.9163 | 0.5022 | 0.5618 | 0.5664 |
| 131 | CYB5R2 | 3.6405 | 3.3541 | 0.2864 | 0.5673 | 0.5582 |
| 132 | ID4 | 3.0331 | 2.6290 | 0.4042 | 0.5714 | 0.5517 |
| 133 | GALNT7 | 3.9990 | 4.3107 | −0.3117 | 0.5736 | 0.5014 |
| 134 | MYOF | 1.8111 | 2.1103 | −0.2992 | 0.5996 | 0.5101 |
| 135 | ARMCX1 | 2.1771 | 2.4448 | −0.2677 | 0.6091 | 0.5191 |
| 136 | SLC43A1 | 4.0889 | 4.3396 | −0.2507 | 0.6181 | 0.5145 |
| 137 | GSTP1 | −3.0483 | −3.1923 | 0.1440 | 0.6255 | 0.5949 |
| 138 | UBC | −5.0439 | −5.1612 | 0.1174 | 0.6264 | 0.6080 |
| 139 | ATP2B4 | 2.6705 | 2.8655 | −0.1950 | 0.6590 | 0.5555 |
| 140 | GPR160 | −0.2467 | −0.5232 | 0.2766 | 0.6606 | 0.5612 |
| 141 | MPPED2 | 4.2824 | 4.5320 | −0.2496 | 0.6682 | 0.5134 |
| 142 | WFDC2 | 1.1016 | 0.7947 | 0.3069 | 0.6804 | 0.5670 |
| 143 | CAPG | −4.2244 | −4.0600 | −0.1644 | 0.6960 | 0.5336 |
| 144 | FBP1 | −1.4394 | −1.5845 | 0.1451 | 0.7462 | 0.5752 |
| 145 | LAPTM4B | 1.9350 | 2.1303 | −0.1953 | 0.7496 | 0.5008 |
| 146 | ILK | −1.0169 | −0.8813 | −0.1356 | 0.7763 | 0.5123 |
| 147 | LGALS8 | 0.1183 | 0.2856 | −0.1673 | 0.7836 | 0.5410 |
| 148 | HSD17B11 | −0.9348 | −0.7994 | −0.1353 | 0.7846 | 0.5112 |
| 149 | EFEMP1 | 3.8914 | 3.7238 | 0.1675 | 0.7919 | 0.5271 |
| 150 | ABCA5 | 3.1438 | 3.2776 | −0.1337 | 0.7969 | 0.5440 |
| 151 | MPZL2 | 3.4752 | 3.3417 | 0.1335 | 0.8065 | 0.5451 |
| 152 | KRT5 | 4.9155 | 4.7976 | 0.1179 | 0.8179 | 0.5243 |
| 153 | GDPD1 | 4.9082 | 4.8331 | 0.0750 | 0.8495 | 0.5473 |
| 154 | BEND4 | 3.5553 | 3.4508 | 0.1045 | 0.8588 | 0.5380 |
| 155 | GALNT3 | 1.8192 | 1.9117 | −0.0925 | 0.8621 | 0.5271 |
| 156 | GUSB | −0.1043 | −0.1395 | 0.0353 | 0.8752 | 0.5796 |
| 157 | TPM2 | 4.9154 | 4.8534 | 0.0620 | 0.8754 | 0.5528 |
| 158 | UAP1 | 0.5894 | 0.6773 | −0.0880 | 0.8931 | 0.5763 |
| 159 | IPO8 | 1.1090 | 1.1557 | −0.0468 | 0.8986 | 0.5839 |
| 160 | CSRP1 | 0.2578 | 0.1863 | 0.0715 | 0.9018 | 0.5446 |
| 161 | PTP4A3 | 3.8405 | 3.8882 | −0.0477 | 0.9060 | 0.5500 |
| 162 | ALKBH3 | 2.7342 | 2.6760 | 0.0582 | 0.9158 | 0.5156 |
| 163 | PEX10 | 1.9629 | 1.9021 | 0.0609 | 0.9298 | 0.5085 |
| 164 | TMTC4 | 3.9149 | 3.9461 | −0.0312 | 0.9552 | 0.5440 |
| 165 | TSPAN13 | 3.0045 | 2.9749 | 0.0296 | 0.9640 | 0.5566 |
| 166 | LAMB3 | 0.6535 | 0.6480 | 0.0055 | 0.9917 | 0.5451 |
| 167 | FASN | 0.8070 | 0.8100 | −0.0030 | 0.9960 | 0.5443 |

TABLE 4A
Performance Characteristics of Prostate Cancer Multi-gene Signatures in Whole Urine Samples

| | Machine Learning | Nb | | DeLong | | | |
|---|---|---|---|---|---|---|---|
| Rank | Method | Gene | AUC | p-value | Sensitivity | Specificity | Accuracy |
| 1 | Random Forest | 8 | 0.850617 | 0.002264 | 82.2 | 82.2 | 82.2 |
| 2 | Random Forest | 8 | 0.864444 | 0.002127 | 84.4 | 77.8 | 81.1 |
| 3 | Random Forest | 8 | 0.857778 | 0.004621 | 82.2 | 77.8 | 80.0 |
| 4 | Naive Bayes | 9 | 0.817284 | 0.021875 | 82.2 | 82.2 | 82.2 |
| 5 | Naive Bayes | 5 | 0.806914 | 0.026091 | 82.2 | 80.0 | 81.1 |
| 6 | Random Forest | 9 | 0.847901 | 0.002293 | 84.4 | 77.8 | 81.1 |
| 7 | Random Forest | 8 | 0.853827 | 0.004829 | 84.4 | 73.3 | 78.9 |
| 8 | Random Forest | 8 | 0.842469 | 0.005408 | 82.2 | 75.6 | 78.9 |
| 9 | Random Forest | 9 | 0.837778 | 0.003190 | 82.2 | 75.6 | 78.9 |
| 10 | Random Forest | 9 | 0.826667 | 0.008667 | 80.0 | 75.6 | 77.8 |
| 11 | Naive Bayes | 8 | 0.817778 | 0.016039 | 80.0 | 80.0 | 80.0 |
| 12 | Naive Bayes | 9 | 0.814321 | 0.029127 | 80.0 | 80.0 | 80.0 |
| 13 | Naive Bayes | 7 | 0.811852 | 0.028601 | 80.0 | 80.0 | 80.0 |
| 14 | Naive Bayes | 9 | 0.811358 | 0.024384 | 80.0 | 80.0 | 80.0 |
| 15 | Random Forest | 9 | 0.826173 | 0.007471 | 84.4 | 77.8 | 81.1 |
| 16 | Random Forest | 8 | 0.823457 | 0.010485 | 86.7 | 71.1 | 78.9 |
| 17 | Naive Bayes | 7 | 0.819259 | 0.016767 | 84.4 | 75.6 | 80.0 |
| 18 | Naive Bayes | 8 | 0.818765 | 0.021170 | 84.4 | 77.8 | 81.1 |
| 19 | Naive Bayes | 8 | 0.818765 | 0.019229 | 84.4 | 75.6 | 80.0 |
| 20 | Naive Bayes | 9 | 0.818272 | 0.018762 | 80.0 | 77.8 | 78.9 |
| 21 | Random Forest | 7 | 0.846420 | 0.005095 | 73.3 | 86.7 | 80.0 |
| 22 | Random Forest | 10 | 0.845185 | 0.002513 | 75.6 | 80.0 | 77.8 |
| 23 | Naive Bayes | 4 | 0.838519 | 0.015898 | 71.1 | 84.4 | 77.8 |
| 24 | Random Forest | 8 | 0.834815 | 0.006329 | 73.3 | 84.4 | 78.9 |
| 25 | Random Forest | 8 | 0.830617 | 0.008529 | 77.8 | 77.8 | 77.8 |
| 26 | Random Forest | 9 | 0.828889 | 0.010240 | 75.6 | 77.8 | 76.7 |
| 27 | Random Forest | 11 | 0.822963 | 0.005734 | 77.8 | 80.0 | 78.9 |
| 28 | Naive Bayes | 2 | 0.821728 | 0.024877 | 80.0 | 77.8 | 78.9 |
| 29 | Random Forest | 8 | 0.820988 | 0.027011 | 80.0 | 75.6 | 77.8 |
| 30 | Random Forest | 9 | 0.820494 | 0.032538 | 80.0 | 77.8 | 78.9 |
| 31 | Random Forest | 6 | 0.820494 | 0.008747 | 75.6 | 84.4 | 80.0 |
| 32 | Naive Bayes | 7 | 0.819753 | 0.017533 | 82.2 | 77.8 | 80.0 |
| 33 | Random Forest | 6 | 0.819259 | 0.019806 | 77.8 | 77.8 | 77.8 |
| 34 | Naive Bayes | 7 | 0.814815 | 0.020641 | 77.8 | 80.0 | 78.9 |
| 35 | Naive Bayes | 9 | 0.813827 | 0.021629 | 77.8 | 80.0 | 78.9 |
| 36 | Naive Bayes | 8 | 0.813333 | 0.023319 | 82.2 | 77.8 | 80.0 |
| 37 | Random Forest | 3 | 0.812840 | 0.027034 | 84.4 | 71.1 | 77.8 |
| 38 | Naive Bayes | 9 | 0.811852 | 0.031236 | 77.8 | 77.8 | 77.8 |
| 39 | Naive Bayes | 10 | 0.810864 | 0.024567 | 82.2 | 75.6 | 78.9 |
| 40 | Naive Bayes | 10 | 0.809383 | 0.025224 | 77.8 | 80.0 | 78.9 |
| 41 | Naive Bayes | 8 | 0.808889 | 0.018651 | 82.2 | 75.6 | 78.9 |
| 42 | Naive Bayes | 8 | 0.808395 | 0.026394 | 84.4 | 71.1 | 77.8 |
| 43 | Naive Bayes | 9 | 0.808395 | 0.025669 | 82.2 | 77.8 | 80.0 |
| 44 | Naive Bayes | 10 | 0.808395 | 0.018862 | 77.8 | 82.2 | 80.0 |
| 45 | Random Forest | 5 | 0.807901 | 0.014359 | 66.7 | 82.2 | 74.4 |
| 46 | Random Forest | 10 | 0.807160 | 0.015324 | 71.1 | 82.2 | 76.7 |
| 47 | Naive Bayes | 8 | 0.804938 | 0.037459 | 84.4 | 75.6 | 80.0 |
| 48 | Random Forest | 7 | 0.803951 | 0.008514 | 66.7 | 91.1 | 78.9 |
| 49 | Random Forest | 7 | 0.803457 | 0.033589 | 75.6 | 82.2 | 78.9 |
| 50 | Naive Bayes | 6 | 0.802469 | 0.049725 | 71.1 | 84.4 | 77.8 |
| 51 | Naive Bayes | 5 | 0.801975 | 0.042480 | 82.2 | 75.6 | 78.9 |
| 52 | Naive Bayes | 6 | 0.800988 | 0.037817 | 71.1 | 84.4 | 77.8 |
| 53 | Random Forest | 4 | 0.791111 | 0.042238 | 66.7 | 82.2 | 74.4 |
| 54 | Random Forest | 4 | 0.806173 | 0.058471 | 73.3 | 75.6 | 74.4 |
| 55 | Random Forest | 3 | 0.776296 | 0.077403 | 80.0 | 77.8 | 78.9 |
| 56 | Naive Bayes | 9 | 0.774321 | 0.129411 | 82.2 | 75.6 | 78.9 |
| 57 | Random Forest | 7 | 0.759506 | 0.407508 | 71.1 | 77.8 | 74.4 |
| 58 | Random Forest | 2 | 0.750864 | 0.298667 | 64.4 | 77.8 | 71.1 |
| 59 | Random Forest | 3 | 0.724691 | 0.656093 | 77.8 | 62.2 | 70.0 |
| 60 | Random Forest | 4 | 0.717531 | 0.656995 | 75.6 | 57.8 | 66.7 |

TABLE 4B
Performance Characteristics of Prostate Cancer Multi-gene Signatures in
Urine Samples with Confirmed Presence of Prostate Cells

| | Machine | | | |
|---|---|---|---|---|
| Rank | Learning Method | Nb Gene | AUC | DeLong p-value |
| 1 | Naive Bayes | 3 | 0.813787 | 0.049765 |
| 2 | Naive Bayes | 8 | 0.773346 | 0.132157 |
| 3 | Naive Bayes | 8 | 0.774449 | 0.134058 |
| 4 | Naive Bayes | 9 | 0.770588 | 0.141174 |
| 5 | Naive Bayes | 9 | 0.768199 | 0.146155 |
| 6 | Naive Bayes | 7 | 0.771875 | 0.146727 |
| 7 | Naive Bayes | 5 | 0.767647 | 0.148872 |
| 8 | Naive Bayes | 8 | 0.765625 | 0.150005 |
| 9 | Random Forest | 10 | 0.760386 | 0.157278 |
| 10 | Naive Bayes | 5 | 0.769485 | 0.163111 |
| 11 | Naive Bayes | 8 | 0.766544 | 0.163878 |
| 12 | Naive Bayes | 8 | 0.766912 | 0.165959 |
| 13 | Naive Bayes | 9 | 0.767279 | 0.172960 |
| 14 | Naive Bayes | 9 | 0.766544 | 0.173379 |
| 15 | Naive Bayes | 9 | 0.769853 | 0.174634 |
| 16 | Naive Bayes | 6 | 0.764154 | 0.181853 |
| 17 | Random Forest | 5 | 0.763603 | 0.185708 |
| 18 | Naive Bayes | 9 | 0.765993 | 0.187185 |
| 19 | Naive Bayes | 10 | 0.765257 | 0.190532 |
| 20 | Naive Bayes | 6 | 0.764338 | 0.194475 |
| 21 | Random Forest | 6 | 0.778585 | 0.207653 |
| 22 | Naive Bayes | 9 | 0.763419 | 0.210162 |
| 23 | Naive Bayes | 9 | 0.764338 | 0.210318 |
| 24 | Naive Bayes | 3 | 0.778860 | 0.214319 |
| 25 | Naive Bayes | 9 | 0.763419 | 0.222629 |
| 26 | Random Forest | 10 | 0.763051 | 0.233504 |
| 27 | Naive Bayes | 4 | 0.758272 | 0.247932 |
| 28 | Random Forest | 8 | 0.762868 | 0.248382 |
| 29 | Naive Bayes | 4 | 0.774265 | 0.250954 |
| 30 | Naive Bayes | 6 | 0.757904 | 0.259289 |
| 31 | Naive Bayes | 7 | 0.756434 | 0.262668 |
| 32 | Naive Bayes | 7 | 0.761765 | 0.270339 |
| 33 | Random Forest | 5 | 0.769301 | 0.282388 |
| 34 | Random Forest | 10 | 0.753676 | 0.286136 |
| 35 | Naive Bayes | 6 | 0.754412 | 0.292353 |
| 36 | Random Forest | 7 | 0.745037 | 0.329950 |
| 37 | Naive Bayes | 7 | 0.749265 | 0.336941 |
| 38 | Random Forest | 6 | 0.746140 | 0.349600 |
| 39 | Random Forest | 7 | 0.753768 | 0.352375 |
| 40 | Random Forest | 6 | 0.760846 | 0.354260 |
| 41 | Random Forest | 9 | 0.742739 | 0.354422 |
| 42 | Random Forest | 6 | 0.743199 | 0.396201 |
| 43 | Random Forest | 5 | 0.736581 | 0.412218 |
| 44 | Random Forest | 8 | 0.738327 | 0.430296 |
| 45 | Naive Bayes | 4 | 0.750551 | 0.471117 |
| 46 | Random Forest | 7 | 0.742188 | 0.511709 |
| 47 | Random Forest | 12 | 0.748254 | 0.517539 |
| 48 | Random Forest | 7 | 0.733180 | 0.536945 |
| 49 | Random Forest | 4 | 0.734099 | 0.554592 |
| 50 | Random Forest | 7 | 0.741544 | 0.560231 |
| 51 | Random Forest | 6 | 0.750551 | 0.615783 |
| 52 | Random Forest | 5 | 0.731985 | 0.644389 |
| 53 | Random Forest | 4 | 0.733824 | 0.646331 |
| 54 | Random Forest | 3 | 0.738511 | 0.654049 |
| 55 | Random Forest | 6 | 0.730423 | 0.670225 |
| 56 | Random Forest | 9 | 0.732353 | 0.671557 |
| 57 | Random Forest | 8 | 0.727206 | 0.784646 |
| 58 | Random Forest | 8 | 0.729596 | 0.809125 |
| 59 | Random Forest | 7 | 0.724908 | 0.809591 |

TABLE 5
List of Selected Prostate Cancer Markers and their Associated Transcripts

| | | | | | Alternative | Frequency |
|---|---|---|---|---|---|---|
| Official | | Accession | | Associated | TaqMan | Of Gene |
| Symbol | Gene Name | Number | TaqMan Assay | Transcripts | Assay | Use (%) |
| KRT15 | keratin 15 | NM_002275 | Hs00267035_m1 | — | — | 68.33% |
| CACNA1D | calcium channel, voltage-dependent, L | NM_000720 | Hs00167753_m1 | NM_001128839; | Hs01073319_m1; | 63.33% |
| | type, alpha 1D subunit | | | NM_001128840 | Hs01073321_m1; | |
| | | | | | Hs01073332_m1; | |
| | | | | | Hs01073331_m1 | |
| ERG | v-ets erythroblastosis virus E26 | NM_182918 | Hs00171666_m1 | NM_001136154; | Hs01554635_m1; | 56.67% |
| | oncogene homolog | | | NM_001136155 | Hs01554630_m1; | |
| | | | | | Hs01554631_m1; | |
| | | | | | Hs01554632_m1 | |
| LAMB3 | laminin, beta 3 | NM_000228 | Hs00165078_m1 | NM_001017402; | Hs00989733_m1; | 53.33% |
| | | | | NM_001127641 | Hs00989725_m1; | |
| | | | | | Hs00989716_m1; | |
| | | | | | Hs00989730_m1 | |
| FLNC | filamin C, gamma | NM_001458 | Hs00155124_m1 | NM_001127487 | Hs01099451_m1; | 38.33% |
| | | | | | Hs01099457_m1; | |
| | | | | | Hs00356200_ml; | |
| | | | | | Hs00356228_m1 | |
| RASSF1A | Ras association domain family | NM_007182 | Hs00945257_m1 | NM_170712; | Hs00200394_m1; | 35.00% |
| | member 1 | | | NM_170713; | Hs00945253_m1; | |
| | | | | NM_170714; | Hs00945679_m1; | |
| | | | | NM_001206957 | Hs00945680_m1 | |
| TMEM178 | transmembrane protein 178 | NM_152390 | Hs00380771_m1 | NM_001167959 | Hs00917498_m1; | 26.67% |
| | | | | | Hs00917497_m1 | |
| HOXC4 | homeobox C4 | NM_014620 | Hs00538088_m1 | NM_153633 | Hs03043989_m1; | 25.00% |
| | | | | | Hs00205994_m1 | |
| RPL22L1 | ribosomal protein L22-like 1 | NM_001099645 | Hs01595625_m1 | — | — | 25.00% |
| EFNA5 | ephrin-A5 | NM_001962 | Hs00157342_m1 | — | Hs01029098_m1; | 23.33% |
| | | | | | Hs01029096_m1 | |
| TRIM29 | tripartite motif containing 29 | NM_012101 | Hs00232590_m1 | — | Hs00988455_m1; | 20.00% |
| | | | | | Hs00988450_m1; | |
| | | | | | Hs00988451_m1; | |
| | | | | | Hs00988448_m1 | |
| HLA-DMB | major histocompatibility complex, class II, | NM_002118 | Hs00988699_m1 | — | Hs00157943_m1; | 16.67% |
| | DM beta | | | | Hs00157941_m1 | |
| HOXC6 | homeobox C6 | NM_004503 | Hs00171690_m1 | NM_153693 | — | 16.67% |
| THBS4 | thrombospondin 4 | NM_003248 | Hs00170261_m1 | — | Hs01007948_m1; | 15.00% |
| | | | | | Hs01007962_m1; | |
| | | | | | Hs01007949_m1; | |
| | | | | | Hs01007954_m1 | |
| CRISP3 | cysteine-rich secretory protein 3 | NM_006061 | Hs00195988_m1 | NM_001190986 | Hs01119228_m1; | 8.33% |
| | | | | | Hs01119230_m1 | |
| SDK1 | sidekick homolog 1, cell adhesion | NM_152744 | Hs00326727_m1 | NR_027816 | Hs01010129_m1; | 8.33% |
| | molecule | | | | Hs01010142_m1; | |
| | | | | | Hs01010156_m1; | |
| | | | | | Hs01010133_m1 | |
| SRD5A2 | steroid-5-alpha-reductase, alpha | NM_000348 | Hs00165843_m1 | — | Hs03003720_m1; | 8.33% |
| | polypeptide 2 | | | | Hs03003722_m1; | |
| | | | | | Hs00936406_m1; | |
| | | | | | Hs03003719_m1 | |
| TAGLN | transgelin | NM_003186 | Hs00162558_m1 | — | Hs01038780_m1; | 8.33% |
| | | | | | Hs01038771_m1 | |
| WFS1 | Wolfram syndrome 1 | NM_006005 | Hs00903605_m1 | NM_001145853 | Hs00903610_m1; | 8.33% |
| | | | | | Hs00903607_m1; | |
| | | | | | Hs00195634_m1 | |
| SNAI2 | snail homolog 2 (Drosophila) | NM_003068 | Hs00161904_m1 | — | Hs00161904_m1 | 6.67% |
| GDPD1 | glycerophosphodiester phosphodiesterase | NM_182569 | Hs01018359_m1 | NM_001165993; | Hs00402246_m1; | 5.00% |
| | domain containing 1 | | | NM_001165994 | Hs01018360_m1; | |
| | | | | | Hs01018358_m1; | |
| | | | | | Hs01018362_m1 | |
| TTK | TTK protein kinase | NM_003318 | Hs00177412_m1 | NM_001166691 | Hs01011319_m1; | 5.00% |
| | | | | | Hs01009887_m1; | |
| | | | | | Hs01009872_m1; | |
| | | | | | Hs01009881_m1 | |
| OR51E1 | olfactory receptor, family 51, subfamily E, | NM_152430 | Hs00379183_m1 | — | — | 3.33% |
| | member 1 | | | | | |
| PDZD2 | PDZ domain containing 2 | NM_178140 | Hs00389477_m1 | — | Hs01054842_m1; | 3.33% |
| | | | | | Hs01054833_m1; | |
| | | | | | Hs01054824_m1; | |
| | | | | | Hs01054838_m1 | |
| | | | | | Hs00229805_m1; | |
| TDRD1 | tudor domain containing 1 | NM_198795 | Hs00229805_m1 | — | Hs00974888_m1; | 3.33% |
| | | | | | Hs00974897_m1; | |
| | | | | | Hs00974894_m1 | |

TABLE 6A
Expression Characteristics of Prostate Cancer Markers in Prostate Tissues

| | | | | | Mean DeltaCt | | | |
|---|---|---|---|---|---|---|---|---|
| | Official | | Accession | Amplicon | Normal | Tumor | Difference | t-test |
| Rank | Symbol | Gene Name | Number | Size | (n = 5) | (n = 4) | in Means | p-value |
| 1 | CRISP3 | cysteine-rich secretory protein 3 | NM_006061 | 111 | 3.9964 | −0.3945 | 4.3908 | 0.0785 |
| 2 | HOXC6 | homeobox C6 | NM_004503 | 87 | 2.5588 | −0.8571 | 3.4159 | 0.0018 |
| 3 | TDRD1 | tudor domain containing 1 | NM_198795 | 67 | 2.9221 | −0.2505 | 3.1726 | 0.1018 |
| 4 | HOXC4 | homeobox C4 | NM_014620 | 85 | 4.3182 | 1.2809 | 3.0373 | 0.0181 |
| 5 | SNAI2 | snail homolog 2 (Drosophila) | NM_003068 | 79 | −1.6726 | 0.9881 | −2.6607 | 0.0091 |
| 6 | TRIM29 | tripartite motif containing 29 | NM_012101 | 82 | −1.5395 | 0.9594 | −2.4989 | 0.0450 |
| 7 | ERG | v-ets erythroblastosis virus E26 oncogene homolog | NM_182918 | 60 | −1.5375 | −3.9131 | 2.3756 | 0.0293 |
| 8 | TMEM178 | transmembrane protein 178 | NM_152390 | 62 | 1.5028 | −0.8619 | 2.3646 | 0.0895 |
| 9 | THBS4 | thrombospondin 4 | NM_003248 | 96 | 0.0119 | −2.2689 | 2.2808 | 0.0109 |
| 10 | RASSF1A | Ras association domain family member 1 | NM_007182 | 55 | −0.0056 | 1.9497 | −1.9553 | 0.0201 |
| 11 | SDK1 | sidekick homolog 1, cell adhesion molecule | NM_152744 | 57 | 2.0229 | 0.2818 | 1.7411 | 0.0152 |
| 12 | LAMB3 | laminin, beta 3 | NM_000228 | 69 | −0.0503 | 1.5020 | −1.5523 | 0.0155 |
| 13 | HLA-DMB | major histocompatibility complex, class II, DM beta | NM_002118 | 75 | −1.0452 | −2.5851 | 1.5399 | 0.0169 |
| 14 | PDZD2 | PDZ domain containing 2 | NM_178140 | 83 | 0.6423 | 2.1038 | −1.4615 | 0.0781 |
| 15 | EFNA5 | ephrin-A5 | NM_001962 | 98 | −0.5672 | 0.8565 | −1.4237 | 0.0178 |
| 16 | GDPD1 | glycerophosphodiester phosphodiesterase domain | NM_182569 | 69 | −0.1360 | −1.4298 | 1.2938 | 0.0208 |
| | | containing 1 | | | | | | |
| 17 | KRT15 | keratin 15 | NM_002275 | 81 | −2.5306 | −1.3971 | −1.1335 | 0.0351 |
| 18 | FLNC | filamin C, gamma | NM_001458 | 71 | −3.5561 | −2.4583 | −1.0978 | 0.0756 |
| 19 | CACNA1D | calcium channel, voltage-dependent, L type, | NM_000720 | 69 | 0.3375 | −0.6480 | 0.9855 | 0.0429 |
| | | alpha 1D subunit | | | | | | |
| 20 | TAGLN | transgelin | NM_003186 | 82 | −7.8099 | −8.7403 | 0.9305 | 0.4180 |
| 21 | SRD5A2 | steroid-5-alpha-reductase, alpha polypeptide 2 | NM_000348 | 83 | 0.2151 | 1.0266 | −0.8115 | 0.2849 |
| 22 | UAP1 | UDP-N-acteylglucosamine pyrophosphorylase 1 | NM_003115 | 99 | −1.1625 | −1.9597 | 0.7972 | 0.0885 |
| 23 | RPL22L1 | ribosomal protein L22-like 1 | NM_001099645 | 136 | 2.3854 | 3.1724 | −0.7870 | 0.1871 |
| 24 | PDLIM5 | PDZ and LIM domain 5 | NM_006457 | 70 | −0.4704 | −1.0520 | 0.5815 | 0.4472 |
| 25 | OR51E1 | olfactory receptor, family 51, subfamily E, member 1 | NM_152430 | 97 | −0.6694 | −1.2118 | 0.5424 | 0.5713 |
| 26 | KLK3 | kallikrein-related peptidase 3 | NM_001648 | 83 | −8.6492 | −9.0756 | 0.4264 | 0.3092 |
| 27 | HSP1D | heat shock 60 kDa protein 1 (chaperonin) | NM_002156 | 89 | −1.3053 | −1.5978 | 0.2924 | 0.7039 |
| 28 | IMPDH2 | inosine 5′-monophosphate dehydrogenase 2 | NM_000884 | 68 | −0.5926 | −0.6473 | 0.0546 | 0.9624 |
| 29 | TTK | TTK protein kinase | NM_003318 | 64 | 1.7554 | 1.7210 | 0.0343 | 0.9405 |
| 30 | WFS1 | Wolfram syndrome 1 | NM_006005 | 81 | −0.6242 | −0.6017 | −0.0225 | 0.9711 |

TABLE 6B
Co-regulation of Prostate Cancer Markers

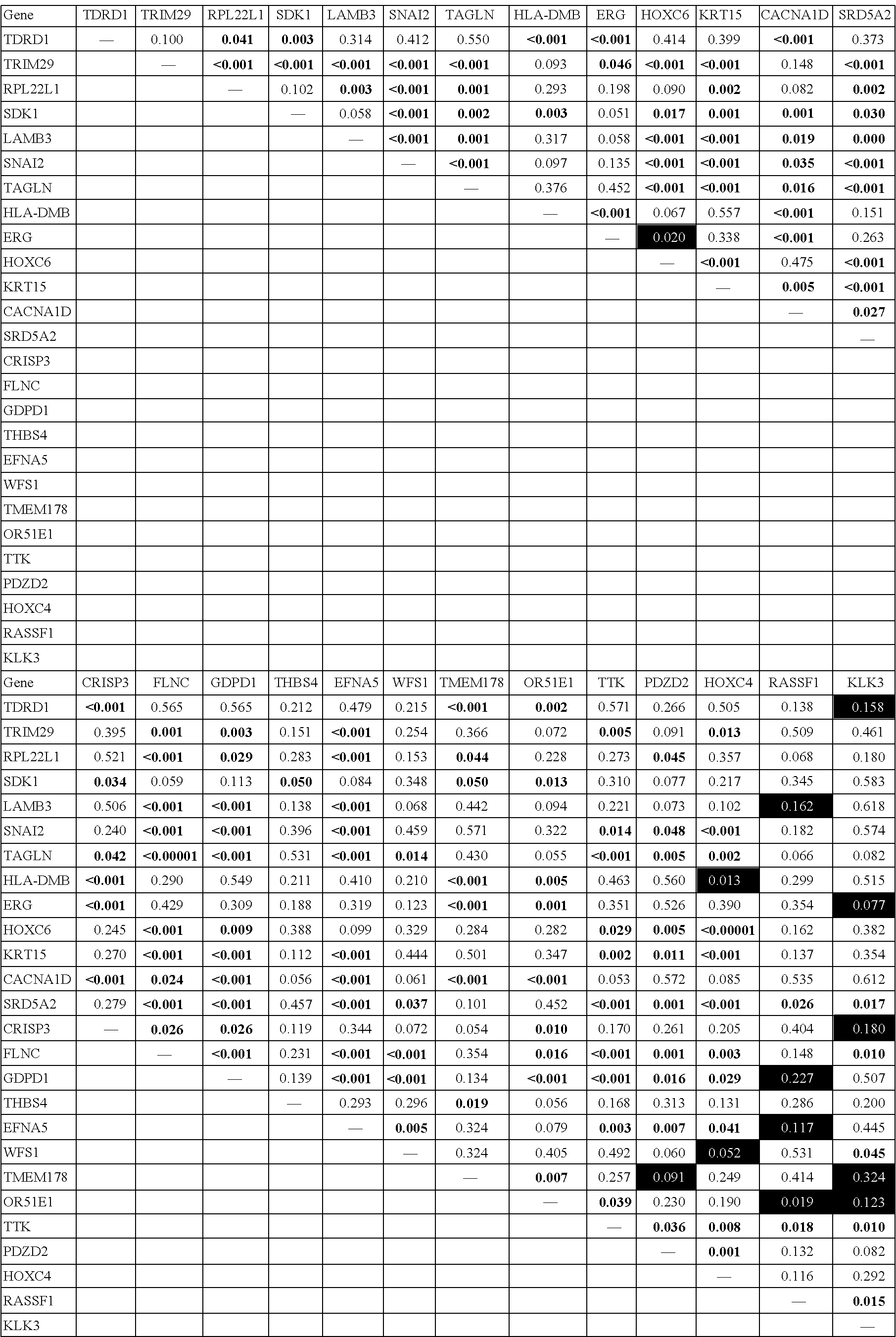

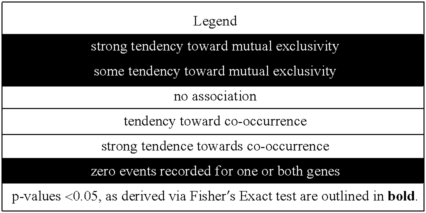

TABLE 7A
Performance Characteristics of Selected Multigene Signatures in Training and Validation Set

| | | | | Training (n = 174; 101N/73T) | | | | | Validation (n = 87, 51N/36T) | | | | |
|---|---|---|---|---|---|---|---|---|---|---|---|---|---|
| | Machine | | | | | Difference | | | | | Difference | | |
| | Learning | | | | | between | DeLong | Gleason | | | between | DeLong | Gleason |
| Classifier | Algorithm | Control Markers | Prostate Cancer Markers | AUC | p-value | areas | p-value | p-value | AUC | p-value | areas | p-value | p-value |
| PCA3 | None | KLK3 | PCA3 | 0.653 | 2.00E−04 | Ref | Ref | 0.602 | 0.714 | 1.66E−04 | Ref | Ref | 0.3260 |
| Class 1 | Naive | KLK3 | ERG, CACNA1D | 0.721 | 1.34E−08 | 0.0680 | 0.1301 | 0.994 | 0.676 | 0.0022 | −0.038 | 0.5277 | 0.5260 |
| | Bayes | | | | | | | | | | | | |
| Class 1 | Naïve | KLK3, IPO8, POLR2A | ERG, CACNA1D | 0.703 | 4.29E−07 | 0.0500 | 0.3684 | 0.472 | 0.685 | 0.0012 | −0.029 | 0.6851 | 0.5600 |
| | Bayes | | | | | | | | | | | | |
| Class 1 | Naïve | IPO8, POLR2A, GUSB, | ERG, CACNA1D | 0.711 | 1.48E−07 | 0.0580 | 0.2955 | 0.609 | 0.721 | 4.93E−05 | 0.007 | 0.9158 | 0.6870 |
| | Bayes | TBP, KLK3 | | | | | | | | | | | |
| Class 2 | Naïve | KLK3 | ERG, HOXC6, TAGLN, TDRD1, CACNA1D, SDK1 | 0.723 | 7.21E−09 | 0.0700 | 0.0932 | 0.997 | 0.693 | 6.15E−04 | −0.021 | 0.7174 | 0.4700 |
| | Bayes | | | | | | | | | | | | |
| Class 2 | Naïve | KLK3, IPO8, POLR2A | ERG, HOXC6, TAGLN, TDRD1, CACNA1D, SDK1 | 0.712 | 1.43E−07 | 0.0590 | 0.2861 | 0.255 | 0.698 | 4.61E−04 | −0.016 | 0.8328 | 0.4920 |
| | Bayes | | | | | | | | | | | | |
| Class 2 | Naïve | IPO8, POLR2A, GUSB, | ERG, HOXC6, TAGLN, TDRD1, CACNA1D, SDK1 | 0.721 | 4.99E−08 | 0.0680 | 0.2147 | 0.394 | 0.743 | 3.84E−06 | 0.029 | 0.6792 | 0.6420 |
| | Bayes | TBP, KLK3 | | | | | | | | | | | |
| Class 3 | Naïve | KLK3 | EFNA5, ERG-SNAI2, ERG-RPL22L1, KRT15, HOXC4 | 0.758 | 1.94E−12 | 0.1050 | 0.0269 | 0.917 | 0.716 | 1.43E−04 | 0.002 | 0.9751 | 0.0471 |
| | Bayes | | | | | | | | | | | | |
| Class 3 | Naïve | KLK3, IPO8, POLR2A | EFNA5, ERG-SNAI2, ERG-RPL22L1, KRT15, HOXC4 | 0.761 | 1.20E−12 | 0.1080 | 0.0300 | 0.998 | 0.740 | 1.23E−05 | 0.026 | 0.6850 | 0.0097 |
| | Bayes | | | | | | | | | | | | |
| Class 3 | Naïve | IPO8, POLR2A, GUSB, | EFNA5,ERG-SNAI2, ERG-RPL22L1, KRT15, HOXC4 | 0.766 | 3.98E−13 | 0.1130 | 0.0254 | 0.897 | 0.755 | 1.78E−06 | 0.041 | 0.5005 | 0.0133 |
| | Bayes | TBP, KLK3 | | | | | | | | | | | |
| Class 4 | Naïve | KLK3 | SRD5A2, ERG-SNAI2, maxERG CACNA1D, LAMB3, | 0.756 | 1.56E−12 | 0.1030 | 0.0246 | 0.448 | 0.710 | 1.82E−04 | −0.004 | 0.9473 | 0.2670 |
| | Bayes | | HOXC4 | | | | | | | | | | |
| Class 4 | Naïve | KLK3, IPO8, POLR2A | SRD5A2, ERG-SNAI2, maxERG CACNA1D, LAMB3, | 0.759 | 8.21E−13 | 0.1060 | 0.0339 | 0.379 | 0.730 | 1.92E−05 | 0.016 | 0.8119 | 0.0442 |
| | Bayes | | HOXC4 | | | | | | | | | | |
| Class 4 | Naïve | IPO8, POLR2A, GUSB, | SRD5A2, ERG-SNAI2, maxERG CACNA1D, LAMB3, | 0.767 | 6.50E−14 | 0.1140 | 0.0221 | 0.348 | 0.748 | 1.94E−06 | 0.034 | 0.5935 | 0.0784 |
| | Bayes | TBP, KLK3 | HOXC4 | | | | | | | | | | |
| Class 5 | Naïve | KLK3 | ERG-SNAI2, ERG, CACNA1D, LAMB3, HOXC4, | 0.763 | 1.45E−13 | 0.1100 | 0.0163 | 0.765 | 0.736 | 1.66E−05 | 0.022 | 0.7329 | 0.0898 |
| | Bayes | | ERG-RPL22L1, KRT15, TRIM29 | | | | | | | | | | |
| Class 5 | Naïve | KLK3, IPO8, POLR2A | ERG-SNAI2, ERG, CACNA1D, LAMB3, HOXC4, | 0.753 | 5.25E−12 | 0.1000 | 0.0501 | 0.611 | 0.748 | 2.44E−06 | 0.034 | 0.6158 | 0.0152 |
| | Bayes | | ERG-RPL22L1, KRT15, TRIM29 | | | | | | | | | | |
| Class 5 | Naïve | IPO8, POLR2A, GUSB, | ERG-SNAI2, ERG, CACNA1D, LAMB3, HOXC4, | 0.759 | 9.17E−13 | 0.1060 | 0.0388 | 0.468 | 0.779 | 1.75E−08 | 0.065 | 0.3103 | 0.0208 |
| | Bayes | TBP, KLK3 | ERG-RPL22L1, KRT15, TRIM29 | | | | | | | | | | |
| Class 6 | Naïve | KLK3 | OR51E1, ERG, CACNA1D, ERG-SNAI2, LAMB3, HOXC4, | 0.759 | 6.71E−13 | 0.1060 | 0.0196 | 0.840 | 0.718 | 1.07E−04 | 0.004 | 0.9435 | 0.0583 |
| | Bayes | | ERG-RPL22L1, KRT15, HOXC6 | | | | | | | | | | |
| Class 6 | Naïve | KLK3, IPO8, POLR2A | OR51E1, ERG, CACNA1D, ERG-SNAI2, LAMB3, HOXC4, | 0.756 | 2.13E−12 | 0.1030 | 0.0379 | 0.947 | 0.740 | 6.32E−06 | 0.026 | 0.6994 | 0.0061 |
| | Bayes | | ERG-RPL22L1, KRT15, HOXC6 | | | | | | | | | | |
| Class 6 | Naïve | IPO8, POLR2A, GUSB, | OR51E1, ERG, CACNA1D, ERG-SNAI2, LAMB3, HOXC4, | 0.762 | 3.97E−13 | 0.1090 | 0.0292 | 0.869 | 0.762 | 3.63E−07 | 0.048 | 0.4595 | 0.0092 |
| | Bayes | TBP, KLK3 | ERG-RPL22L1, KRT15, HOXC6 | | | | | | | | | | |

TABLE 7B
AUC of ROC Curves Analysis using Selected Classifiers with different Prostate-specific
Control Markers

| Prostate | | | | | | | |
|---|---|---|---|---|---|---|---|
| Specific | | | | | | | |
| Control | | Classifiers | | | | | |
| Markers | Other Control Markers | Class 1 | Class 2 | Class 3 | Class 4 | Class 5 | Class 6 |
| KLK3 | — | 0.728 | 0.755 | 0.749 | 0.759 | 0.723 | 0.761 |
| KLK3 | IPO8 POLR2A | 0.716 | 0.772 | 0.779 | 0.785 | 0.730 | 0.783 |
| KLK3 | IPO8 POLR2A GUSB TBP | 0.726 | 0.799 | 0.792 | 0.804 | 0.742 | 0.803 |
| FOLH1 | — | 0.711 | 0.750 | 0.743 | 0.757 | 0.693 | 0.758 |
| FOLH1 | IPO8 POLR2A | 0.709 | 0.779 | 0.773 | 0.783 | 0.718 | 0.786 |
| FOLH1 | IPO8 POLR2A GUSB TBP | 0.721 | 0.800 | 0.793 | 0.803 | 0.725 | 0.804 |
| FOLH1B | — | 0.710 | 0.757 | 0.752 | 0.759 | 0.665 | 0.747 |
| FOLH1B | IPO8 POLR2A | 0.715 | 0.784 | 0.768 | 0.777 | 0.712 | 0.782 |
| FOLH1B | IPO8 POLR2A GUSB TBP | 0.721 | 0.800 | 0.790 | 0.797 | 0.720 | 0.796 |
| PCGEM1 | — | 0.668 | 0.758 | 0.736 | 0.753 | 0.603 | 0.731 |
| PCGEM1 | IPO8 POLR2A | 0.715 | 0.794 | 0.779 | 0.787 | 0.709 | 0.797 |
| PCGEM1 | IPO8 POLR2A GUSB TBP | 0.719 | 0.809 | 0.799 | 0.806 | 0.720 | 0.812 |
| PMEPA1 | — | 0.721 | 0.769 | 0.748 | 0.765 | 0.730 | 0.768 |
| PMEPA1 | IPO8 POLR2A | 0.718 | 0.789 | 0.774 | 0.780 | 0.720 | 0.783 |
| PMEPA1 | IPO8 POLR2A GUSB TBP | 0.723 | 0.805 | 0.792 | 0.799 | 0.728 | 0.799 |
| OR51E1 | — | 0.559 | 0.713 | 0.707 | 0.710 | 0.465 | 0.720 |
| OR51E1 | IPO8 POLR2A | 0.653 | 0.765 | 0.752 | 0.759 | 0.656 | 0.764 |
| OR51E1 | IPO8 POLR2A GUSB TBP | 0.689 | 0.784 | 0.771 | 0.789 | 0.681 | 0.785 |
| OR51E2 | — | 0.515 | 0.723 | 0.699 | 0.700 | 0.656 | 0.691 |
| OR51E2 | IPO8 POLR2A | 0.655 | 0.754 | 0.746 | 0.748 | 0.635 | 0.753 |
| OR51E2 | IPO8 POLR2A GUSB TBP | 0.682 | 0.773 | 0.766 | 0.786 | 0.662 | 0.777 |
| PSCA | — | 0.646 | 0.757 | 0.699 | 0.741 | 0.590 | 0.698 |
| PSCA | IPO8 POLR2A | 0.706 | 0.782 | 0.760 | 0.783 | 0.697 | 0.772 |
| PSCA | IPO8 POLR2A GUSB TBP | 0.705 | 0.800 | 0.781 | 0.804 | 0.707 | 0.794 |

TABLE 8
Performance Characteristics of Prostate Cancer Classifiers in Men Treated for BPH Versus Participants Without any Medication

| | | | Under BPH Medication | | | Without Medication | | |
|---|---|---|---|---|---|---|---|---|
| | | | (n = 51; 37N/14T) | | | (n = 202; 112N/90T) | | |
| Classifier | Control Markers | Prostate Cancer Markers | AUC | SE | 95% Cl | AUC | SE | 95% Cl |
| Class 1 | KLK3 | ERG, CACNA1D | 0.707 | 0.0917 | 0.562-0.826 | 0.696 | 0.0366 | 0.628-0.759 |
| Class 1 | KLK3, IPO8, POLR2A | ERG, CACNA1D | 0.680 | 0.1020 | 0.534-0.803 | 0.700 | 0.0365 | 0.632-0.762 |
| Class 1 | IPO8, POLR2A, GUSB, | ERG, CACNA1D | 0.674 | 0.1030 | 0.528-0.798 | 0.718 | 0.0359 | 0.651-0.779 |
| | TBP, KLK3 | | | | | | | |
| Class 2 | KLK3 | ERG, HOXC6, TAGLN, | 0.699 | 0.0887 | 0.554-0.819 | 0.712 | 0.0359 | 0.644-0.773 |
| | | TDRD1, CACNA1D, SDK1 | | | | | | |
| Class 2 | KLK3, IPO8, POLR2A | ERG, HOXC6, TAGLN, | 0.681 | 0.0987 | 0.536-0.805 | 0.714 | 0.0363 | 0.646-0.775 |
| | | TDRD1, CACNA1D, SDK1 | | | | | | |
| Class 2 | IPO8, POLR2A, GUSB, | ERG, HOXC6, TAGLN, | 0.680 | 0.0996 | 0.534-0.803 | 0.736 | 0.0356 | 0.670-0.796 |
| | TBP, KLK3 | TDRD1, CACNA1D, SDK1 | | | | | | |
| Class 3 | KLK3 | EFNA5, ERG-SNAI2, ERG-RPL22L1, | 0.855 | 0.0574 | 0.728-0.938 | 0.714 | 0.0364 | 0.647-0.775 |
| | | KRT15, HOXC4 | | | | | | |
| Class 3 | KLK3, IPO8, POLR2A | EFNA5, ERG-SNAI2, ERG-RPL22L1, | 0.840 | 0.0662 | 0.710-0.927 | 0.729 | 0.0360 | 0.662-0.789 |
| | | KRT15, HOXC4 | | | | | | |
| Class 3 | IPO8, POLR2A, GUSB, | EFNA5, ERG-SNAI2, ERG-RPL22L1, | 0.826 | 0.0707 | 0.694-0.918 | 0.745 | 0.0353 | 0.679-0.804 |
| | TBP, KLK3 | KRT15, HOXC4 | | | | | | |
| Class 4 | KLK3 | SRD5A2, ERG-SNAI2, maxERG | 0.813 | 0.0707 | 0.679-0.908 | 0.719 | 0.0356 | 0.652-0.780 |
| | | CACNA1D, LAMB3, HOXC4 | | | | | | |
| Class 4 | KLK3, IPO8, POLR2A | SRD5A2, ERG-SNAI2, maxERG | 0.790 | 0.0771 | 0.653-0.891 | 0.727 | 0.0351 | 0.660-0.787 |
| | | CACNA1D, LAMB3, HOXC4 | | | | | | |
| Class 4 | IPO8, POLR2A, GUSB, | SRD5A2, ERG-SNAI2, maxERG | 0.797 | 0.0769 | 0.661-0.897 | 0.741 | 0.0344 | 0.675-0.800 |
| | TBP, KLK3 | CACNA1D, LAMB3, HOXC4 | | | | | | |
| Class 5 | KLK3 | ERG-SNAI2, ERG, CACNA1D, LAMB3, | 0.830 | 0.0684 | 0.699-0.921 | 0.733 | 0.0350 | 0.667-0.793 |
| | | HOXC4, ERG-RPL22L1, KRT15, TRIM29 | | | | | | |
| Class 5 | KLK3, IPO8, POLR2A | ERG-SNAI2, ERG, CACNA1D, LAMB3, | 0.805 | 0.0780 | 0.670-0.903 | 0.731 | 0.0351 | 0.664-0.790 |
| | | HOXC4, ERG-RPL22L1, KRT15, TRIM29 | | | | | | |
| Class 5 | IPO8, POLR2A, GUSB, | ERG-SNAI2, ERG, CACNA1D, LAMB3, | 0.799 | 0.0782 | 0.664-0.898 | 0.750 | 0.0341 | 0.684-0.808 |
| | TBP, KLK3 | HOXC4, ERG-RPL22L1, KRT15, TRIM29 | | | | | | |
| Class 6 | KLK3 | OR51E1, ERG, CACNA1D, ERG-SNAI2, | 0.820 | 0.0734 | 0.688-0.914 | 0.714 | 0.0360 | 0.646-0.775 |
| | | LAMB3, HOXC4, ERG-RPL22L1, | | | | | | |
| | | KRT15, HOXC6 | | | | | | |
| Class 6 | KLK3, IPO8, POLR2A | OR51E1, ERG, CACNA1D, ERG-SNAI2, | 0.807 | 0.0807 | 0.672-0.904 | 0.723 | 0.0354 | 0.656-0.784 |
| | | LAMB3, HOXC4, ERG-RPL22L1, | | | | | | |
| | | KRT15, HOXC6 | | | | | | |
| Class 6 | IPO8, POLR2A, GUSB, | OR51E1, ERG, CACNA1D, ERG-SNAI2, | 0.805 | 0.0800 | 0.670-0.903 | 0.737 | 0.0346 | 0.671-0.796 |
| | TBP, KLK3 | LAMB3, HOXC4, ERG-RPL22L1, | | | | | | |
| | | KRT15, HOXC6 | | | | | | |

TABLE 9
Performance Characteristics of Selected Prostate Cancer Multigene Signatures

| | | | High Grade Cancer | | | Prior First Biopsy | | |
|---|---|---|---|---|---|---|---|---|
| | | | (n = 204; 152N/52T) | | | (n = 220; 122N/98T) | | |
| Classifier | Control Markers | Prostate Cancer Markers | AUC | SE | 95% Cl | AUC | SE | 95% Cl |
| Class 1 | KLK3 | ERG, CACNA1D | 0.702 | 0.0400 | 0.634 to 0.764 | 0.701 | 0.0348 | 0.635 to 0.760 |
| Class 1 | KLK3, IPO8, POLR2A | ERG, CACNA1D | 0.718 | 0.0406 | 0.651 to 0.779 | 0.676 | 0.0361 | 0.610 to 0.737 |
| Class 1 | IPO8, POLR2A, GUSB, | ERG, CACNA1D | 0.731 | 0.0404 | 0.664 to 0.790 | 0.692 | 0.0357 | 0.626 to 0.752 |
| | TBP, KLK3 | | | | | | | |
| Class 2 | KLK3 | ERG, HOXC6, TAGLN, | 0.711 | 0.0384 | 0.644 to 0.773 | 0.717 | 0.0342 | 0.653 to 0.776 |
| | | TDRD1, CACNA1D, SDK1 | | | | | | |
| Class 2 | KLK3, IPO8, POLR2A | ERG, HOXC6, TAGLN, | 0.744 | 0.0396 | 0.678 to 0.802 | 0.691 | 0.0361 | 0.626 to 0.752 |
| | | TDRD1, CACNA1D, SDK1 | | | | | | |
| Class 2 | IPO8, POLR2A, GUSB, | ERG, HOXC6, TAGLN, | 0.759 | 0.0386 | 0.695 to 0.816 | 0.708 | 0.0358 | 0.643 to 0.767 |
| | TBP, KLK3 | TDRD1, CACNA1D, SDK1 | | | | | | |
| Class 3 | KLK3 | EFNA5, ERG-SNAI2, ERG-RPL22L1, | 0.772 | 0.0355 | 0.708 to 0.827 | 0.753 | 0.0331 | 0.690 to 0.808 |
| | | KRT15, HOXC4 | | | | | | |
| Class 3 | KLK3, IPO8, POLR2A | EFNA5, ERG-SNAI2, ERG-RPL22L1, | 0.779 | 0.0362 | 0.715 to 0.834 | 0.749 | 0.0334 | 0.687 to 0.805 |
| | | KRT15, HOXC4 | | | | | | |
| Class 3 | IPO8, POLR2A, GUSB, | EFNA5, ERG-SNAI2, ERG-RPL22L1, | 0.791 | 0.0358 | 0.729 to 0.845 | 0.757 | 0.0331 | 0.695 to 0.812 |
| | TBP, KLK3 | KRT15, HOXC4 | | | | | | |
| Class 4 | KLK3 | SRD5A2, ERG-SNAI2, maxERG | 0.778 | 0.0340 | 0.714 to 0.833 | 0.740 | 0.0332 | 0.677 to 0.797 |
| | | CACNA1D, LAMB3, HOXC4 | | | | | | |
| Class 4 | KLK3, IPO8, POLR2A | SRD5A2, ERG-SNAI2, maxERG | 0.792 | 0.0325 | 0.730 to 0.846 | 0.736 | 0.0332 | 0.672 to 0.793 |
| | | CACNA1D, LAMB3, HOXC4 | | | | | | |
| Class 4 | IPO8, POLR2A, GUSB, | SRD5A2, ERG-SNAI2, maxERG | 0.800 | 0.0326 | 0.738 to 0.852 | 0.747 | 0.0326 | 0.684 to 0.803 |
| | TBP, KLK3 | CACNA1D, LAMB3, HOXC4 | | | | | | |
| Class 5 | KLK3 | ERG-SNAI2, ERG, CACNA1D, LAMB3, | 0.790 | 0.0335 | 0.727 to 0.843 | 0.753 | 0.0325 | 0.690 to 0.808 |
| | | HOXC4, ERG-RPL22L1, KRT15, TRIM29 | | | | | | |
| Class 5 | KLK3, IPO8, POLR2A | ERG-SNAI2, ERG, CACNA1D, LAMB3, | 0.794 | 0.0339 | 0.732 to 0.847 | 0.737 | 0.0333 | 0.674 to 0.794 |
| | | HOXC4, ERG-RPL22L1, KRT15, TRIM29 | | | | | | |
| Class 5 | IPO8, POLR2A, GUSB, | ERG-SNAI2, ERG, CACNA1D, LAMB3, | 0.806 | 0.0333 | 0.744 to 0.857 | 0.751 | 0.0325 | 0.689 to 0.807 |
| | TBP, KLK3 | HOXC4, ERG-RPL22L1, KRT15, TRIM29 | | | | | | |
| Class 6 | KLK3 | OR51E1, ERG, CACNA1D, ERG-SNAI2, | 0.774 | 0.0343 | 0.710 to 0.829 | 0.740 | 0.0332 | 0.677 to 0.797 |
| | | LAMB3, HOXC4, ERG-RPL22L1, | | | | | | |
| | | KRT15, HOXC6 | | | | | | |
| Class 6 | KLK3, IPO8, POLR2A | OR51E1, ERG, CACNA1D, ERG-SNAI2, | 0.785 | 0.0345 | 0.723 to 0.840 | 0.733 | 0.0335 | 0.670 to 0.790 |
| | | LAMB3, HOXC4, ERG-RPL22L1, | | | | | | |
| | | KRT15, HOXC6 | | | | | | |
| Class 6 | IPO8, POLR2A, GUSB, | OR51E1, ERG, CACNA1D, ERG-SNAI2, | 0.797 | 0.0336 | 0.735 to 0.850 | 0.743 | 0.0329 | 0.680 to 0.800 |
| | TBP, KLK3 | LAMB3, HOXC4, ERG-RPL22L1, | | | | | | |
| | | KRT15, HOXC6 | | | | | | |

TABLE 10
Sequence Listing

| Marker | Exemplary SEQ ID NO: |
|---|---|
| GUSB | 1 |
| HPRT1 | 2 |
| IPO8 | 3 |
| POLR2A | 4 |
| TBP | 5 |
| KLK3 | 6 |
| FOLH1 | 7 |
| FOLH1B | 8 |
| OR51E1 | 9 |
| OR51E2 | 10 |
| PCGEM1 | 11 |
| PMEPA1 | 12 |
| PSCA | 13 |
| KRT15 | 14 |
| CACNA1D | 15 |
| ERG | 16 |
| LAMB3 | 17 |
| FLNC | 18 |
| RASSF1A | 19 |
| TMEM178 | 20 |
| HOXC4 | 21 |
| RPL22L1 | 22 |
| EFNA5 | 23 |
| TRIM29 | 24 |
| HLA-DMB | 25 |
| HOXC6 | 26 |
| THBS4 | 27 |
| CRISP3 | 28 |
| SDK1 | 29 |
| SDR5A2 | 30 |
| TAGLN | 31 |
| WFS1 | 32 |
| SNAI2 | 33 |
| GDPD1 | 34 |
| TTK | 35 |
| PDZD2 | 36 |
| TDRD1 | 37 |

The present invention is illustrated in further details by the following non-limiting examples.

### Example 1

### Gene Expression Profile Analysis of Whole Urine Samples

We determined the technical feasibility of gene expression profiling in whole urine samples in men having or suspected of having prostate cancer. Urine samples were collected from 90 men having undergone a digital rectal exam (DRE) prior to a transrectal ultrasound-guided prostate biopsy, the results of which were used to categorize subjects into two groups: (1) men having prostate cancer; and (2) men not having prostate cancer with or without benign prostate conditions. Biopsy results were used to assign subjects into either of these two categories. Benign prostate cancer conditions include: benign prostatic hyperplasia (BPH), high-grade prostatic intraepithelial neoplasia (HG-PIN), atypical small acinar proliferation (ASAP), and/or atypical prostatic cells (Atypia). In all cases, categorization or stratification of the samples was based on interpretation of the biopsy as assessed by a pathologist. Following stratification based upon biopsy results, 45 urine samples were identified as being from men having prostate cancer with confirmed positive biopsy, and 45 urine samples were identified as being from men with negative biopsy results.

Before the biopsy, subjects underwent an attentive DRE performed by a physician who was given instructions to perform a thorough prostate palpation for 15 to 30 seconds. After the DRE, the first 20 to 30 mL of voided urine was collected and mixed with an equal volume of a buffer containing guanidine thiocyanate. Total RNA was extracted from whole urine samples based on the denaturing properties of chaotropic agents, binding of nucleic acid to silica particles, and finally eluting in buffered water.

Gene expression levels were measured by RT-qPCR using TagMan® Gene Expression Assays (Applied Biosystems). A panel of candidate markers was preselected based on their reported expression in either prostate or prostate-cancer cells. A list of these candidate markers used for gene expression profiling in this study is given in Table 1. All TaqMan® assays were selected to perform standard gene expression experiments as they can detect the maximum number of transcripts for the gene of interest without detecting gene products with similar sequence, such as homologs. Most assays were designed across an exon-exon junction, targeting a short amplicon without detecting off-target sequences, thus increasing the efficiency and specificity of the PCR reaction. Based on an evaluation of each of the assays with the Entrez SNP database at NCBI, single-nucleotide polymorphisms (SNPs) were found to be located under certain probe or primer sequences for some assays used in this study. Reference sequence (RS) numbers for each associated SNPs are also listed in Table 1.

About 20 μL of RNA were transcribed into single-stranded cDNA using nucleic acids extracted from whole urine samples and the High-Capacity Archive Kit (Applied Biosystems, Foster City, Calif.) with random hexamers as primers in a final volume of 100 μL as described in the manufacturer's protocol. Quantitative real-time PCR (qPCR) reactions were performed using 5 μL of a 1:10 (v/v) dilution of the cDNA reaction in DNase/RNase free water, the TaqMan® Fast Advanced Master Mix (Applied Biosystems) and TaqMan® Gene Expression Assays (Applied Biosystems) for each candidate marker listed in Table 1 in a final volume of 20 μL on an 7900HT Fast PCR System (Applied Biosystems) as recommended by the manufacturer. TaqMan® Exogenous Internal Positive Control (VIC Probe) was used in duplex as an internal positive control (IPC) in all qPCR reactions to distinguish samples identified as negative because they lack the target sequence from samples identified as negative or because of the presence of a PCR inhibitor.

Raw data were recorded with the Sequence Detection System (SDS) software of the instrument. Cycle threshold (Ct) values were determined for each candidate prostate cancer marker. Furthermore, normalized gene expression values were calculated based on the delta Ct method, in which the difference between the Ct of each prostate cancer marker and the mean Ct value of five (5) control markers listed in Table 2, namely HPRT1, TBP, IPO8, POLR2A and GUSB, is established. The data were normalized to correct for potential technical variability and deviation in RNA integrity and quantity in each PCR reaction. The normalized gene expression value was compared between normal and prostate cancer subjects. For each individual prostate cancer marker, the difference in mean expression value (delta Ct) between non-cancer and cancer subjects is presented in Table 3A. Prostate cancer markers were ranked according to their significant change between non-cancer and cancer subjects based on Student's T-test. A p-value <0.05 was considered statistically significant. The top-scoring prostate cancer markers ERG, PCA3 and CACNA1 D were found to be highly over-expressed in whole urine from subjects with prostate cancer as compared to that from subjects lacking prostate cancer.

In addition to gene expression analysis, the performance of the individual prostate cancer markers was evaluated using the area under the receiver operating characteristic curves (hereinafter referred to as AUC and ROC curves) to identify genes associated with the presence of prostate cancer cells in whole urine samples. Table 3A provides performance characteristics on whole urine samples. As can be observed, the top-scoring genes, based on normalized expression, are also those that best discriminate whether a urine sample is from a non-prostate cancer subject or a prostate cancer subject.

### Example 2

### Gene Expression Profile Analysis of Urine Sediments

The study shown in Example 1 was repeated on urine samples from a group of 77 subjects that were obtained after DRE and analyzed by quantitative RT-PCR for the genes listed in Table 1, with the exception that instead of using whole urine, the urine samples were centrifuged to pellet cells prior to nucleic acid extraction. The entire procedure took about 15 minutes and was carried out in a clinical centrifuge at 2,500 rpm. The resulting urine sediments containing epithelial cells from the urogenital tract were then extracted as described in Example 1. Table 3B provides mean normalized expression values in normal subjects and cancer subjects for individual genes, as well as performance characteristics based on ROC curve analysis. The genes significantly associated with the presence of prostate cancer cells were either up-regulated or down-regulated. It was determined that the genes whose expression values were significantly different between normal subjects and prostate cancer subjects could be used to predict presence of cancer or cancer development in an individual.

### Example 3

### Machine Learning Methods Used to Study Genes Significantly Associated with Prostate Cancer

Here, we analyzed normalized gene expression data from the 90 whole urine samples of Example 1 using machine learning methods to select and weight individual genes, gene pairs or set of genes according to their ability to separate prostate cancer patients from non-prostate cancer individuals. There are many different methods to combine genes that individually best classify large data sources, one being the design of class predictor (a.k.a. classifier) based on a pre-selected subset of genes. We complemented this set of individual gene features by a set of pair gene features obtained by taking the maximum of the two delta Cts (e.g., “maxERG CACNA1D”) or by subtracting the delta Cts of two pairs of genes (e.g., ERG-SNAI2). While connections of some of the selected genes were found to cancer and/or prostate in Example 1 and 2, their relationship to the prostate-cancer marker PCA3 was not previously documented.

We selected five machine learning algorithms: Naïve Bayes, linear discriminant analysis (LDA), quadratic discriminant analysis (QDA), Random Forest, support vector machine using radial and linear kernels (SVM). These different machine learning algorithms are all well accepted and widely used in the field, but differ so significantly in their design that they enable us to cover a wide range of mathematical models ensuring us to find at least one optimal model. By training a computational model using a machine learning algorithm on a dataset containing normalized gene expression values (e.g., delta Ct) for a set of candidate markers, we were able to define multi-gene signatures capable of providing a clinical assessment of prostate cancer with optimal parameters tuned to achieve the best clinical performance.

To assess the performance of the model, a two-samples-out cross-validation was used. Briefly, one cancer and one non-cancer sample were removed from the dataset and the parameters of the model were trained on the remaining dataset. After the training phase, the model was then applied on the left-out samples. Using cross-validation, it was possible to get an unbiased estimation of the performance of the multigene signature because the samples on which the model was tested had not been used for training. The result of this cross-validation step was a cross-validated receiver operating characteristic (ROC) curve for which we were able to calculate the area under the ROC curve (AUC). Table 4A presents the top scoring machine learning algorithms with their corresponding clinical performances for each multigene signature. Data normalization using the delta Ct calculation method based on mean expression value of five (5) endogenous control genes selected from Table 2 allowed us to generate multigene signatures using machine learning algorithms. We observed that Random forest and Naïve Bayes classifiers represent the two best performing machine learning approaches. The change in AUC in comparison with that of a ratio of PCA3 over PSA was also quantified and p-values were generated using DeLong's test. P-values <0.05 were considered to provide statistical evidence of the best overall test.

In total, 53 multi-gene prostate cancer signatures were found to outperform the PCA3 over PSA test, for some signatures using as little as two prostate cancer markers (Table 4A). Using the same approach, we then applied the selected machine learning algorithms to a group of samples comprising whole urine samples and urine sediments with confirmed presence of prostate cells as assessed by KLK3 gene expression level (Table 4B). The results of this analysis were used to validate that the selected prostate cancer signatures generated through the use of machine learning algorithms can accurately provide a clinical assessment of prostate cancer in a biological sample (e.g., whole urine or urine sediments) containing a background of contaminating prostate cells that are not necessary from prostate cancer cells.

Table 5 provides a list of 25 individual genes which can act as prostate cancer markers within various prostate cancer signatures. Interestingly, we observed the repeated presence of KRT15, ERG, CACNA1D and LAMB3 in the top-scoring prostate cancer signatures.

### Example 4

### Expression Profiling of Selected Genes in Prostate Tissue

The development of diagnostic assays in a rapidly changing technology environment is challenging. There is an urgent need for new markers capable of distinguishing between normal, benign and malignant prostate tissue and for predicting the extent and malignancy of prostate cancer. Although urine-based markers would be particularly desirable for screening prior to biopsy, gene expression evaluation in biopsied prostate tissues or in surgically-resected prostate could also be useful to diagnose and prognosticate prostate diseases. This study therefore examined gene expression levels of a 36-gene panel of reference (Table 2) and prostate cancer-related genes (Table 6A) using quantitative RT-PCR. In total, nine (9) samples from prostatectomy were used for this study; five (5) from normal tissues and four (4) from prostate cancer tissues. Classification of samples was based on interpretation of the Gleason score, TNM staging system and percentage of tumor involvement as assessed by pathologists. RNA from fresh frozen prostate tissues was extracted using twenty (20) sections of 5 μm resuspended in 1 mL of Trizol® reagent (Invitrogen, Carlsbad, Calif.). Extraction of nucleic acids (RNA and to a lesser extent DNA) was performed as recommended by the manufacturer and resuspended in 60 μL of DNase/RNase free water.

Quantity and quality of nucleic acids extracted was evaluated using the Quant-iT™ RNA Assay Kit (Invitrogen, Carlsbad, Calif.) and the Nanodrop™ ND-1000 spectrophotometer (Thermo Scientific, Wilmington, Del.). RNAs were transcribed into single-stranded cDNAs using a minimum of 250 ng of nucleic acids extracted from prostate tissues and the High-Capacity Archive Kit (Applied Biosystems, Foster City, Calif.) with random hexamers as primers in a final volume of 50 μL, as described in the manufacturer's protocol. Gene expression levels were measured using TaqMan® gene expression assays. Quantitative real-time PCR reactions were performed using 5 μL of a 1:10 (v/v) dilution of the cDNA reaction in DNase/RNase free water, the TaqMan® Fast Advanced Master Mix (Applied Biosystems), the TaqMan® Gene Expression Assays (Applied Biosystems) listed in Table 2 and Table 6A in duplex with the TaqMan® Exogenous Internal Positive Control in a final volume of 20 μL on an 7900HT Fast PCR System (Applied Biosystems) as recommended by the manufacturer. All analyses were conducted on normalized gene expression levels using the average Ct values from 5 reference genes (HPRT1, TBP, IPO8, POLR2A and GUSB).

For each individual gene, difference in mean expression value (delta Ct) between normal prostate tissue and prostate cancer tissue is presented in Table 6A. Genes were ranked according to their significant change between normal subjects and cancer subjects based on Student's T-test. Gene expression analysis showed that members of the homeobox gene family HOXC6 and HOXC4 were up-regulated in prostate cancer. Homeobox genes are a large family of similar genes that direct the formation of many body structures during early embryonic development. Genes in the homeobox family are involved in a wide range of critical activities during development and their overexpression promote cellular transformation in cultured cells. Differences in expression were also observed for CRISP3, TDRD1 and PCA3, but the differences were not significant. Furthermore, a number of genes were also found to be significantly down-regulated in prostate cancer tissue. Among these were several known prostate cancer relevant genes, such as TRIM29, EFNA5 and LAMB3. The transcriptional repressor SNAI2 involved in oncogenic transformation of epithelial cells was also found significantly down-regulated in prostate cancer.

We hereby provide subsets of genes (or classifiers) whose expression level is capable of distinguishing prostate cancer, and normal prostate tissue from benign prostate conditions. It was also observed that genes often worked together and that their expression can be co-regulated in a concerted way, a process also referred to as co-occurrence (or co-regulation). Co-regulated genes identified for a disease process like cancer can serve as biomarkers for tumor status and can thus be used in lieu of, or in addition to, the assayed gene with which it is co-expressed. Mutual exclusivity and co-expression analysis of 26 selected genes associated with the presence of prostate cancer was performed using a public dataset (GSE21032) containing log 2 whole transcript mRNA expression values from 150 patients with prostate cancer (Table 6B). Gene expression profile of primary and metastatic prostate cancer tissues was performed using the GeneChip® Human Exon 1.0 ST Array (Affymetix, Santa Clara, Calif.).

Certain cancer genes contribute to tumorigenesis in a manner which is either co-occurring or mutually exclusive. Here, one goal was to identify sets of connected genes that are up- or down-regulated across multiple patients and belong to the same biological process, such as cancer development and progression. The underlying rationale was that genes regulated by similar pathway should co-occur more frequently than expected in pre-configured gene sets that have been grouped according to various measures of similarity. Thus, genes whose expression is governed by similar signals are expected to co-occur significantly in distinct gene expression signatures and to form a strongly interconnected network with different biological pathways. Gene sets that exhibit these properties are very likely to drive cancer progression. The algorithm accessible via the cBio Cancer Genomics Portal (http://cbioportal.org) computed mutual exclusivity or co-occurrence between all pairs of genes and generated a binary matrix with p-values for all target genes (Table 6B) by applying the Fisher Exact test to each individual gene pair. Using this approach, individual genes as well as entire signatures can be assigned to pathways such as cancer development and progression, whose composition of the gene signatures is entirely determined by common genomic features that are consistent with the pathway assignment. Following this procedure, we identified two pairs of genes, FLNC:TAGLN and HOXC4:HOXC6, that exhibited a statistically significant strong tendency towards co-occurrence with p-values <0.00001. A large number of genes also exhibited a significant tendency toward co-occurrence. As an example, one of the top-scoring genes, CRISP3, was found to be co-expressed with 9 other genes. The strongest association observed for CRISP3 was with TDRD1, ERG, and CACNA1D (all p-values <0.001). Although being only minimally down-regulated in cancer tissues, the SRD5A2 gene involved in the androgen metabolism pathway was one of the most commonly co-regulated genes and was found to be significantly co-expressed with 18 other genes tested. In searching for mutually exclusive gene sets, only 6 genes were found to have a strong tendency toward mutual exclusivity. The PCA3/KLK3 gene pair had the highest p-value for mutual exclusivity (p=0.0045). The two other high-scoring pairs included ERG:HOXC6 (p=0.02) and OR51E1:RASSF1 (p=0.018).

### Example 5

### Selection of Genes for Accurate Normalization of Large Gene Expression Data in Urine Samples

To minimize errors and sample-to-sample variation, gene expression analysis from quantitative RT-PCR is usually performed based on relative quantification of specific nucleic acid sequences with an internal standard. Evaluation of stable control markers in clinical samples is desirable for precise and accurate normalization of relative gene expression using an RT-qPCR platform or other related amplification methods. The endogenous control markers to be used in conjunction with prostate cancer markers for the detection of prostate cells in a patient's sample, shall ideally have an expression that is not significantly affected by the presence of cancer cells in a tissue or body fluid, and a similar behavior in samples taken from different individuals or under stress factors such as alkaline conditions.

To identify suitable control markers having stable expression in samples that may contain prostate cells, expression of 10 candidate endogenous reference genes was determined in whole urine samples from 152 non-prostate cancer subjects, 109 prostate cancer subjects and 9 frozen prostate tissues (5 non-cancers and 4 cancers). The RT-qPCR was performed as described above in Example 1 and each reaction plate included an exogenous control reaction using a commercial human universal RNA (Clonetech).

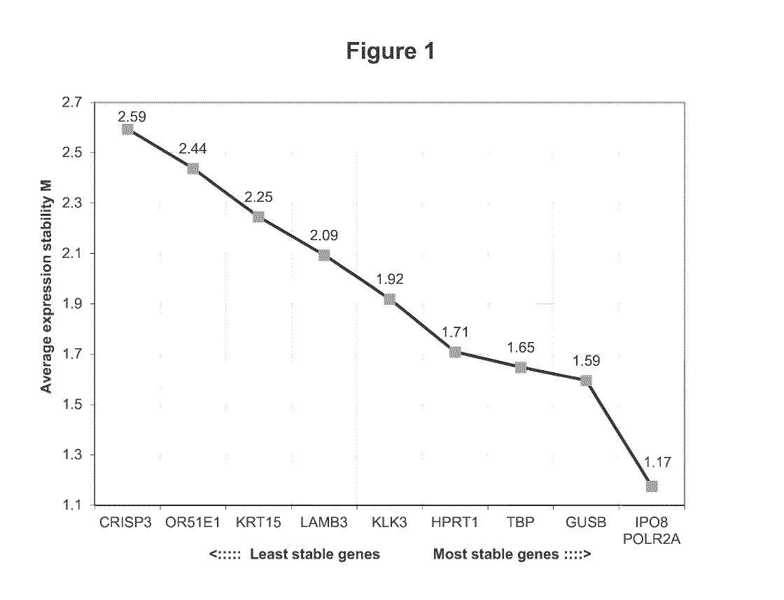

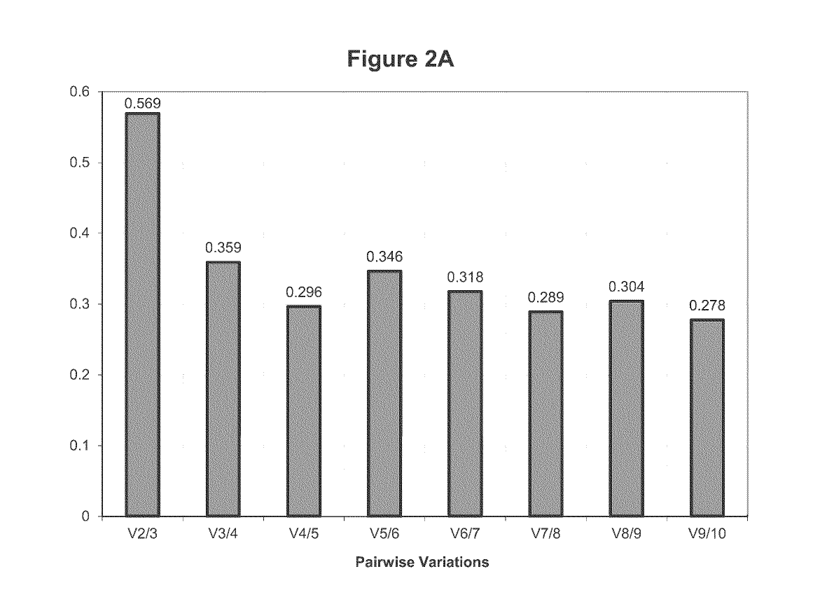

An ideal reference gene should maintain constant expression in urine samples from both prostate cancer and non-prostate cancer subjects. Expression stability was analyzed using the geNorm™ software. In general, geNorm™ uses a pair-wise comparison model to select the gene pair showing the least variation in expression ratio across samples. The software computes a measure of gene stability (M) for each endogenous reference gene. FIG. 1 shows the M values for some of the tested genes. Two genes (IPO8 and POLR2A) demonstrated M values lower than the geNorm™ default threshold of 1.5. Although the reference genes selected have M values that vary, their expression was not de-regulated per se in prostate cancer. Furthermore, while POLR2A and IPO8 were identified as the most stable gene pair, TBP and GUSB showed less variability in their mRNA expression in the urine samples (FIG. 1).

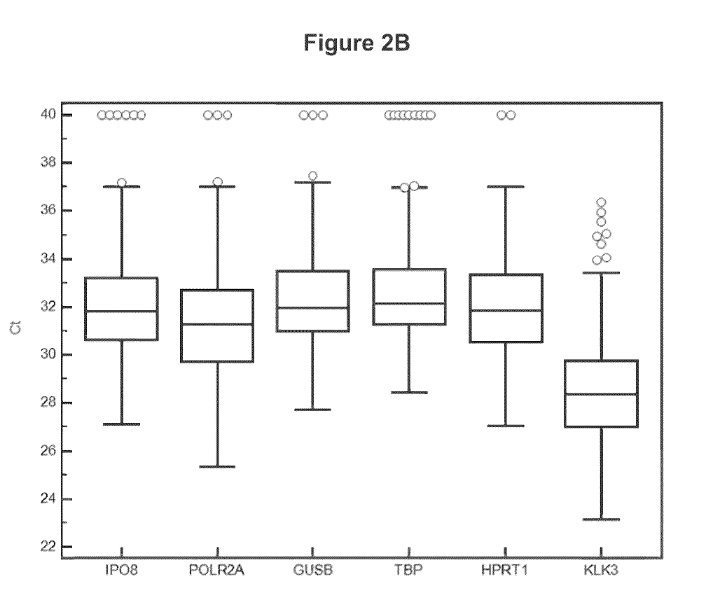

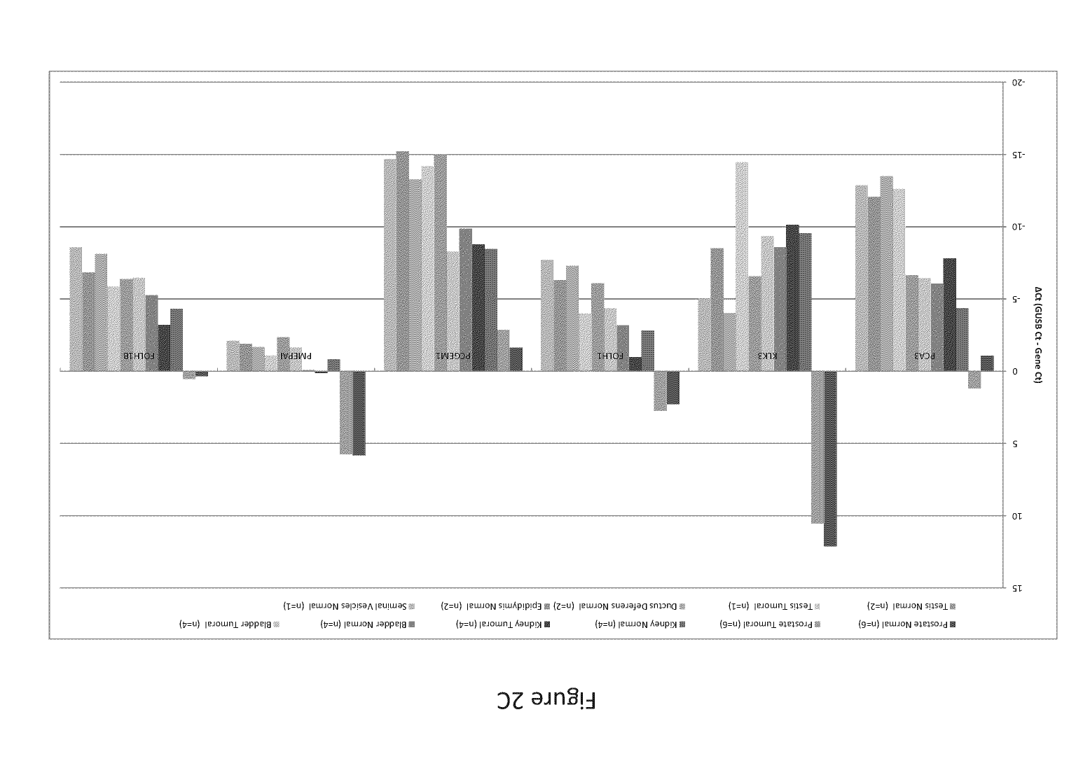

It has been a standard practice in quantitative PCR to use a single reference gene for RNA expression normalization. However, our studies revealed that reference gene expression can vary considerably. This suggested that the use of multiple reference genes may improve accuracy in relative quantification studies. Therefore, it was desirable to identify the appropriate combination of control markers to be used for the sample being tested (e.g., urine). To determine the optimal number of reference genes required for quantitative PCR normalization, the geNorm software calculates a pairwise variation V for each sequentially increasing number of reference genes added. FIG. 2A shows a graph of the pairwise variation calculated by the geNorm software. The geNorm V value of 0.3 was used as a cutoff to determine the optimal number of genes. This analysis revealed that, in the conditions used, the optimal number of endogenous reference genes was four (POLR2A, IPO8, GUSB and TBP) when using RNA extracted from whole urine sample (FIG. 2A).

As an example, the control markers listed in Table 2 do not exhibit an expression level that is significantly different in cancerous prostate tissues compared to non-cancerous prostate tissues, and their expression is also quite constant among the same tissue type taken from different patients (FIG. 2B). Although gene expression profiling of one or more genes is usually measured in tissue samples, the expression level of altered genes may also be measured in cells recovered from sites distant from the primary tumor tissue, for example distant organs, circulating tumor cells and body fluids such as urine, semen, blood and blood fraction. For this purpose, we further evaluated reference gene expression levels in cell lines derived from other malignancies than prostate using a human universal RNA composed of total RNA from 10 human cell lines. This human universal RNA is designed to be used for gene profiling experiments.

Beside these four (4) endogenous reference genes, the use of markers that are specific to prostate cells, such as PSA (a.k.a. KLK3), was desirable to control for the presence of nucleic acid originating from prostate cells in the sample. To demonstrate the possibility of using prostate specific markers for the normalization of gene expression data in urine samples, tissue specificity of five (5) prostate specific control markers listed in table 2 were characterized in tumor and non-tumor tissues of the male genitourinary tract (FIG. 2C). All genes demonstrated a level of expression in prostatic tissues at many orders of magnitude higher than all the other tissues tested. The high specificity of these prostate-specific control markers has made it possible to identify the presence of nucleic acid originating from prostate epithelial cells among non-prostate cells. The use of these prostate-specific control markers can thus be used in addition to or in lieu of PSA (a.k.a KLK3) for gene expression level normalization where the sample may contain nucleic acid from non-prostate cells.

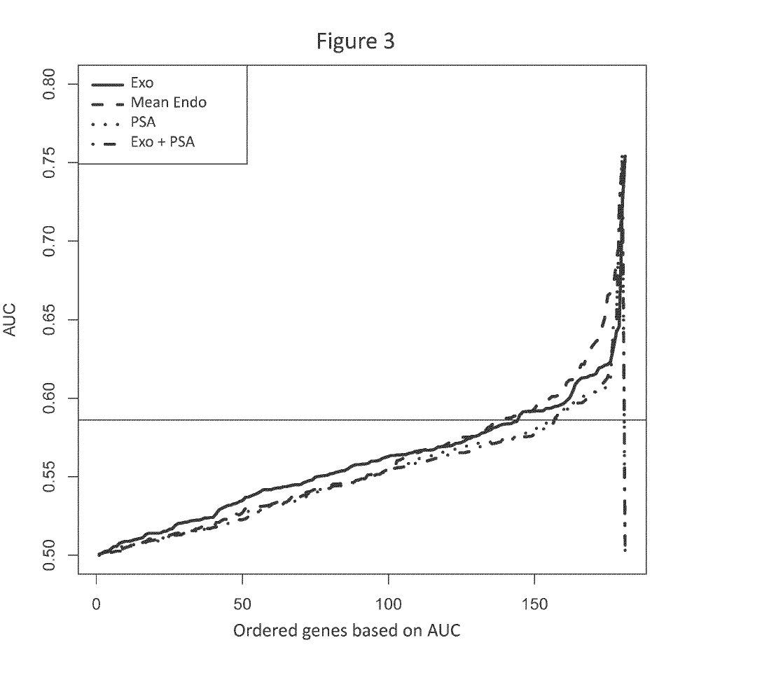

The second step was thus to test different normalization approaches and evaluate the effect on AUC for individual prostate specific control markers. We tested the normalization using four different approaches: (1) using the Ct of the exogenous internal positive control duplex PCR (“Exo”); (2) using the mean of the 5 endogenous reference genes (“Mean Endo”); (3) using PSA (“PSA”); and (4) using both PSA and the exogenous internal positive control (“Exo+PSA”). We verified the difference in performance by plotting sorted AUC of the individual markers as a function of the different normalization approaches in FIG. 3. The horizontal line corresponds to the 95% expected random performance, meaning that all markers over this line have a performance that is significantly higher than a random predictor. Under such conditions, we observed that the normalization approach using the mean of five (5) endogenous reference genes gives more reproducible AUC for individual genes when testing large gene expression data set (e.g., 150 genes or more).

### Example 6

### Validation of Prostate Cancer Classifiers on Whole Urine Samples Analyzed by RT-gPCR, Including Urine from Patients Undergoing Treatment

The selection of the prostate cancer markers listed in Table 5 was based on different thresholds of t-test p-values and by the area under the ROC curve (AUC). The AUC was used as a performance measure to determine if genes have a pattern of expression which is positively or negatively associated with a clinical assessment of prostate cancer from urine samples. Once the gene subset had been established, the top prostate cancer markers (as sorted based on the detection of prostate cancer from urine samples) were combined using the Bayes rule. To validate the multigene prostate cancer signatures defined by the first approach we combined two datasets to evaluate the performance of a selected number of multigene prostate cancer signatures and randomly assigned a set of samples as the training set and the remaining sample as the validation set. The resulting Naïve Bayes classifier, which was trained using 174 whole urine samples (comprising 73 samples from prostate cancer subjects patients, and 101 samples from non-prostate cancer subjects), was then used to predict the likelihood of prostate cancer in a biological sample. The Naïve Bayes classifier selects the most likely classification V_{nb} (e.g., Normal or Tumor) given the attribute values a_{1′}, a_{2′}, . . . aₙ. In this example, V_{nb} could be either tumor or normal and the attributes values aᵢ represent real values corresponding normalized gene expression level (delta Ct) as provided by RT-qPCR. This results in the corresponding classifier:

V_{nb}=(a₁, a₂, . . . , aₙ)=argmaxᵥ_{ⱼ}_{ε}_{^{v}}P(vⱼ)ΠP(aᵢ|vⱼ)

We generally estimate P(aᵢ|vⱼ) using normal distribution for which mean μᵥⱼ and standard deviation σᵥⱼ are estimated from the training set for every class and gene as in:

$$P ( {a}_{i} \vert {v}_{j} ) = \frac{1}{\sqrt{2 {πσ}_{vj}^{2}}} {e}^{\frac{- {( {a}_{i} - {μ}_{vj} )}^{2}}{2 {σ}_{vj}^{2}}}$$

### Where

aᵢ=the delta Ct of gene i

vⱼ=either tumor or normal

μᵥⱼ=the mean of class vⱼ and gene i

σᵥⱼ=the standard deviation of class vⱼ and gene i

For example, for a 5-gene Naïve Bayes classifier we need to estimate 2×5×2 (for mean and standard deviation)=20 parameters from the training set. When applying such machine learning algorithms, it is highly recommended to add a cross-validation step because, in some instances, algorithms may be able to classify well the sample in the training set, and yet yield poorer results on an independent test set. This phenomenon is called over-fitting. To avoid over-fitting during model selection, the selection of prostate cancer markers was performed using 20 repeats of a 10-fold cross validation within the training set. For the present analyses, we used “leave-two-out” cross-validation, which involves removing one cancer and one non-cancer sample to train the algorithm, and then testing back with the samples that were left-out. The performances of the different models were compared using the AUC. The number of parameters was selected to maximize AUC and minimize random variation across batch using 200 iterations. The best parameters were identified as the ones giving the highest mean cross-validated AUC computed on the training set. Real values used as Naïve Bayes parameters are normalized expression level of prostate cancer makers (deltaCt) or a parameter computed from a pair of genes. For example, classifier 3 included pairs of genes as Naïve Bayes parameters. In this particular example, the ERG-SNAI2 parameter represents the differential expression between the most up-regulated gene, ERG, and the most down-regulated gene, SNAI2 among the tested cohort and was calculated by subtracting the deltaCt value of SNAI2 from the deltaCt value of ERG. In another classifier, a Naïve Bayes parameters was the most overexpressed genes selected from a group consisting of the co-regulated genes ERG and CACNA1D and referred herein as maxERG CACNA1 D in classifier 4.

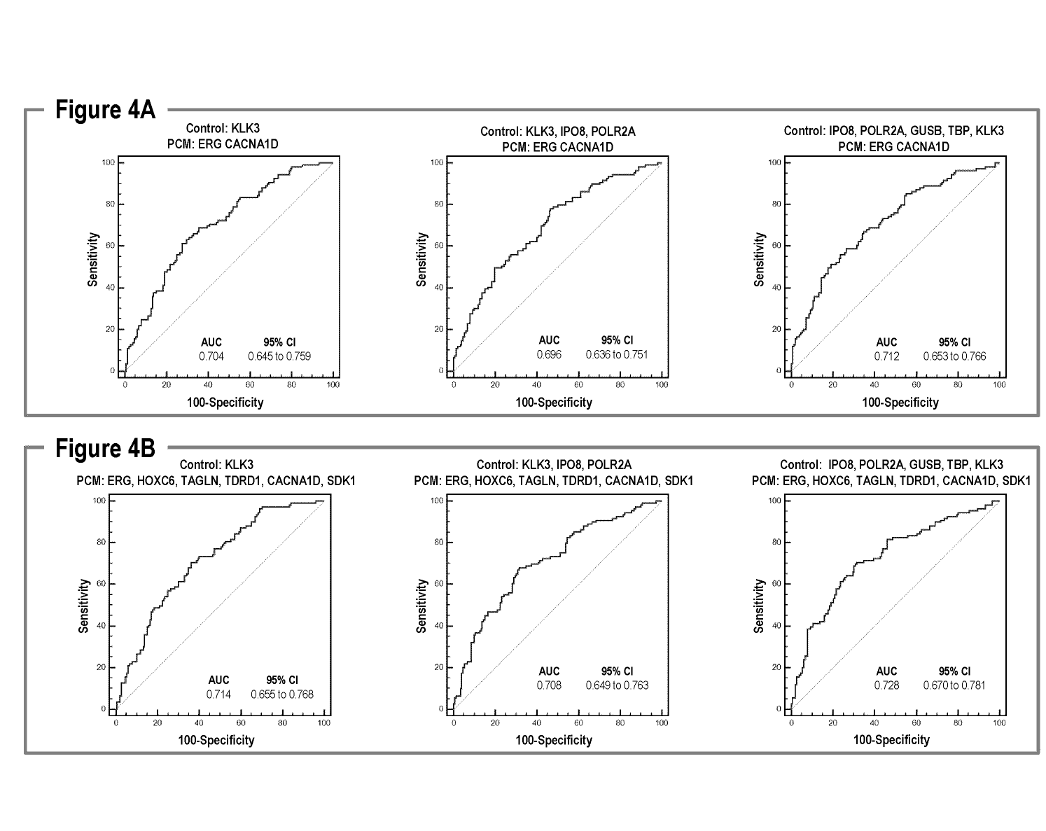

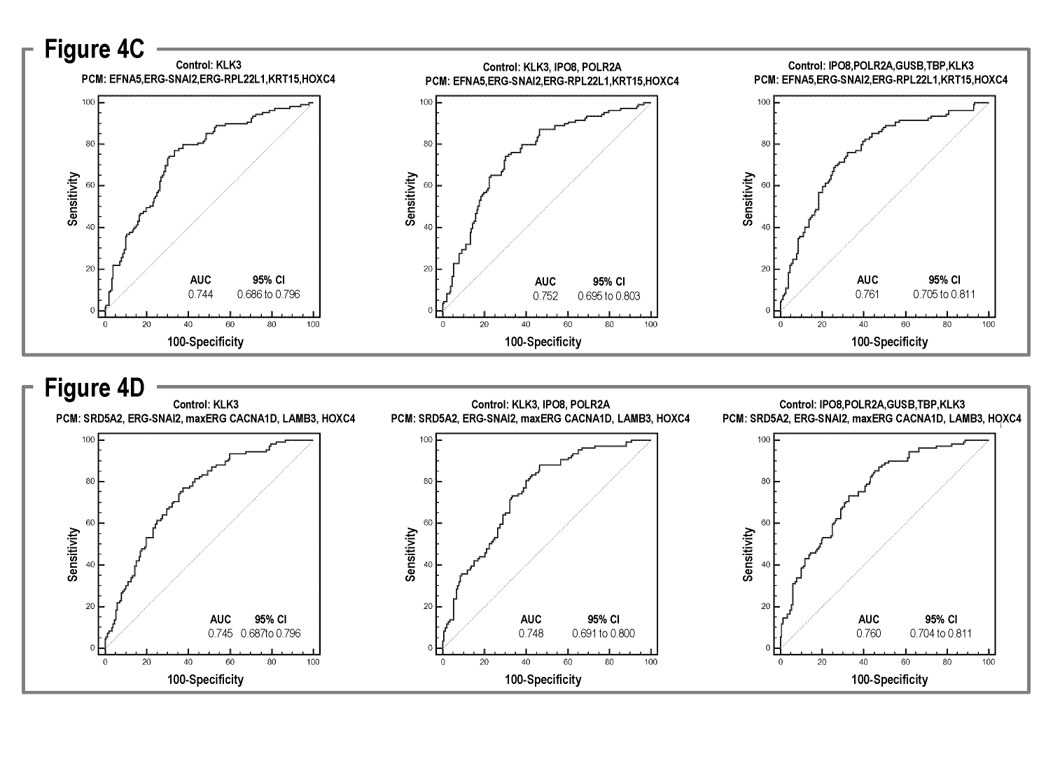

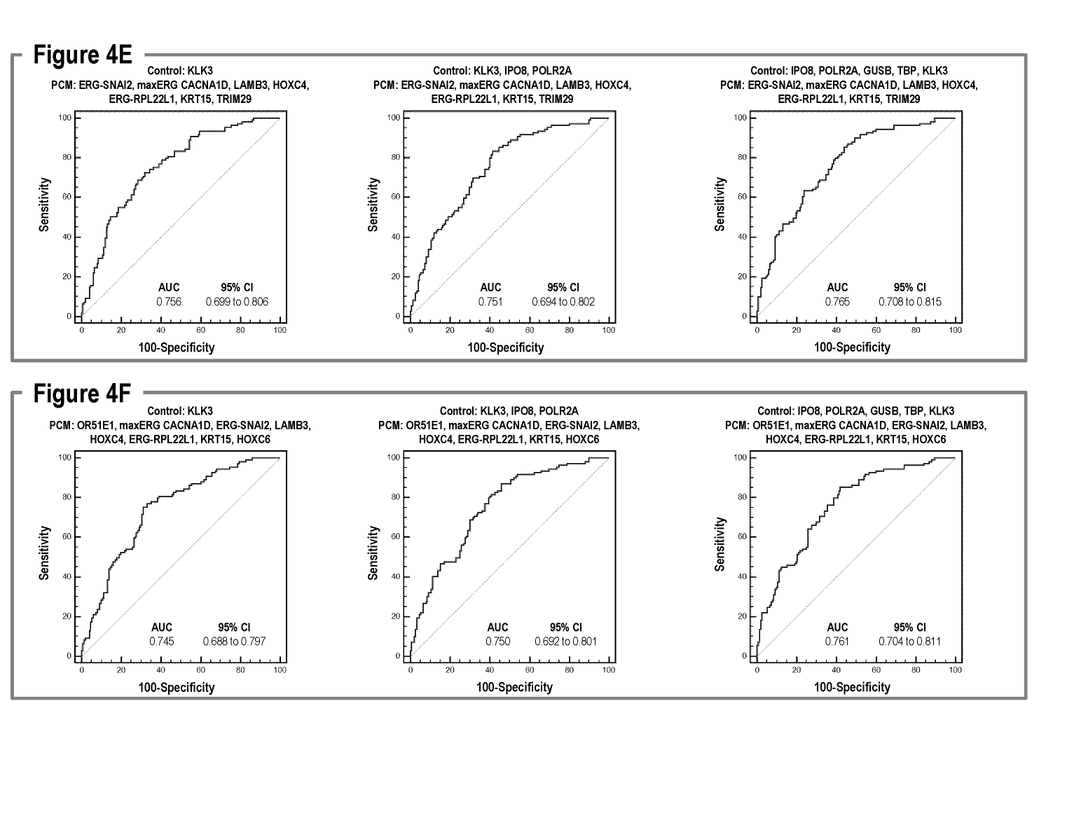

Finally, a selection of classifiers qualified on the training set was applied to the 87 biological samples in the validation set. Table 7A shows the performance characteristics of the 18 prostate cancer signatures in a training set of 174 whole urine samples and a validation set of 87 whole urine samples from men having or suspected of having prostate cancer. We also used the DeLong's test to verify the difference in AUC observed for a given classifier compared to the PCA3/PSA ratio in the training and validation set. The performance of each individual was also analyzed in relation to prostate cancer aggressiveness defined by high Gleason score in the biopsies samples. P-value for the association with the Gleason score is presented in Table 7A. All selected multigene signatures generated with this approach were able to significantly discriminate subjects according to the presence or absence of prostate cancer (FIG. 4A-F). AUC scores illustrate how accurately the 18 prostate cancer signatures were able to detect prostate cancer versus all other conditions in both the training and the validation set.

Herein, we evaluated 3 different normalization approaches wherein a prostate specific marker such as PSA is used as a control marker to normalize gene expression data in relation with the presence of prostate epithelial cells in the urine sample. Our results suggest that increasing the number of normalization genes increased the overall performance of a classifier (Table 7A). As mentioned in Example 5, prostate specific markers other than PSA, can be used in a normalization step to control for the presence of nucleic acid originating from prostate cells in the sample. Table 7B shows the performance characteristics of the selected classifiers using prostate specific control makers other than PSA. Analysis of receiver-operating characteristic (ROC) curves confirmed the improved diagnostic accuracy afforded by incorporating the prostate specific control marker to the other control marker (Table 7B).

We also wanted to validate that the prostate cancer classifiers of the present invention can also be used in a population of men undergoing treatment for benign conditions other than prostate cancer, such as BPH. Thus, ROC curve analysis were performed on a group of 51 individuals taking either a 5-alpha-reductase inhibitor, such as Dutasteride (Avodart™) or Finasteride (Proscar™, Propecia™), or an alpha-1 adrenergic receptor antagonist such as Tamsulosin (Flomax™) or alfuzosin (Xatral™). Table 8 provides performance characteristics of prostate cancer classifiers using urine samples from 14 patients with confirmed prostate cancer, as compared with 37 specimens from non-prostate cancer subjects, all of which are taking BPH medication. For comparison purposes, results from a similar cohort not known to take BPH medication were provided. Performance characteristics of the 18 prostate cancer signatures were better in the group under BPH medication than in the cohort not known to take BPH medication.

It has been reported in the literature that BPH medication (e.g., 5-alpha-reductase inhibitors) could reduce the likelihood of developing prostate cancer. This potential additional effect of BPH medication might explain the better overall performance of the selected classifiers in this cohort, as compared to individuals not under BPH medication. These results suggest that screening for prostate cancer using gene signatures of the present invention in men under BPH medication is a practical approach for prevention of prostate cancer development.

Additionally, the signature seem to also have clinical applications among men with Gleason 7, by further estimating their risk of lethal prostate cancer and thereby guiding therapy decisions to improve outcomes and reduce overtreatment. A comparison was made between whole urine samples from: (1) non-prostate cancer subjects; and (2) prostate cancer subjects with the highest Gleason score (≧7) pattern. Each of the 18 prostate cancer signatures were analyzed using this subset of 204 urine specimens. Table 9 provides performance characteristics of prostate cancer classifiers using Naïve Bayes algorithms in whole urine samples from 52 patients with Gleason score ≧7, compared with 152 specimens from non-prostate cancer subjects. Using the same experimental setup as described above, each classifier was able to accurately separate cancer subjects with high Gleason score (≧7) from non-prostate cancer subjects based on urine sample analysis. Increasing the number of normalization genes again increased the overall performance of the classifiers.

Table 9 also provides performance characteristics of prostate cancer classifiers in a subset of individuals in which the test was performed on the first 20 to 30 mL of voided urine collected after DRE but before the first biopsy. In total, 220 individuals were screened and 122 had subsequent negative biopsy results, while 98 had a confirmed diagnosis of prostate cancer. Of importance, all classifiers were able to accurately identify patients with increased risk of having a first positive biopsy result with performance characteristics presented in Table 9.

### Example 7

### Prognostic Abilities of Genes Significantly Associated with the Presence of Prostate Cancer

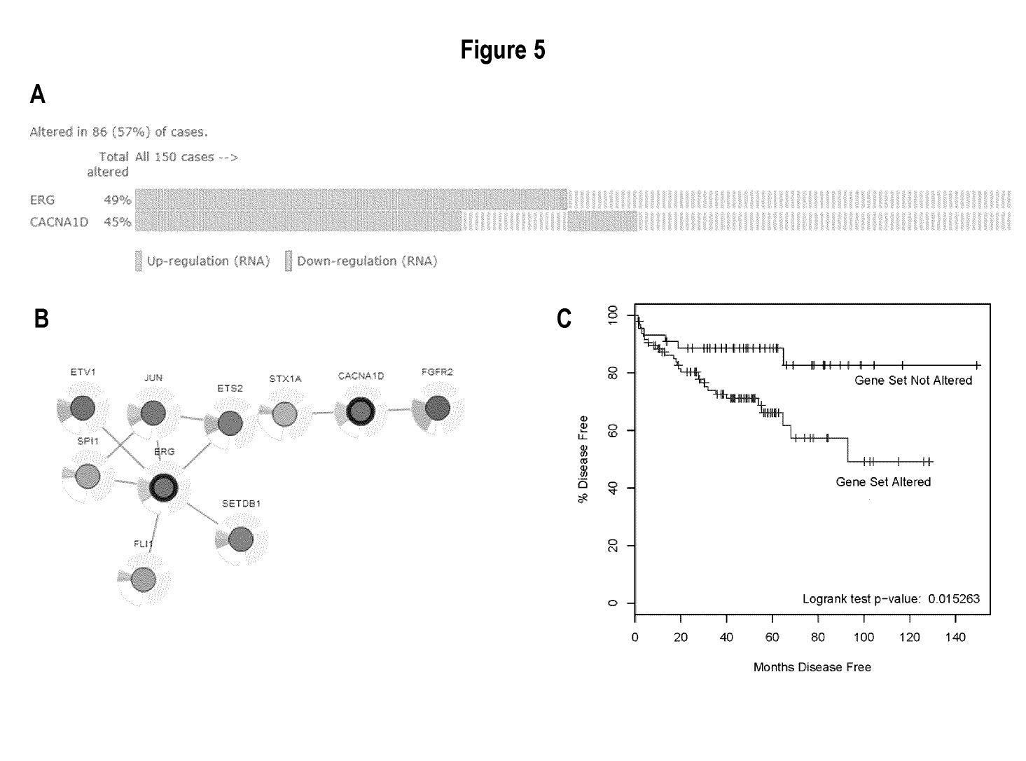

For some applications, it would be useful not only to diagnose the presence of cancer in a subject based on a probability score, but also to be able to use the same score to predict the subject's outcome after therapy. As noted in Example 6, some of the prostate cancer markers selected in certain classifiers were associated with high Gleason Score (Table 7A and Table 9) and could thus be used to predict disease progression and poor outcome. Accordingly, we selected a subset of genes from five (5) classifiers and tested if they had prognostic abilities, by testing prostate cancer subjects having undergone radical prostatectomy. We used a publicly available dataset (GSE21032) containing gene expression data from 150 prostate cancer tissue samples to test whether gene expression level alteration of this subset of genes is associated with an increased risk of developing aggressive cancer and hence, associated with poor outcome. Gene expression data for each of the subjects were generated using the GeneChip® Human Exon 1.0 ST Array (Affymetix, Santa Clara, Calif.) and included clinical data annotations for each subject. We performed a disease-free survival analysis based on 5 selected gene signatures associated with the presence of prostate cancer via the cBio Cancer Genomics Portal (http://cbioportal.org). As an illustrative example, FIG. 5A shows the OncoPrint™ for the two prostate cancer markers included in classifier 1. In this case, we observed that mRNA expression alteration of genes within this classifier was present in more than 50% of the cases. The portal also supports visualization of network interaction among genes present in the classifier and those reported as belonging to a common pathway (FIG. 5B).

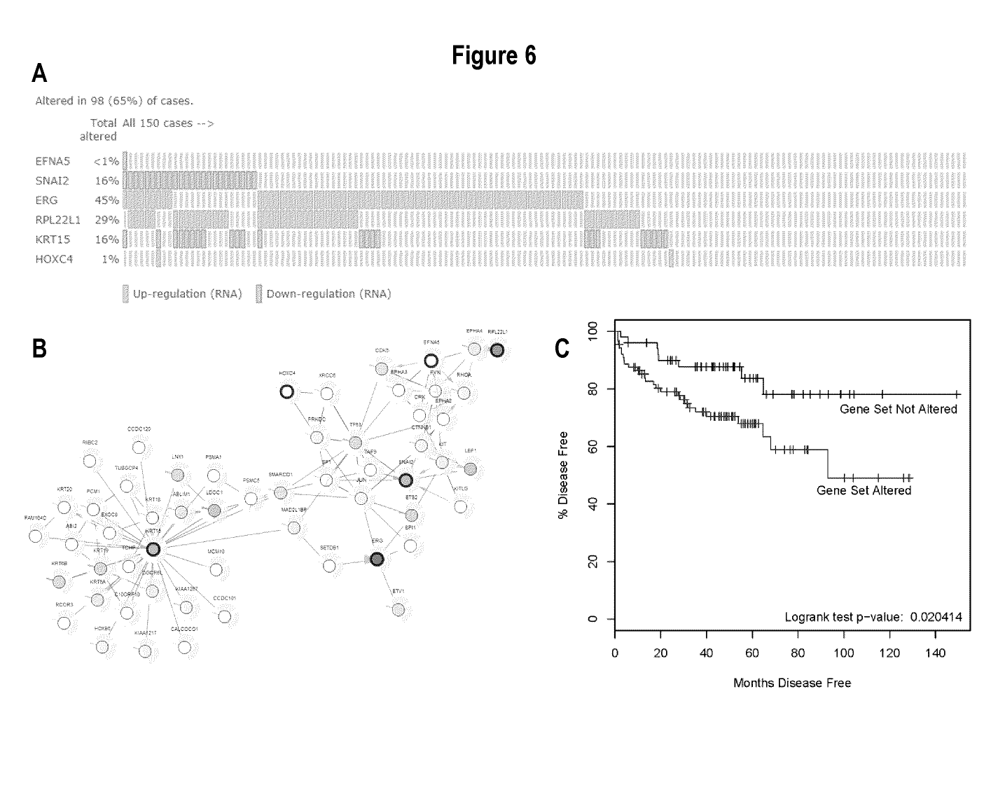

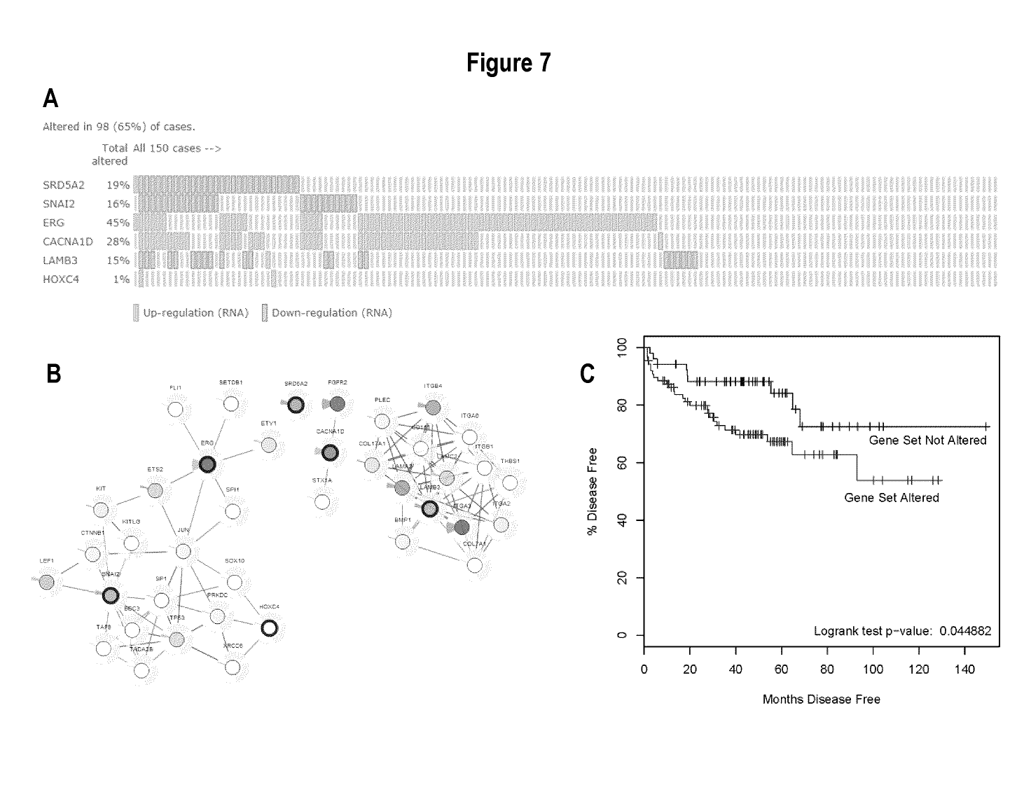

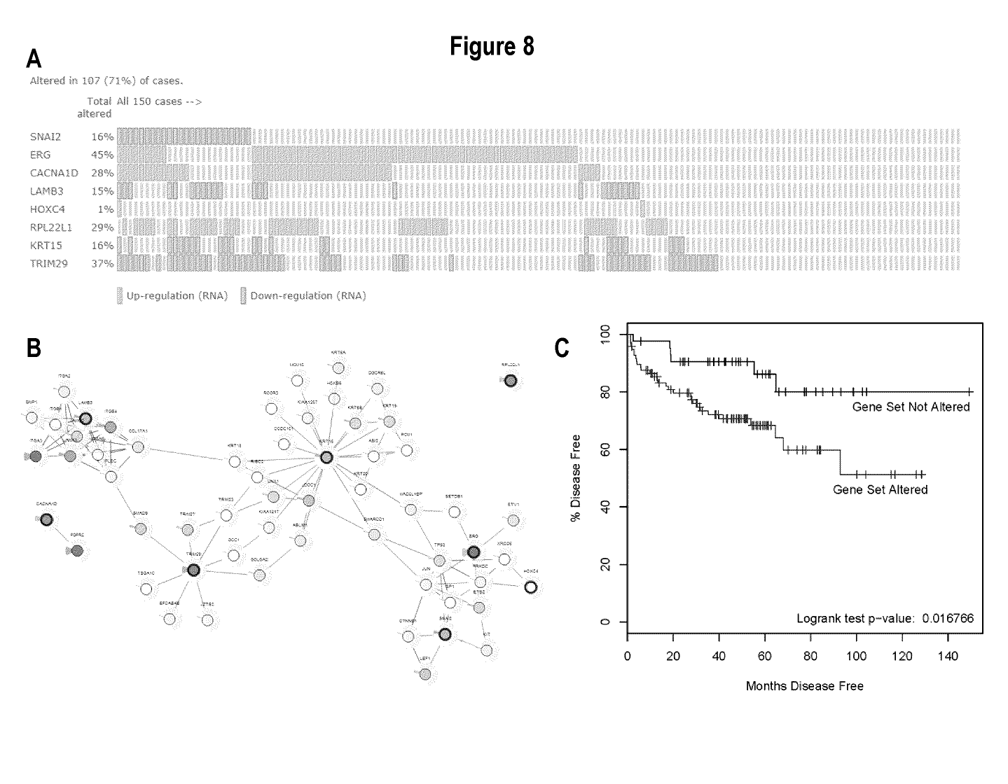

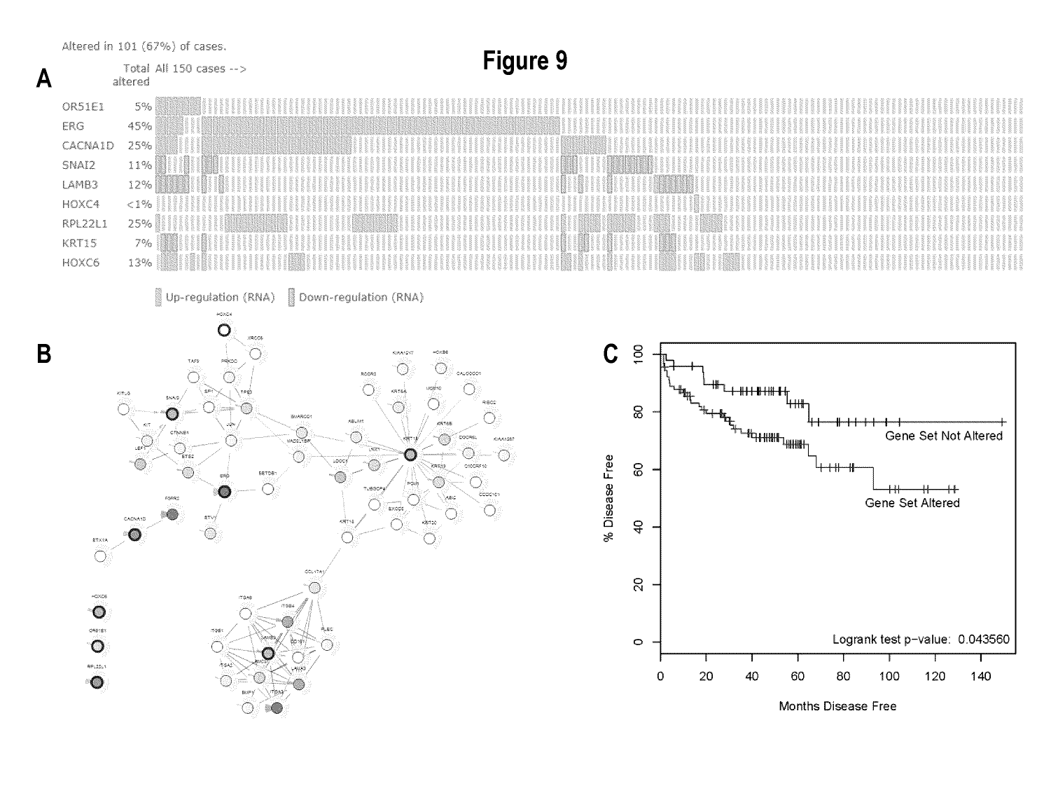

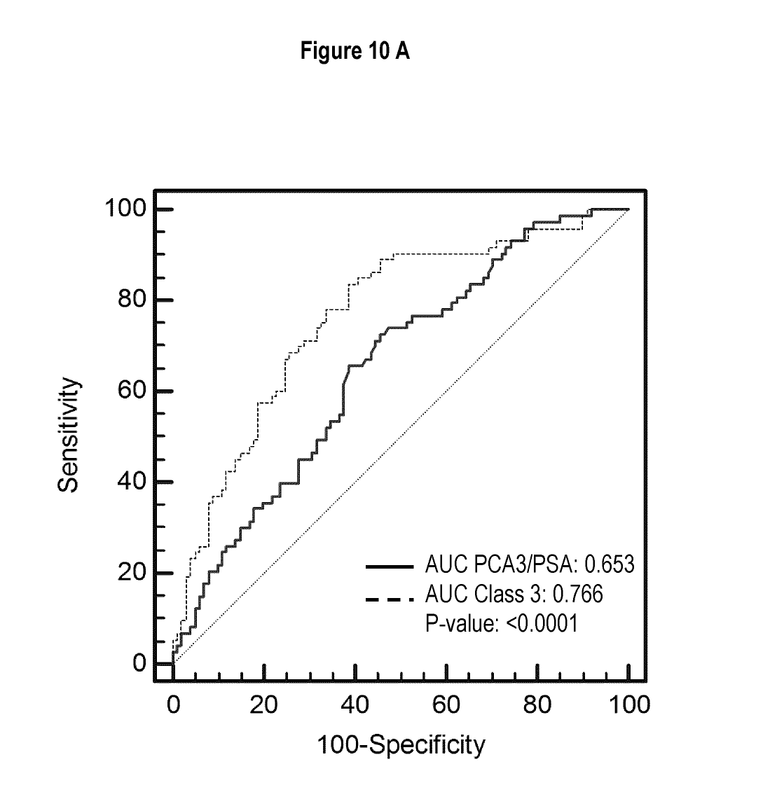

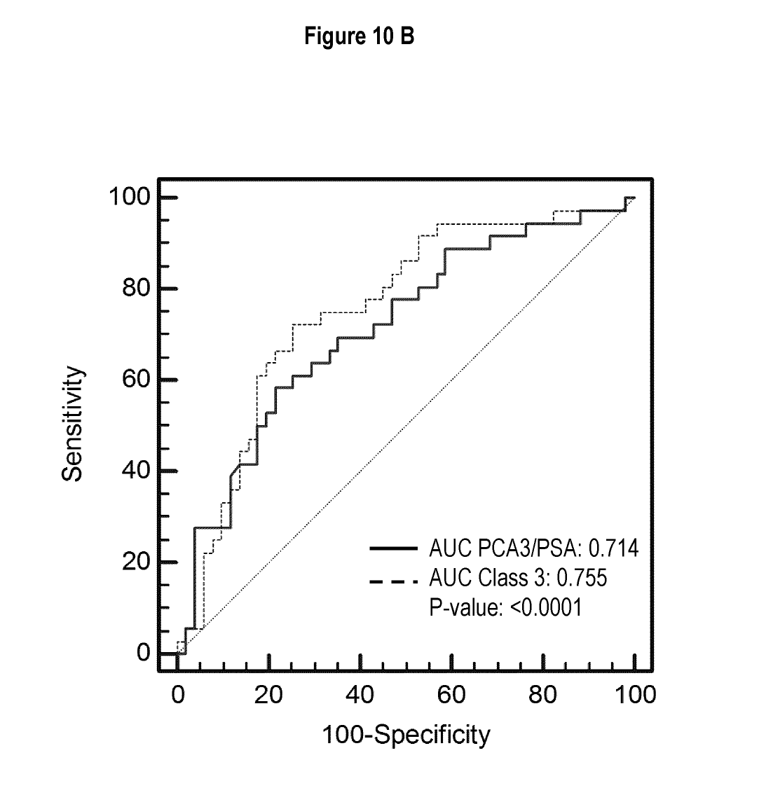

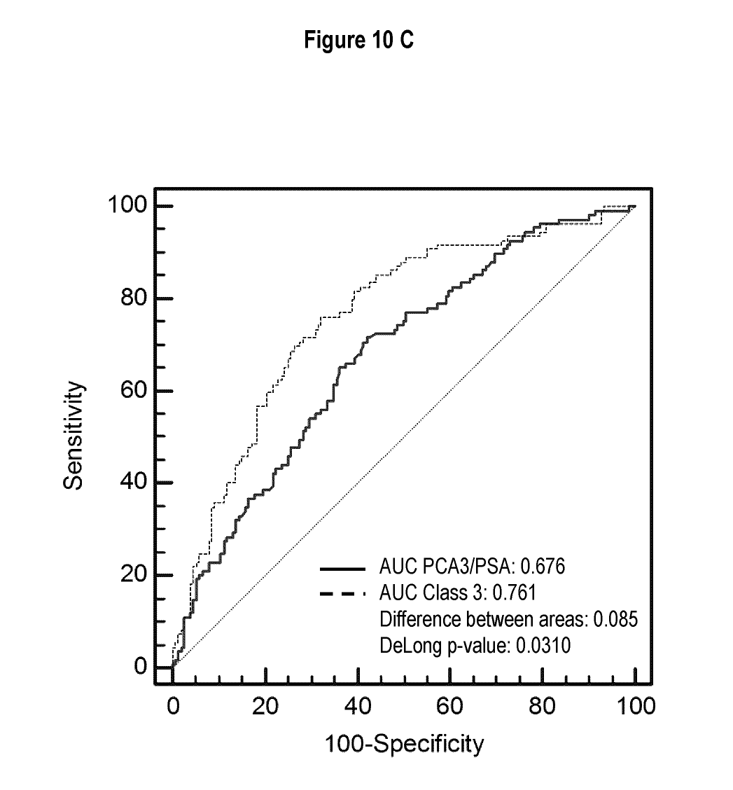

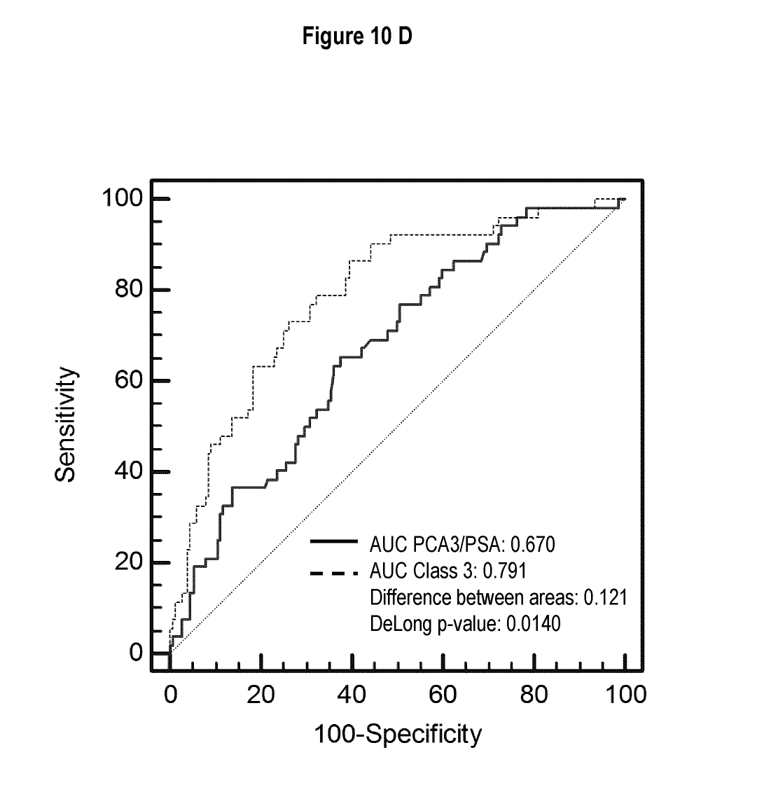

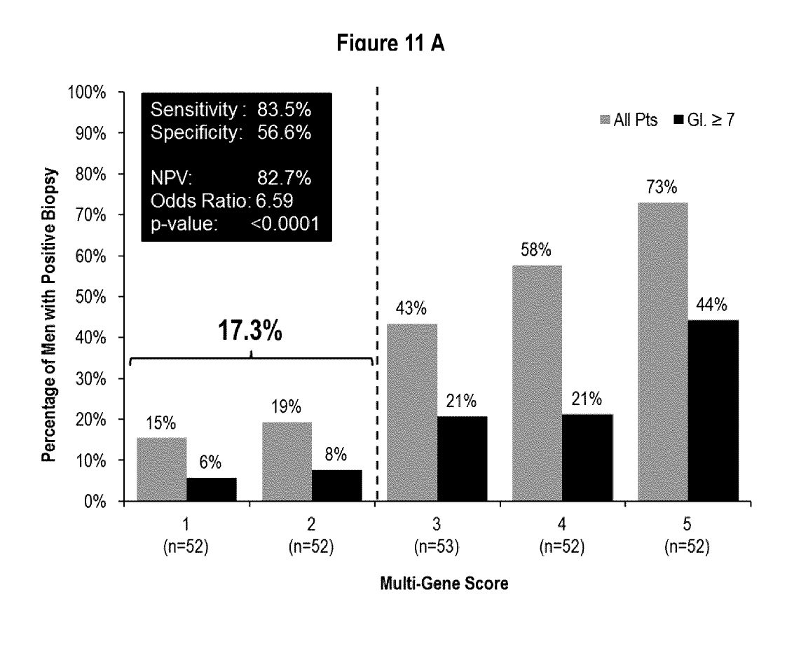

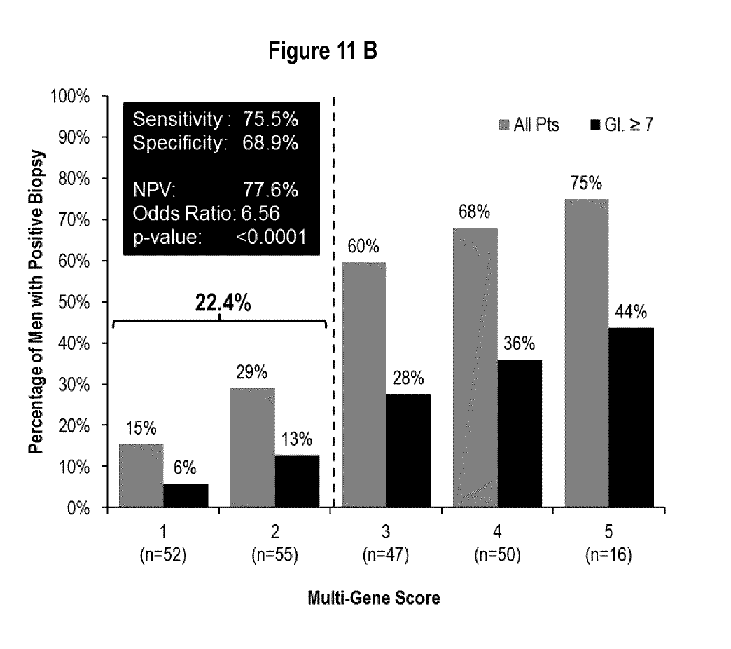

Panel C of FIGS. 5-9 show Kaplan-Meier curves of disease-free survival after prostatectomy. For each selected classifier, disease-free survival analysis was performed in subjects with gene expression altered as compared to patients with gene set not altered, based on mRNA expression Z-score. All five classifiers were able to predict significant worse survival in patients with altered mRNA expression. For the five examined classifiers, genes were altered in at least half of the cases with some classifiers having more than 100 cases with altered gene expression out of 150 prostate cancer patients. Overall, gene sets selected in these classifiers were either up- or down-regulated in prostate cancer and were found to be useful predictors of outcome after prostatectomy. The present invention highlights and demonstrates the potential value of selected multi-gene signature-based diagnostics, as well as tools for improved prognostication and treatment stratification in prostate cancer.

Thus, the classifiers and signatures of the present invention not only relate to diagnosis of prostate cancer, they also relate to prognosis, grade determination, patient outcome, etc. The classifiers and signatures of the present invention are thus extremely powerful clinical assessment tools for prostate cancer.

### Example 8

### Performance Characteristics of a Prostate Cancer Multigene Signature Incorporating PCA3 Marker

Using the same experimental setup as mentioned above, a set of experiments was conducted to determine the effect on performance characteristics of incorporating the PCA3 marker into a prostate cancer multigene signature of the present invention that lacks PCA3. The performance criterion was the area under the ROC curve (AUC), where the ROC curve is a plot of the sensitivity as a function of the specificity. The AUC measures how well the classifiers monitor the sensitivity/specificity tradeoff without imposing a particular threshold. For this analysis, we used the classifier 3 (class 3; Table 7A) multigene signature with 5 control markers (IPO8, POLR2A, GUSB, TBP, KLK3) to evaluate the effect of incorporating the PCA3 marker. The difference between the two approaches is solely based on the addition of PCA3 non-coding RNA as a known prostate cancer marker into the multigene signature to predict the likelihood of prostate cancer in a biological sample.

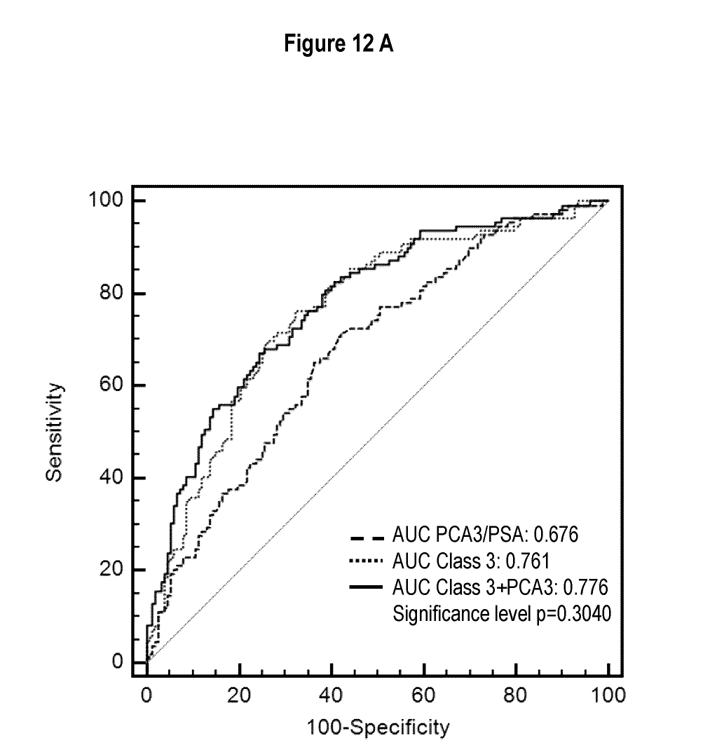

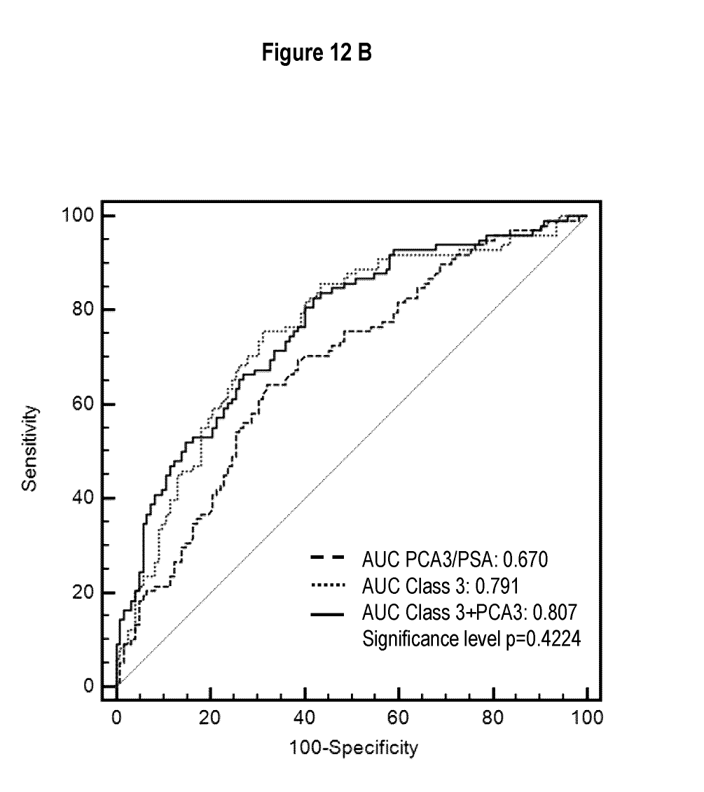

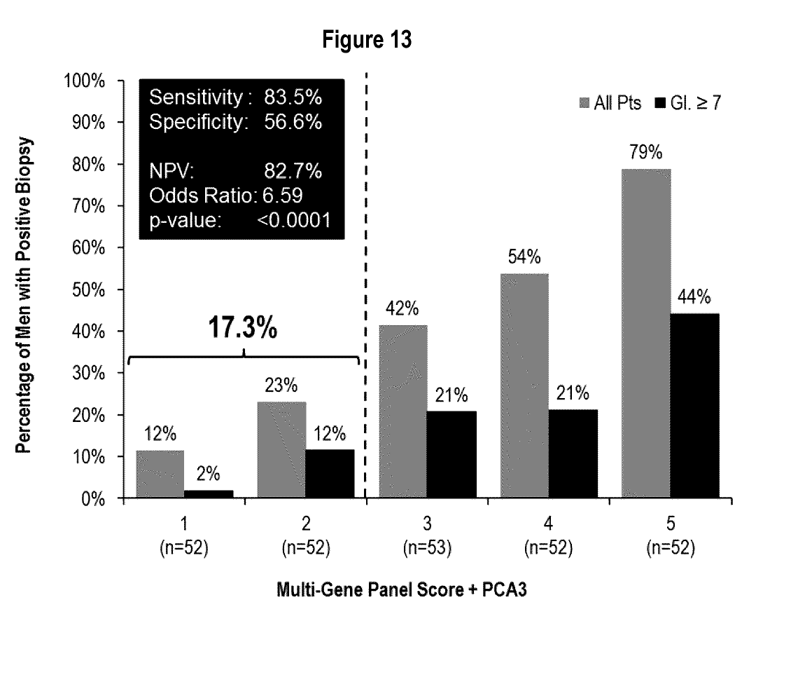

Surprisingly, our results demonstrate that incorporating PCA3 non-coding RNA into a prostate cancer classifier of the present invention does not increase the overall performance of the classifier (FIG. 12A). Overall, the difference between areas did not result in increased sensitivity of specificity in the total cohort (FIG. 13). As mentioned in Example 6, the classifier was able to accurately separate cancer subjects with high Gleason score 7) from non-prostate cancer subjects based on urine sample analysis. Again, inclusion of PCA3 non-coding RNA to the set of prostate cancer markers did not result in a statistically significant improvement in AUC at 0.807 compared to 0.791 without PCA3 (DeLong p-value=0.4224) (FIG. 12B).

Although the present invention has been described hereinabove by way of specific embodiments thereof, it can be modified, without departing from the spirit and nature of the subject invention as defined in the appended claims.

### REFERENCES

de la Taille A, Irani J, Graefen M, Chun F, de RT, Kil P, et al. Clinical Evaluation of the PCA3 Assay in Guiding Initial Biopsy Decisions. J Urol 2011; 185: 2119-25
Laxman B, Morris D S, Yu J, Siddiqui J, Cao J, Mehra R, Lonigro R J, Tsodikov A, Wei J T, Tomlins S A, Chinnaiyan A M. A first-generation multiplex biomarker analysis of urine for the early detection of prostate cancer. Cancer Res., 2008, 68: 645-649
Nam R K, Saskin R, Lee Y, Liu Y, Law C, Klotz L H, et al. Increasing hospital admission rates for urological complications after transrectal ultrasound guided prostate biopsy. J Urol 2010; 183:963-8
Schroder F H, Hugosson J, Roobol M J, Tammela T L, Ciatto S, Nelen V, et al. Prostate-cancer mortality at 11 years of follow-up. N Engl J Med 2012; 366: 981-90

1. A method for providing a clinical assessment of prostate cancer in a subject, said method comprising:
(a) determining the expression of at least two prostate cancer markers listed in Table 5 or 6A, or a marker co-regulated therewith in prostate cancer, in a biological sample from said subject;
(b) normalizing the expression of said at least two prostate cancer markers using one or more control markers;
(c) performing a mathematical correlation of the normalized expression levels of said at least two prostate cancer markers;
(d) deriving a score from said mathematical correlation; and
(e) providing said clinical assessment of prostate cancer based on said derived score.

2. The method of claim 1, wherein said
at least two prostate cancer markers are validated as such, based on their expression profile in urines of a population of patients known to have or lack prostate cancer.

3. The method of claim 1, wherein said at least two prostate cancer markers is at least three prostate cancer markers; at least four prostate cancer markers; at least five prostate cancer markers; at least six prostate cancer markers; at least seven prostate cancer markers; at least eight prostate cancer markers; or at least nine prostate cancer markers.

4. The method of claim 1, wherein said at least two prostate cancer markers are selected from:
(1) CACNA1D or a marker co-regulated therewith in prostate cancer;
(2) ERG or a marker co-regulated therewith in prostate cancer;
(3) HOXC4 or a marker co-regulated therewith in prostate cancer;
(4) ERG-SNAI2 prostate cancer marker pair;
(5) ERG-RPL22L1 prostate cancer marker pair;
(6) KRT 15 or a marker co-regulated therewith in prostate cancer;
(7) LAMB3 or a marker co-regulated therewith in prostate cancer;
(8) HOXC6 or a marker co-regulated therewith in prostate cancer;
(9) TAGLN or a marker co-regulated therewith in prostate cancer;
(10) TDRD1 or a marker co-regulated therewith in prostate cancer;
(11) SDK1 or a marker co-regulated therewith in prostate cancer;
(12) EFNA5 or a marker co-regulated therewith in prostate cancer;
(13) SRD5A2 or a marker co-regulated therewith in prostate cancer;
(14) maxERG CACNA1D prostate cancer marker pair;
(15) TRIM29 or a marker co-regulated therewith in prostate cancer;
(16) OR51E1 or a marker co-regulated therewith in prostate cancer; and
(17) HOXC6 or a marker co-regulated therewith in prostate cancer.

5. The method of claim 1, wherein said at least two prostate cancer markers comprise:
(a) CACNA1D or a prostate cancer marker co-regulated therewith in prostate cancer; or
(b) CACNA1D or a prostate cancer marker co-regulated therewith in prostate cancer, and ERG or a prostate cancer marker co-regulated therewith in prostate cancer.

6. (canceled)

7. The method of claim 4, wherein said prostate cancer markers are combined in classifiers as defined in Tables 7-9.

8. The method of claim 1, wherein one or more of said marker co-regulated therewith in prostate cancer is as defined in Table 6B.

9. The method of claim 1, wherein said one or more control markers comprise:
(a) endogenous reference genes;
(b) at least one prostate-specific control marker;
(c) one or more control markers are as defined in Table 2, Table 7A and/or Table 7B;
(d) one or more of KLK3, FOLH1, FOLH1B, PCGEM1, PMEPA1, OR51E1, OR51E2, and PSCA;
(e) one or more of KLK3, IPO8, and POLR2A; or
(f) one or more of IPO8, POLR2A, GUSB, TBP, and KLK3.

10-14. (canceled)

15. The method of claim 1, wherein said clinical assessment of prostate cancer comprises:
(i) a diagnosis of prostate cancer;
(ii) a prognosis of prostate cancer;
(iii) a staging assessment of prostate cancer;
(iv) a prostate cancer aggressiveness classification;
(v) an assessment of therapy effectiveness;
(vi) as assessment of the need for a prostate biopsy; or
(vii) any combination of (i) to (vi).

16. The method of claim 1, wherein said marker is a gene or a protein.

17. (canceled)

18. The method of claim 1, wherein said determining the expression of said at least two prostate cancer markers comprises;
(a) determining RNA expression by performing a hybridization and/or amplification reaction which comprises:
(i) polymerase chain reaction (PCR);
(ii) nucleic acid sequence-based amplification assay (NASBA);
(iii) transcription mediated amplification (TMA);
(iv) ligase chain reaction (LCR);
(v) strand displacement amplification (SDA);
(vi) direct sequencing of said at least two prostate cancer markers; or
(vii) any combination of (i) to (vi); and/or
(b) determining protein expression.

19-21. (canceled)

22. The method of claim 1, wherein said biological sample is urine, whole or crude urine, urine sediment, urine obtained with or without prior digital rectal examination, prostate tissue resection, prostate tissue biopsy, ejaculate or bladder washing.

23-25. (canceled)

26. A prostate cancer diagnostic composition comprising:
(a) urine, or a fraction thereof having markers of prostate origin, from a subject having or suspected of having prostate cancer; and
(b) reagents enabling the detection and/or amplification of at least two prostate cancer markers from Table 5 or 6A, or a marker co-regulated therewith.

27. (canceled)

28. The prostate cancer diagnostic composition of claim 26, wherein:
(a) said at least two prostate cancer markers are selected from:
(1) CACNA1D or a marker co-regulated therewith in prostate cancer;
(2) ERG or a marker co-regulated therewith in prostate cancer;
(3) HOXC4 or a marker co-regulated therewith in prostate cancer;
(4) ERG-SNAI2 prostate cancer marker pair;
(5) ERG-RPL22L1 prostate cancer marker pair;
(6) KRT 15 or a marker co-regulated therewith in prostate cancer;
(7) LAMB3 or a marker co-regulated therewith in prostate cancer;
(8) HOXC6 or a marker co-regulated therewith in prostate cancer;
(9) TAGLN or a marker co-regulated therewith in prostate cancer;
(10) TDRD1 or a marker co-regulated therewith in prostate cancer;
(11) SDK1 or a marker co-regulated therewith in prostate cancer;
(12) EFNA5 or a marker co-regulated therewith in prostate cancer;
(13) SRD5A2 or a marker co-regulated therewith in prostate cancer;
(14) maxERG CACNA1 D prostate cancer marker pair;
(15) TRIM29 or a marker co-regulated therewith in prostate cancer;
(16) OR51E1 or a marker co-regulated therewith in prostate cancer; and
(17) HOXC6 or a marker co-regulated therewith in prostate cancer;
(b) said at least two prostate cancer markers comprise CACNA1D or a prostate cancer marker co-regulated therewith in prostate cancer; or
(c) said at least two prostate cancer markers comprise CACNA1D or a prostate cancer marker co-regulated therewith in prostate cancer, and ERG or a prostate cancer marker co-regulated therewith in prostate cancer.

29-32. (canceled)

33. The prostate cancer diagnostic composition of claim 26, further comprising reagents enabling the detection and/or amplification of one or more control markers, wherein said one or more control markers comprise:
(a) endogenous reference genes;
(b) at least one prostate-specific control marker;
(c) one or more control markers are as defined in Table 2, Table 7A and/or Table 7B;
(d) one or more of KLK3, FOLH1, FOLH1B, PCGEM1, PMEPA1, OR51E1, OR51E2, and PSCA;
(e) one or more of MAKI IPO8, and POLR2A; or
(f) one or more of IPO8, POLR2 GUSB, TBP, and KLK3.

34-40. (canceled)

41. The prostate cancer diagnostic composition of claim 26, wherein said marker is a gene or a protein, and wherein said reagents enable the determination of RNA expression and/or protein expression.

42-44. (canceled)

45. The prostate cancer diagnostic composition of claim 26, wherein said reagents enabling the detection and/or amplification of said at least two markers comprises oligonucleotides enabling the detection and/or amplification of said at least two markers, or said marker co-regulated therewith.

46-48. (canceled)

49. A kit for providing a clinical assessment of prostate cancer in a subject from a biological sample therefrom, said kit comprising:
(a) reagents enabling the detection and/or amplification of at least two prostate cancer markers from Table 5 or 6A, or a marker co-regulated therewith; and
(b) a suitable container.

50. (canceled)

51. The kit of claim 49, wherein:
(a) said at least two prostate cancer markers are selected from:
(1) CACNA1D or a marker co-regulated therewith in prostate cancer;
(2) ERG or a marker co-regulated therewith in prostate cancer;
(3) HOXC4 or a marker co-regulated therewith in prostate cancer;
(4) ERG-SNAI2 prostate cancer marker pair;
(5) ERG-RPL22L1 prostate cancer marker pair;
(6) KRT 15 or a marker co-regulated therewith in prostate cancer;
(7) LAMB3 or a marker co-regulated therewith in prostate cancer;
(8) HOXC6 or a marker co-regulated therewith in prostate cancer;
(9) TAGLN or a marker co-regulated therewith in prostate cancer;
(10) TDRD1 or a marker co-regulated therewith in prostate cancer;
(11) SDK1 or a marker co-regulated therewith in prostate cancer;
(12) EFNA5 or a marker co-regulated therewith in prostate cancer;
(13) SRD5A2 or a marker co-regulated therewith in prostate cancer;
(14) maxERG CACNA1D prostate cancer marker pair;
(15) TRIM29 or a marker co-regulated therewith in prostate cancer;
(16) OR51E1 or a marker co-regulated therewith in prostate cancer; and
(17) HOXC6 or a marker co-regulated therewith in prostate cancer
(b) said at least two prostate cancer markers comprise CACNA1D or a prostate cancer marker co-regulated therewith in prostate cancer; or
(c) said at least two prostate cancer markers comprise CACNA1D or a prostate cancer marker co-regulated therewith in prostate cancer, and ERG or a prostate cancer marker co-regulated therewith in prostate cancer.

52-55. (canceled)

56. The kit of claim 49, further comprising reagents enabling the detection and/or amplification of one or more control markers, wherein said one or more control markers comprise:
(a) endogenous reference genes;
(b) at least one prostate-specific control marker;
(c) one or more control markers are as defined in Table 2, Table 7A and/or Table 7B;
(d) one or more of KLK3, FOLH1, FOLH1B, PCGEM1, PMEPA1, OR51E1, OR51E2, and PSCA;
(e) one or more of KLK3, IPO8, and POLR2A: or
(f) one or more of IPO8, POLR2A, GUSB, TBP, and KLK3.

57-63. (canceled)

64. The kit of claim 49, wherein said marker is a gene or a protein, and wherein said reagents enable the determination of RNA expression and/or protein expression.

65-67. (canceled)

68. The kit of claim 49, wherein said reagents enabling the detection and/or amplification of said at least two markers comprises oligonucleotides enabling the detection and/or amplification of said at least two markers, or said marker co-regulated therewith.

69.-75. (canceled)
